(19) United States
(12) Patent Application Publication
(10) Pub. No.: US 2026/0060970 A1
(43) Pub. Date: Mar. 5, 2026

(54) Use of a Therapeutic Agent with Phosphodiesterase-7 Inhibitory Activity for the Treatment and Prevention of Diseases Associated with Chronic Fatigue, Exhaustion and/or Exertional Intolerance

(71) Applicant: MITODICURE GMBH, Kriftel (DE)

(72) Inventor: Klaus WIRTH, Kriftel (DE)

(21) Appl. No.: 19/103,774

(22) PCT Filed: Aug. 16, 2023
(86) PCT No.: PCT/EP2023/072568

§ 371 Date: Feb. 13, 2025

(30) Foreign Application Priority Data
Aug. 18, 2022 (EP) 22191022.7
Mar. 8, 2023 (EP) 23160645.0

(51) Int. Cl. A61K 31/4439; A61K 31/145; A61K 31/4162; A61K 31/4178; A61K 31/496; A61K 31/517; A61K 31/519; A61K 31/522; A61K 31/527; A61K 31/5377; A61P 21/00

(52) U.S. Cl. CPC A61K 31/4439; A61K 31/145; A61K 31/4162; A61K 31/4178; A61K 31/496; A61K 31/517; A61K 31/519; A61K 31/522; A61K 31/527; A61K 31/5377; A61P 21/00

(57) ABSTRACT

The instant invention relates to the use of a substance with phosphodiesterase-7 inhibitory activity (PDE7 inhibitor) as active ingredient in a therapeutic agent with phosphodiesterase-7 inhibitory activity for the treatment and prevention of different diseases, syndromes, disease states, or conditions associated with chronic fatigue, exhaustion and/or exertional intolerance using PDE7 inhibitors alone or in combination with other therapeutic agents.

The instant invention relates to the use of a substance with phosphodiesterase-7 inhibitory activity (PDE7 inhibitor) as active ingredient in a therapeutic agent with phosphodiesterase-7 inhibitory activity for the treatment and prevention of different diseases, syndromes, disease states, or conditions associated with chronic fatigue, exhaustion and/or exertional intolerance using PDE7 inhibitors alone or in combination with other therapeutic agents. These diseases, syndromes, disease states, or conditions will subsumed by the term “diseases associated with chronic fatigue” and abbreviated as (DACF) include diseases diagnosed as Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac syndrome) after COVID-19 vaccinations (Long Vax) and vaccinations against other germs, virus and pathogenic agents, postinfectious fatigue after viral, bacterial, or fungal infections, in particular ME/CFS of non-infectious, non-inflammatory and non-immunological cause, cancer-related fatigue (wherein chronic fatigue and exhaustion are symptoms of or are associated with cancer), and fatigue associated with fibromyalgia, Ehlers-Danlos syndrome, Marfan syndrome, Gulf War illness and the autoimmune diseases Rheumatoid Arthritis, ANCA vasculitis (anti-neutrophil cytoplasmic antibodies (ANCA)-associated vasculitis) and Sjögren's syndrome, and other autoimmune diseases with fatigue and exhaustion as debilitating symptoms.

PDE7 inhibitors are known and treatments have been described in the context of inflammation, immunomodulation, neoplastic diseases related to the immune system, respiratory diseases, cardiovascular diseases (acute myocardial infarction, stroke), neurodegenerative disorders (multiple sclerosis, Alzheimer's disease, Parkinson's disease), other neurological disorders (spinal cord injury, schizophrenia) and weaning from different addictions, for example in U.S. Pat. Nos. 11,207,275, 8,846,654. The claim for treating neurodegenerative and neurological diseases is based on the assumption that PDE7 inhibition exerts anti-inflammatory effects and treats autoimmune mechanisms in the nervous system.

The pathophysiology of diseases associated with chronic fatigue (DACF) had been totally enigmatic so that no rationally based pharmacological or therapeutic strategies could be derived or developed. Therefore, no specific effective treatment exists due to a lack of understanding of these diseases and their pathophysiology.

The instant invention has been established by a unifying and comprehensive disease hypothesis elaborated by the instant inventor who found that DACF can be treated by stimulating the sodium-potassium ATPase (Na⁺/K⁺ATPase) in skeletal muscle. The instant inventor found that a rise in cAMP (cyclic adenosine monophosphate) via PDE7-inhibition in skeletal muscle (myocytes) stimulates the sodium-potassium ATPase (Na⁺/K⁺ATPase) to prevent unfavorable ionic changes in intracellular sodium (Na⁺) and subsequently intracellular and mitochondrial calcium (Ca²⁺), which play a strong pathophysiological role in the development and perpetuation of chronic fatigue, exhaustion and/or exertional intolerance. A second transporter equally important for the prevention of mitochondrial calcium overload is the mitochondrial sodium-calcium exchanger abbreviated as NCLX that exports calcium from the mitochondrium in exchange for sodium to limit and prevent mitochondrial calcium overload. The activity of the NCLX is equally stimulated by cAMP. Thus, a rise in cAMP by PDE7-inhibition has a synergistic action on two ion transporters that prevent cellular and mitochondrial calcium overload and damage. A rise in cAMP can be achieved by administering PDE7 inhibitors, which inhibit the hydrolysis of cAMP to raise its levels. Additionally, a rise in cAMP by PDE7-inhibition also occurs in blood vessels of skeletal muscles and of the brain and enhances organ blood flow for further improvements. Since the PDE7A variant has a much higher expression in skeletal muscle than the variant PDE7B, a PDE7-inhibitor is with a particularly strong inhibitory effect on PDE7A is preferred.

As of today, eleven families of phosphodiesterases (PDE) have been identified. They are distinguished by their primary structure, their substrate specificity and their sensitivity with regard to various effectors and inhibitors specific for PDEs. Each family is composed of one or more genes which are expressed in various tissues in the form of splicing variants. For example, PDE4, PDE7 and PDE8 specifically hydrolyse cAMP and PDE5, PDE6 and PDE9 specifically hydrolyse cGMP.

The family PDE7 is represented by the isoforms PDE7A with their variants PDE7A1, PDE7A2 and PDE7A3 and PDE7B with their variants PDE7B1, PDE7B2 and PDE7B3 originating from two distinct genes. PDE7 is highly selective for cAMP. The Michaelis constant Kₘ values of PDE7B for cAMP ranges from 0.13 μM to 0.2 μM. Kₘ values of PDE7A1 and PDE7A2 for cAMP are 0.2 M and 0.1 μM, respectively. The catalytic part of PDE7B exhibits approximately 70% homology with that of PDE7A.

In conclusion, inhibition of PDE7 will only raise cAMP and the action of PDE7 inhibitors will to a great extent be restricted to tissues in which PDE7 is expressed.

The presence of PDE7 isoenzymes in skeletal muscle (in myocytes) is known, and there is a higher expression of PDE7A variant versus the PDE7B variant. Inhibition of PDE7 by appropriate inhibitors raises the concentration of the second messenger cAMP which is a key second messenger for appropriate functioning of skeletal muscle. Of additional importance for the treatment of DACF is the presence of PDE7 in blood vessels. Inhibition of PDE7 in blood vessels raises cAMP which has vasodilator actions and improves organ blood flow to improve the metabolic situation and performance. Altogether, raising cAMP in skeletal muscle (myocytes) and in the blood vessels supplying skeletal muscle and inter alia the brain will improve or cure DACF or prevent relapses or prevent chronic fatigue, exhaustion and/or exertional intolerance prophylactically after an infection. Due to the tissue distribution of PDE7, particularly its expression in skeletal muscle (myocytes), and its role in degrading cAMP in skeletal muscle (myocytes) and blood vessels of skeletal muscles and brain, PDE7 inhibitors can treat and prevent diseases of the DACF with minimal side effects.

Among the diseases associated with fatigue, exhaustion, and/or exertional intolerance, Myalgic Encephalomyelitis/Chronic Fatigue Syndrome (ME/CFS) is the most debilitating condition. Although ME/CFS was frequent affecting approximately 0.3% of the population even before Covid-19 it is not very well-known to most physicians. ME/CFS is characterized by profound fatigue, postexertional malaise (PEM), exertional intolerance, cognitive dysfunction, orthostatic intolerance including postural tachycardia (POTS) and associated with a number of different symptoms including muscle weakness and pain. Despite the fact that non-refreshing sleep is a typical symptom, too, and non-refreshing sleep may worsen the patient's situation, the sleep disturbance is not the initial cause for the profound fatigue and exhaustion, but a consequence of ME/CFS that may further worsen the condition.

Prior to the instant invention, no specific treatment had been available and current symptomatic treatments only provide some symptomatic relief and do not cure. Many patients with ME/CFS are bedridden or wheel chair bound; most are incapacitated for work. ME/CFS is often but not always triggered by infections with various viruses, like Epstein-Barr virus (EBV), enteroviruses, influenza virus, dengue fever and as a recent example Corona virus (SARS-CoV-2). Persisting or newly arising complaints after a past acute Corona-Virus infection are referred to as Long COVID or post-COVID-19 syndrome (PCS) or post-acute Covid syndrome (PACS) or post-Acute Sequelae of SARS-CoV-2 Infection (PASC). Studies into PCS show that there is broadest overlap in symptomatology and mechanisms between both conditions. A fraction of PCS patients develops the full picture of ME/CFS. With regard to symptoms and fall in cerebral blood flow in orthostatic tolerance testing, ME/CFS patients and ME/CFS patients with PCS showed indistinguishable symptomatology. The prevalence of ME/CFS is expected to strongly rise by Covid-19.

Hence, there is a strong demand for a drug or therapeutic agent which can be used for the treatment and prevention of chronic fatigue, exhaustion and/or exertional intolerance associated with different diseases, syndromes, disease states (DACF).

Clearly, there is a strong need in the art for new methods for treating and preventing chronic fatigue, exhaustion and/or extertional intolerance associated with different diseases, syndromes, disease states (DACF).

The present invention provides for a new pathway for treating and preventing DACF by the use of a substance with phosphodiesterase-7 inhibitory activity (PDE7 inhibitors) as active ingredient in a therapeutic agent with phosphodiesterase-7 inhibitory activity.

As used herein, the term “PDE7 inhibitor” includes chemical compounds, proteins or polypeptides, nucleic acids, ribozymes, DNAzymes, protein degraders, gene therapies, or other such agents, which directly or indirectly inhibit or block the phosphodiesterase 7 activity of the PDE7 protein (PDE7A, PDE7B) in a selective or non-selective way. In some cases, the agent may bind or interact directly with PDE7 protein. An agent that binds to PDE7 may act to inhibit or block the PDE7 activation by any suitable means, such as by inhibiting the binding of cAMP or substrate ligand with PDE7. In other cases, the PDE7 inhibitory agent may inhibit PDE7 activity indirectly, such as by decreasing expression of the PDE7 protein. In some cases, the PDE7 inhibitory agent may inhibit PDE7 activity by altering the cellular distribution of PDE7, for example, by interfering with the association between PDE7 and an intracellular anchoring protein.

As used herein, the term diseases associated with chronic fatigue includes different diseases, syndromes, disease states, or conditions associated with chronic fatigue, exhaustion and/or exertional intolerance. It includes Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac-syndrome) after Covid-19 vaccinations and vaccinations against other germs, virus and pathogenic agents postinfectious fatigue and exhaustion after viral, bacterial, or fungal infections, in particular ME/CFS of non-infectious, non-inflammatory and non-immunological cause. Furthermore, this term includes diseases in which chronic fatigue and exhaustion are symptoms of or associated with cancer (cancer-related fatigue), fibromyalgia, Ehlers-Danlos syndrome, Marfan syndrome, Gulf War illness, the autoimmune diseases Rheumatoid Arthritis, ANCA vasculitis and Sjögren's syndrome, and other autoimmune diseases with fatigue and exhaustion as debilitating symptoms. In the following these diseases, syndromes, disease states, syndromes or conditions are subsumed as diseases associated with chronic fatigue (DACF).

In a preferred embodiment of the instant invention the PDE7 inhibitory agent is a compound that is sufficiently potent to inhibit the enzymatic activity of PDE7 (PDE7A, PDE7B, or PDE7A and PDE7B) at an IC₅₀<50 μM, preferably less than or about 1 μM. Even more preferred, the PDE7 inhibitory agent is sufficiently potent to inhibit the enzymatic activity of PDE7 (PDE7A, PDE7B, or PDE7A and PDE7B) at an IC₅₀ of from about 0.1 to about 600 nM, in particular, the PDE7 inhibitory agent is potent to inhibit the enzymatic activity of PDE7 (PDE7A, PDE7B, or PDE7A and PDE7B) at an IC₅₀ of from about 0.2 to about 100 nM, preferrably at an IC₅₀ of from about 1 to about 100 nM.

In the instant invention, the PDE7 inhibitors stimulate the Na⁺/K⁺-ATPase and the NCLX in skeletal muscle to prevent unfavorable ionic changes that cause mitochondrial and energetic dysfunction in skeletal muscle. The harmful rise in sodium (Na⁺) and ensuing changes in calcium (Ca²⁺) are inhibited by stimulation of the Na⁺/K⁺-ATPase and the NCLX via PDE7-inhibition. PDE7-inhibition improves the energetic situation in skeletal muscle by a dual effect, by preventing the key ionic, metabolic and energetic disturbance in skeletal muscle caused by a rise in sodium (Na⁺) and by preventing subsequent unfavorable changes in cellular and mitochondrial calcium as well as by improving blood flow to the muscles. Furthermore, PDE7 inhibition ameliorates the energetic situation in the brain by raising cerebral blood flow to improve the metabolic situation and performance.

The improvements of the energetic situation in skeletal muscle resulting from PDE7-inhibition prevent the release of vasoactive algesic (painful) mediators in skeletal muscles. Normally, these mediators are released to compensate for a poor metabolic situation and are physiologically meant to act only locally in the skeletal muscle. Since excessively produced in the disease state due to the very poor metabolic situation in the large muscle mass of the body, spillover into the systemic circulation occurs so that these mediators with algesic properties can reach and act on every organ in the body. These mediators cause many of the most different symptoms. Apart from fatigue and exhaustion symptoms associated include brain fog, impaired cognition, disturbed sleep, orthostatic intolerance, muscle weakness, pain, post-exertional malaise (PEM), which is an aggravation of the typical symptoms after small or moderate levels of exercise, and many other complaints.

By these combined beneficial effects, PDE7 inhibition improves the symptoms of DACF and potentially cures the diseases of DACF and prevents the relapses and prevents the diseases in a prophylactic manner.

Specific examples for PDE7 inhibitors used in the instant invention are the hereinafter described materials of the formula 1A, formula 1B, compound 1, compound 2, formula 2A, formula 2B, formula 2C, compound 3, formula 3, compound 4, formula 4A, formula 4B, formula 5, formula 6, formula 6A, formula 6B, formula 6C, formula 6D, formula 6E, formula 6F, formula 6G, formula 6H, formula 7A, formula 7B, formula 8, formula 8A, formula 9, formula 10, formula 11, formula 12, formula 13, formula 14, formula 15A, formula 15B, formula 16, formula 16A, formula 17A, formula 17B, formula 18, formula 19, formula 20, formula 21, formula 22, formula 23, formula 24, formula 25, formula 26, formula 27A, formula 27B, formula 27C, formula 27D, formula 28, formula 29, formula 30, formula 31, formula 32, formula 33, formula 34, formula 35, formula 35A, formula 36, formula 37, formula 38, formula 39, formula 40, formula 41, formula 42, formula 42A, formula 43A, formula 43B, formula 44, formula 44A, formula 44B, formula 45, formula 46, formula 47, formula 48, formula 49, formula 50, formula 51, formula 52, formula 53, formula 54, formula 55, formula 55A, formula 55B, formula 55C, formula 55D, formula 55E, formula 55F, formula 55G, formula 55H, formula 55I, formula 55J, formula 56, formula 56B, formula 56C, formula 56D, formula 56E, formula 56F, formula 56G, formula 56H, formula 57, formula 57A, formula 57B, formula 57C, formula 57D, formula 57E, formula 58, formula 59, formula 60, formula 61, formula 62, formula 63, formula 63A, formula 64, dipyridamole, pentoxifylline, SUN11817, S14, S.14, and IBMX.

Methods of Treating and Preventing Diseases Associated with Chronic Fatigue (DACF) Using PDE7 Inhibitor(s):

Thus, the present invention includes methods of treating or preventing DACF, comprising administering one or more PDE7 inhibitors to a subject having DACF or at risk for developing DACF. As used herein, unless the context makes clear otherwise, “treat” and similar word such as “treatment” or “treating” etc., is an approach for obtaining beneficial or desired results, including and preferably clinical results. Treatment can involve optionally either the reducing or amelioration of a disease or condition, e.g., DACF, or the delaying of the progression of the disease or condition, e.g., DACF.

As used herein, unless the context makes clear otherwise, “prevent,” and similar word such as “prevention,” “preventing” etc., is an approach for preventing the onset or recurrence of a disease or condition, (e.g., DACF) or preventing the occurrence, e.g., after a viral infection like with the Epstein-Barr virus, or recurrence of the symptoms of a disease or condition, or optionally an approach for delaying the onset or recurrence of a disease or condition or delaying the occurrence or recurrence of the symptoms of a disease or condition.

As used herein the term “PDE7” is used generically to refer to all translation products coded by transcripts of either or both of these two genes: PDE7A and/or PDE7B.

As used herein, an “effective amount” or a “therapeutically effective amount” of a substance, e.g., a PDE7 inhibitor, is that amount sufficient to affect a desired biological or psychological effect, such as beneficial results, including clinical results. For example, in the context of treating DACF using the methods of the present invention, an effective amount of a PDE7 inhibitor is that amount sufficient to treat and prevent DACF. Generally, a subject is provided with an effective amount of a PDE7 inhibitor.

### Pharmaceutical Compositions, Routes of Administration, Unit Dosage Forms, Kits:

The compositions of the present invention may be administered to a subject as a pharmaceutical composition or formulation. In particular embodiments, pharmaceutical compositions of the present invention may be in any form which allows for the composition to be administered to a subject. For example, the composition may be in the form of a solid, liquid or gas (aerosol). Typical routes of administration include, without limitation, oral, topical, parenteral, sublingual, rectal, vaginal, and intranasal. The term parenteral as used herein includes subcutaneous injections, intravenous, intramuscular, epidural, intrasternal injection or infusion techniques.

Pharmaceutical compositions used according to the present invention comprise a PDE7 inhibitor, another therapeutic agent, and a pharmaceutically acceptable diluent, excipient, or carrier.

“Pharmaceutically acceptable carriers” for therapeutic use are well known in the pharmaceutical art. For example, sterile saline and phosphate buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

Pharmaceutical compositions of the invention are generally formulated so as to allow the active ingredients contained therein to be bioavailable upon administration of the composition to a subject. Compositions that will be administered to a subject may take the form of one or more dosage units, where for example, a tablet, capsule or cachet may be a single dosage unit, and a container comprising a combination of agents according to the present invention in aerosol form may hold a plurality of dosage units. In particular embodiments, the composition comprising a PDE7 inhibitor and eventually another therapeutic agent is administered in one or more doses of a tablet formulation, typically for oral administration. The tablet formulation may be, e.g., an immediate release formulation, a controlled-release formulation, or an extended-release formulation. In one embodiment, a tablet formulation comprises an effective amount of a composition comprising a PDE7 inhibitor and eventually another therapeutic agent. In particular embodiments, a tablet comprises about 1, 5, 10, 20, 30, 50 100, 150, 200, 250, or 300 mg of a PDE7 inhibitor, and eventually about 1, 5, 10, 20, 30, 50 100, 150, 200, 250, or 300 mg of another therapeutic agent if used in combination.

By way of example, a unit administration form of a compound in accordance with the invention in the form of a tablet can comprise the following components: Compound with formula 55E 50 mg, mannitol 223.75 mg, croscarmellose sodium 6 mg, maize starch 15 mg, hydroxypropylmethylcellulose 2.25 mg, and magnesium stearate 3 mg.

Alternatively, in accordance with the invention a capsule formulation can comprise, by way of example, 50 mg of a compound with formula 55E and pharmaceutically acceptable excipients, wherein said excipients can comprise one or more selected from the group consisting of disintegrators, fillers, and lubricants and can comprise an effective amount of binder.

Preferably, the PDE7 inhibitor is provided to a subject in an amount in the range of 0.1-1000 mg/day, 1-1000 mg/day, 10-100 mg/day, or 25-50 mg/day.

Certain combinations of PDE7 inhibitors and other therapeutic agents may not be readily adaptable to coformulation. For example, one of the agents may be more amenable to intravenous administration, while another of the agents may be more amenable to oral administration. Or, the serum half-life of the two agents may be such that one must be administered more frequently than the other. Accordingly, the present invention contemplates kits comprising one or more unit dosage forms of a PDE7 inhibitor and one or more unit dosage forms of another therapeutic agent, such that the two unit dosage forms may be provided to a subject in a therapeutically effective manner.

### PDE7 Proteins and Inhibitory Agents

Cyclic nucleotide phosphodiesterase type 7 (PDE7) is identified as a unique family based on its primary amino acid sequence and distinct enzymatic activity. The PDE genes identified as PDE7 (PDE7A and PDE7B), code for cAMP-specific PDEs. The biochemical and pharmacological characterization of PDE7 shows that PDE7 is a high-affinity cAMP-specific PDE-isoenzyme that does not change levels of cGMP. PDE7 is not inhibited by selective inhibitors of other PDE-isoenzymes. The PDE7 enzyme selectively hydrolyzes cAMP and is characterized as an enzyme that is not inhibited by rolipram, a selective inhibitor of PDE4, which is another distinct, cAMP-specific PDE family. Two sub-types have been identified within the PDE7 family, PDE7A and PDE7B. PDE7A and

PDE7B are encoded by two separate genes located on chromosomes 8, g13-q22 and 6q23-q24, respectively. The two gene products exhibit 70% amino acid sequence identity in their C-terminal catalytic domains.

PDE7A (Uniprot ID: Q13946) has three splice variants (PDE7A1, PDE7A2 and PDE7A3) in humans; these variants are generated via alternative splicing at the N- or C-termini. PDE7A1 and PDE7A2 display 97% sequence identity and vary in their N-terminal regions, while PDE7A3 is a C-terminal variant of PDE7A1 but is a shorter protein that still displays 99% sequence identity with PDE7A1. The nucleotide sequence of PDE7A, transcript variant 1, is accessible in public databases. Human PDE7A1 protein has 482 amino acids. The nucleotide sequence of PDE7A, transcript variant 2, is accessible in public databases. Human PDE7A2 protein has 456 amino acids. Human PDE7A3 protein has 424 amino acids. The nucleotide sequence of PDE7A, transcript variant 3, is accessible in public databases. The PDE7A protein has a region of about 270 amino acids at the carboxy terminal end that displays significant similarity (˜23% homology) to the analogous regions of other cAMP-hydrolyzing PDEs. This region serves as the catalytic domain. The amino-terminal region of this protein is divergent from that of other PDEs and presumably mediates the distinctive and regulatory properties unique to this enzyme family. In skeletal muscle PDE7 is the predominant PDE isoenzyme. The expression of variant PDE7A is much higher than that of the variant PDE7B.

Three splice variants of PDE7B have been reported: PDE7B1, PDE7B2 and PDE7B3 with unique N-terminal sequences. The protein sequence of human PDE7B (Uniprot ID: Q9NP56) is accessible in public databases. Similar to the PDE7A protein, the amino-terminal region of PDE7B protein is divergent and presumably accounts for the distinctive and regulatory properties unique to the individual PDE families. The PDE7B protein shows homology to other cAMP-dependent PDEs (^{˜}23%) within the catalytic domain. The PDE7B polypeptide is 59% homologous to PDE7A.

PDE7 is also uniquely localized in mammalian subjects relative to other PDE families. PDE7A expression has been detected in the majority of tissues analyzed, including the skeletal muscle, heart, kidney, and spleen. PDE7B expression has been detected in the: brain, peripheral blood mononuclear cells, liver, heart, kidney, small intestine, and skeletal muscle. In skeletal muscle PDE7A expression is much higher than that of the variant PDE7B while the latter shows higher expression in the brain.

In the practice of the methods of the invention, representative PDE7 inhibitory agents that inhibit the phosphodiesterase activity of PDE7 include: Molecules that bind to PDE7 and inhibit the enzyme activity of PDE7 (such as small molecule inhibitors or blocking peptides or proteins/protein fragments/fusion proteins that bind to PDE7 and reduce enzymatic activity), molecules that decrease the expression of PDE7 at the transcriptional and/or translational level (such as PDE7 antisense nucleic acid molecules, PDE7 specific RNAi molecules and PDE7 ribozymes, DNAzymes), and PDE7-directed gene therapies, thereby preventing PDE7 from cleaving cAMP. The PDE7 inhibitory agents can be used alone as a primary therapy or in combination with other therapeutics as an adjuvant therapy to enhance the therapeutic benefits, as discussed here.

The inhibition of PDE7 is characterized by at least one of the following changes that occur as a result of administration of a PDE7 inhibitory agent in accordance with the methods of the invention: the inhibition of PDE7-dependent enzymatic cleavage of the 3′-phosphodiester bond in cAMP to form 5′-adenosine monophosphate (5′-AMP), a reduction in the gene or protein expression level of PDE7, measured, for example, by gene expression analysis (e.g., RT-PCR analysis) or protein analysis (e.g., Western blot).

In some embodiments, a PDE7 inhibitory agent is a molecule or composition that inhibits the expression of PDE7A, PDE7B, or both PDE7A and PDE7B, such as an antisense or small inhibitory nucleotide (e.g., siRNA) that specifically hybridizes with the cellular mRNA and/or genomic DNA corresponding to the gene(s) of the target PDE7 so as to inhibit their transcription and/or translation, or a ribozyme that specifically cleaves the mRNA of a target PDE7.

### Potency of PDE7 Inhibitory Agents:

In one embodiment, a PDE7 inhibitory agent useful in the methods of the invention is a compound that is sufficiently potent to inhibit the enzymatic activity of PDE7 (PDE7A, PDE7B, or PDE7A and PDE7B) at an IC₅₀<50 μM, preferably less than or about 1 μM. In one embodiment, the PDE7 inhibitory agent is sufficiently potent to inhibit the enzymatic activity of PDE7 (PDE7A, PDE7B, or PDE7A and PDE7B) at an IC₅₀ of from about 0.1 to about 600 nM. In one embodiment, the PDE7 inhibitory agent is potent to inhibit the enzymatic activity of PDE7 (PDE7A, PDE7B, or PDE7A and PDE7B) at an IC₅₀ of from about 0.2 to about 100 nM, preferrably at an IC₅₀ of from about 1 to about 100 nM.

Representative methods for determining the IC₅₀ for a PDE7 (PDE7A or PDE7B) inhibitory agent are well known in the art, such as the Scintillation Proximity Assay (SPA).

### PDE7A or PDE7B Selective Inhibitory Agents:

In one embodiment, the PDE7 inhibitor useful in the method of the invention is a PDE7A inhibitory agent. In one embodiment, the PDE7A inhibitory agent is potent to inhibit the enzymatic activity of PDE7A at an IC₅₀ of from about 0.1 to about 600 nM.

In some embodiments, the PDE7 inhibitory agent exhibits isozyme-selective activity against PDE7A. A PDE7A selective inhibitory agent reduces PDE7A activity at least two-fold more than PDE7B activity, more preferably at least 10-fold, at least 20-fold, at least 50-fold, or at least 100-fold. In some embodiments, the PDE7A inhibitory agent is an inhibitory agent that is at least 10-fold (such as at least 20-fold, or at least 50-fold or at least 100-fold) more selective for inhibiting PDE7A activity than for the enzyme activity of any other PDE (PDE1-6, 7B, and 8-11).

In one embodiment, the PDE7B inhibitory agent is potent to inhibit the enzymatic activity of PDE7B at an IC₅₀ of from about 0.1 to about 600 nM.

In some embodiments, the PDE7 inhibitor exhibits isozyme-selective activity against PDE7B. A PDE7B selective inhibitory agent reduces PDE7B activity at least two-fold more than PDE7A activity, more preferably at least 10-fold, at least 20-fold, at least 50-fold, or at least 100-fold. In some embodiments, the PDE7B inhibitory agent is an inhibitory agent that is at least 10-fold (such as at least 20-fold, or at least 50-fold or at least 100-fold) more selective for inhibiting PDE7B activity than for the enzyme activity of any other PDE (PDE1-6, 7A, and 8-11).

### Types of PDE7 Inhibitory Agents:

The PDE7 inhibitory agent can be any type of agent including, but not limited to, a chemical compound, a protein or polypeptide, a peptidomimetic, a nucleic acid molecule, a ribozyme, a DNAzyme, a protein degrader, or a gene therapy. In some embodiments, PDE7 inhibitory agents are small molecule inhibitors including natural and synthetic substances that have a low molecular weight, such as, for example, peptides, peptidomimetics and nonpeptide inhibitors such as chemical compounds.

### Chemical Compounds:

The PDE7 inhibitors useful in the methods of the invention include agents that are administered by a conventional route (e.g., oral, intramuscular, subcutaneous, transdermal, transbuccal, intravenous, etc.) into the bloodstream and are ultimately transported through the vascular system to inhibit PDE7 in skeletal muscles and the vasculature.

The following is a description of exemplary PDE7 inhibitors useful in the methods of the invention. They can be orthosteric, allosteric or other inhibitors. If it is not explicitly defined later, the following terms mean:
i) aryl: a functional group derived from an aromatic ring or ring system with or without hetero atoms when one hydrogen is removed from such ring structure,
ii) alkyl: in a broad definition the hydrocarbon group formed when a hydrogen atom is removed from an alkane, alkene, oralkyne group,
iii) alkoxy: functional group containing an alkyl group bonded to oxygen,
iv) acyl: functional group with the formula R—C═O and R being aryl, alkyl or other structures.

In one embodiment, PDE7 inhibitors useful in the methods of the invention are selected from those compounds generally or specifically disclosed in EP-A 1454897, WO 2003/053975, and US 2005/0148604, U.S. Pat. No. 7,268,128, each expressly incorporated herein by reference in its entirety. In one embodiment, PDE7 inhibitors useful in the methods of the invention have the formulas 1A or 1B:

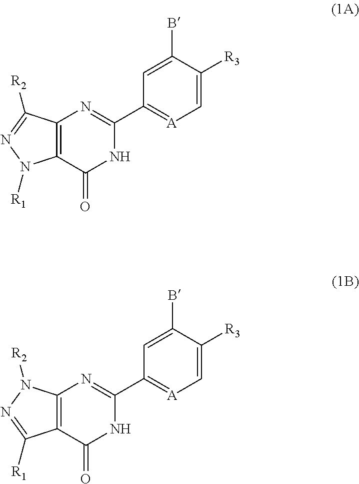

((1A), (1B): Also referred to as pyrazolopyrimidinone derivatives.)

The substituents for the above compounds 1A and 1B are defined as follows:
A represents N or CR₄,
B′ represents a hydrogen atom or a halogen atom,
R₁ represents optionally substituted C₃₋₇ cycloalkyl or tert-butyl,
R₂ represents hydrogen, methyl, or ethyl,
R₃ represents a hydrogen, nitro, cyano or halogen atom, NR₅R₆, C(═X)R₇, SO₂NR₅R₆, OR₈, NR₈CONR₅R₆, NR₈SO₂R₉, NR₈CO₂R₉, a heteroaryl group, optionally substituted C₁₋₃ alkyl, optionally substituted C₁₋₆ alkenyl, or optionally substituted saturated or unsaturated heterocycloalkyl,
R₄ represents hydrogen, or C₁₋₃ alkoxy substituted, if desired, by one or more fluorine atoms,
R₅ and R₆ are the same or different, and represent a hydrogen atom, optionally substituted C₁₋₆ alkyl, optionally substituted heterocycloalkyl, or optionally substituted acyl or, together with the nitrogen atom which they are bound to, form azetidinyl, pyrrolidinyl, piperidinyl, morpholino, thiomorpholino, piperazinyl, or homopiperazinyl, each of these groups being optionally substituted by optionally substituted C₁₋₄ alkyl, OH, C₁₋₃ alkoxy, CO₂H, NR₅R₆, an oxo group, NR₉COR₇, or C(═O)R₇,
R₇ represents optionally substituted C₁₋₆ alkyl, OH, ORs, or NR₅R₆,
R₈ represents hydrogen, an optionally substituted C₁₋₆ alkyl group, or optionally substituted heterocycloalkyl,
R₉ represents an optionally substituted C₁₋₆ alkyl group, and
X represents O, S, or NH.

In regard to the above compounds, “optionally substituted” refers to optionally substituted linear, branched or cyclic alkyl group such as methyl, ethyl, propyl or cyclohexyl; a hydroxyl group; a cyano group; an alkoxy group such as methoxy or ethoxy; an optionally substituted amino group such as amino, methylamino or dimethylamino; an optionally substituted acyl group such as acetyl or propionyl; a carboxyl group; an optionally substituted aryl group such as phenyl or naphthyl; an optionally substituted heteroaryl group such as pyridinyl, thiazolyl, imidazolyl or pyrazyl; an optionally substituted saturated or unsaturated heterocycloalkyl group such as piperazinyl or morphonyl; an optionally substituted carbamoyl group; an optionally substituted amido group; a halogen atom such as chlorine, fluorine or bromine; a nitro group; an optionally substituted sulfone group; an optionally substituted sulfonylamido group; an oxo group; a urea group; and an optionally substituted linear, branched or cyclic alkenyl group such as ethenyl, propenyl or cyclohexenyl. Examples of the heteroaryl group as R³ include a 5- to 7-membered monocyclic heteroaryl group having 2 to 8 carbon atoms and containing 1 to 4 hetero atoms consisting of oxygen atoms, nitrogen atoms or sulfur atoms, and a polycyclic heteroaryl group comprising two or more such identical or different monocyclic compounds fused together, examples of the monocyclic and polycyclic heteroaryl groups being pyrrole, furyl, thienyl, imidazolyl, thiazolyl, pyridyl, pyrazyl, indolyl, quinolyl, isoquinolyl, and tetrazolyl.

In one embodiment, a PDE7 inhibitor useful in the invention has the formula:

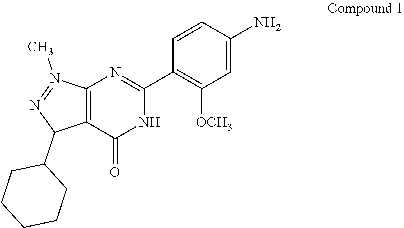

CAS Registry Number assigned by the Chemical Abstracts Service (CAS): 553669-13-9.

In other embodiments, PDE7 inhibitors useful in the methods of the invention have the formulas:

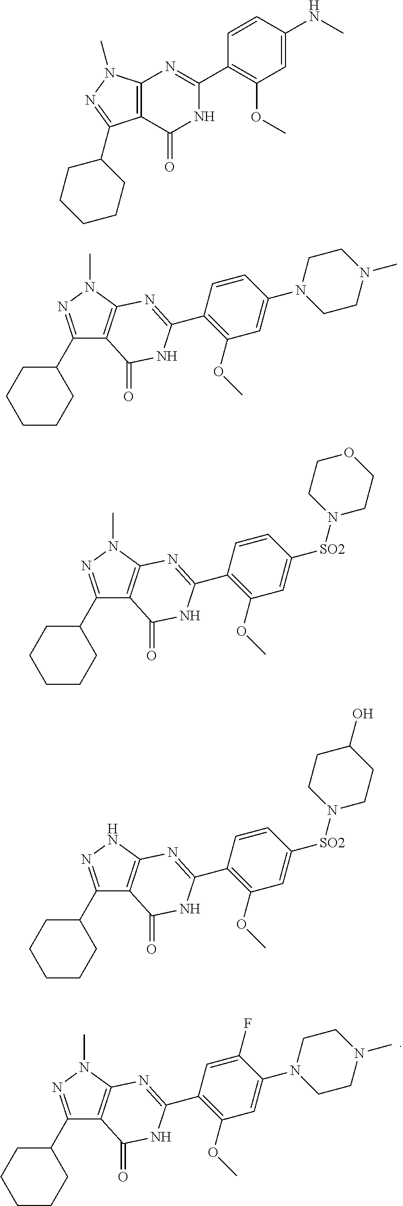

In another embodiment, a PDE7 inhibitor useful in the methods of the invention has the formula:

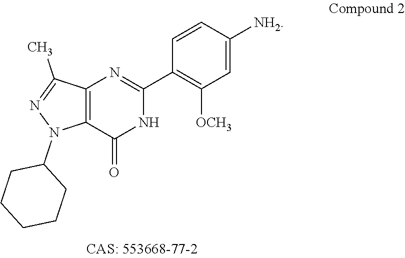 [SMILES: COc1cc(N)ccc1-c1nc2c(C)nn(C3CCCCC3)c2c(=O)[nH]1]

In other embodiments, PDE7 inhibitors useful in the methods of the invention have the formulas:

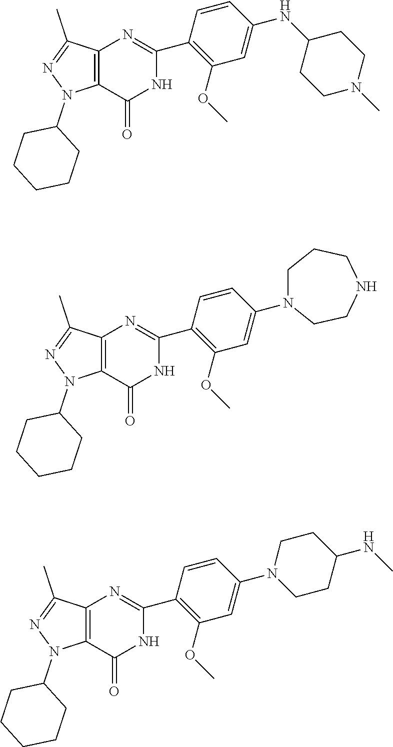 [SMILES: COc1cc(NC2CCN(C)CC2)ccc1-c1nc2c(C)nn(C3CCCCC3)c2c(=O)[nH]1 ; COc1cc(N2CCCNCC2)ccc1-c1nc2c(C)nn(C3CCCCC3)c2c(=O)[nH]1 ; CNC1CCN(c2ccc(-c3nc4c(C)nn(C5CCCCC5)c4c(=O)[nH]3)c(OC)c2)CC1]

The preparation of the above compounds is described in EPA-1454897, WO 2003/053975, and US 2005/0148604.

The above formulas 1A, 1B, Compound 1, Compound 2 are also referred to in U.S. Pat. No. 11,207,275 and WO 2012/064667, U.S. Pat. No. 9,220,715, US 2012/0115849, WO 2013/176877, and US 2022/0062194. The formulas 1A, 1B, Compound 1, Compound 2 are also referred to in U.S. Pat. Nos. 9,119,822, 8,637,528 and WO 2010/129036, U.S. Pat. No. 9,119,822, and US 2019/0183824 and WO 2008/119057.

In another embodiment, PDE7 inhibitors useful in the methods of the invention are selected from those compounds generally or specifically disclosed in US 2002/0198198, WO 2002/076953, WO 2002/074754, WO 2006/092691, Bioorganic & Medicinal Chemistry Letters 14 (2004) 4623-4626, US 2009/0111837 and Bioorganic & Medicinal Chemistry Letters 14 (2004) 4627-4631, each expressly incorporated herein by reference in its entirety. In one embodiment, PDE7 inhibitors useful in the methods of the invention have the formulas:

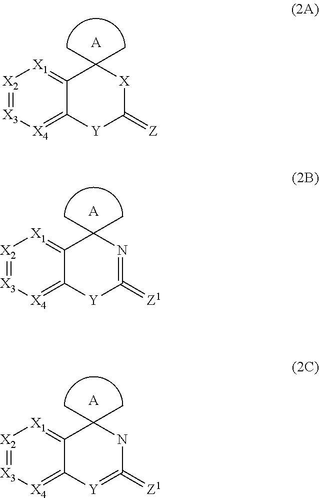

((2A), (2B), (2C): Also referred to as spirocyclic derivatives/spirocycyles.)

The substituents for the above compounds 2A, 2B and 2C are defined as follows:
(a) X₁, X₂, X₃, and X₄ are the same or different and are selected from:
N, provided that not more than two of the groups X₁, X₂, X₃, and X₄ simultaneously represent a nitrogen atom, or, C—R₁, in which R₁ is selected from: Q₁, or lower alkyl, lower alkenyl, or lower alkynyl, these groups being unsubstituted or substituted with one or several groups Q₂; the group X₅—R₅ in which, X₅ is selected from: a single bond, lower alkylene, lower alkenylene, or lower alkynylene; optionally interrupted with 1 or 2 heteroatoms chosen from O, S, S(═O), SO₂, or N, the carbon atoms of these groups being unsubstituted or substituted with one or several groups, identical or different, selected from SR₆, OR₆, NR₆R₇, ═O, ═S, or ═NR₆ in which R₆ and R₇ are the same or different and are selected from hydrogen or lower alkyl, and, R₅ is selected from aryl, heteroaryl, cycloalkyl optionally interrupted with C(═O) or with 1, 2, or 3 heteroatoms chosen from O, S, S(═O), SO₂, or N, cycloalkenyl optionally interrupted with C(═O) or with 1, 2, or 3 heteroatoms chosen from O, S, S(═O), SO₂ or N, or a bicyclic group, these groups being unsubstituted or substituted with one or several groups selected from Q₃, heteroaryl, or lower alkyl optionally substituted with Q₃; in which Q₁, Q₂, and Q₃ are the same or different and are selected from: hydrogen, halogen, CN, NO₂, SO₃H, P(═O)(OH)₂, OR₂, OC(═O)R₂, C(═O)OR₂, SR₂, S(═O)R₂, NR₃R₄, Q-R₂, Q-NR₃R₄, NR₂-Q-NR₃R₄, or NR₃-Q-R₂ in which Q is selected from C(═NR), C(═O), C(═S), or SO₂, R is selected from hydrogen, or lower alkyl, and R₂, R₃, and R₄ are the same or different and are selected from: hydrogen, lower alkyl optionally interrupted with C(═O), (CH₂)ₙ-aryl, (CH₂)ₙ-heteroaryl, (CH₂)ₙ-cycloalkyl optionally interrupted with C(═O) or with 1 or 2 heteroatoms chosen from O, S, S(═O), SO₂, or N, in which n is an integer selected from 0, 1, 2, 3 or 4; these groups being unsubstituted or substituted with one or several groups selected from lower alkyl, halogen, CN, CH₃, SO₃H, SO₂CH₃, CF₃, C(═O)NHSO₂CH₃, OR₆, COOR₆, C(═O)R₆, NR₆R₇, C(═O)NR₆R₇, or SO₂NR₆R₇, in which R₆ and R₇ are the same or different and are selected from hydrogen or lower alkyl optionally substituted with one or two groups selected from OR, COOR or NRR₈ in which R and R₈ are hydrogen or lower alkyl, and, R₆ and R₇, and/or, R₃ and R₄, together with the nitrogen atom to which they are linked, can form a 4- to 8-membered heterocyclic ring, which may contain one or two heteroatoms selected from O, S, S(═O), SO₂, or N, and which may be substituted with, a 4- to 8-membered heterocyclic ring, which may contain one or two heteroatoms selected from O, S, or N, and which may be substituted with a lower alkyl, or, a lower alkyl optionally substituted with OR′, NR′R″, C(═O)NR′R′ or COOR′ in which R′ and R″ are the same or different and are selected from H, lower alkyl optionally substituted with OR or COOR in which R is hydrogen or lower alkyl, and R′ and R″ together with the nitrogen atom to which they are linked, can form a 4- to 8-membered heterocyclic ring, which may contain one or two heteroatoms selected from O, S, or N; or, (b) X is O, S, or NR₉, in which R₉ is selected from hydrogen, CN, OH, NH₂, lower alkyl, lower alkenyl, or lower alkynyl, these groups being unsubstituted or substituted with cycloalkyl optionally interrupted with 1 or 2 heteroatoms chosen from O, S, S(═O), SO₂, or N, cycloalkenyl optionally interrupted with 1 or 2 heteroatoms chosen from O, S, S(═O), SO₂, or N, aryl, heteroaryl, OR₁₀, or NR₁₀R₁₁ in which R₁₀ and R₁₁ are the same or different and are selected from hydrogen or lower alkyl;
(c) Y is selected from O, S, or N—R₁₂, in which R₁₂ is selected from hydrogen, CN, OH, NH₂, lower alkyl, lower alkenyl, or lower alkynyl, these groups being unsubstituted or substituted with cycloalkyl optionally interrupted with 1 or 2 heteroatoms chosen from O, S, S(═O), SO₂, or N, cycloalkenyl optionally interrupted with 1 or 2 heteroatoms chosen from O, S, S(═O), SO₂, or N, aryl, heteroaryl, OR₁₀, or NR₁₀R₁₁ in which R₁₀ and R₁₁ are the same or different and are selected from hydrogen or lower alkyl;
(d) Z is chosen from CH—NO₂, O, S, or NR₁₃ in which R₁₃ is selected from hydrogen, CN, OH, NH₂, aryl, heteroaryl, cycloalkyl optionally interrupted with one or several heteroatoms chosen from O, S, S(═O), SO₂, or N, cycloalkenyl optionally interrupted with one or several heteroatoms chosen from O, S, S(═O), SO₂, or N, C(═O)R₁₄, C(═O)NR₁₄R₁₅, OR₁₄, or, lower alkyl, unsubstituted or substituted with one or several groups which are the same or different and which are selected OR₁₄ or NR₁₄R₁₅; R₁₄ and R₁₅ being independently selected from hydrogen or lower alkyl, or, R₁₄ and R₁₅, together with the nitrogen atom to which they are linked, can form a 4- to 8-membered heterocyclic ring which may contain one or two heteroatoms chosen from O, S, or N, and which may be substituted with a lower alkyl;
(e) Z¹ is chosen from H, CH₃, or NR₁₆R₁₇ in which R₁₆ and R₁₇ are the same or different and are selected from hydrogen, CN, aryl, heteroaryl, cycloalkyl optionally interrupted with one or several heteroatoms chosen from O, S, S(═O), SO₂, or N, cycloalkenyl optionally interrupted with one or several heteroatoms chosen from O, S, S(═O), SO₂, or N, C(═O)R₁₄, C(═O)NR₁₄R₁₅, OR₁₄, or, lower alkyl unsubstituted or substituted with one or several groups selected from OR₁₄ or NR₁₄R₁₅, R₁₄ and R₁₅ being chosen from hydrogen or lower alkyl, and, R₁₄ and R₁₅, and/or, R₁₆ and R₁₇, together with the nitrogen atom to which they are linked, can form a 4- to 8-membered heterocyclic ring which may contain one or two heteroatoms chosen from O, S, or N, and which may be substituted with a lower alkyl;
(f) A is a cycle selected from:

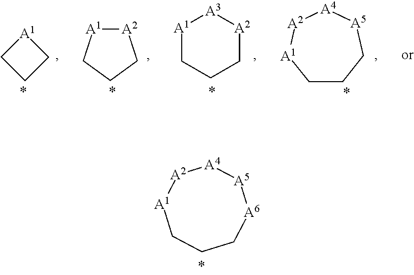

in which
A¹, A², A³, A⁴, A⁵, and A⁶ are the same or different and are selected from O, S, C, C(═O), SO, SO₂, or NR₁₈ in which R₁₈ is selected from hydrogen, aryl, heteroaryl, cycloalkyl optionally interrupted with one or several heteroatoms chosen from O, S, S(═O), SO₂, or N, cycloalkenyl optionally interrupted with one or several heteroatoms chosen from O, S, S(═O), SO₂, or N, lower alkyl unsubstituted or substituted with aryl, heteroaryl, cycloalkyl optionally interrupted with one or several heteroatoms chosen from O, S, S(═O), SO₂, or N, cycloalkenyl optionally interrupted with one or several heteroatoms chosen from O, S, S(═O), SO₂, or N, CN, NR₁₉R₂₀, C(═O)NR₁₉R₂₀, OR₁₉, C(═O)R₁₉ or C(═O)OR₁₉ in which R₁₉ and R₂₀ are identical or different and are selected from hydrogen or lower alkyl;
represents the carbon atom which is shared between the cycle A and the backbone cycle containing X and/or Y;
each carbon atom of the cycle A is unsubstituted or substituted with 1 or 2 groups, identical or different, selected from lower alkyl optionally substituted with OR₂₁, NR₂₁R₂₂, COOR₂₁, or CONR₂₁R₂₂, lower haloalkyl, CN, F, ═O, SO₂NR₁₉R₂₀, OR₁₉, SR₁₉, C(═O)OR₁₉, C(═O)NR₁₉R₂₀, or NR₁₉R₂₀ in which R₁₉ and R₂₀ are identical or different and are selected from hydrogen or lower alkyl optionally substituted with OR₂₁, NR₂₁R₂₂, COOR₂₁, or CONR₂₁R₂₂, in which R₂₁ and R₂₂ are identical or different and are selected from hydrogen or lower alkyl, and, R₁₉ and R₂₀, and/or, R₂₁ and R₂₂, together with the nitrogen atom to which they are linked, can form a 4- to 8-membered heterocyclic ring;
two atoms of the cycle A, which are not adjacent, may be linked by a 2, 3 or 4 carbon atom chain which may be interrupted with 1 heteroatom chosen from O, S or N; provided that not more than two of the groups A¹, A², A³, A⁴, A⁵, and A⁶ simultaneously represent a heteroatom; and
their tautomeric forms, their racemic forms, their isomers, and their pharmaceutically acceptable derivatives.

In regard to the above compounds, halogen includes fluoro, chloro, bromo, and iodo. Preferred halogens are F and C₁. Lower alkyl includes straight and branched carbon chains having from 1 to 6 carbon atoms. Examples of such alkyl groups include methyl, ethyl, isopropyl, and tert-butyl. Lower alkenyl includes straight and branched hydrocarbon radicals having from 2 to 6 carbon atoms and at least one double bond. Examples of such alkenyl groups are ethenyl, 3-buten-1-yl, 2-ethenylbutyl, and 3-hexen-1-yl. Lower alkynyl includes straight and branched hydrocarbon radicals having from 2 to 6 carbon atoms and at least one triple bond. Examples of such alkynyl groups are ethynyl, 3-butyn-1-yl, propynyl, 2-butyn-1-yl, and 3-pentyn-1-yl. Lower haloalkyl includes a lower alkyl as defined above, substituted with one or several halogens. An example of haloalkyl is trifluoromethyl. Aryl is understood to refer to an aromatic carbocycle containing between 6 and 10 carbon atoms. An example of an aryl group is phenyl. Heteroaryl includes aromatic cycles which have from 5 to 10 ring atoms, from 1 to 4 of which are independently selected from the group consisting of O, S, and N. Representative heteroaryl groups have 1, 2, 3 or 4 heteroatoms in a 5- or 6-membered aromatic ring. Examples of such groups are tetrazole, pyridyl, and thienyl. Representative cycloalkyl contain from 3 to 8 carbon atoms. Examples of such groups are cyclopropyl, cyclobutyl, cyclopentyl, cyclohexyl, cycloheptyl and cyclooctyl. The term “interrupted” means that in a backbone chain, a carbon atom is replaced by a heteroatom or a group as defined herein. For example, in “cycloalkyl or cycloalkenyl optionally interrupted with C(═O) or with 1 heteroatom chosen from O, S, S(═O), SO₂ or N”, the term “interrupted” means that C(═O) or a heteroatom can replace a carbon atom of the ring. Example of such groups are morpholine or piperazine. Cycloalkenyl includes 3- to 10-membered cycloalkyl containing at least one double bond. Heterocyclic rings include heteroaryl as defined above and cycloalkyl or cycloalkenyl, as defined above, interrupted with 1, 2 or 3 heteroatoms chosen from O, S, S(═O), SO₂, or N. Bicyclic substituents refer to two cycles, which are the same or different and which are chosen from aryl, heterocyclic ring, cycloalkyl or cycloalkenyl, fused together to form said bicyclic substituents. An example of a bicyclic substituent is indolyl.

In one embodiment, a PDE7 inhibitor useful in the methods of the invention has the formula:

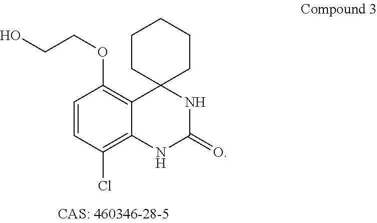 [SMILES: O=C1Nc2c(Cl)ccc(OCCO)c2C2(CCCCC2)N1]

The above formulas of 2A, 2B, 2C, and Compound 3 are also referred to in U.S. patent Ser. No. 11/207,275 and WO 2012/064667, U.S. Pat. No. 9,220,715, US 2012/0115849, WO 2013/176877, and US 2022/0062194. The formulas of 2A, 2B, 2C, and Compound 3 are also referred to in U.S. Pat. Nos. 9,119,822, 8,637,528 and WO 2010/129036, and US 2019/0183824 and WO 2008/119057.

In other embodiments, PDE7 inhibitors useful in the methods of the invention have the formulas:

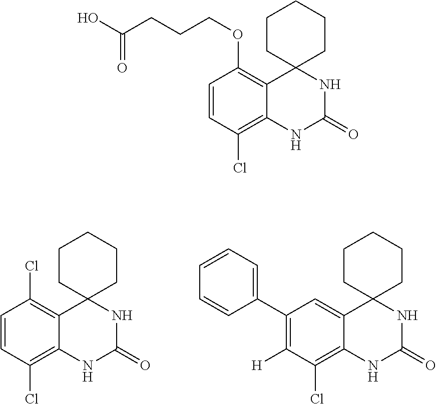 [SMILES: O=C(O)CCCOc1ccc(Cl)c2c1C1(CCCCC1)NC(=O)N2 ; O=C1Nc2c(Cl)ccc(Cl)c2C2(CCCCC2)N1 ; O=C1Nc2c(Cl)cc(-c3ccccc3)cc2C2(CCCCC2)N1]

Any such and all chloro-derivatives of formula 2A are referred to as 8′-chloro-2′,3′-dihydro-2′-oxospiro[cyclohexane-1,4′(1′H)-quinazolin]-5′-yl derivatives.

The preparation of the above compounds is described in US 2002/0198198, WO 2002/076953, WO 2002/074754, WO 2006/092691, Bioorganic & Medicinal Chemistry Letters 14 (2004) 4623-4626, and Bioorganic & Medicinal Chemistry Letters 14 (2004) 4627-4631.

In other embodiments, PDE7 inhibitors that may be useful in the methods of the invention can be derived from compounds with the formula 2A where in the X₁ and X₂ position another heterocycle with substituents is annuleted with X₁ and X₂ being a carbon atom (also referred to a as spirocyclic quinazoline derivatives).

In another embodiment, PDE7 inhibitors useful in the methods of the invention are selected from those compounds generally or specifically disclosed in EP-A-1193261, WO 2002/28847, US 2003/0045557, U.S. Pat. No. 7,122,565, Bioorganic & Medicinal Chemistry Letters 14 (2004) 4607-4613, and Bioorganic & Medicinal Chemistry Letters 14 (2004) 4615-4621, each expressly incorporated herein by reference in its entirety. In one embodiment, PDE7 inhibitors useful in the methods of the invention have the formula (3):

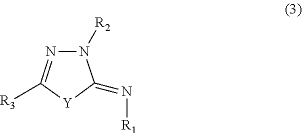

((3): Also referred to as thiadiazole and oxadiazole derivatives.)

The substituents for the above formula (3) are defined as follows:
Y is S or O;
R₁ is C₁-C₁₀ alkyl, C₂-C₁₀ alkenyl, C₂-C₁₀ alkynyl, cycloalkyl, cycloalkenyl, heterocycle, aryl, or a polycyclic group; each optionally substituted with one or several groups X₁—R₄, identical or different, in which X₁ is a single bond, lower alkylene, C₂-C₆ alkenylene, cycloalkylene, arylene, or divalent heterocycle, and R₄ is:
(1) H, ═O, NO₂, CN, halogen, lower haloalkyl, lower alkyl, carboxylic acid bioisostere;
(2) COOR₅, C(═O)R₅, C(═S)R₅, SO₂R₅, SOR₅, SO₃R₅, SR₅, OR₅;
(3) C(═O)NR₇R₈, C(═S)NR₇R₈, C(═CH—NO₂)NR—R₃, C(═N—CN)NR₇R₈, C(═N—SO₂NH₂)NR₇R₃, C(═NR₇) NHR₃, C(═NR₇)R₈, C(═NR₉) NHR₈, C(═NR₉)R₈, SO₂NR₇R₈, or NR₇R₈, wherein R₇ and R₈ are the same or different and are selected from OH, R₅, R₆, C(═O)NR₅R₆, C(═O)R₅, SO₂R₅, C(═NR₉) NHR₁₀, C(═NR₉)R₁₀, C(═CH—NO₂)NR₉R₁₀, C(═N—SO₂NH₂)NR₉R₁₀, C(═N—CN)NR₉R₁₀, or C(═S)NR₉R₁₀;
R₂ is lower alkyl, C₂-C₁₀ alkenyl, C₂-C₁₀ alkynyl, cycloalkyl, cycloalkenyl, heterocycle, aryl; each optionally substituted with one or several groups which are the same or different and which are selected from:
(1) H, carboxylic acid bioisostere, lower haloalkyl, halogen,
(2) COOR₅, OR₅, SO₂R₅,
(3) SO₂NR₁₁R₁₂, C(═O)NR₁₁R₁₂, NR₁₁R₁₂, wherein R₁₁ and R₁₂ are the same or different and are selected from OH, R₅, R₆, C(═O)NR₅R₆, C(═O)R₅, SO₂R₅, C(═S)NR₉R₁₀, C(═CH—NO₂)NR₉R₁₀, C(═N—CN)NR₉R₁₀, C(═N—SO₂NH₂)NR₉R₁₀, C(═NR₉) NHR₁₀, or C(═NR₉)R₁₀;
R₃ is X₂—R′3, wherein X₂ is a single bond or, a group selected from C₁-C₄ alkylene, C₂-C₆ alkenylene, C₂-C₆ alkynylene, each optionally substituted with one or several groups which are the same or different and which are selected from:
(1) H, C₁-C₃ alkyl, C₃-C₄ cycloalkyl, aryl, heterocycle, ═O, CN,
(2) OR₅, ═NR₅; or
(3) NR₁₃R₁₄, wherein R₁₃ and R₁₄ are the same or different and are selected from R₅, R₆, C(═O)NR₅R₆, C(═O)R₅, SO₂R₅, C(═S)NR₉R₁₀, C(═CH—NO₂)NR₉R₁₀, C(═NR₉) NHR₁₀, or C(═NR₉)R₁₀;
R′₃ is cycloalkyl, cycloalkenyl, aryl, heterocycle, or a polycyclic group; each optionally substituted with one or several groups X₃—R₁₇ wherein X₃ is a single bond, lower alkylene, C₂-C₆ alkenylene, C₂-C₆ alkynylene, cycloalkylene, arylene, divalent heterocycle or a divalent polycyclic group, and, R₁₇ is:
(1) H, ═O, NO₂, CN, lower haloalkyl, halogen, carboxylic acid bioisostere, cycloalkyl,
(2) COOR₅, C(═O)R₅, C(═S)R₅, SO₂R₅, SOR₅, SO₃R₅, SR₅, OR₅;
(3) C(═O)NR₁₅R₁₆, C(═S)NR₁₅R₁₆, C(═N—CN)NR₁₅R₁₆, C(═N—SO₂NH₂)NR₁₅R₁₆, C(═CH—NO₂)NR₁₅R₁₆, SO₂NR₁₅R₁₆, C(═NR₁₅) NHR₁₆, C(═NR₁₅)R₁₆, C(═NR₉) NHR₁₆, C(═NR₉)R₁₆, or NR₁₅R₁₆ wherein R₁₅ and R₁₆ are the same or different and are selected from OH, R₅, R₆, C(═O)NR₅R₆, C(═O)R₅, SO₂R₅, C(═S)NR₉R₁₀, C(═CH—NO₂)NR₉R₁₀, C(═N—CN)NR₉R₁₀, C(═N—SO₂NH₂)NR₉R₁₀, C(═NR₉) NHR₁₀ or C(═NR₉)R₁₀,
(4) heterocycle optionally substituted with one or several groups R₅;
wherein R₅ and R₆ are the same or different and are selected from H, lower alkyl, C₂-C₆ alkenyl, C₂-C₆ alkynyl, X₄-cycloalkyl, X₄-cycloalkenyl, X₄-aryl, X₄-heterocycle or X₄-polycyclic group, wherein X₄ is a single bond, lower alkylene, or C₂-C₆ alkenylene; each optionally substituted with one or several groups that are the same or different and selected from halogen, ═O, COOR₂₀, CN, OR₂₀, O-lower alkyl optionally substituted with OR₂₀, C(═O)-lower alkyl, lower haloalkyl,

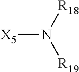

in which X₅ is a single bond or lower alkylene and R₁₈, R₁₉, and R₂₀, are the same or different and are selected from H or lower alkyl;
X₆-heterocycle, X₆-aryl, X₆-cycloalkyl, X₆-cycloalkenyl, or X₆-polycyclic group, wherein X₆ is a single bond or lower alkylene, these groups being optionally substituted with one or several groups, identical or different, selected from halogens, COOR₂₁, OR₂₁, or (CH₂)ₙNR₂₁R₂₂ in which n is 0, 1, or 2 and R₂₁ and R₂₂ are the same or different and are selected from H or lower alkyl;
R₉ is selected from H, CN, OH, lower alkyl, O-lower alkyl, aryl, heterocycle, SO₂NH₂, or

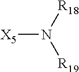

in which X₅ is a single bond or lower alkylene and R₁₈ and R₁₉ are the same or different and are selected from H or lower alkyl;
R₁₀ is selected from hydrogen, lower alkyl, cyclopropyl, or heterocycle;
or their pharmaceutically acceptable derivatives.

In regard to the above compounds, aryl refers to an unsaturated carbocycle, exclusively comprising carbon atoms in the cyclic structure, the number of which is between 5 and 10, including phenyl, naphthyl, or tetrahydronaphthyl. Heterocycle refers to a nonsaturated or saturated monocycle containing between 1 and 7 carbon atoms in the cyclic structure and at least one heteroatom in the cyclic structure, such as nitrogen, oxygen, or sulfur, preferably from 1 to 4 heteroatoms, identical or different, selected from nitrogen, sulfur and oxygen atoms. Suitable heterocycles include morpholinyl, piperazinyl, pyrrolidinyl, piperidinyl, pyrimidinyl, 2- and 3-furanyl, 2- and 3-thienyl, 2-pyridyl, 2- and 3-pyranyl, hydroxypyridyl, pyrazolyl, isoxazolyl, tetrazole, imidazole, triazole, and the like. Polycyclic groups include at least two cycles, identical or different, selected from aryl, heterocycle, cycloalkyl, cycloalkenyl groups fused together to form said polycyclic group such as 2- and 3-benzothienyl, 2- and 3-benzofuranyl, 2-indolyl, 2- and 3-quinolinyl, acridinyl, quinazolinyl, indolyl benzo[1,3]dioxolyl, and 9-thioxantanyl. Bicyclic groups refer to two cycles, which are the same or different and which are chosen from aryl, heterocycle, cycloalkyl or cycloalkenyl, fused together to form said bicyclic groups. Halogen refers to fluorine, chlorine, bromine, or iodine. Lower alkyl refers to an alkyl is linear or branched and contains 1 to 6 carbon atoms. Examples of lower alkyl groups include methyl, ethyl, propyl, butyl, isopropyl, tert-butyl, isobutyl, n-butyl, pentyl, hexyl and the like. Alkenyl refers to a linear or branched unsaturated carbon atom chain, comprising one or several double bonds, preferably one or two double bonds. Alkynyl refers to a linear or branched unsaturated carbon atom chain, comprising one or several triple bonds, preferably one or two triple bonds. Lower haloalkyl refers to a lower alkyl substituted with one or several halogens; preferred lower haloalkyl groups include perhaloalkyl groups such as CF₃. Cycloalkyl refers to saturated monocarbocyle containing from 3 to 10 carbon atoms; including cyclopropyl, cyclobutyl, cyclopentyl, cyclohexyl, and cycloheptyl. Cycloalkenyl refers to unsaturated monocarbocyle containing from 3 to 10 carbon atoms. Examples of suitable cycloalkenyl are 3-cyclohexene, and 3-cycloheptene. Carboxylic acid bioisostere has the classical meaning; common carboxylic acid bioisostere are tetrazole-5-yl, C(═O)N(H)OH, isoxazol-3-yl, hydroxythiadiazolyl, sulfonamido, sulfonylcarboxamido, phosphonic acid, phosphonamido, phosphinic acid, sulfonic acids, acyl sulfonamido, mercaptoazole, acyl cyanamides.

In one embodiment, a PDE7 inhibitor useful in the methods of the invention has the formula: Compound 4:

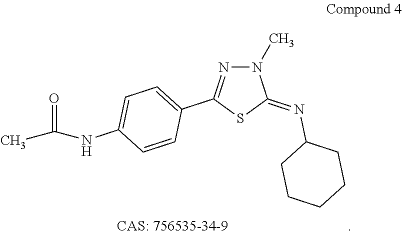 [SMILES: CC(=O)Nc1ccc(-c2nn(C)/c(=N/C3CCCCC3)s2)cc1]

The above formulas of 3 and Compound 4 are also referred to in U.S. Pat. No. 11,207,275 and WO 2012/064667, U.S. Pat. No. 9,220,715, US 2012/0115849, WO 2013/176877, and US 2022/0062194. The formulas of 3 and Compound 4 are also referred to in U.S. Pat. Nos. 9,119,822, 8,637,528 and WO 2010/129036, US 2019/0183824 and WO 2008/119057.

In other embodiments, PDE7 inhibitors useful in the methods of the invention have the formulas:

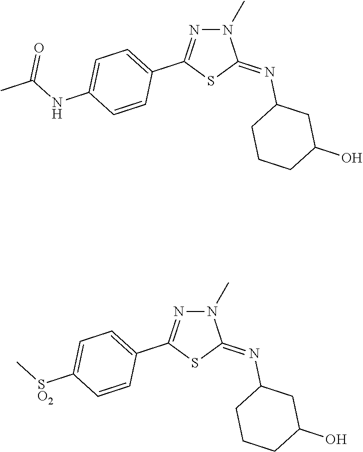 [SMILES: CC(=O)Nc1ccc(-c2nn(C)/c(=N/C3CCCC(O)C3)s2)cc1 ; Cn1nc(-c2ccc(S(C)(=O)=O)cc2)s/c1=N\C1CCCC(O)C1]

The preparation of the above compounds is described in EP-A-1193261, WO 02/28847, US 2003/0045557, U.S. Pat. No. 7,122,565, Bioorganic & Medicinal Chemistry Letters 14 (2004) 4607-4613, and Bioorganic & Medicinal Chemistry Letters 14 (2004) 4615-4621.

In another embodiment, PDE7 inhibitors useful in the methods of the invention are selected from those compounds generally or specifically disclosed in WO 2004/111054, US20060128728, and US 20070270419, each expressly incorporated herein by reference in its entirety. In one embodiment, PDE7 inhibitors useful in the methods of the invention have the formulas 4A and/or 4B:

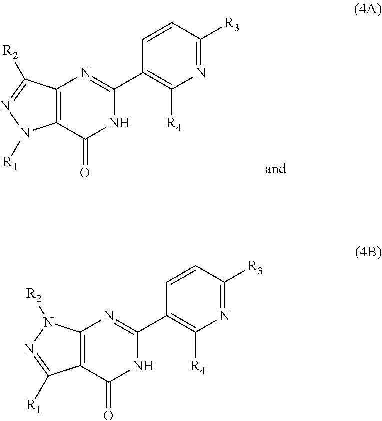

((4A), (4B): Also referred to as pyridinylpyrazolopyrimidinone derivatives)

The substituents for the above compounds 4A and 4B are defined as follows:
R₁ is a substituted or unsubstituted C₃₋₈ cycloalkyl group or tert-butyl group;
R₂ is a hydrogen atom or C₁₋₃ alkyl group;
R₃ is a group: NR₅R₆, C(═O)R₇, or S(O)₀₋₂R₈;
R₄ is a hydrogen atom or C₁₋₃ alkoxyl group which is unsubstituted or substituted by one or more fluorine atom(s);
R₅ and R₆ are, same or different from each other, a hydrogen atom, substituted or unsubstituted C₁₋₆ alkyl group, substituted or unsubstituted acyl group, substituted or unsubstituted heterocycloalkyl group, and substituted or unsubstituted heterocycloalkyl ring formed with a nitrogen atom which is binding R₅ and R₆;
R₇ is a group: OR₉ or NR₅R₆;
R₈ is a hydrogen atom, a halogen atom, a group: NR₅R₆, substituted or unsubstituted C₁₋₆ alkyl group, or substituted or unsubstituted aryl group;
R₉ is a hydrogen atom or substituted or unsubstituted C₁₋₆ alkyl group;
or pharmaceutically acceptable salts or solvates thereof.

In regard to the above compounds, the term “C₁-C₃ alkyl group” includes a straight or branched-chained alkyl group having 1 to 3 carbon atoms. The term “C₃-C₈ cycloalkyl group” includes a cycloalkyl group having 3 to 8 carbon atoms such as cyclopropyl, cyclobutyl, cyclopentyl, cyclohexyl and cyclooctyl. The term “heterocycloalkyl group” is 3 to 7 membered heterocyclic group containing the same or different 1 to 4 hetero atom(s) such as oxygen, nitrogen or sulfur atom(s), and examples may include pyrrolidinyl, piperidinyl, piperazinyl, homopiperazinyl, tetrahydrofuryl, tetrahydrophyranyl, morpholinyl and azetidinyl. The term “C₁-C₃ alkoxy group” means alkoxy group having 1 to 3 carbon atoms. The term “acyl group” means acyl group having 1 to 8 carbon atoms. The term “aryl group” is phenyl, naphthyl, biphenyl group, having 6 to 12 carbon atoms, and the term “heteroaryl group” is 5 to 7 membered monocyclic or polycyclic group thereof containing 2 to 8 carbon atoms and the same or different 1 to 4 hetero atom(s) such as oxygen, nitrogen, sulfur atom(s). The examples include pyrrole, furyl, thienyl, imidazolyl, thiazolyl, pyrazinyl, indolyl, quinolinyl, isoquinolinyl, tetrazolyl, pyridinyl, pyrazolyl pyridazinyl and pyrimidinyl. Examples of suitable substituent of “substituted or unsubstituted C₁-C₆ alkyl group” include hydroxyl group and halogen atom, and examples of suitable substituent of “substituted or unsubstituted acyl group” include halogen atom and nitro group. Further, examples of suitable substituent of “substituted or unsubstituted aryl group” include C₁-C₃ alkyl, halogen atom, amino group, acyl group, amide group, hydroxyl group, acylamino group, carboxyl group and sulfonyl group. Examples of suitable substituent of “substituted or unsubstituted C₃-C₈ cycloalkyl group” is C₁-C₃ alkyl, hydroxyl group and oxo group, and examples of suitable substituent of “substituted or unsubstituted heterocycloalkyl group” may include carboxy group, acyl group, alkoxy group, amino group, alkylamino group, acylamino group, hydroxyl group, oxo group, ethylenedioxy group, methyl group, ethyl group and hydroxyethyl group.

In other embodiments, PDE7 inhibitors useful in the methods of the invention have the formulas:

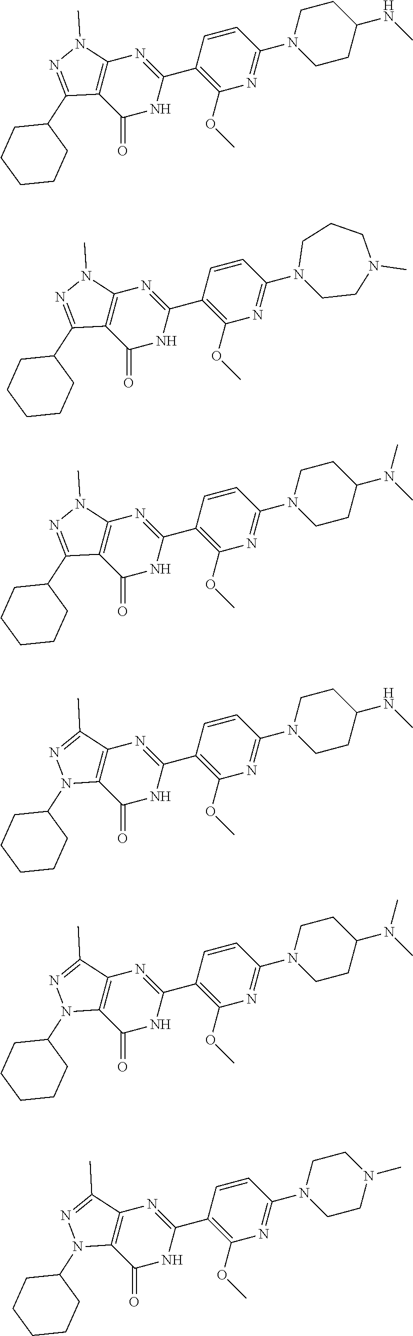 [SMILES: CNC1CCN(c2ccc(-c3nc4c(c(C5CCCCC5)nn4C)c(=O)[nH]3)c(OC)n2)CC1 ; COc1nc(N2CCCN(C)CC2)ccc1-c1nc2c(c(C3CCCCC3)nn2C)c(=O)[nH]1 ; COc1nc(N2CCC(N(C)C)CC2)ccc1-c1nc2c(c(C3CCCCC3)nn2C)c(=O)[nH]1 ; CNC1CCN(c2ccc(-c3nc4c(C)nn(C5CCCCC5)c4c(=O)[nH]3)c(OC)n2)CC1 ; COc1nc(N2CCC(N(C)C)CC2)ccc1-c1nc2c(C)nn(C3CCCCC3)c2c(=O)[nH]1 ; COc1nc(N2CCN(C)CC2)ccc1-c1nc2c(C)nn(C3CCCCC3)c2c(=O)[nH]1]

The preparation of the above compounds is described in WO 2004/111054, US 2006/0128728, and US 2007/0270419.

In another embodiment, PDE7 inhibitors useful in the methods of the invention are selected from those compounds generally or specifically disclosed in U.S. Pat. No. 6,903,109, US 2004/0082578, WO 2003/088963, and US 2006/0154949, each expressly incorporated herein by reference in its entirety. In one embodiment, PDE7 inhibitors useful in the methods of the invention have the formula (5):

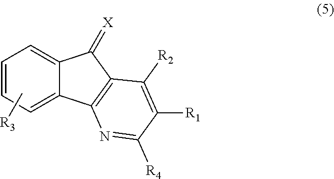

((5): Also referred to as arylindenopyridine derivatives.)

The substituents for the above compounds (5) are defined as follows:
(a) R₁ is selected from the group consisting of:
(i) COR₅, wherein R₅ is selected from H, optionally substituted C₁₋₈ straight or branched chain alkyl, optionally substituted aryl and optionally substituted arylalkyl; wherein the substituents on the alkyl, aryl and arylalkyl group are selected from C₁₋₈ alkoxy, phenylacetyloxy, hydroxy, halogen, p-tosyloxy, mesyloxy, amino, cyano, carboalkoxy, or NR₂₀R₂₁ wherein R₂₀ and R₂₁ are independently selected from the group consisting of hydrogen, C₁₋₈ straight or branched chain alkyl, C₃-7 cycloalkyl, benzyl, or aryl;
(ii) COOR₆, wherein R₆ is selected from H, optionally substituted C₁₋₈ straight or branched chain alkyl, optionally substituted aryl and optionally substituted arylalkyl; wherein the substituents on the alkyl, aryl and arylalkyl group are selected from C₁₋₈ alkoxy, phenylacetyloxy, hydroxy, halogen, p-tosyloxy, mesyloxy, amino, cyano, carboalkoxy, or NR₂₀R₂₁ wherein R₂₀ and R₂₁ are independently selected from the group consisting of hydrogen, C₁₋₈ straight or branched chain alkyl, C₃-7 cycloalkyl, benzyl, or aryl;
(iii) cyano;
(iv) a lactone or lactam formed with R₄;
(v) CONR₇R wherein R₇ and R₈ are independently selected from H, C₁₋₈ straight or branched chain alkyl, C₃-7 cycloalkyl, trifluoromethyl, hydroxy, alkoxy, acyl, alkylcarbonyl, carboxyl, arylalkyl, aryl, heteroaryl, and heterocyclyl; wherein the alkyl, cycloalkyl, alkoxy, acyl, alkylcarbonyl, carboxyl, arylalkyl, aryl, heteroaryl, and heterocyclyl groups may be substituted with carboxyl, alkyl, aryl, substituted aryl, heterocyclyl, substituted heterocyclyl, heteroaryl, substituted heteroaryl, hydroxamic acid, sulfonamide, sulfonyl, hydroxy, thiol, alkoxy, or arylalkyl;
or R₇ and R₈ taken together with the nitrogen to which they are attached form a heterocyclyl or heteroaryl group;
(vi) a carboxylic ester or carboxylic acid bioisostere including optionally substituted heteroaryl groups;
(b) R₂ is selected from the group consisting of optionally substituted alkyl, optionally substituted aryl, optionally substituted heteroaryl, optionally substituted C₃₋₇ cycloalkyl, optionally substituted heterocyclyl, wherein the heterocyclyl is 1,3-dioxolane or furan, or R₂ is;

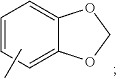

(c) R₃ is from one to four groups independently selected from the group consisting of:
(i) hydrogen, halo, C₁₋₈ straight or branched chain alkyl, arylalkyl, C₃₋₇ cycloalkyl, C₁₋₈ alkoxy, cyano, C₁₋₄ carboalkoxy, trifluoromethyl, C₁₋₈ alkylsulfonyl, halogen, nitro, hydroxy, trifluoromethoxy, C₁₋₈ carboxylate, aryl, heteroaryl, and heterocyclyl;
(ii) NR₁₀R₁₁ wherein R₁₀ and R₁₁ are independently selected from H, C₁₋₈ straight or branched chain alkyl, arylalkyl, C₃₋₇ cycloalkyl, carboxyalkyl, aryl, heteroaryl, or heterocyclyl, or R₁₀ and R₁₁ taken together with the nitrogen to which they are attached form a heterocyclyl or heteroaryl group;
(iii) NR₁₂COR₁₃ wherein R₁₂ is selected from hydrogen or alkyl and R₁₃ is selected from hydrogen, alkyl, substituted alkyl, C₁₋₃ alkoxyl, carboxyalkyl, R₃₀R₃₁N(CH₂)ₚ, R₃₀R₃₁NCO(CH₂)ₚ, aryl, arylalkyl, heteroaryl, or heterocyclyl, or R₁₂ and R₁₃ taken together with the carbonyl group form a carbonyl containing heterocyclyl group, wherein R₃₀ and R₃₁ are independently selected from H, OH, alkyl, and alkoxy, and p is an integer from 1-6, wherein the alkyl group may be substituted with carboxyl, alkyl, aryl, substituted aryl, heterocyclyl, substituted heterocyclyl, heteroaryl, substituted heteroaryl, hydroxamic acid, sulfonamide, sulfonyl, hydroxy, thiol, alkoxy, or arylalkyl;
(d) R₄ is selected from the group consisting of
(i) hydrogen,
(ii) C₁₋₃ straight or branched chain alkyl,
(iii) benzyl, and (iv) NR₁₃R₁₄, wherein R₁₃ and R₁₄ are independently selected from hydrogen and C₁₋₆ alkyl; wherein the C₁₋₃ alkyl and benzyl groups are optionally substituted with one or more groups selected from C₃₋₇ cycloalkyl, C₁₋₈ alkoxy, cyano, C₁₋₄ carboalkoxy, trifluoromethyl, C₁₋₈ alkylsulfonyl, halogen, nitro, hydroxy, trifluoromethoxy, C₁₋₈ carboxylate, amino, NR₁₃R₁₄, aryl, and heteroaryl; and
(e) X is selected from S and O;
and the pharmaceutically acceptable salts, esters and pro-drug forms thereof.

In an alternative embodiment, R₁, R₃, and R₄ are as above and R₂ is NR₁₅R₁₆, where R₁₅ and R₁₆ are independently selected from hydrogen, C₁₋₈ straight or branched chain alkyl, arylalkyl, C₃₋₇ cycloalkyl, aryl, heteroaryl, and heterocyclyl, or R₁₅ and R₁₆ taken together with the nitrogen to which they are attached form a heterocyclyl or heteroaryl group.

In regard to the above compounds, “alkyl” refers to straight, cyclic and branched-chain alkyl. The alkyl group may be optionally substituted with one or more groups such as halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, carboxamide, hydroxamic acid, sulfonamide, sulfonyl, thiol, aryl, aryl(c₁-c₈)alkyl, heterocyclyl, and heteroaryl. The term “bioisostere” is defined as “groups or molecules which have chemical and physical properties producing broadly similar biological properties.” (Burger's Medicinal Chemistry and Drug Discovery, M. E. Wolff, ed. Fifth Edition, Vol. 1, 1995, Pg. 785). The term “acyl” as used herein, whether used alone or as part of a substituent group, means an organic radical having 2 to 6 carbon atoms (branched or straight chain) derived from an organic acid by removal of the hydroxyl group. “Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-)halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. The term “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. The terms “heterocycle,” “heterocyclic,” and “heterocycle” refer to an optionally substituted, fully or partially saturated cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom.

In other embodiments, PDE7 inhibitors useful in the methods of the invention have the formulas:

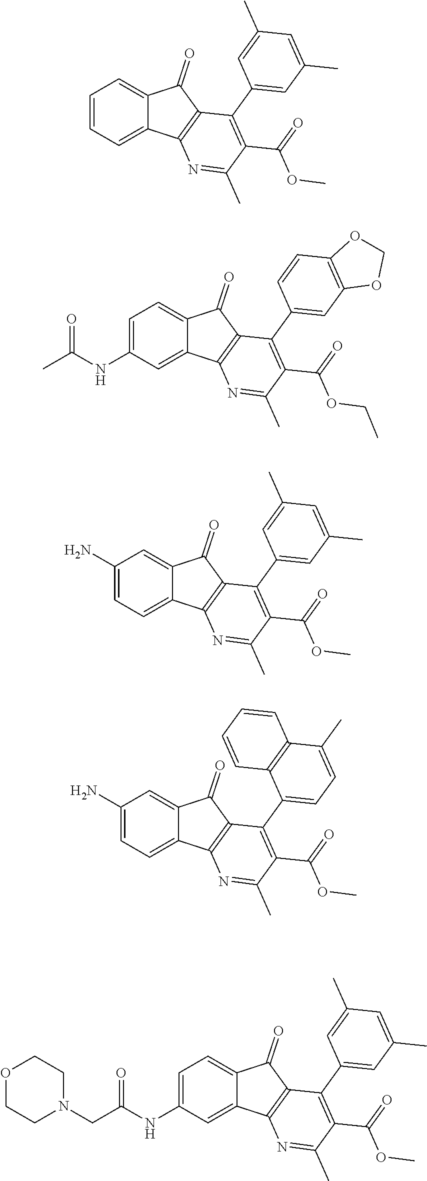

The preparation of the above compounds is described in U.S. Pat. No. 6,903,109, US 2004/0082578, WO 2003/088963, and US 2006/0154949.

In another embodiment, PDE7 inhibitors useful in the methods of the invention are selected from those compounds generally or specifically disclosed in U.S. Pat. No. 6,958,328, WO 2002/085894, and US 2003/0212089, each expressly incorporated herein by reference in its entirety. These PDE7 inhibitors have the same formula as those described above (e.g., U.S. Pat. No. 6,903,109), except that R₁ is not a carboxylic ester or carboxylic acid bioisostere. The preparation of these compounds is described in U.S. Pat. No. 6,958,328, US 2003/0212089, and WO 2002/085894.

In another embodiment, PDE7 inhibitors useful in the methods of the invention are selected from those compounds generally or specifically disclosed in WO 2006/004040, U.S. Pat. No. 7,932,250, and EP-A-1775298, each expressly incorporated herein by reference in its entirety.

In one embodiment, PDE7 inhibitors useful in the methods of the invention have the formula (6):

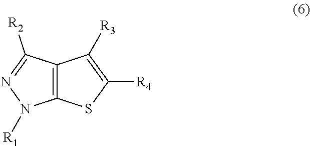

((6): Also referred to as thienopyrazole derivatives.)

The substituents for the above compound (6) are defined as follows:
R₁ is substituted or unsubstituted C₃₋₈ alkyl group, substituted or unsubstituted cycloalkyl group, or substituted or unsubstituted heterocycloalkyl group (e.g., cyclohexyl, cycloheptyl, or tetrahydropyranyl);
R₂ is a hydrogen atom or substituted or unsubstituted C₁₋₃ alkyl group (e.g., methyl);
R₃ is a hydrogen atom, substituted or unsubstituted C₁₋₃ alkyl group, or a halogen atom; and
R₄ is substituted or unsubstituted aryl group, substituted or unsubstituted heteroaryl group, or a group CONR₅R₆, or CO₂R₇,
wherein R₅ and R₆ are, same or different from each other, a hydrogen atom; C₁₋₆ alkyl group which may be substituted by a halogen atom, substituted or unsubstituted aryl group, substituted or unsubstituted heteroaryl group, substituted or unsubstituted heterocycloalkyl group, substituted or unsubstituted cycloalkyl group, a group NR₇COR₈, COR₈, NR₉R₁₀; substituted or unsubstituted cycloalkyl group; substituted or unsubstituted heterocycloalkyl group; substituted or unsubstituted aryl group; substituted or unsubstituted heteroaryl group; or substituted or unsubstituted heterocycloalkyl group in which the ring is formed together with the nitrogen atom binding R₅ and R₆;
wherein R₇ is a hydrogen atom or substituted or unsubstituted C₁₋₃ alkyl group;
wherein R₈ is substituted or unsubstituted heterocycloalkyl group, or a group OH, OR₇, or NR₉R₁₀;
wherein R₉ and R₁₀ are, same or different from each other, a hydrogen atom; substituted or unsubstituted C₁₋₃ alkyl group, substituted or unsubstituted heterocycloalkyl group; substituted or unsubstituted acyl; a group SO₂R₇, or substituted or unsubstituted heterocycloalkyl group in which the ring is formed together with the nitrogen atom binding R₅ and R₆;
or pharmaceutically acceptable salts or solvates thereof.

In regard to the above compounds, the term “cycloalkyl group” means cycloalkyl group having 3 to 8 carbon atoms. The term “heterocycloalkyl group” may be 3 to 7 membered monocyclic or polycyclic heterocyclic group containing the same or different 1 to 4 hetero atom(s) such as oxygen, nitrogen or sulfur atom(s), and examples may include piperidinyl, pyrrolidinyl, piperazinyl, tetrahydrofuryl, tetrahydropyranyl, morpholinyl, azetidinyl, imidazolidinyl, oxazolidinyl, hexahydropyrrolidinyl, octahydroindolidinyl, octahydroquinolidinyl, octahydroindolyl, and oxo-derivatives thereof. The term “aryl group” may be aromatic hydrocarbon group, which consists of mono-benzene ring, or binding or condensed benzene ring, such as phenyl, naphthyl, biphenyl and the like; and dicyclic or tricyclic group, which consists of benzene ring condensed with cycloalkyl or heterocyclic ring, such as 1,2,3,4-tetrahydronaphthalene, 2,3-dihydroindene, indoline, coumarone and the like. The term “heteroaryl group” may be 5 to 7 membered monocyclic heteroaryl group or polycyclic heteroaryl group, and having 2 to 8 carbon atoms with 1 to 4 hetero atom(s) such as oxygen, nitrogen, sulfur atom(s), in which the polycyclic heteroaryl group has condensed ring system by the same or different monocyclic heteroaryl or benzene ring each other; or polycyclic group which is consisted of heteroaryl group condensed with cycloalkyl or heterocycloalkyl ring. Examples of suitable substituent of the present invention may include straight, branched-chained or cyclic C₁-C₈ alkyl group, which may be substituted by one or more methyl, ethyl, propyl, isopropyl, n-butyl, t-butyl, cyclohexyl, cycloheptyl, methoxymethyl, hydroxymethyl, trifluoromethyl, C₁-C₃ alkoxy group, halogen atom, and hydroxyl group; hydroxyl group; cyano group; substituted or unsubstituted alkoxy group such as methoxy, ethoxy group; amino group which may be substituted by C₁-C₆ alkyl group or acyl group such as amino, methylamino, ethylamino, dimethylamino, acylamino and the like; carboxylic group; substituted or unsubstituted ester group; phosphate group; sulfonic group; substituted or unsubstituted aryl group; substituted or unsubstituted heteroaryl group; saturated or unsaturated heterocycloalkyl group which may be substituted; substituted or unsubstituted carbamoyl group; substituted or unsubstituted amide group; substituted or unsubstituted thioamide group; halogen atom; nitro group; substituted or unsubstituted sulfone group; substituted or unsubstituted sulfonylamide group; oxo group; substituted or unsubstituted urea group; straight, branched-chained or cyclic alkenyl group such as ethenyl, propenyl, cyclohexenyl and the like.

In other embodiments, PDE7 inhibitors useful in the methods of the invention have the formulas 6A, 6B, 6C, 6D, 6E, 6F, 6G and 6H:

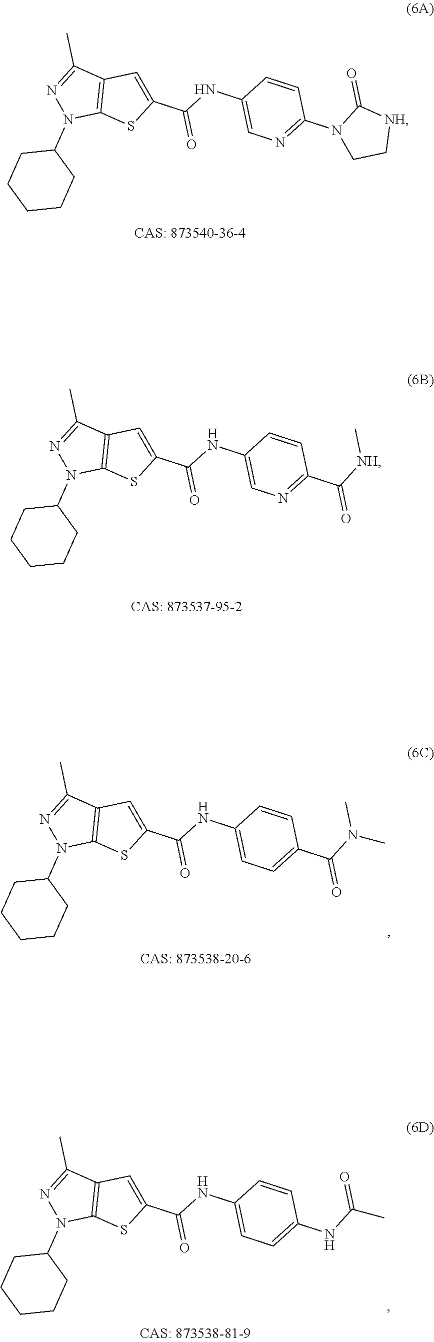 [SMILES: Cc1nn(C2CCCCC2)c2sc(C(=O)Nc3ccc(N4CCNC4=O)nc3)cc12 ; CNC(=O)c1ccc(NC(=O)c2cc3c(C)nn(C4CCCCC4)c3s2)cn1 ; Cc1nn(C2CCCCC2)c2sc(C(=O)Nc3ccc(C(=O)N(C)C)cc3)cc12 ; CC(=O)Nc1ccc(NC(=O)c2cc3c(C)nn(C4CCCCC4)c3s2)cc1]

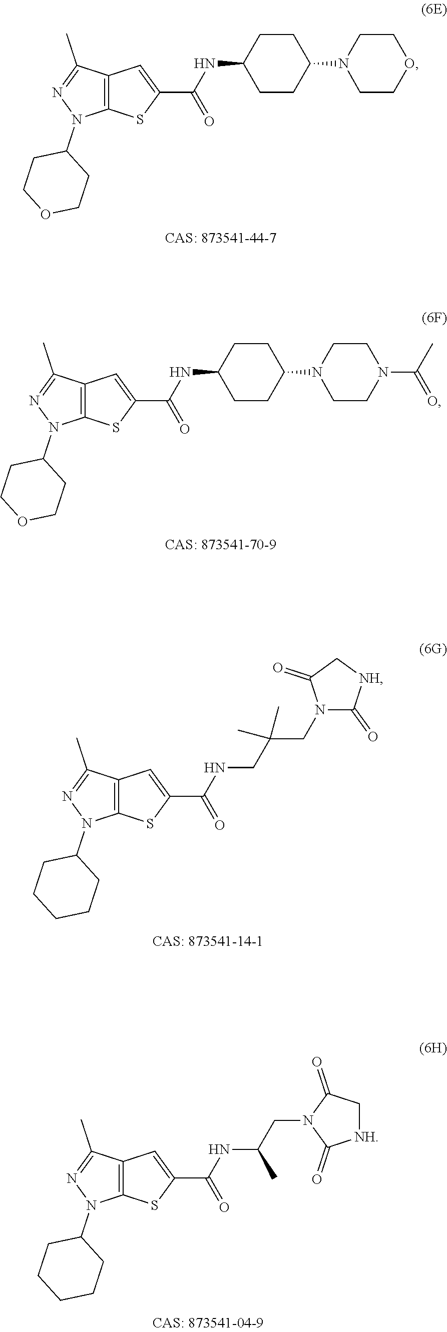 [SMILES: Cc1nn(C2CCOCC2)c2sc(C(=O)N[C@H]3CC[C@H](N4CCOCC4)CC3)cc12 ; CC(=O)N1CCN([C@H]2CC[C@H](NC(=O)c3cc4c(C)nn(C5CCOCC5)c4s3)CC2)CC1 ; Cc1nn(C2CCCCC2)c2sc(C(=O)NCC(C)(C)CN3C(=O)CNC3=O)cc12 ; Cc1nn(C2CCCCC2)c2sc(C(=O)N[C@H](C)CN3C(=O)CNC3=O)cc12]

(6A, 6B, 6C, 6D, 6E, 6F, 6G, 6H): Also referred to as 3-methyl-1H-thieno[2,3-c]pyrazole-5-carboxamide derivatives.)
(6E, 6F): Also referred to as 3-methyl-1-(tetrahydro-2H-pyran-4-yl)-1H-thieno[2,3-c]pyrazole-5-carboxamide derivatives.)

The preparation of the above compounds 6A to 6H is described in EP-A-1775298, U.S. Pat. No. 7,932,250, and WO 2006/004040 and EP-A-2433943, and U.S. Pat. No. 8,901,315.

The formulas 6, 6A, 6B, 6C, 6D, 6E, 6F, 6G, and 6H are also referred to in U.S. Pat. No. 11,207,275 and WO 2012/064667, U.S. Pat. No. 9,220,715, US 2012/0115849, WO 2013/176877, and US 2022/0062194. In these descriptions, especially in U.S. Pat. No. 11,207,275, reference is also made to formula 6 and OMS182399 (OMS527 respectively). Reference is also made in JP 6082058, KR 101741853, AU 2010245250, CA 2760786, CN115192711, and RU 2600869 ((6E) and (6F)).

The formulas 6, 6A, 6B, 6C, 6D, 6E, 6F, 6G, and 6H are also referred to in U.S. Pat. Nos. 9,119,822, 8,637,528 and WO 2010/129036, U.S. Pat. No. 9,119,822, and US 2019/0183824 and WO 2008/119057.

In another embodiment, PDE7 inhibitors useful in the methods of the invention are selected from those compounds generally or specifically disclosed in WO 2004/111053 and US 2006/0128707, each expressly incorporated herein by reference in its entirety.

In one embodiment, PDE7 inhibitors useful in the methods of the invention have the formulas (7A) and (7B):

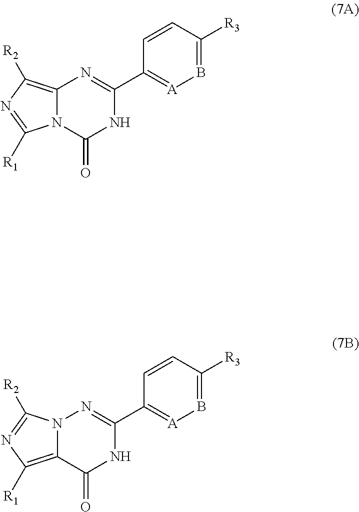

((7A), (7B): Also referred to as imidazotriazinone derivatives.)

The substituents for the above compounds 7A and 7B are defined as follows:
A is N or CR₄;
B is N or CH;
R₁ is substituted or unsubstituted C₃₋₈ cycloalkyl group or tert-butyl group;
R₂ is a hydrogen atom or C₁₋₆ alkyl group;
R₃ is a hydrogen atom; nitro group; cyano group; a halogen atom; heteroaryl group; substituted or unsubstituted C₁₋₆ alkyl group; substituted or unsubstituted C₂₋₆ alkenyl group; saturated or unsaturated heterocycloalkyl group which is substituted or unsubstituted; a group: NR₅R₆, C(O)R₇, SO₂R₇, OR₈, NR₈COR₇, NR₈SO₂R₇;
R₄ is a hydrogen atom or C₁₋₃ alkoxy group which is unsubstituted or substituted by one or more fluorine atom(s);
R₅ and R₆ are, same or different from each other, a hydrogen atom; substituted or unsubstituted C₁₋₆ alkyl group; substituted or unsubstituted acyl group; or substituted or unsubstituted heterocycloalkyl group;
R₇ is a hydrogen atom; substituted or unsubstituted C₁₋₆ alkyl group; substituted or unsubstituted heterocycloalkyl group; OH; OR₈ or NR₅R₆;
R₈ is a hydrogen atom, substituted or unsubstituted C₁₋₆ alkyl group; or substituted or unsubstituted heterocycloalkyl group;
or pharmaceutically acceptable salts or solvates thereof.

In regard to the above compounds, the term “C₁-C₆ alkyl group” refers to a straight or branched-chained alkyl group having 1 to 6 carbon atoms, and the term “C₂-C₆ alkenyl group” refers to a straight or branched-chained alkenyl group having 2 to 6 carbon atoms. The term “cycloalkyl group” refers to a cycloalkyl group having 3 to 8 carbon atoms such as cyclopropyl, cyclobutyl, cyclopentyl, cyclohexyl, cycloheptyl, and cyclooctyl. The term “heterocycloalkyl group” is 3 to 7 membered heterocyclic group containing the same or different 1 to 4 hetero atom(s) such as oxygen, nitrogen or sulfur atom(s), and examples may include piperidinyl, pyrrolidinyl, piperazinyl, tetrahydrofuryl, tetrahydropyranyl, morpholinyl, azetidinyl, and homopiperazinyl. The term “heteroaryl group” is 5 to 7 membered monocyclic or polycyclic group thereof containing 2 to 8 carbon atoms and the same or different 1 to 4 hetero atom(s) such as oxygen, nitrogen or sulfur atom(s). The examples include pyrrole, furyl, thienyl, imidazolyl, thiazolyl, pyrazinyl, indolyl, quinolinyl, isoquinolinyl, tetrazolyl, pyridinyl, pyrazolyl, pyridazinyl, and pyrimidinyl. The “halogen atom” includes fluorine, chlorine, bromine and iodine. Examples of the suitable substituent of “substituted or unsubstituted C₁-C₆ alkyl group”, “substituted or unsubstituted C₃-C₈ cycloalkyl group”, “substituted or unsubstituted alkenyl group”, “substituted or unsubstituted heterocycloalkyl group” and “substituted or unsubstituted acyl group” include a straight or branched-chained, or substituted or unsubstituted alkyl group such as methyl, ethyl, propyl, isopropyl, n-butyl, tert-butyl, substituted or unsubstituted cycloalkyl group such as cyclopropyl, cyclobutyl, cyclopentyl, cyclohexyl, and cycloheptyl; hydroxyl group; cyano group; alkoxy group such as methoxy and ethoxy; substituted or unsubstituted amino group such as amino, methylamino, ethylamino, and dimethylamino; substituted or unsubstituted acyl group such as acetyl, and propionyl; substituted or unsubstituted aryl group; substituted or unsubstituted heteroaryl group; saturated or unsaturated heterocycloalkyl group which is substituted or unsubstituted; substituted or unsubstituted carbamoyl group; substituted or unsubstituted amide group; halogen atom; nitro group; substituted or unsubstituted sulfone group; oxo group; urea group; a straight or branched-chained, or cyclic alkenyl group which is substituted or unsubstituted such as ethenyl, propenyl, and cyclohexenyl.

In other embodiments, PDE7 inhibitors useful in the methods of the invention have the formulas:

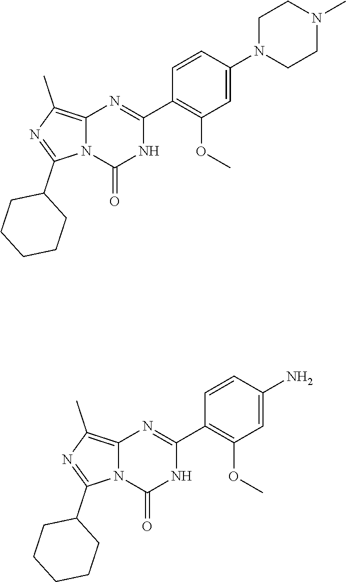 [SMILES: COc1cc(N2CCN(C)CC2)ccc1-c1nc2c(C)nc(C3CCCCC3)n2c(=O)[nH]1 ; COc1cc(N)ccc1-c1nc2c(C)nc(C3CCCCC3)n2c(=O)[nH]1]

The preparation of the above compounds is described in US 2006/0128707 and WO 2004/111053.

In another embodiment, PDE7 inhibitors useful in the methods of the invention are selected from those compounds generally or specifically disclosed in U.S. Pat. No. 6,617,357, US 2002/0156064, and Molecular Pharmacology 66:1679-1689, 2004, each expressly incorporated herein by reference in its entirety.

In one embodiment, PDE7 inhibitors useful in the methods of the invention have the formula (8):

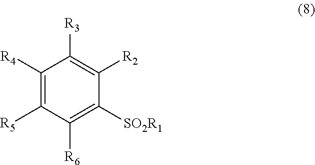

((8): Also referred to as benzenesulfonamide derivatives.)

The substituents for the above formula (8) are defined as follows:
R₁ is NRₐR_{b} where Rₐ and R_{b} are independently H or C₁₋₆ alkyl, or represents a 5 to 7 member ring comprised of carbon or carbon and one or more additional heteroatoms selected from O, N, or S;
R₂ is H, C₁₋₈ alkyl, C₁₋₃ alkyl-Ar, C₁₋₃ alkyl-C₃₋₆ cycloalkyl, C₂₋₈ alkenyl, C₂₋₄ alkenyl-Ar, or C₂₋₄ alkenyl-C₃₋₆ cycloalkyl, wherein Ar is substituted or unsubstituted phenyl;
R₃ is NO₂, halo, CN, C(O)OR₇, COR₁, or NRₐR_{b} where Rₐ and R_{b} are independently H or C₁₋₆ alkyl;
R₄ is H, OC₁₋₆ alkyl, halo, C(O)NRₐR_{b}, C(O)OR₇, C₁₋₈ alkyl, OCHF₂, CH₂OR₈, OC₁₋₃ alkyl-Ar, or CH₂NHC(O) CH₃;
R₅ is H, halo, or alkyl;
R₆ is C₁₋₈ alkyl, OC₁₋₄ alkyl, or halo;
R₇ is hydrogen or an ester or amide-forming group;
R₈ is hydrogen or C₁₋₆ alkyl;
or a pharmaceutically acceptable salt or solvate thereof.

In one embodiment, a PDE7 inhibitor useful in the methods of the invention has the formula 8A:

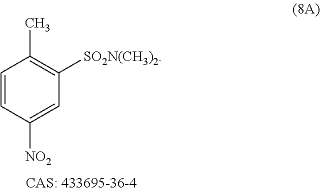

The preparation of the above compounds 8A is described in U.S. Pat. No. 6,617,357, US 2002/0156064, and Molecular Pharmacology 66:1679-1689, 2004.

In another embodiment, PDE7 inhibitors useful in the methods of the invention are selected from those compounds generally or specifically disclosed in U.S. Pat. No. 6,852,720, EP-A-1348433, and WO 2003/082277, each expressly incorporated herein by reference in its entirety.

In one embodiment, PDE7 inhibitors useful in the methods of the invention have the formula (9):

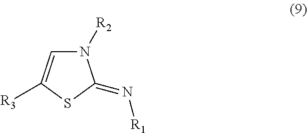

((9): Also referred to as thiazol-2-yl-imine derivatives.)

The substituents for the above formula (9) are defined as follows:
R₁ is a group selected from cycloalkyl, heterocycloalkyl, aryl and heteroaryl, those groups being optionally substituted by one or more groups, identical or different, selected independently of each other from halogen, trifluoromethyl, nitro, cyano, oxo, NR₄R₅, CO₂R₄, CONR₄R₅, OR₄, S(O)ₙR₄, S(O)ₙNR₄R₅, tetrazolyl and (C₁-C₆) alkyl which is optionally substituted by 1 to 3 groups, identical or different, selected independently of each other from OR₄, NR₄R₅, and CO₂R₄; wherein n is an integer from 0 to 2 inclusive, R₄ and R₅ are identical or different and independently of each other are a hydrogen atom or a group of formula X₁—Rₐ, wherein X₁ is a single bond or a (C₁-C₆) alkylene group, and Rₐ is a group selected from (C₁-C₆) alkyl, cycloalkyl, heterocycloalkyl, aryl, and heteroaryl,
R₂ is a group selected from (C₁-C₆) alkyl, (C₂-C₆) alkenyl, (C₂-C₆) alkynyl, aryl, and cycloalkyl,
R₃ is a group selected from cycloalkyl, heterocycloalkyl, aryl and heteroaryl, these groups being optionally substituted by one or more groups, identical or different, selected independently of each other from halogen, nitro, cyano, trifluoromethyl, oxo, (C₁-C₆) alkyl, OR₆, NR₆R₇, COR₆, CO₂R₆, CONHOH, CONR₆R₇, S(O)ₘR₆, S(O)ₘNR₆R₇, NR₆COR₇, NR₆SO₂R₇, N(SO₂R₇)₂, NR₆CONR₇R₈, C(═NCN)NR₆R₇, NR₈C(═NCN)NR₆R₇, and tetrazolyl optionally substituted with a (C₁-C₄) alkyl, wherein m is an integer from 0 to 2 inclusive, R₆ and R₇ are identical or different and independently of each other are a hydrogen atom or a group of formula X₂R_{b}, wherein X₂ is a single bond or a (C₁-C₆) alkylene group, R_{b} is a group selected from (C₁-C₆) alkyl, cycloalkyl, heterocycloalkyl, aryl and heteroaryl, these groups being optionally substituted by 1 to 3 groups, identical or different, selected independently of each other from hydroxy, (C₁-C₆)alkoxy, (C₁-C₆) alkyl, amino, mono(C₁-C₆) alkylamino, di(C₁-C₆) alkylamino (each alkyl amino being identical or different, independently of each other), carboxy, (C₁-C₆)alkoxycarbonyl, and benzyl, and R₈ represents a hydrogen atom or a (C₁-C₆) alkyl group;
a racemic form thereof, an isomer thereof, an N-oxide thereof, or a pharmaceutically acceptable acid or base salt thereof.

The preparation of the above compounds is described in U.S. Pat. No. 6,852,720, EP-A-1348433, and WO 2003/082277.

In another embodiment, PDE7 inhibitors useful in the methods of the invention are selected from those compounds generally or specifically disclosed in U.S. Pat. No. 6,753,340, US 2003/0191167, EP-A-1348701, and WO 2003/082839, each expressly incorporated herein by reference in its entirety.

In one embodiment, PDE7 inhibitors useful in the methods of the invention have the formula (10):

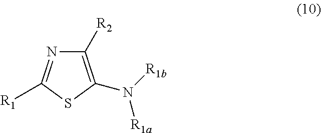

((10): Also referred to as (4,2-disubstituted-thiazol-5-yl)amine derivatives.)

The substituents for the above formula (10) are defined as follows:
R₁ₐ is a group selected from hydrogen, (C₁-C₆)alkyl and aryl(C₁-C₆) alkyl, R_{1b} is a group selected from cycloalkyl, heterocycloalkyl, aryl and heteroaryl, those groups being optionally substituted by one or more groups, identical or different, selected independently of each other from halogen, trifluoromethyl, nitro, cyano, oxo, NR₄R₅, CO₂R₄, CONR₄R₅, OR₄, S(O)ₙR₄, S(O)ₙNR₄R₅, tetrazolyl, and (C₁-C₆) alkyl which is optionally substituted by 1 to 3 groups, identical or different, selected independently of each other from OR₄, NR₄R₅, and CO₂R₄, wherein n is an integer from 0 to 2 inclusive, R₄ and R₅ are identical or different and independently of each other are a hydrogen atom or a group of formula X₁—Rₐ, wherein X₁ is a single bond or a (C₁-C₆) alkylene group, and Rₐ is a group selected from (C₁-C₆) alkyl, cycloalkyl, heterocycloalkyl, aryl and heteroaryl,
R₂ is a group selected from (C₁-C₆) alkyl, (C₂-C₆) alkenyl, (C₂-C₆) alkynyl, aryl and cycloalkyl,
R₃ is a group selected from cycloalkyl, heterocycloalkyl, aryl and heteroaryl, these groups being optionally substituted by one or more groups, identical or different, selected independently of each other from halogen, nitro, cyano, trifluoromethyl, oxo, (C₁-C₆) alkyl, OR₆, NR₆R₇, COR₆, CO₂R₆, CONHOH, CONR₆R₇, S(O)ₘR₆, S(O)ₘNR₆R₇, NR₆COR₇, NR₆SO₂R₇, N(SO₂R₇)₂, NR₆CONR₇R₈, C(═N—CN)NR₆R₇, NR₈C(═N—CN)NR₆R₇, and tetrazolyl optionally substituted with a (C₁-C₄) alkyl, wherein m is an integer from 0 to 2 inclusive, R₆ and R₇ are identical or different and independently of each other are a hydrogen atom or a group of formula X₂—R_{b}, wherein X₂ is a single bond or a (C₁-C₆) alkylene group, R_{b} is a group selected from (C₁-C₆) alkyl, cycloalkyl, heterocycloalkyl, aryl and heteroaryl, these groups being optionally substituted by 1 to 3 groups, identical or different, selected independently of each other from hydroxy, (C₁-C₆)alkoxy, (C₁-C₆) alkyl, amino, mono(C₁-C₆) alkylamino, di(C₁-C₆) alkylamino (each alkyl amino being identical or different, independently of each other), carboxy, (C₁-C₆)alkoxycarbonyl, and benzyl, and R₈ is a hydrogen atom or a (C₁-C₆) alkyl group, or a racemic form thereof, an isomer thereof, an N-oxide thereof or a pharmaceutically acceptable acid or base salt thereof.

The preparation of these compounds is described in U.S. Pat. No. 6,753,340, US 2003/0191167, EP-A-1348701, and WO 2003/082839.

In another embodiment, PDE7 inhibitors useful in the methods of the invention are selected from those compounds generally or specifically disclosed in U.S. Pat. No. 6,849,638, US20030119829, and WO 2002/088138, each expressly incorporated herein by reference in its entirety.

In one embodiment, PDE7 inhibitors useful in the methods of the invention have the formula (11):

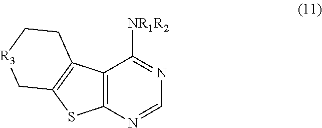

((11): Also referred to as amine-substituted thiopheno[2,3-d]pyrimidine derivatives.)

The substituents for the above formula (11) are defined as follows:
R₁ and R₂ are independently selected from the group consisting of hydrogen, alkyl of 1-8 carbon atoms, alkenyl of 2-8 carbon atoms, alkynyl of 2-8 carbon atoms, cycloalkyl of 3-7 carbon atoms, fully saturated heterocycle of 2-6 carbon atoms and 1-2 heteroatoms selected from NH, S and O, aryl of 6-12 carbon atoms, that may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1, 2 heteroatoms selected from N, S, and O, heteroaryl of 4-11 carbon atoms and 1-2 heteroatoms selected from N, S and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S and O, and R₄-R₅, or R₁ and R₂ combine to form, together with the nitrogen atom to which they are attached, a 5-7 membered saturated ring which may contain 1-2 additional heteroatoms selected from the group consisting of NH, NR₈, S and O, or combine to form, together with the nitrogen atom to which they are attached, a 5-7 membered unsaturated ring that may contain 1-2 additional heteroatoms selected from the group consisting of N, S and O,
wherein said saturated or unsaturated ring may be substituted with 1-2 substituents selected from the group consisting of OH, alkyl of 1-6 carbon atoms, alkenyl of 2-6 carbon atoms, alkynyl of 2-6 carbon atoms, cycloalkyl of 3-7 carbon atoms, fully saturated heterocycle of 2-6 carbon atoms and 1-2 heteroatoms selected from NH, S, and O, halogen, haloalkyl of 1-2 carbon atoms and a number of halogen atoms up to the perhalo level, alkoxy of 1-6 carbon atoms, haloalkoxy of 1-6 carbon atoms and a number of halogen atoms up to the perhalo level, and R₉-R₁₀; or
R₁ and R₂ combine to form, together with the nitrogen atom to which they are attached, an 8-10 membered bicyclic saturated ring;
R₃ is selected from the group consisting of NH, S, S(═O)₂, and O;
R₄ is selected from alkyl of 1-8 carbon atoms, alkenyl of 2-8 carbon atoms, alkynyl of 2-8 carbon atoms, C(═C), S(═O)₂, and C(═O)O;
R₅ is selected from hydrogen, OH, alkyl of 1-8 carbon atoms, alkenyl of 2-8 carbon atom, alkynyl of 2-8 carbon atoms, alkoxy of 1-8 carbon atoms, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkyl of 1-6 carbon atoms and a number of halogen atoms up to the perhalo level, aryl of 6-12 carbon atoms and heteroaryl of 4-11 carbon atoms and 1-2 heteroatoms selected from N, S, and O, heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms and heteroaryl of 4-11 carbon atoms and 1-2 heteroatoms selected from N, S, and O, cycloalkyl of 3-7 carbon atoms, fully saturated heterocycle of 2-6 carbon atoms and 1-2 heteroatoms selected from NH, S and O, and NR₆R₇,
R₆ and R₇ are independently selected from hydrogen, alkyl of 1-8 carbon atoms, alkenyl of 2-8 carbon atoms, and alkynyl of 2-8 carbon atoms, or R₆ and R₇ combine together with the nitrogen atom to which they are attached to form a 5-7 membered, unsaturated ring which may contain 1-2 additional heteroatoms selected from N, S and O or to form a 5-7 membered, saturated ring which may contain 1-2 additional heteroatoms selected from NH, S, and O;
R₈ is selected from alkyl of 1-8 carbon atoms, alkenyl of 2-8 carbon atoms, alkynyl of 2-8 carbon atoms, R₁₁-R₁₂, cycloalkyl of 3-7 carbon atoms, fully saturated heterocycle of 2-6 carbon atoms and 1-2 heteroatoms selected from NH, S, and O, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O, heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O;
R₉ is selected from alkyl of 1-8 carbon atoms, alkenyl of 2-8 carbon atoms, and alkynyl of 2-8 carbon atoms,
R₁₀ is selected from OH, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O, and heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O;
R₁₁ is selected from alkyl of 1-8 carbon atoms, alkenyl of 2-8 carbon atoms, and alkynyl of 2-8 carbon atoms; and
R₁₂ is selected from cycloalkyl of 3-7 carbon atoms, fully saturated heterocycle of 2-6 carbon atoms and 1-2 heteroatoms selected from NH, S, and O, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O, and heteroaryl of 4-11 carbon atoms and 1-2 heteroatoms selected from N, S and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S and O;
and pharmaceutically acceptable salts thereof.

The preparation of these compounds is described in U.S. Pat. No. 6,849,638, US 2003/0119829, and WO 2002/088138.

In another embodiment, PDE7 inhibitors useful in the methods of the invention are selected from those compounds generally or specifically disclosed in US2005222138 and WO 2003/064389, each expressly incorporated herein by reference in its entirety.

In one embodiment, PDE7 inhibitors useful in the methods of the invention have the formula (12):

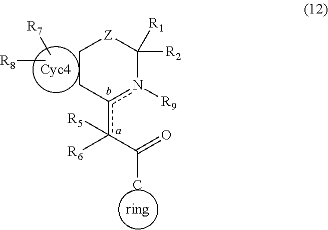

((12): Also referred to as nitrogen-containing bicyclic compound derivatives.)

The substituents for the above formula (12) are defined as follows:
R₁ and R₂ are each independently, (1) hydrogen atom, or (2) C₁₋₈ alkyl, or
R₁ and R₂ may be taken together with the carbon atom to which they are attached to form Cyc1, wherein R₁ and R₂ do not represent hydrogen atom at the same time;
Z is (1) CR₃R₄, (2) O, (3) S, or (4) a bond;
R₃ and R₄ are each independently, (1) hydrogen atom, (2) C₁₋₈ alkyl, (3) C₁₋₈ alkoxy, or (4) hydroxy, or
R₃ and R₄ may be taken together with the carbon atom to which they are attached to form Cyc1 or C(O);
R₅ and R₆ are each independently, (1) hydrogen atom, or (2) C₁₋₈ alkyl, or
R₅ and R₆ may be taken together with the carbon atom to which they are attached to form Cyc1;
Cyc1, which is represented by R₁ and R₂, R₃ and R₄, R₅ and R₆ is, each independently, (1) C₃₋₁₀ cycloalkyl, or (2) 3-10 membered monocyclic hetero-ring comprising 1-2 of heteroatom selected from oxygen, nitrogen and sulfur, and Cyc1 may be substituted with R₁₀;
R₁₀ is (1) C₁₋₈ alkyl, (2) C₁₋₈ alkoxy, (3) hydroxy, (4) COOR₁₁, (5) oxo, (6) SO₂R₁₂, or (7) COR₁₃;
R₁₁ is hydrogen atom, or C₁₋₈ alkyl;
R₁₂ and R₁₃ are (1) C₁₋₈ alkyl, or (2) phenyl which may be substituted with C₁₋₈ alkyl;
R₇ and R₈ are each independently, (1) hydrogen atom, (2) C₁₋₈ alkyl, (3) C₁₋₈ alkoxy, (4) hydroxy, (5) cyano, (6) halogen atom, (7) COOR₁₄, (8) CONR₁₅R₁₆, (9) Cyc2, (10) C₂₋₈ alkenyl, (11) C₂₋₈ alkynyl, (12) NR₅₁R₅₂, (13) nitro, (14) formyl, (15) C₂₋₈ acyl, (16) C₁₋₈ alkyl substituted with hydroxy, C₁₋₈ alkoxy, Cyc2, NR₅₁R₅₂, or NR₅₃-Cyc2, (17) NR₅₄COR₅₅, (18) NR₅₆SO₂R₅₇, (19) SO₂NR₅₈R₅₉, (20) C₂₋₈ alkenyl substituted with COOR₁₄, (21) CH═N—OH, (22) C₁₋₈ alkylene-NR₆₀—(C₁₋₈ alkylene)-R₆₁, (23) C₁₋₈ alkylthio, (24) C₁₋₈ alkyl substituted with 1-3 of halogen atom, (25) C₁₋₈ alkoxy substituted with 1-3 of halogen atom, (26) C₁₋₈ alkoxy substituted with Cyc2, (27) 0-Cyc2, (28) OSO₂R₆₅, or (29) CH═N—OR₁₃₇;
R₁₄ is hydrogen atom, or C₁₋₈ alkyl;
R₁₅ and R₁₆ are each independently hydrogen atom or C₁₋₈ alkyl;
R₅₁ and R₅₂, R₅₈ and R₅₉ are each independently, hydrogen atom, or C₁₋₈ alkyl;
R₅₃, R₅₄, R₅₆, and R₆₀ are each independently, hydrogen atom, or C₁₋₈ alkyl;
R₅₅ is hydrogen atom, C₁₋₈ alkyl, or C₁₋₈ alkoxy; R₅₇ is C₁₋₈ alkyl;
R₆₁ is NR₆₂R₆₃ or hydroxy;
R₆₂ and R₆₃ are each independently, hydrogen atom, or C₁₋₈ alkyl;
R₆₅ is C₁₋₈ alkyl;
R₁₃₇ is C₁₋₈ alkyl;

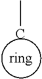

(hereinafter it is abbreviated as ring) is Cyc2 wherein the group which attaches to carbonyl is carbon;
R₇, R₈, and Cyc2 represented by ring are each independently, (1) C₃₋₁₅ mono-, bi- or tri-cyclic (fused or spiro)carboring, or (2) 3-15 membered mono-, bi- or tri-cyclic (fused or spiro) heteroring comprising 1-4 of heteroatom selected from oxygen, nitrogen and sulfur;
Cyc2 may be substituted with 1-5 of R₁₇ or R_{17′};
R₁₇ is (1) C₁₋₈ alkyl, (2) C₂₋₈ alkenyl, (3) C₂₋₈ alkynyl, (4) C₁₋₈ alkoxy, (5) C₁₋₈ alkylthio, (6) hydroxy, (7) halogen atom, (8) nitro, (9) oxo, (10) carboxy, (11) formyl, (12) cyano, (13) NR₁₈R₁₉, (14) phenyl, phenoxy or phenylthio, which may be substituted with 1-5 of R₂₀, (15) C₁₋₈ alkyl, C₂₋₈ alkenyl, C₁₋₈ alkoxy or C₁₋₈ alkylthio, which may be substituted with 1-5 of R₂₁, (16) OCOR₂₂, (17) CONR₂₃R₂₄, (18) SO₂NR₂₅R₂₆, (19) COOR₂₇, (20) COCOOR₂₈, (21) COR₂₉, (22) COCOR₃₀, (23) NR₃₁COR₃₂, (24) SO₂R₃₃, (25) NR₃₄SO₂R₃-5, or (26) SOR₆₄;
R₁₈ and R₁₉, R₃₁ and R₃₄ are each independently, hydrogen atom, or C₁₋₈ alkyl;
R₂₀ and R₂₁ are C₁₋₈ alkyl, C₁₋₈ alkoxy, hydroxy, halogen atom, nitro, or COOR₃₆;
R₂₂ and R₆₄ are each independently C₁₋₈ alkyl; R₂₃, R₂₄, R₂₅ and R₂₆ are each independently hydrogen atom, C₁₋₈ alkyl, or phenyl;
R₂₇, R₂₈, R₂₉, R₃₀, R₃₂, R₃₃ and R₃₅ are (1) C₁₋₈ alkyl, (2) C₂₋₈ alkenyl, (3) C₁₋₈ alkyl substituted with 1-5 of R₃₇, (4) diphenylmethyl, (5) triphenylmethyl, (6) Cyc3, (7) C₁₋₈ alkyl or C₂₋₈ alkenyl substituted with Cyc3, (8) C₁₋₈ alkyl substituted with O-Cyc3, S-Cyc3 or SO₂-Cyc3;
R₃₆ is hydrogen atom, or C₁₋₈ alkyl;
R₃₇ is C₁₋₈ alkoxy, C₁₋₈ alkylthio, benzyloxy, halogen atom, nitro or COOR₃₈;
R₃₈ is hydrogen atom, C₁₋₈ alkyl or C₂₋₈ alkenyl;
Cyc3 is (1) C₃₋₁₅ mono-, bi- or tri-cyclic (fused or spiro)carboring, or (2) 3-15 membered mono-, bi- or tri-cyclic (fused or spiro) heteroring comprising 1-4 of heteroatom selected from oxygen, nitrogen and sulfur;
Cyc3 may be substituted with 1-5 of R₃₉;
R₃₉ is (1) C₁₋₈ alkyl, (2) C₂₋₈ alkenyl, (3) C₂₋₈ alkynyl, (4) C₁₋₈ alkoxy, (5) C₁₋₈ alkylthio, (6) hydroxy, (7) halogen atom, (8) nitro, (9) oxo, (10) cyano, (11) benzyl, (12) benzyloxy, (13) C₁₋₈ alkyl, C₁₋₈ alkoxy or C₁₋₈ alkylthio substituted with 1-5 of R₄₀, (14) phenyl, phenoxy, phenylthio, phenylsulfonyl or benzoyl which may be substituted with 1-5 of R₄₁, (15) OCOR₄₂, (16) SO₂R₄₃, (17) NR₄₄COR₄₅, (18) SO₂NR₄₆R₄₇, (19) COOR₄₈, or (20) NR₄₉R₅₀;
R₄₀ is halogen atom;
R₄₁ is C₁₋₈ alkyl, C₁₋₈ alkoxy, halogen atom, or nitro;
R₄₂, R₄₃ and R₄₅ are C₁₋₈ alkyl;
R₄₄ and R₄₈ are hydrogen atom or C₁₋₈ alkyl;
R₄₆ and R₄₇, R₄₉ and R₅₀ are each independently, hydrogen atom or C₁₋₈ alkyl;
R₁₇ is (1) SH, (2) NR₆₆CHO, (3) Cyc5, (4) C₁₋₈ alkyl, C₂₋₈ alkenyl or C₂₋₈ alkynyl substituted with Cyc5, (5) CO—(NH-amino acid residue-CO)ₙ—OH, (6) NR₆₇CONR₆₈R₆₉, (7) CONR₇₀NR₇₁R₇₂, (8) CONR₇₃OR₇₄, (9) CONR₇₅COR₇₆, (10) C(S)NR₇₇R₇₈, (11) CONR₇₉C(S) COOR₈₀, (12) NR₈₁COCOOR₈₂, (13) NR₈₃COOR₈₄, (14) CONR₈₅C(S)R₈₆, (15) OCOR₈₇, (16) SOR₈₈, (17) CONR₃₉R₉₀, (18) SO₂NR₉₁R₉₂, (19) COOR₉₃, (20) COCOOR₉₄, (21) COR₉₅, (22) COCOR₉₆, (23) NR₉₇COR₉₈, (24) SO₂R₉₉, (25) NR₁₀₀SO₂R₁₀₁, or (26) NR₁₀₂R₁₀₃;
n is an integer of 1 or 2;
R₆₆, R₇₃, R₇₅, R₇₇, R₇₉, R₈₁, R₈₃, R₈₅, R₉₇, R₁₀₀ and R₁₀₂ are hydrogen atom, or C₁₋₈ alkyl;
R₆₇ and R₆₈, R₇₀ and R₇₁ are each independently, hydrogen atom, or C₁₋₈ alkyl;
R₈₉ and R₉₁ are (1) hydrogen atom, (2) C₁₋₈ alkyl, (3) phenyl, or (4) C₁₋₈ alkyl substituted with cyano or C₁₋₈ alkoxy; R₁₀₃ is Cyc6;
R₆₉, R₇₂, R₇₄, R₇₆, R₇₈, R₈₀, R₈₂, R₈₄, R₈₆, R₈₇, R₈₈, R₉₀ and R₉₂ are (1) hydrogen atom, (2) C₁₋₈ alkyl, (3) C₂₋₈ alkenyl, (4) C₂₋₈ alkynyl, (5) C₁₋₈ alkyl substituted with 1-5 of R₁₀₄, (6) diphenylmethyl, (7) triphenylmethyl, (8) Cyc6, (9) C₁₋₈ alkyl or C₂₋₈ alkenyl substituted with Cyc6, or (10) C₁₋₈ alkyl substituted with O-Cyc6, S-Cyc6 or SO₂-Cyc6;
R₁₀₄ is (1) C₁₋₈ alkoxy, (2) C₁₋₈ alkylthio, (3) benzyloxy, (4) halogen atom, (5) nitro, (6) COOR₁₀₅, (7) cyano, (8) NR₁₀₆R₁₀₇, (9) N₁₀₈COR₁₀₉, (10) hydroxy, (11) SH, (12) SO₃H, (13) S(O)OH, (14) OSO₃H, (15) C₂₋₈ alkenyloxy, (16) C₂₋₈ alkynyloxy, (17) COR₁₁₀, (18) SO₂R₁₁₁, or (19) C₁₋₈ alkoxy or C₁₋₈ alkylthio substituted with hydroxy;
R₁₀₅ is hydrogen atom, C₁₋₈ alkyl, or C₂₋₈ alkenyl;
R₁₀₆ and R₁₀₇ are each independently, hydrogen atom, or C₁₋₈ alkyl;
R₁₀₈ is hydrogen atom, or C₁₋₈ alkyl;
R₁₀₉ and R₁₁₁ are C₁₋₈ alkyl;
R₁₁₀ is C₁₋₈ alkyl, or halogen atom;
R₉₃, R₉₄, R₉₅, R₉₆, R₉₈, R₉₉ and R₁₀₁ are (1) C₂₋₈ alkynyl, (2) C₁₋₈ alkyl substituted with R₁₂₈ which may be substituted with 1-4 of R₂₉, (3) Cyc8, (4) C₁₋₈ alkyl or C₂₋₈ alkenyl substituted with Cyc8, or (5) C₁₋₈ alkyl substituted with O-Cyc8, S-Cyc8 or SO₂-Cyc8; R₁₂₈ is (1) cyano, (2) NR₁₀₆R₁₀₇, (3) NR₁₀₈COR₁₀₉, (4) hydroxy, (5) SH, (6) SO₃H, (7) S(O)OH, (8) OSO₃H, (9) C₂₋₈ alkenyloxy, (10) C₂₋₈ alkynyloxy, (11) COR₁₁₀, (12) SO₂R₁₁₁, or (13) C₁₋₈ alkoxy or C₁₋₈ alkylthio substituted with hydroxy;
R₁₂₉ has the same meaning as R₁₀₄;
Cyc5 and Cyc6 may be substituted with 1-5 of R₁₁₂;
R₁₁₂ is (1) C₁₋₈ alkyl, (2) C₂₋₈ alkenyl, (3) C₂₋₈ alkynyl, (4) C₁₋₈ alkoxy, (5) C₁₋₈ alkylthio, (6) hydroxy, (7) halogen atom, (8) nitro, (9) oxo, (10) cyano, (11) benzyl, (12) benzyloxy, (13) C₁₋₈ alkyl, C₁₋₈ alkoxy or C₁₋₈ alkylthio substituted with 1-5 of R₁₁₃, (14) phenyl, phenoxy, phenylthio or benzoyl, which may be substituted with 1-5 of R₁₁₄, (15) COR₁₁₅, (16) SO₂R₁₁₆, (17) NR₁₁₇COR₁₁₈, (18) SO₂NR₁₁₉R₁₂₀, (19) COOR₁₂₁, (20) NR₁₂₂R₁₂₃, (21) COR₁₂₄, (22) CONR₁₂₅R₁₂₆, (23) SH, (24) C₁₋₈ alkyl substituted with hydroxy or NR₁₂₇-benzoyl, or (25) Cyc7;
R₁₁₃ is halogen atom;
R₁₁₄ is C₁₋₈ alkyl, C₁₋₈ alkoxy, halogen atom, or nitro;
R₁₁₅, R₁₁₆ and R₁₁₈ are C₁₋₈ alkyl;
R₁₁₇, R₁₂₁, R₁₂₄ and R₁₂₇ are hydrogen atom, or C₁₋₈ alkyl;
R₁₁₉ and R₁₂₀, R₁₂₂ and R₁₂₃, R₁₂₅ and R₁₂₆ are each independently, hydrogen atom or C₁₋₈ alkyl;
Cyc7 may be substituted with 1-5 group selected from (1) C₁₋₈ alkyl, (2) C₁₋₈ alkoxy, (3) halogen atom, or (4) nitro;
Cyc8 may be substituted with R₁₃₀, and it further may be substituted with 1-4 of R₁₃₁;
R₁₃₀ is (1) COR₁₂₄, (2) CONR₁₂₅R₁₂₆, (3) SH, (4) C₁₋₈ alkyl substituted with hydroxy or NR₁₂₇-benzoyl, or (5) Cyc7;
R₁₃₁ has the same meaning as R₁₁₂;
Cyc5, Cyc6, Cyc7 and Cyc8 are (1) C₃₋₁₅ mono-, bi- or tri-cyclic (fused or spiro)carboring, or (2) 3-15 membered mono-, bi- or tri-cyclic (fused or spiro) heteroring comprising 1-4 of heteroatom selected from 1-4 of oxygen, nitrogen or sulfur;
wherein when R₁₇, is Cyc5, Cyc5 is not phenyl which may be substituted with 1-5 selected from C₁₋₈ alkyl, C₁₋₈ alkoxy, hydroxy, halogen atom, nitro, COOH, or COO(C₁₋₈ alkyl);
wherein Cyc7 is not phenyl;
Cyc4 is (1) C₅₇ monocyclic carboring, or (2) 5-7 membered monocyclic heteroring comprising 1-2 of heteroatom selected from oxygen, nitrogen and sulfur; (abbreviated as dashed line a hereafter) and (abbreviated as dashed line b hereafter) are (1) a bond, or (2) a double bond;
R₉ (1) absent or (2) is hydrogen atom;
wherein
(1) when dashed line a is a bond, dashed line b is a double bond, and R₉ is absent,
(2) when dashed line a is a double bond, dashed line b is a bond, and R₉ is hydrogen atom and R₆ is absent, and
(3) 2-(3,3-dimethyl-3,4-dihydro-(2H)-isoquinolin-1-ylidene)-1-phenylethan-1-one is excluded, or a pharmacologically acceptable salt thereof.

The preparation of these compounds is described in US 2005/222138 and WO 2003/064389.

In another embodiment, PDE7 inhibitors useful in the methods of the invention are selected from those compounds generally or specifically disclosed in WO 2003/057149, expressly incorporated herein by reference in its entirety.

In one embodiment, PDE7 inhibitors useful in the methods of the invention have the formula (13):

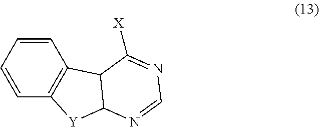

((13): Also referred to as 4-substituted fused heteropyrimidine and fused hetero-4-pyrimidone derivatives.)

The substituents for the above formula (13) are defined as follows:
(1) X is selected from halogen and NR₁R₂,
(2) Y is selected from NR₃, S, and O, with the proviso that Y is not S when X is C₁,
(3) R₁ and R₂ are independently selected from hydrogen, alkyl of 1-8 carbon atoms, alkenyl of 2-8 carbon atoms, alkynyl of 2-8 carbon atoms, cycloalkyl of 3-7 carbon atoms, polycycloalkyl of 5-9 carbon atoms, heterocycloalkyl of 2-6 carbon atoms and 1-2 heteroatoms selected from NH, S, and O, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms, or heteroaryl of 4-11 carbon atoms and 1-2 heteroatoms selected from N, S, and O, heteroaryl of 4-11 carbon atoms and 1-2 heteroatoms selected from N, S and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms, or heteroaryl of 4-11 carbon atoms and 1-2 heteroatoms selected from N, S, and O, and R₄R₅, or R₁ and R₂ combine to form, together with the nitrogen atom to which they are attached, a 5-7 membered monocyclic saturated ring, which optionally contains 1-2 additional heteroatoms selected from the group consisting of NH, NR₆, S, and O, or combine to form, together with the nitrogen atom to which they are attached, a 6-10 membered fused polycyclic saturated ring, which optionally contains 1-2 additional heteroatoms selected from the group consisting of NH, NR₆, S, and O, or combine to form, together with the nitrogen atom to which they are attached, a 5-7 membered unsaturated ring, which optionally contains 1-2 additional heteroatoms selected from the group consisting of N, S, and O, wherein said monocyclic saturated ring, polycyclic saturated ring or unsaturated ring may be substituted with 1-2 substituents selected from the group consisting of OH, alkyl of 1-6 carbon atoms, alkenyl of 2-6 carbon atoms, alkynyl of 2-6 carbon atoms, cycloalkyl of 3-7 carbon atoms, heterocycloalkyl of 2-6 carbon atoms and 1-2 heteroatoms selected from NH, S, and O, halogen, haloalkyl of 1-2 carbon atoms and a number of halogen atoms up to the perhalo level, alkoxy of 1-6 carbon atoms, haloalkoxy of 1-6 carbon atoms and a number of halogen atoms up to the perhalo level, and R₇R₈,
(4) R₃ is selected from hydrogen, alkyl of 1-8 carbon atoms, alkenyl of 2-8 carbon atoms, alkynyl of 2-8 carbon atoms, cycloalkyl of 3-7 carbon atoms, and heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atom sup to the perhalo level, haloalkoxy of 1-6 carbon atoms and a number of halogen atoms up to the perhalo level, aryl of 6-12 carbon atoms, or heteroaryl of 4-11 carbon atoms and 1-2 heteroatoms selected from N, S, and O,
(5) R₄ is selected from alkyl of 1-8 carbon atoms, alkenyl of 2-8 carbon atoms, alkynyl of 2-8 carbon atoms, C(═O), S(═O)₂, and C(═O)O,
(6) R₅ is selected from hydrogen, OH, alkyl of 1-8 carbon atoms, alkenyl of 2-8 carbon atoms, alkynyl of 2-8 carbon atoms, alkoxy of 1-8 carbon atoms, thioxy of 1-8 carbon atoms, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms, or heteroaryl of 4-11 carbon atoms and 1-2 heteroatoms selected from N, S, and O, heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms, or heteroaryl of 4-11 carbon atoms and 1-2 heteroatoms selected from N, S, and O, cycloalkyl of 3-7 carbon atoms, heterocycloalkyl of 2-6 carbon atoms and 1-2 heteroatoms selected from NH, S, and O, and NR₉R₁₀,
(7) R₆ and R₇ are independently selected from alkyl of 1-8 carbon atoms, alkenyl of 2-8 carbon atoms, and alkynyl of 2-8 carbon atoms,
(8) R₈ is selected from OH, aryl of 6-12 carbon atoms, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, haloalkoxy of 1-6 carbon atoms and a number of halogen atoms up to the perhalo level, aryl of 6-12 carbon atoms or heteroaryl of 4-11 carbon atoms and 1-2 heteroatoms selected from N, S, and O, and heteroaryl of 4-11 carbon atoms and 1-2 heteroatoms selected from N, S, and O, which may be substituted with alkyl of 1-6 carbon atoms, alkenyl of 2-6 carbon atoms, alkynyl of 2-6 carbon atoms, alkoxy of 1-6 carbon atoms, halogen, haloalkyl of 1-6 carbon atoms and a number of halogen atoms up to the perhalo level, aryl of 6-12 carbon atoms or heteroaryl of 4-11 carbon atoms and 1-2 heteroatoms selected from N, S, and O;
(9) R₉ and R₁₀ are independently selected from hydrogen, alkyl of 1-8 carbon atoms, alkenyl of 2-8 carbon atoms, and alkynyl of 2-8 carbon atoms, or R₉ and R₁₀ combine together with the nitrogen atom to which they are attached to form a 5-7 membered, unsaturated ring which may contain 1-2 additional heteroatoms selected from N, S, and O, or to form a 5-7 membered, saturated ring which may contain 1-2 additional heteroatoms selected from NH, NR₁₁, S, and O;
(10) R₁ is selected from alkyl of 1-8 carbon atoms, alkenyl of 2-8 carbon atoms, and alkynyl of 2-8 carbon atoms, and pharmaceutically acceptable salts thereof.

The preparation of these compounds is described in WO 2003/057149.

In another embodiment, PDE7 inhibitors useful in the methods of the invention are selected from those compounds generally or specifically disclosed in US 2003/0092721, U.S. Pat. No. 7,022,849, WO 2002/102315, and US 2006/116516, U.S. Pat. No. 7,601,836, each expressly incorporated herein by reference in its entirety.

In one embodiment, PDE7 inhibitors useful in the methods of the invention have the formula (14):

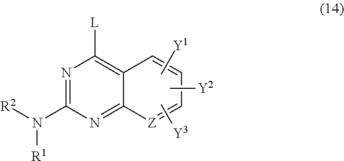

((14): Also referred to as 2-amine-substituted quinazoline and pyrido[2,3-d]pyrimidine derivatives.)

The substituents for the above formula (14) are defined as follows:
R₁ is H or alkyl;
R₂ is (a) heteroaryl or heterocyclo, either of which may be optionally substituted with one to three groups T1, T2, T3; or (b) aryl fused to a heteroaryl or heterocyclo ring wherein the combined ring system may be optionally substituted with one to three groups T1, T2, T3;
L is (a) OR₄, C(O)R₄, C(O)OR₄, SR₄, NR₃R₄, C(O)NR₃R₄, NR₃SO₂R_{4b}, halogen, nitro, or haloalkyl; or (b) alkyl, aryl, heteroaryl, heterocyclo, or cycloalkyl any of which may be optionally substituted with one to three groups T1a, T2a and/or T3a;
Y¹, Y² and Y³ are independently (a) hydrogen, halo, or —OR₄ₐ; or (b) alkyl, alkenyl, or alkynyl, any of which may be optionally substituted with one to three groups T1b, T2b and/or T3b;
R₃ and R₄ are independently H, alkyl, alkenyl, aryl, (aryl) alkyl, heteroaryl, (heteroaryl) alkyl, cycloalkyl, (cycloalkyl) alkyl, heterocyclo, or (heterocyclo) alkyl, any of which may be optionally substituted with one to three groups T1a, T2a and/or T3a; or
R₃ and R₄ together with the nitrogen atom to which they are attached may combine to form a 4- to 8-membered heterocyclo ring optionally substituted with one to three groups T1a, T2a and/or T3a;
R₄ₐ is hydrogen, alkyl, alkenyl, aryl, heteroaryl, (aryl) alkyl, (heteroaryl) alkyl, heterocyclo, (heterocyclo) alkyl, cycloalkyl, or (cycloalkyl) alkyl, any of which may be optionally substituted with one to three groups T1b, T2b and/or T3b;
R_{4b} is alkyl, alkenyl, aryl, (aryl) alkyl, heteroaryl, (heteroaryl) alkyl, cycloalkyl, (cycloalkyl) alkyl, heterocyclo, or (heterocyclo) alkyl, any of which may be optionally substituted with one to three groups T1a, T2a and/or T3a;
Z is N or CH;
T1-1b, T2-2b, and T3-3b are each independently;
(1) hydrogen or T6, where T6 is (i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl, or (heteroaryl)alkyl; (ii) a group (i) which is itself substituted by one or more of the same or different groups (i); or (iii) a group (i) or (ii) which is independently substituted by one or more of the following groups (2) to (13) of the definition of T1-1b, T2-2b and T3-3b;
(2) —OH or —OT6;
(3) —SH or —ST6;
(4) —C(O)ₜH, —C(O)ₜT6, or —O—C(O)T6, where t is 1 or 2;
(5) —SO₃H, —S(O)ₜT6, or S(O)ₜN(T9)T6;
(6) halo;
(7) cyano;
(8) nitro;
(9) -T4-NT7T8;
(10) -T4-N(T9)-T5-NT7T8;
(11) -T4-N(T10)-T5-T6;
(12) -T4-N(T10)-T5-H; and
(13) oxo;
T4 and T5 are each independently a single bond, T11S(O)ₜT12-, T11C(O)T12-, T11C(S)T12, T110T12, T11ST12, T110C(O)T12, T11C(O)OT12, T11C(═NT9a)T12, or T11C(O)C(O)T12;
T7, T8, T9, T9a and T10 are:
(1) each independently hydrogen or a group provided in the definition of T6, or
(2) T7 and T8 may together be alkylene or alkenylene, completing a 3- to 8-membered saturated or unsaturated ring together with the atoms to which they are attached, which ring is unsubstituted or substituted with one or more groups listed in the description of T1-1b, T2-2b and T3-3b, or
(3) T7 or T8, together with T9, may be alkylene or alkenylene completing a 3- to 8-membered saturated or unsaturated ring together with the nitrogen atoms to which they are attached, which ring is unsubstituted or substituted with one or more groups listed in the description of T1-1b, T2-2b and T3-3b, or
(4) T7 and T8 or T9 and T10 together with the nitrogen atom to which they are attached may combine to form a group N=CT13T14 where T13 and T14 are each independently H or a group provided in the definition of T6; and T11 and T12 are each independently a single bond, alkylene, alkenylene, or alkynylene.

The preparation of these compounds is described in US 2003/0092721, U.S. Pat. No. 7,022,849, WO 2002/102315, and US 2006/116516, U.S. Pat. No. 7,601,836.

In another embodiment, PDE7 inhibitors useful in the methods of the invention are selected from those compounds generally or specifically disclosed in U.S. Pat. No. 6,838,559, U.S. 2003/0100571, and WO 2002/102314, each expressly incorporated herein by reference in its entirety.

In one embodiment, PDE7 inhibitors useful in the methods of the invention have the formulas 15A and/or 15B:

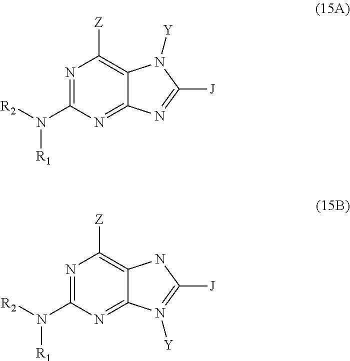

((15A), (15B): Also referred to as purine derivatives.)

The substituents for the above formulas 15A and 15B are defined as follows:
R₁ is H or alkyl;
R₂ is (a) heteroaryl, or heterocyclo, either of which may be optionally substituted with one to three groups T1, T2, T3; (b) aryl substituted with one to three groups T1, T2, T3 provided that at least one of T1, T2, T3 is other than H; or (c) aryl fused to a heteroaryl or heterocyclo ring wherein the combined ring system may be optionally substituted with one to three groups T1, T2, T3;
Y is alkyl, alkenyl, alkynyl, cycloalkyl, aryl, heterocyclo, heteroaryl, (aryl)alkyl or (heteroaryl)alkyl any of which may be optionally substituted with one to three groups T1a, T2a, T3a;
J is (a) hydrogen, halo, or OR₄, or (b) alkyl, alkenyl, alkynyl, aryl, heteroaryl, heterocyclo, or, cycloalkyl any of which may be optionally substituted with one to three groups T1b, T2b, T3b;
Z is (a) OR₄, SR₄, NR₃R₄, NR₃SO₂R₄ₐ halogen, nitro, haloalkyl; or (b) alkyl, aryl, heteroaryl, heterocyclo, or cycloalkyl any of which may be optionally substituted with one to three groups T1c, T2c, T3c;
R₃ is H, alkyl, alkenyl, aryl, (aryl)alkyl, heteroaryl, (heteroaryl)alkyl, cycloalkyl, (cycloalkyl)alkyl, heterocyclo or (heterocyclo)alkyl any of which may be optionally independently substituted where valance allows with one to three groups T1c, T2c, T3c;
R₄ is alkyl, alkenyl, aryl, (aryl)alkyl, heteroaryl, (heteroaryl)alkyl, cycloalkyl, (cycloalkyl)alkyl, heterocyclo or (heterocyclo)alkyl any of which may be optionally independently substituted where valance allows with one to three groups T1d, T2d, or T3d; or
R₃ and R₄ together with the nitrogen atom to which they are attached may combine to form a 4 to 8 membered heterocyclo ring optionally substituted with one to three groups T1c, T2c, or T3c;
R₄ₐ is hydrogen, alkyl, alkenyl, aryl, heteroaryl, (aryl)alkyl, (heteroaryl)alkyl, heterocyclo, (heterocyclo)alkyl, cycloalkyl or (cycloalkyl)alkyl any of which may be optionally substituted with one to three groups T1d, T2d or T3d; T1, T1a, T1b, T1c, T1d, T2, T2a, T2b, T2c, T2d, T3, T3a, T3b, T3c, and T3d (hereinafter abbreviated as T1-1d, T2-2d, and T3-3d) are independently
(1) hydrogen or T6, where T6 is
(a) alkyl, (hydroxy)alkyl, (alkoxy) alkyl, alkenyl, alkynyl, cycloalkyl, (cycloalkyl) alkyl, cycloalkenyl, (cycloalkenyl) alkyl, aryl, (aryl) alkyl, heterocyclo, (heterocyclo) alkyl, heteroaryl, or (heteroaryl) alkyl;
(b) a group (a) which is itself substituted by one or more of the same or different groups (a); or
(c) a group (a) or (b) which is independently substituted by one or more (preferably 1 to 3) of the following groups (2) to (13) of the definition of T1-1d, T2-2d and T3-3d,
(2) OH or OT6,
(3) SH or ST6,
(4) C(O)ₜH, C(O)ₜT6, or OC(O)T6, where t is 1 or 2;
(5) SO₃H, S(O)ₜT6, or S(O)ₜN(T9)T6,
(6) halo,
(7) cyano,
(8) nitro,
(9) T4NT7 T8,
(10) T4N(T9)-T5NT7 T8,
(11) T4N(T10)-T5-T6,
(12) T4N(T10)-T5H,
(13) oxo,
T4 and T5 are each independently a single bond, T11-S(O)ₜ-T12, T11-C(O)-T12, T11-C(S)-T12, T11-O-T12, -T11S-T12, -T11OC(O)-T12, -T11-C(O)O-T12, -T11C(═NT9a)-T12, or T11-C(O)—C(O)-T12; T7, T8, T9, T9a and T10 are
(1) each independently hydrogen or a group provided in the definition of T6, or
(2) T7 and T8 may together be alkylene or alkenylene, completing a 3- to 8-membered saturated or unsaturated ring together with the atoms to which they are attached, which ring is unsubstituted or substituted with one or more groups listed in the description of T1-1d, T2-2d and T3-3d, or
(3) T7 or T8, together with T9, may be alkylene or alkenylene completing a 3- to 8-membered saturated or unsaturated ring together with the nitrogen atoms to which they are attached, which ring is unsubstituted or substituted with one or more groups listed in the description of T1-1d, T2-2d and T3-3d, or
(4) T7 and T8 or T9 and T10 together with the nitrogen atom to which they are attached may combine to form a group N=CT13T14 where
T13 and T14 are each independently H or a group provided in the definition of T6; and
T11 and T12 are each independently a single bond, alkylene, alkenylene, or alkynylene.

The preparation of these compounds is described in U.S. Pat. No. 6,838,559, U.S. 2003/0100571, and WO 2002/102314.

In another embodiment, PDE7 inhibitors useful in the methods of the invention are selected from those compounds generally or specifically disclosed in U.S. Pat. No. 7,087,614, U.S. 2003/0162802, and WO 2002/102313, each expressly incorporated herein by reference in its entirety.

In one embodiment, PDE7 inhibitors useful in the methods of the invention have the formula (16):

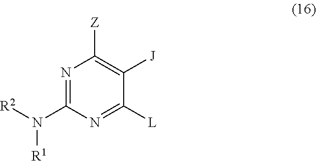

The substituents for the above formula (16) are described below as for formula (16A).

In a related embodiment, PDE7 inhibitors useful in the methods of the invention have the formula (16A):

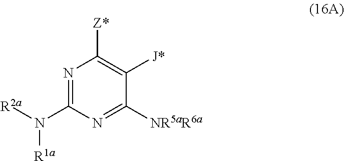

((16), (16A), (54): Also referred to as amine-substituted pyrimidine derivatives.)

The substituents for the above formulas (16) and (16A) are defined as follows:
R^{1a} is hydrogen or alkyl; R^{2a} is

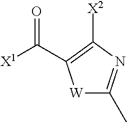

W is O or S; X¹ is alkoxy; and X² is alkyl;
Z* is halogen, haloalkyl, oxazolyl, NR₃ₐR₄ₐ, C(O)—N(H)-alkylene-COOH, or phenyl which is unsubstituted or substituted with heteroaryl, COₜH, or COₜT₆;
R₃ₐ is hydrogen or alkyl;
R₄ₐ is alkyl, alkoxy, unsubstituted or substituted (heteroaryl) alkyl, unsubstituted or substituted heterocyclo, unsubstituted or substituted (heterocyclo) alkyl, or (aryl)alkyl wherein the aryl group is substituted with one or two groups T1 and/or T2 and/or further substituted with a group T3; or R₃ₐ and R₄ₐ together with the nitrogen atom to which they are attached combine to form an unsubstituted or substituted heterocyclo ring;
R^{5a} is an unsubstituted or substituted (heteroaryl) alkyl, or (aryl)alkyl wherein the aryl group is substituted with one or two groups T1 and/or T2 and/or further substituted with a group T3; or R₅ₐ and R₆ₐ together with the nitrogen atom to which they are attached combine to form an unsubstituted or substituted heterocyclo ring;
R^{6a} is hydrogen or alkyl;
J* is hydrogen or alkyl; T1 and T2 are independently alkoxy, alkoxycarbonyl, heteroaryl, SO₃H, or SO₂R₈ₐ where R₈ₐ is alkyl, amino, alkylamino or dialkylamino; or T1 and T2 together with the aryl ring to which they are attached combine to form a bicyclic ring; T3 is H, alkyl, halo, haloalkyl, or cyano; t is 1 or 2; and T6 is alkyl, haloalkyl, cycloalkyl, alkoxy, or heteroaryl.

The preparation of these compounds is described in U.S. Pat. No. 7,087,614, U.S. 2003/0162802, and WO 2002/102313.

In another embodiment, PDE7 inhibitors useful in the methods of the invention are selected from those compounds generally or specifically disclosed in US 2003/0104974, WO 2002/088080, and WO 2002/088079, each expressly incorporated herein by reference in its entirety.

In one embodiment, PDE7 inhibitors useful in the methods of the invention have the formulas (17A) and/or (17B):

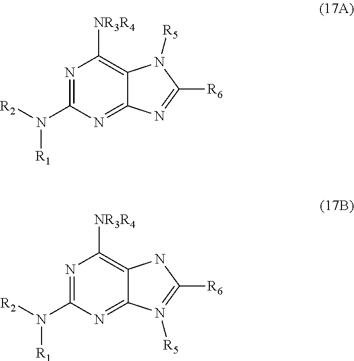

((17A), (17B): Also referred to as amine-substituted purine derivatives.)

The substituents for the above formulas (17A) and (17B) are defined as follows:
R₁ is H or alkyl; R₂ is optionally substituted heteroaryl, or 4-substituted aryl; R₃ is hydrogen or alkyl; R₄ is alkyl, optionally substituted (aryl) alkyl, optionally substituted (heteroaryl) alkyl, optionally substituted heterocyclo, or optionally substituted (heterocyclo) alkyl; or R₃ and R₄ together with the nitrogen atom to which they are attached may combine to form an optionally substituted heterocyclo ring; R₅ is alkyl, optionally substituted (aryl) alkyl, or optionally substituted (heteroaryl) alkyl; and R₆ is hydrogen or alkyl.

In a related embodiment, PDE7 inhibitors useful in the methods of the invention have the formula:

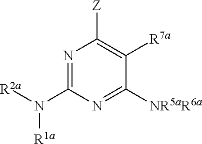

wherein R^{1a} is H or alkyl; R^{2a} is optionally substituted heteroaryl; Z is halogen, alkyl, substituted alkyl, haloalkyl, or NR₃ₐR₄ₐ; R₃ₐ is hydrogen or alkyl; R₄ₐ is alkyl, optionally substituted (heteroaryl) alkyl, optionally substituted heterocyclo, optionally substituted (heterocyclo) alkyl, or (aryl)alkyl wherein the aryl group is substituted with one or two groups T1 and T2 and optionally further substituted with a group T3; or R₃ₐ and R₄ₐ together with the nitrogen atom to which they are attached may combine to form an optionally substituted heterocyclo ring; R^{5a} is (aryl)alkyl wherein the aryl group is substituted with one or two groups T1 and T2 and optionally further substituted with a group T3; R^{6a} is hydrogen or alkyl; R^{7a} is hydrogen or alkyl; T1 and T2 are independently alkoxy, alkoxycarbonyl, heteroaryl or SO₂R₈ₐ where R₈ₐ is alkyl, amino, alkylamino or dialkylamino; or T1 and T2 together with the atoms to which they are attached may combine to form a ring (e.g., benzodioxole); T3 is H, alkyl, halo, haloalkyl or cyano.

In another related embodiment, PDE7 inhibitors useful in the methods of the invention have the formula:

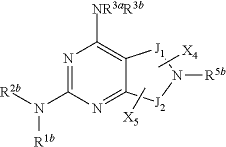

wherein R^{1b} is H or alkyl; R^{2b} is optionally substituted heteroaryl; R^{3a} is H or alkyl; R^{3b} is optionally substituted (aryl)alkyl; R^{5b} is H, alkyl, or C(O)(CH₂)ᵥOYR_{6b}, where Y is a bond or C(O), R_{6b} is hydrogen or alkyl, and v is an integer from 0 to 2; J₁ and J₂ are independently optionally substituted C₁₋₃ alkylene, provided that J₁ and J₂ are not both greater than C₂ alkylene; X₄ and X₅ are optional substituents bonded to any available carbon atom in one or both of J₁ and J₂, independently selected from hydrogen, OR₇, NR₈R₉, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl; R₇ is hydrogen, alkyl, substituted alkyl, alkenyl, alkynyl, cycloalkyl, substituted cycloalkyl, C(O)alkyl, C(O)substituted alkyl, C(O)cycloalkyl, C(O) substituted cycloalkyl, C(O)aryl, C(O) substituted aryl, C(O)O-alkyl, C(O)O-substituted alkyl, C(O)heterocycloalkyl, C(O)heteroaryl, aryl, substituted aryl, heterocycloalkyl and heteroaryl; and R₈ and R₉ are independently selected from the group consisting of hydrogen, alkyl, substituted alkyl, cycloalkyl, substituted cycloalkyl, alkenyl, alkynyl, C(O) alkyl, C(O) substituted alkyl, C(O) cycloalkyl, C(O) substituted cycloalkyl, C(O) aryl, C(O) substituted aryl, C(O)O alkyl, C(O)O substituted alkyl, C(O) heterocycloalkyl, C(O) heteroaryl, S(O)₂alkyl, S(O)₂ substituted alkyl, S(O)₂ cycloalkyl, S(O)₂ substituted cycloalkyl, S(O)₂aryl, S(O)₂ substituted aryl, S(O)₂ heterocycloalkyl, S(O)₂ heteroaryl, aryl, substituted aryl, heterocycloalkyl, and heteroaryl, or R₈ and R₉ taken together with the nitrogen atom to which they are attached complete an optionally substituted heterocycloalkyl or heteroaryl ring.

In a further related embodiment, PDE7 inhibitors useful in the methods of the invention have the formula:

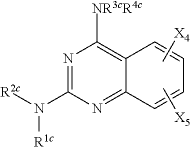

wherein R^{1c} is H or alkyl; R^{2c} is optionally substituted heteroaryl; R^{3c} is H or alkyl; R^{4c} is optionally substituted (aryl)alkyl; and X₄ and X₅ are optional substituents bonded to any available carbon atom in one or both of J₁ and J₂, independently selected from hydrogen, OR₇, NR₈R₉, alkyl, substituted alkyl, alkenyl, substituted alkenyl, alkynyl, substituted alkynyl, cycloalkyl, substituted cycloalkyl, aryl, substituted aryl, heterocycloalkyl, or heteroaryl.

The preparation of these compounds is described in US 2003/0104974, WO 2002/088080, and WO 2002/088079.

In another embodiment, PDE7 inhibitors useful in the methods of the invention are selected from those compounds generally or specifically disclosed in US 2003/0092908 and WO 2002/087513, each expressly incorporated herein by reference in its entirety.

In one embodiment, PDE7 inhibitors useful in the methods of the invention have the formula (18):

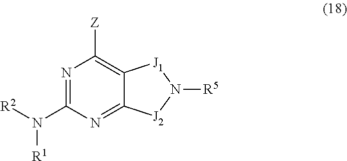

((18): Also referred to as fused heterocyclic derivatives containing a 2-amine-substituted pyrimidine.)

The substituents for the above formula (18) are defined as follows:
R₁ is hydrogen or alkyl;
R₂ is (a) heteroaryl, or heterocyclo, either of which may be optionally substituted with one to three groups T1, T2, T3; (b) aryl substituted with one to three groups T1, T2, T3 provided that at least one of T1, T2, T3 is other than H; or (c) aryl fused to a heteroaryl or heterocyclo ring wherein the combined ring system may be optionally substituted with one to three groups T1, T2, T3;
Z is NR₃R₄, NR₃SO₂R₄ₐ, OR₄, SR₄, haloalkyl, or halogen;
R₃ and R₄ are independently H, alkyl, alkenyl, aryl, (aryl)alkyl, heteroaryl, (heteroaryl)alkyl, cycloalkyl, (cycloalkyl)alkyl, heterocyclo or (heterocyclo)alkyl any of which may be optionally independently substituted where valence allows with one to three groups T1a, T2a, or T3a; or
R₃ and R₄ may be taken together with the nitrogen atom to which they are attached to form a heterocyclo or heteroaryl ring optionally independently substituted where valance allows with one to three groups T1a, T2a, or T3a;
R₄ₐ is alkyl, alkenyl, aryl, (aryl)alkyl, heteroaryl, (heteroaryl)alkyl, cycloalkyl, (cycloalkyl)alkyl, heterocyclo or (heterocyclo)alkyl any of which may be optionally independently substituted where valance allows with one to three groups T1a, T2a, or T3a;
R_{3b} and R_{4b} are independently H, alkyl, alkenyl, aryl, (aryl)alkyl, heteroaryl, (heteroaryl)alkyl, cycloalkyl, (cycloalkyl)alkyl, heterocyclo or (heterocyclo)alkyl;
R₅ is
(1) hydrogen, or cyano;
(2)alkyl, alkenyl, alkynyl, cycloalkyl, (cycloalkyl)alkyl, aryl, (aryl)alkyl, heterocyclo, (heterocyclo)alkyl, heteroaryl or (heteroaryl)alkyl, any of which may be optionally independently substituted where valance allows with one to three groups T1b, T2b, or T3b; or
(3) C(O)R₆, C(O)OR₆, C(O)—C(O)OR, or SO₂R₆ₐ;
R₆ is H, alkyl, alkenyl, NR_{3b}R_{4b}, heterocyclo, (heterocyclo)alkyl, (hydroxy)alkyl, (alkoxy)alkyl, (aryloxy)alkyl, (NR_{3b}R_{4b})alkyl, heteroaryl, aryl or (aryl)alkyl, any of which may be optionally independently substituted where valance allows with one to three groups T1b, T2b, or T3b;
R₆ₐ is alkyl, alkenyl, NR_{3b}R_{4b}, heterocyclo, (heterocyclo)alkyl, (hydroxy)alkyl, (alkoxy)alkyl, (aryloxy)alkyl, (NR_{3b}R_{4b})alkyl, heteroaryl, aryl or (aryl)alkyl, any of which may be optionally independently substituted where valance allows with one to three groups T1b, T2b, or T3b;
J₁ and J₂ are independently optionally substituted C₁₋₃ alkylene, provided that J₁ and J₂ are not both greater than C₂ alkylene; and
T1-1b, T2-2b, and T3-3b are each independently
(1) hydrogen or T6, where T6 is (i)alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl, or (heteroaryl)alkyl; (ii) a group (i) which is itself substituted by one or more of the same or different groups (i); or (iii) a group (i) or (ii) which is independently substituted by one or more (preferably 1 to 3) of the following groups (2) to (13) of the definition of T1-1b, T2-2b, and T3-3b,
(2) OH or OT6,
(3) SH or ST6,
(4) C(O)tH, C(O)tT6, or OC(O)T6, where t is 1 or 2,
(5) SO₃H, S(O)ₜT6, or S(O)ₜN(T9)T6,
(6) halo,
(7) cyano,
(8) nitro,
(9) T4-NT7T8,
(10) T4-N(T9)-T5-NT7T8,
(11) T4-N(T10)-T5-T6,
(12) T4-N(T10)-T5H,
(13) oxo,
T4 and T5 are each independently (1) a single bond, (2) T11-S(O)-T12, (3) T11-C(O)-T12, (4) T11-C(S)-T12, (5) -T11-O-T12, (6) T11-S-T12, (7) T11-O—C(O)-T12, (8) T11-C(O)—O-T12, (9) T11-C(═NT9a)-T12, or (10) T11-C(O)—C(O)-T12,
T7, T8, T9, T9a and T10,
(1) are each independently hydrogen or a group provided in the definition of T6, or
(2) T7 and T8 may together be alkylene or alkenylene, completing a 3- to 8-membered saturated or unsaturated ring together with the atoms to which they are attached, which ring is unsubstituted or substituted with one or more groups listed in the description of T1-1b, T2-2b, and T3-3b, or
(3) T7 or T8, together with T9, may be alkylene or alkenylene completing a 3- to 8-membered saturated or unsaturated ring together with the nitrogen atoms to which they are attached, which ring is unsubstituted or substituted with one or more groups listed in the description of T1-1b, T2-2b, and T3-3b, or
(4) T7 and T8 or T9 and T10 together with the nitrogen atom to which they are attached may combine to form a group N=CT13T14 where T13 and T14 are each independently H or a group provided in the definition of T6; and T11 and T12 are each independently a single bond, alkylene, alkenylene, or alkynylene.

The preparation of these compounds is described in US20030092908 and WO 2002/087513.

In another embodiment, PDE7 inhibitors useful in the methods of the invention are selected from those compounds generally or specifically disclosed in US20040127707 and WO 2002/085906, each expressly incorporated herein by reference in its entirety.

In one embodiment, PDE7 inhibitors useful in the methods of the invention have the formula (19):

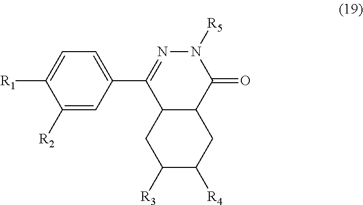

((19): Also referred to as phthalazinone derivatives.)

The substituents for the above formula (19) are defined as follows:
R₁ is 1-2C-alkoxy or 1-2C-alkoxy which is completely or predominantly substituted by fluorine,
R₂ is fluorine, bromine, or chlorine,
R₃ and R₄ are both hydrogen or together form an additional bond,
R₅ is R₆, CₘH₂ₘ—R₇, CₙH₂ₙ—C(O)R₈, CH(R₉)₂, CₚH₂ₚ—Y-Aryl1, R₁₂ or R₂₆, wherein
R₆ 1-8C-alkyl, 3-10C-cycloalkyl, 3-7C-cycloalkylmethyl, 3-7C-alkenyl, 3-7C-alkinyl, phenyl-3-4C-alkenyl, 7-10C-polycycloalkyl, naphthyl, pyridyl, pyrazinyl, pyridazinyl, pyrimidyl, quinazolinyl, quinoxalinyl, cinnolinyl, isoquinolinyl, quinolinyl, indanyl, indazolyl, benzoxazolyl, benzothiazolyl, oxazolyl, thiazolyl, N-methylpiperidyl, tetrahydropyranyl, 6-methyl-3-trifluoromethyl-pyridin-2-yl, 1,3,4-trimethyl-1H-pyrazolo[3,4-b]pyridin-6-yl, 3-thiophen-2-yl[1,2,4]thiadiazol-5-yl, 1,1-dioxide-tetrahydrothiophen-3-y-1,1-oxo-1,3-dihydro-isobenzofuran-5-yl, 4-(4-yl-but-1-oxy)benzoic acid, or an unsubstituted or by R₆₁ and/or R₆₂ substituted phenyl radical, wherein
R₆₁ is hydroxyl, 1-4C-alkyl, 1-4C-alkoxy, nitro, cyano, halogen, carboxyl, hydroxycarbonyl-1-4C-alkyl, 1-4C-alkoxycarbonyl, hydroxy-1-4C-alkyl, amino, mono- or di-1-4C-alkylamino, 1-4C-alkylcarbonylamino, aminocarbonyl, mono- or di-1-4C-alkylaminocarbon-yl, aminosulfonyl, mono- or di-1-4C-alkylaminosulfonyl, 4-methylphenylsulfonamido, imidazolyl; tetrazol-5-yl, 2-(1-4C-alkyl)tetrazol-5-yl or 2-benzyltetrazol-5-yl and
R₆₂ is 1-4C-alkyl, 1-4C-alkoxy, nitro, or halogen,
R₇ is hydroxyl, halogen, cyano, nitro, nitroxy (O—NO₂), carboxyl, carboxyphenyloxy, phenoxy, 1-4C-alkoxy, 3-7C-cycloalkoxy, 3-7C-cycloalkylmethoxy, 1-4C-alkylcarbonyl, 1-4C-alkylcarbonyloxy, 1-4C-alkylcarbonylamino, 1-4C-alkoxycarbonyl, aminocarbonyl, mono- or di-1-4C-alkylaminocarbonyl, amino, mono- or di-1-4C-alkylamino, or an unsubstituted or by R₇₁ and/or R₇₂ substituted piperidyl, piperazinyl, pyrrolidinyl or morpholinyl radical, wherein
R₇₁ is hydroxyl, 1-4C-alkyl, hydroxy-1-4C-alkyl or 1-4C-alkoxycarbonyl, and
R₇₂ is 1-4C-alkyl, carboxyl, aminocarbonyl or 1-4C-alkoxycarbonyl,
R₈ is an unsubstituted or by R₈₁ and/or R₈₂ substituted phenyl, naphthyl, phenanthrenyl or anthracenyl radical, wherein
R₈₁ is hydroxyl, halogen, cyano, 1-4C-alkyl, 1-4C-alkoxy, carboxyl, aminocarbonyl, mono- or di-1-4C-alkylaminocarbonyl, 1-4C-alkylcarbonyloxy, 1-4C-alkoxycarbonyl, amino, mono- or di-1-4C-alkylamino, 1-4C-alkylcarbonylamino, or 1-4C-alkoxy which is completely or predominantly substituted by fluorine, and
R₈₂ is hydroxyl, halogen, 1-4C-alkyl, 1-4C-alkoxy or 1-4C-alkoxy which is completely or predominantly substituted by fluorine,
R₉ is C_{q}H_{2q}-phenyl,
Y is a bond or O (oxygen),
Aryl₁ is an unsubstituted phenyl, naphthyl, pyridyl, pyrazinyl, pyridazinyl, pyrimidinyl, quinazolinyl, quinoxalinyl, cinnolinyl, isoquinolyl, quinolyl, coumarinyl, benzimidazolyl, benzoxazolyl, benzothiazolyl, benzotriazolyl, N-benzosuccinimidyl, imidazolyl, pyrazolyl, oxazolyl, thiazolyl, furyl, thienyl, pyrrolyl, a 2-(1-4C-alkyl)-thiazol-4-yl radical, or a phenyl radical substituted by R₁₀ and/or R₁₁, wherein
R₁₀ is hydroxyl, halogen, nitro, cyano, 1-4C-alkyl, trifluoromethyl, 1-4C-alkoxy, carboxyl, hydroxycarbonyl-1-4C-alkyl, 1-4C-alkylcarbonyloxy, 1-4C-alkoxycarbonyl, amino, mono- or di-1-4C-alkylamino, 1-4C-alkylcarbonylamino, aminocarbonyl, mono- or di-1-4C-alkylamino-carbonyl, imidazolyl or tetrazolyl, and R₁₁ is hydroxyl, halogen, nitro, 1-4C-alkyl or 1-4C-alkoxy,
m is an integer from 1 to 8, n is an integer from 1 to 4, p is an integer from 1 to 6,
q is an integer from 0 to 2,
R₁₂ is a radical of formula (a) N

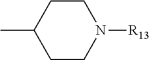

wherein R₁₃ is S(O)₂—R₁₄, S(O)₂—(CH₂)ᵣ—R₁₅, (CH₂)ₛ—S(O)₂R₁₆, C(O)R₁₇, C(O)—(CH₂)ᵣ—R₁₈, (CH₂)ₛ—C(O)—R₁₉, Hetaryl1, Aryl₂ or Aryl₃-1-4C-alkyl, R₁₄ is 1-4C-alkyl, 5-dimethylaminonaphthalin-1-yl, N(R₂₀)R₂₁, phenyl or phenyl substituted by R₂₂ and/or R₂₃, R₁₅ is N(R₂₀)R₂₁, R₁₆ is N(R₂₀)R₂₁,
R₁₇ is 1-4C-alkyl, hydroxycarbonyl-1-4C-alkyl, phenyl, pyridyl, 4-ethyl-piperazin-2,3-dion-1-yl, 2-oxo-imidazolidin-1-yl or N(R₂₀)R₂₁, R₁₈ is N(R₂₀)R₂₁, R₁₉ is N(R₂₀)R₂₁, phenyl, phenyl substituted by R₂₂ and/or R₂₃ and/or R₂₄, R₂₀ and R₂₁ are independent from each other hydrogen, 1-7C-alkyl, 3-7C-cycloalkyl, 3-7C-cycloalkylmethyl or phenyl, or R₂₀ and R₂₁ together and with inclusion of the nitrogen atom to which they are bonded, form a 4-morpholinyl-ring, 1-pyrrolidinyl-ring, 1-piperidinyl-ring, 1-hexahydroazepino-ring or a 1-piperazinyl-ring of formula (b)

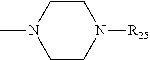

wherein R₂₅ is pyrid-4-yl, pyrid4-ylmethyl, 1-4C-alkyl-dimethylamino, dimethylaminocarbonylmethyl, N-methyl-piperidin-4-yl, 4-morpholino-ethyl or tetrahydrofuran-2-ylmethyl-, R₂₂ is halogen, nitro, cyano, carboxyl, 1-4C-alkyl, trifluoromethyl, 1-4C-alkoxy, 1-4C-alkoxycarbonyl, amino, mono- or di-1 4C-alkylamino, aminocarbonyl 1-4C-alkylcarbonylamino or mono- or di-1-4C-alkyiaminocarbon-yl, R₂₃ is halogen, amino, nitro, 1-4C-alkyl or 1-4C-alkoxy, R₂₄ is halogen,
Hetaryl₁ is pyrimidin-2-yl, thieno-[2,3-d]pyrimidin-4-yl, 1-methyl-1H-pyrazolo-[3,4-d]pyrimidin-4-yl, thiazolyl, imidazolyl or furanyl, Aryl₂ is pyridyl, phenyl or phenyl substituted by R₂₂ and/or R₂₃, Aryl₃ is pyridyl, phenyl, phenyl substituted by R₂₂ and/or R₂₃, 2-oxo-2H-chromen-7-yl or 4-(1,2,3-thiadiazol-4-yl)phenyl,
r is an integer from 1 to 4, s is an integer from 1 to 4,
R₂₆ is a radical of formula (c)

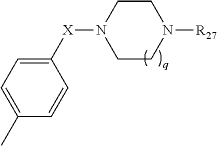

wherein R₂₇ is C(O)R₂₈, (CH₂)ₜ—C(O)R₂₉, (CH₂)ᵤR₃₀, Aryl₄, Hetaryl₂, phenylprop-1-en-3-yl or 1-methylpiperidin-4-yl, R₂₈ hydrogen, 1-4C-alkyl, OR₃₁, furanyl, indolyl, phenyl, pyridyl, phenyl substituted by R₃₄ and/or R₃₅ or pyridyl substituted by R₃₆ and/or R₃₇, R₂₉ is N(R₃₂)R₃₃, R₃₀ is N(R₃₂)R₃₃, tetrahydrofuranyl or pyridinyl, R₃₁ is 1-4C-alkyl, R₃₂ is hydrogen, 1-4C-alkyl, 3-7C-cycloalkyl or 3-7C-cycloalkylmethyl, R₃₃ is hydrogen, 1-4C-alkyl, 3-7C-cycloalkyl or 3-7C-cycloalkylmethyl, or
R₃₂ and R₃₃ together and with inclusion of the nitrogen atom to which they are bonded, form a 4-morpholinyl-, 1-pyrrolidinyl-, 1-piperidinyl- or 1-hexahydroazepinyl-ring,
Aryl₄ is phenyl, pyridyl, pyrimidinyl, phenyl substituted by R₃₄ and/or R₃₅, pyridyl substituted by R₃₆ and/or R₃₇, R₃₄ is halogen, nitro, 1-4C-alkyl, trifluoromethyl or 1-4C-alkoxy, R₃₅ is halogen or 1-4C-alkyl, R₃₆ is halogen, nitro, 1-4C-alkyl, trifluoromethyl or 1-4C-alkoxy, R₃₇ is halogen or 1-4C-alkyl,
Hetaryl₂ is indol-4-yl, 2-methyl-quinolin-4-yl, 5-chloro-6-oxo-1-phenyl-1,6-dihydro-pyridazin-4-y-1,3-phenyl-1,2,4-thiadiazol-5-yl or 3-o-tolyl-1,2,4-thiadiazol-5-yl,
t is an integer from 1 to 4, u is an integer from 1 to 4, v is an integer from 1 to 2, X is —C(O)— or —S(O)₂—, and the salts of these compounds.

The preparation of these compounds is described in US 2004/0127707 and WO 2002/085906.

In another embodiment, PDE7 inhibitors useful in the methods of the invention are selected from those compounds generally or specifically disclosed in U.S. Pat. No. 6,818,651, US 2004/0044212, and WO 2002/040450, each expressly incorporated herein by reference in its entirety.

In one embodiment, PDE7 inhibitors useful in the methods of the invention have the formula (20):

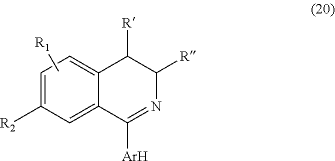

((20): Also referred to as 3,4-dihydroisoquinoline and isoquinoline derivatives.)

The substituents for the above formula (20) are defined as follows:
either R₁ denotes hydrogen, and R₂ denotes fluorine, chlorine, bromine, cyano, trifluoromethyl or phenoxy, or R₁ denotes hydrogen, fluorine, chlorine, bromine, trifluoromethyl or cyano, and R₂ denotes hydrogen, R′ and R″ both denote hydrogen or together represent a bond, and Ar represents a phenyl radical of the formulae IIa, IIb, or IIc

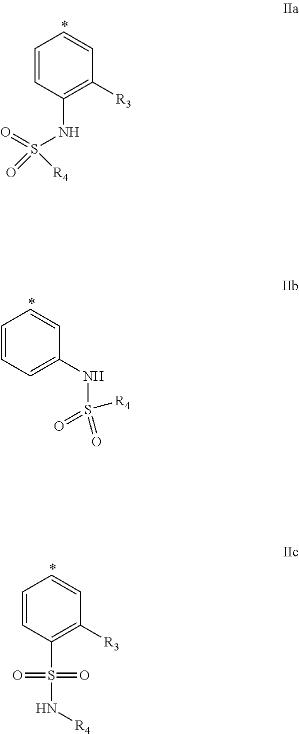

wherein R₃ denotes hydrogen, hydroxyl, nitro, amino, carboxyl, aminocarbonyl, 1-4C-alkoxy, trifluoromethoxy, 1-4C-alkoxycarbonyl or mono- or di-1-4C-alkylaminocarbonyl,
R₄ represents 1-4C-alkyl, naphthalenyl, 5-dimethylaminonaphthalen-1-yl, phenylethen-2-yl, 3,5-dimethylisoxazol-4-yl, 5-chloro-3-methylbenzo[b]thiophen-2-yl, 6-chloro-imidazo[2,1b]-thiazol-5-yl, or represents a phenyl or thiophene radical which is unsubstituted or is substituted by one or more identical or different radicals selected from the group halogen, cyano, 1-4C-alkyl, trifluoromethyl, 1-4C-alkoxy which is substituted entirely or mainly by fluorine, 1-4C-alkoxy, 1-4C-alkylcarbonylamino, 1-4C-alkoxycarbonyl, phenylsulfonyl or isoxazolyl, or
a hydrate, solvate, salt, hydrate of a salt, or solvate of a salt thereof.

The preparation of these compounds is described in U.S. Pat. No. 6,818,651, US 2004/0044212, and WO 2002/040450.

In another embodiment, PDE7 inhibitors useful in the methods of the invention are selected from those compounds generally or specifically disclosed in WO 2002/040449, expressly incorporated herein by reference in its entirety.

In one embodiment, PDE7 inhibitors useful in the methods of the invention have the formula (21):

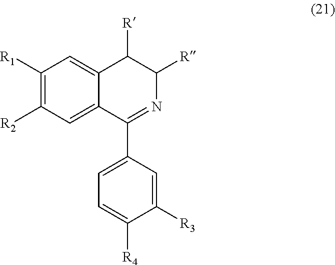

((21): Also referred to as 1-phenyl-derivative-substituted 3,4-dihydroisoquinoline derivatives.)

The substituents for the above formula (21) are defined as follows:
either R₁ denotes hydrogen and R₂ denotes fluorine, chlorine, bromine, cyano, trifluoromethyl or phenoxy, or
R₁ denotes hydrogen, fluorine, chlorine, bromine, trifluoromethyl or cyano and R₂ denotes hydrogen,
R′ and R″ both denote hydrogen or together represent a bond,
R₃ denotes hydrogen, hydroxyl, nitro, amino, carboxyl, aminocarbonyl, 1-4C-alkoxy, trifluoromethoxy, 1-4C-alkoxycarbonyl or mono- or di-1-4C-alkylaminocarbonyl and R₄ denotes C(O)—X—R₅, N(H)—C(O)—R₆ or N(H)—C(O)—N(H)—R₂, wherein
X denotes O or N(H),
R₅ denotes hydrogen, 1-4C-alkyl, 3-7C-cycloalkylmethyl, 6,6-dimethylbicyclo[3,3,l]hept-2-yl, 3-7C-alkynyl, 1-4C-alkylcarbonyl-1-4C-alkyl, aminocarbonyl-1-4C-alkyl, furan-2-ylmethyl, 2-pyridin-2-yleth-1-yl, 2-pyridin-3-ylmethyl, N-methylpiperidin-3-yl, 1-benzylpiperidin-4-yl, morpholin-4-yl-eth-2-yl, morpholin-4-yl-eth-1-yl, 2-benzo[1,3]dioxol-4-yl-eth-1-yl, chroman-4-yl, 1-methoxycarbonyl-2-indol-3-yl-eth-1-yl, 1,3-bis-methoxycarbonylprop-1-yl, 1-methoxycarbonyl-3-methylsulfanyl-eth-1-yl, 1-methoxycarbonyl-2-thiazol-2-yl-eth-1-yl, or 4-methylthiazol-5-yl-eth-2-yl, or represents a benzyl-, phenyl-eth-1-yl or 1-methoxycarbonyl-2-phenyl-eth-2-yl radical which is unsubstituted or substituted by one or more radicals selected from the group halogen, trifluoromethyl and phenyl,
R₆ denotes 2,4-dichlorophenoxymethyl, 2-tert-butoxycarbonylamino-eth-1-yl, 1-acetylpiperidin-4-yl, Ar1 or Ar2-CH═CH—,
where Ar1 represents 3-chlorophenyl, 4-trifluoromethoxyphenyl, 3-phenoxyphenyl, indol5-yl, 2-methylpyridin-5-yl, quinolin-6-yl or 2-benzothiazol-6-yl, Ar2 represents furan-2-yl, furan-3-yl, thiophen-2-yl, indol-3-yl, 3-trifluoromethylphenyl, 3-methoxyphenyl or pyridin-3-yl,
R₇ represents 1-4C-alkyl, 3-7C-alkenyl, 3-7C-cycloalkyl, 1-ethoxycarbonyl-2-phenyl-eth-1-yl, thiophen-2-yleth-1-yl or a phenyl radical which is unsubstituted or substituted by one or more radicals selected from the group halogen, cyano, 1-4C-alkyl, trifluoromethyl, 1-4C-alkylthio, 1-4C-alkoxy, 1-4C-alkoxy which is entirely or predominantly substituted by fluorine, 1-4C-alkylcarbonyl and phenoxy, or
a salt thereof.

The preparation of these compounds is described in WO 2002/040449.

In another embodiment, PDE7 inhibitors useful in the methods of the invention are selected from those compounds generally or specifically disclosed in WO 2001/098274, expressly incorporated herein by reference in its entirety.

In one embodiment. PDE7 inhibitors useful in the methods of the invention have the formula (22):

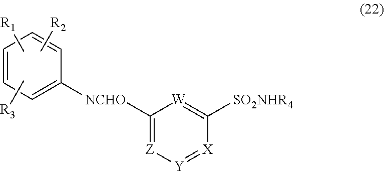

((22): Also referred to as sulphonamide derivatives.)

The substituents for the above formula (22) are defined as follows:
W, X, Y and Z, which may be the same or different, each represents a nitrogen atom or a C(R₅) group [wherein R₅ is a hydrogen or halogen atom or an alkyl, haloalkyl, alkoxy, haloalkoxy, hydroxy, —NO₂ or —CN group] provided that two or more of W, X, Y, and Z are C(R₅) groups;
R₁, R₂ and R₃, which may be the same or different, each is an atom or group -L₁(Alk₁)ᵣL₂(R₆)ₛ wherein L₁ and L₂, which may be the same or different, is each a covalent bond or a linker atom or group, r is zero or the integer 1, Alk₁ is an aliphatic or heteroaliphatic chain, s is an integer 1, 2 or 3 and R₆ is a hydrogen or halogen atom or a group selected from alkyl, —OR₇ [where R₇ is a hydrogen atom or an optionally substituted alkyl group], —SR₇, NR₇R₈ [where R₈ is as just defined for R₇ and may be the same or different], —NO₂, CN, CO₂R₇, SO₃H, S(O)R₇, SO₂R₇, OCO₂R₇, CONR₇R₈, OCONR₇R₈, CSNR₇R₈, OCR₇, OCOR₇, N(R₇) COR₈, N(R₇)CSR₈, S(O)NR₇R₈, SO₂NR₇R₈, N(R₇)SO₂R₈, N(R₇) CON(R₈)(R₉) [where R₉ is a hydrogen atom or an optionally substituted alkyl group], N(R₇) CSN(R₈)(R₈), N(R₇)SO₂N(R₈)(R₉), C(R₇)═NO(R₈), cycloaliphatic, heterocycloaliphatic, aryl or heteroaryl group]; provided that one or more of R₁, R₂, or R₃ is a substituent other than a hydrogen atom;
R₄ represents an optionally substituted phenyl, 1- or 2-naphthyl, pyridyl, pyrimidinyl, pyridazinyl, or pyrazinyl group; and
the salts, solvates, hydrates and N-oxides thereof.

The preparation of these compounds is described in WO 2001/098274.

In another embodiment, PDE7 inhibitors useful in the methods of the invention are selected from those compounds generally or specifically disclosed in WO 2001/074786, expressly incorporated herein by reference in its entirety.

In one embodiment PDE7 inhibitors useful in the methods of the invention have the formula (23):

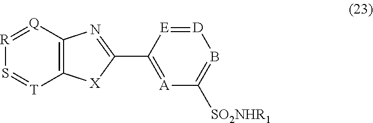

((23): Also referred to as heterobiaryl sulphonamide derivatives.)

The substituents for the above formula (23) are defined as follows:
R₁ represents an aryl or heteroaryl group;
A, B, P, and E, which may be the same or different, each represents a nitrogen atom or a C(R₂) group [wherein R₂ is a hydrogen or halogen atom or an alkyl, haloalkyl, alkoxy, haloalkoxy, hydroxy, —NO₂ or —CN group] provided that two or more of A, B, D, and E are C(R₂) groups; X represents an oxygen or sulphur atom or a N(R₃) group wherein R₃ is a hydrogen atom or an alkyl group;
Q, R, S, and T, which may be the same or different each represents a nitrogen atom or a group C(R₄) [wherein R₄ is an atom or group -L₁(Alk₁)rL₂(R₅)ₛ wherein L₁ and L₂, which may be the same or different, is each a covalent bond or a linker atom or group, r is zero or the integer 1, Alkyl is an aliphatic or heteroaliphatic chain, s is an integer 1, 2 or 3 and R₅ is a hydrogen or halogen atom or a group selected from alkyl, OR₆ [where R₆ is a hydrogen atom or an optionally substituted alkyl group], SR₆, NR₆R₇ [where R₇ is as just defined for R₆ and may be the same or different], NO₂, CN, CO₂R₆, SO₃H, S(O)R₆, SO₂R₆, OCO₂R₆, CONR₆R₇, OCONR₆R₇, CSNR₆R₇, OCR₆, OCOR₆, N(R₆) COR₇, N(R₆)CSR₇, S(O)NR₆R₇, SO₂NR₆R₇, N(R₆)SO₂R₇; N(R₆) CON(R₇)(R₈) [where R₈ is a hydrogen atom or an optionally substituted alkyl group], N(R₆) CSN(R₇)(R₃), N(R₆)SO₂N(R₇)(R₈), C(R₆)═NO(R₇) cycloaliphatic, heterocycloaliphatic, aryl or heteroaryl group] provided that two or more of Q, R, S, and T are C(R₄) groups; and the salts, solvates, hydrates and N-oxides thereof.

The preparation of these compounds is described in WO 2001/074786.

In another embodiment, PDE7 inhibitors useful in the methods of the invention are selected from those compounds generally or specifically disclosed in WO 2000/068230, expressly incorporated herein by reference in its entirety.

In one embodiment, PDE7 inhibitors useful in the methods of the invention have the formula (24):

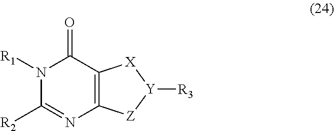

((24): Also referred to as naphthalenyl derivatives of dihydropurin-6-one.)

The substituents for the above formula (24) are defined as follows:
X—Y—Z represents NR₄—C═N or N═C—NR₄;
R₁ represents H, alkyl, cycloalkyl, cycloalkylalkyl, arylalkyl, heteroarylalkyl or heterocycloalkyl;
R₂ represents OR₈, NR₈R₉, SR₁₃, alkyl or CF₃;
R₃ represents halogen, alkyl, CF₃ or OR₈;
R₄, which can be attached to either X or Z, is a residue selected from

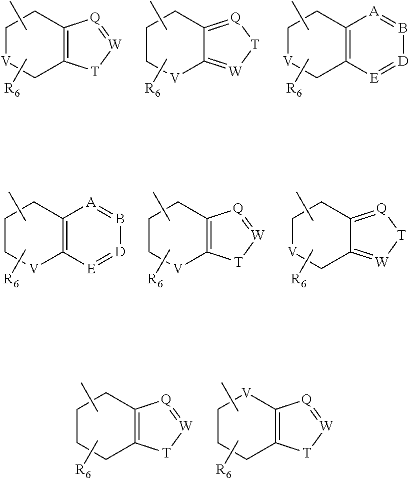

wherein attachment is through any position on the saturated ring, provided the attachment is not at a position adjacent to V, and the saturated ring may be substituted at any position with one or more R₆;
A, B, D, and are the same or different and each represents ClₙR₅, N or N—O;
V represents O, S, NR₇ or C(L¹ₘR₁₄)(L²ₙR₁₄);
Q and W are the same or different and each represents CLₙR₅ or N;
T represents O, S or NR₇;
L¹ and L² are the same or different and each represents C(R₁₅)₂;
m and n are the same or different and each represents 0, 1, 2, 3, 4 or 5;
the R₅s are the same or different and each represents H, halogen, alkyl, cycloalkyl, OR₈, NR₈R₉, CO₂R₁₀, CONR₁₁R₁₂, CONHOH, SO₂NR₁₁R₁₂, SON₁₁R₁₂, COR₁₃, SO₂R₁₃, SOR₁₃, SR₁₃, CF₃, NO₂ or CN;
R₆ represents H, alkyl, cycloalkyl, OR₈, NR₈R₉, CO₂R₁₀, CONR₁₁R₁₂, SO₂NR₁₁R₁₂, SON₁₁R₁₂, COR₁₃, SO₂R₁₃, SOR₁₃, SR₁₃, CF₃, CN or ═O;
R₇ represents H or alkyl;
R₈ represents H, alkyl, cycloalkyl, cycloalkylalkyl, aryl, arylalkyl, heteroaryl, heteroarylalkyl, heterocyclo or heterocycloalkyl;
R₉ represents R₈ or alkylcarbonyl, alkoxycarbonyl, alkylsulphonyl, cycloalkylcarbonyl, cycloalkoxycarbonyl, cycloalkylsulphonyl, cycloalkylalkylcarbonyl, cycloalkylalkoxycarbonyl, cycloalkylalkylsulphonyl, arylcarbonyl, arylsulphonyl, heteroarylcarbonyl, heteroarylsulphonyl, heterocyclocarbonyl, heterocyclosulphonyl, arylalkylcarbonyl, arylalkoxycarbonyl, arylalkylsulphonyl, heteroarylalkylcarbonyl, heteroarylalkoxycarbonyl, heteroarylsulphonyl, heterocycloalkylcarbonyl, heterocycloalkoxycarbonyl or heterocycloalkylsulphonyl; or
NR₈R₉ represents a heterocyclic ring such as morpholine;
R₁₀ represents H, alkyl, cycloalkyl, cycloalkylalkyl, arylalkyl, heteroarylalkyl or heterocycloalkyl;
R₁₁ and R₁₂ are the same or different and are each R₈, or NR₁₁R₁₂ represents a heterocyclic ring such as morpholine;
R₁₃ represents alkyl, cycloalkyl, cycloalkylalkyl, aryl, arylalkyl, heteroaryl, heteroarylalkyl, heterocyclo or heterocycloalkyl;
the R₁₄s are the same or different and are each selected from H alkyl, cycloalkyl, OR₈, NR₈R₉, CO₂R₁₀, CONR₁₁R₁₂, CONHOH, SO₂NR₁₁R₁₂, SON₁₁R₁₂, COR₁₃, SO₂R₁₃, SOR₁₃, SR₁₃, CF₃, NO₂ and CN, provided that when both m and n represent 0, if one R₁₄ is OR₈, NR₈R₉ or SR₁₃, the other is not OR₈, NR₈R₉ or SR₁₃; and
R₁₅ represents H, alkyl or F; or
a pharmaceutically acceptable salt thereof.

The preparation of these compounds is described in WO 2000/068230, incorporated herein by reference in its entirety.

In another embodiment, PDE7 inhibitors useful in the methods of the invention are selected from those compounds generally or specifically disclosed in US 2004/0106631, U.S. Pat. No. 7,429,598, EP-A-1400244, and WO 2004/026818, each expressly incorporated herein by reference in its entirety.

In one embodiment, PDE7 inhibitors useful in the methods of the invention have the formula (25):

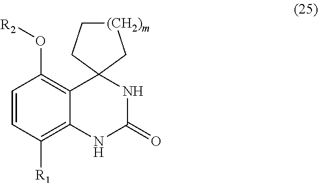

((25): Also referred to as spiroc derivatives.)

The substituents for the above formula (25) are defined as follows:
m is 1, 2 or 3; R₁ is methyl, chloro, bromo or fluoro; R₂ is -Q₁-Q₂-Q₃-Q₄ or (C₁-C₆) alkyl, said (C₁-C₆) alkyl is substituted with one to three OR₄, COOR₄, NR₄R₅, NRC(═O)R₄, C(═O)NR₄R₅ or SO₂NR₄R₅;
R₄ is (C₁-C₆) alkyl substituted with one to three F, CN, S(═O)R₆, SO₃H, SO₂R₆, SR₇, C(═O)—NH—SO₂—CH₃, C(═O)R₇, NR′C(═O)R₇, NR′SO₂R₆, C(═O)NR₇R₈, O—C(═O)NR₇R₈ or SO₂NR₇R₈;
R₅ is H or (C₁-C₆) alkyl optionally substituted with one to three F, CN, S(═O)R₆, SO₃H, SO₂R₆, SR₇, C(═O)—NH—SO₂—CH₃, C(═O)R₇, NR′C(═O)R₇, NR′SO₂R₆, C(═O)NR₇R₈, O—C(═O)NR₇R₈ or SO₂NR₇R₈; or said (C₁-C₆) alkyl is
(1) substituted with one to three OC(═O)R₄ₐ, SR₄ₐ, S(═O)R₃, C(═NR₉)R₄ₐ, C(═NR₉)—NR₄ₐR₅ₐ, NR—C(═NR₉)—NR₄ₐR₅ₐ, NRCOOR₄ₐ, NR—C(═O)NR₄ₐR₅ₐ, NR—SO₂—NR₄ₐR₅ₐ, NR—C(═NR₉)—R₄ₐ or NR—SO₂—R₃; and
(2) optionally substituted with one or two OR₄ₐ, COOR₄ₐ, C(═O)—R₄ₐ, NR₄ₐR₅ₐ, NRC(═O)R₄ₐ, C(═O)NR₄R₅ₐ or SO₂NR₄ₐR₅ₐ;
R₉ is H, CN, OH, OCH₃, SO₂CH₃, SO₂NH₂ or (C₁-C₆)alkyl; and R₃ is (C₁-C₆) alkyl optionally substituted with one to three F, CN, S(═O)R₆, SO₃H, SO₂R₆, C(═O)—NH—SO₂—CH₃, OR₇, SR₇, COOR₇, C(═O)R₇, O—C(═O)NR₇R₈, NR₇R₈, NR′C(═O)R₇, NR′SO₂R₆, C(═O)NR₇R₈ or SO₂NR₇R₃;
R₄ₐ and R₅ₐ are the same or different and are H or (C₁-C₆) alkyl optionally substituted with one to three F, CN, S(═O)R₆, SO₃H, SO₂R₆, C(═O)—NH—SO₂—CH₃, OR₇, SR₇, COOR₇, C(═O)R₇, O—C(═O)NR₇R₈, NR₇R₈, NR′C(═O)R₇, NR′SO₂R₆, C(═O)NR₇R₈ or SO₂NR₇R₈;
Q¹ is a single bond or (C₁-C₆) alkylene; Q² is a saturated 4- to 6-membered heterocyclyl comprising one or two O or N; Q³ is (C₁-C₅) alkylene; Q⁴ is a 4 to 8-membered, aromatic or non aromatic, heterocyclyl comprising 1 to 4 O, S, S(═O), SO₂, or N, said heterocyclyl being optionally substituted with one to three OR, NRR′, —CN or (C₁-C₆) alkyl;
R is H or (C₁-C₆) alkyl;
R₆ is (C₁-C₆) alkyl optionally substituted with one or two OR′;
R₇ and R₈ are the same or different and are H or (C₁-C₆) alkyl optionally substituted with one or two OR′;
R₉ is H, CN, OH, OCH₃, SO₂CH₃, SO₂NH₂ or (C₁-C₆) alkyl;
R′ is H or (C₁-C₆) alkyl; and R″ is H or (C₁-C₆) alkyl;
provided that (1) the atom of Q² bound to Q¹ is a carbon atom; and (2) the atom of Q⁴ bound to Q³ is a carbon atom;
or a racemic form, isomer, pharmaceutically acceptable derivative thereof.

The preparation of these compounds is described in US 2004/0106631, U.S. Pat. No. 7,429,598, EP-A-1400244, and WO 2004/026818.

In another embodiment, PDE7 inhibitors useful in the methods of the invention are selected from those compounds generally or specifically disclosed in U.S. Pat. No. 6,936,609 and US 2004/0249148, and WO 2003/000693 each expressly incorporated herein by reference in its entirety.

In one embodiment, PDE7 inhibitors useful in the methods of the invention have the formula (26):

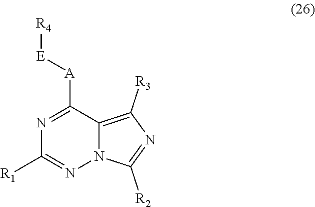

((26): Also referred to as imidazotriazine derivatives.)

The substituents for the above formula (26) are defined as follows:
R₁ represents (C₆-C₁₀)-aryl, which is optionally identically or differently substituted by radicals selected from the group consisting of halogen, formyl, carbamoyl, cyano, hydroxyl, trifluoromethyl, trifluoromethoxy, nitro, (C₁-C₆)-alkyl or (C₁-C₆)-alkoxy, and optionally by a radical of the formula SO₂NR₅R₆, wherein R₅ and R₆ independently of one another denote hydrogen or (C₁-C₆)-alkyl, or NR₅R₆ denotes 4- to 8-membered heterocyclyl, bonded via a nitrogen atom, optionally identically or differently substituted by radicals selected from the group consisting of oxo, halogen, (C₁-C₆)-alkyl and (C₁-C₆)-acyl,
R₂ represents a saturated or partially unsaturated hydrocarbon radical having 1 to 10 carbon atoms,
R₃ represents methyl or ethyl,
A represents O, S, or NR₇, wherein R₇ denotes hydrogen or (C₁-C₆)-alkyl optionally substituted by (C₁-C₃)-alkoxy,
E represents a bond or (C₁-C₃)-alkanediyl,
R₄ represents (C₆-C₁₀)-aryl or 5- to 10-membered heteroaryl, where aryl and heteroaryl are optionally identically or differently substituted by radicals selected from the group consisting of halogen, formyl, carboxyl, carbamoyl, —SO₃H, aminosulphonyl, cyano, hydroxyl, trifluoromethyl, trifluoromethoxy, nitro, (C₁-C₆)-alkyl, (C₁-C₆)-alkoxy, 1,3-dioxa-propane-1,3-diyl, (C₁-C₆)-alkylthio, (C₁-C₆)-alkylsulphinyl and (C₁-C₆)-alkyl-sulphonyl, —NR₈R₉ end optionally methyl-substituted, 5- to 6-membered heteroaryl or phenyl,
wherein R₈ and R₉ independently of one another denote hydrogen, (C₁-C₆)-alkyl or (C₁-C₆)-acyl, or salt thereof.

The preparation of these compounds is described in U.S. Pat. No. 6,936,609 and US 2004/0249148, and WO 2003/000693.

In another embodiment, PDE7 inhibitors useful in the methods of the invention are selected from those compounds generally or specifically disclosed in WO 2006/092692, expressly incorporated herein by reference in its entirety.

In one embodiment, PDE7 inhibitors useful in the methods of the invention have the formulas (27A), (27B, (27C) and (27D):

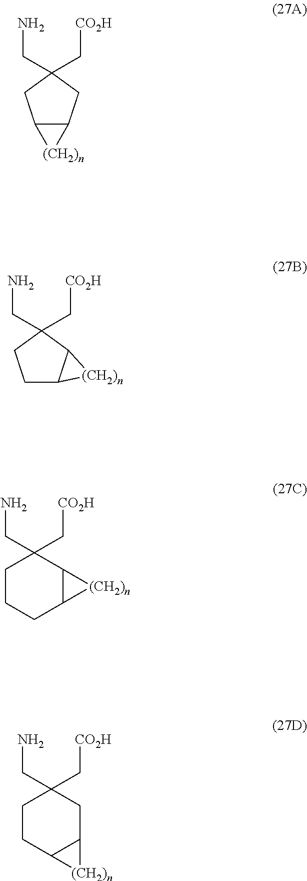

wherein n is an integer of from 1 to 4, and where there are stereocenters, each center may be independently R or S.
((27A), (27B), (27C), (27D): Also referred to as spirocondensed 4-amino-butanecarboxylic adic derivatives.)

The preparation of these compounds is described in WO 2006/092692.

In another embodiment, PDE7 inhibitors useful in the methods of the invention are selected from those compounds generally or specifically disclosed in US 2006/229306 and WO 2004/065391, each expressly incorporated herein by reference in its entirety.

In one embodiment, PDE7 inhibitors useful in the methods of the invention have the formula (28):

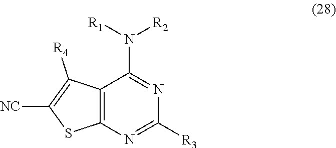

((28): Also referred to as 4-amine-substituted thieno[2,3-d]pyrimidine-6-carbonitrile derivatives.)

The substituents for the above formula (28) are defined as follows:
R₁ and R₂ either
(1) independently represent: (a) a hydrogen atom; (b) a group selected from alkyl, alkenyl and alkynyl groups, wherein each alkyl, alkenyl and alkynyl group is independently optionally substituted by one or more substituents selected from halogen atoms, hydroxy, alkoxy, aryloxy, alkylthio, hydroxycarbonyl, alcoxycarbonyl, mono- and di-alkylaminoacyl, oxo, amino, and mono- and di-alkylamino groups; or (c) a group of formula (CH₂)ₙ—R₆, wherein n is an integer from 0 to 4 and R₆ represents a cycloalkyl or cycloalkenyl group;
(2) R₁ and R₂ form, together with the nitrogen atom to which they are attached, a 3- to 8-membered ring comprising from 1 to 4 heteroatoms selected from nitrogen, oxygen and sulphur, which ring is saturated or unsaturated and optionally substituted by one or more substituents selected from halogen atoms, alkyl, hydroxy, alkoxy, acyl, hydroxycarbonyl, alkoxycarbonyl, alkylenedioxy, amino, mono- and di-alkylamino, mono- and di-alkylaminoacyl, nitro, cyano and trifluoromethyl groups;
R₃ is a group of formula (CH₂)_{n-G}, wherein n is an integer from 0 to 4 and G represents a monocyclic or bicyclic aryl or heteroaryl group comprising from zero to four heteroatoms which group is optionally substituted by one or more substituents selected from:
(1) halogen atoms;
(2) alkyl and alkylene groups, wherein each alkyl and alkylene group is independently optionally substituted by one or more substituents selected from halogen atoms; and
(3) phenyl, hydroxy, hydroxyalkyl, alkoxy, alkylenedioxy, aryloxy, alkylthio, amino, mono- and di-alkylamino, acylamino, nitro, acyl, hydroxycarbonyl, alkoxycarbonyl, cyano, difluoromethoxy and trifluoromethoxy groups;
R₄ represents a hydrogen atom, an alkyl or an aryl group.

The preparation of these compounds is described in US2006229306 and WO 2004/065391.

Other compounds useful in the methods of the invention include imidazopyridine derivatives (WO 2001/034601), dihydropurine derivatives (WO 2000/068230), pyrrole derivatives (WO 2001/032618), benzothiopyranoimidazolone derivatives (DE 19950647), guanine derivatives (Bioorg. Med. Chem. Lett. 11:1081-1083, 2001), heterocyclic derivatives (WO 2012/150369), arylindenopyridine and arylindenopyrimidine derivatives (U.S. Pat. No. 7,754,730), sulfonylbenzene derivatives (JP 2006290791), trans-aconitic acids (U.S. Pat. No. 9,610,268), thienopyrimidine/thienopyrimidinone derivatives (Org Biomol Chem 12(24): 4233-4242, 2014), 2-(isopropylamino) thieno[3,2-d]pyrimidin-4(3H)-one derivatives (Bioorg Med Chem Lett 25(9): 1910-1914, 2015), isothiazole and isoxazole fused pyrimidone dervivatives (Bioorg Med Chem Lett 22(9): 3223-3228, 2012), imidazopyridazinone derivatives (Bioorg Med Chem Lett 22(19): 6286-6291, 2012), sulfide derivatives (J Med Chem 57(20): 8590-8607, 2014), dihydronaphthyridinedione derivatives (Bioorg Med Chem Lett 21(22): 6652-6656, 2011), furan derivatives (J Med Chem 55(7): 3274-3284, 2012), S-substituted quinazolines (U.S. Pat. No. 9,796,687), 1,2-dihydro-3-phenyl-2-thioxo-4(3H)-quinazolinone (S14, S.14, CAS: 18741-24-7, Curr Med Chem 24(7): 673-700, 2017), SUN11817 (CAS: 873539-34-5, Curr Med Chem 24(7): 673-700, 2017), 3-phenyl-2,4(1H,3H)-quinazolinedithione (TC3.6, CAS: 16081-93-9, Curr Med Chem 24(7): 673-700, 2017), benzothienothiadiazine derivatives (Eur. J. Med. Chem. 36:333-338, 2001), the PDE7 inhibitors listed in Liras and Bell, Phosphodiesterases and Their Inhibitors, Wiley-VCH (pages 165-190), steroids, podocarpanes, and fused thiophenes (Journal of Biomolecular Screening, 15(4), 359-367), and other derivatives which also include non-selective PDE inhibitors with significant inhibitory properties against PDE7 like IBMX (3-isobutyl-1-methylxanthine, CAS: 28822-58-4) and dipyridamole (2,2′,2″,2′″-[[4,8-Di(piperidin-1-yl)pyrimido[5,4-d]pyrimidine-2,6-diyl]dinitrilo]tetraethanol, CAS: 58-32-2) (Curr Med Chem 24(7): 673-700, 2017) and ibudilast (CAS: 50847-11-5) and, in addition to IBMX, other methylxanthine derivatives like caffeine, theophylline, theobromine, paraxanthine, 7-methylxanthine, aminophylline, pentoxifylline (CAS: 6493-05-6). The disclosure of each published patent application and journal article listed above is expressly incorporated herein by reference in its entirety.

In another embodiment, PDE7 inhibitors useful in the methods of the invention are selected from those compounds generally or specifically disclosed in WO 2008/130619, expressly incorporated herein by reference in its entirety.

In one embodiment, PDE7 inhibitors useful in the methods of the invention have the formula (29):

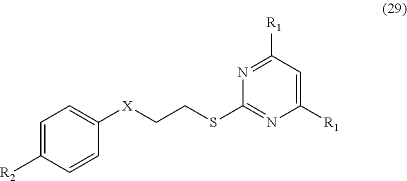

((29): Also referred to as pyrimidin-2-yl sulfide derivatives.)

The substituents for the above formula (29) are defined as follows:
X is SO, or SO₂,
R₁ is H, or alkyl,
R₂ is alkyl, or halogen.

In specific embodiments, R₁ is Me. In other specific embodiments R₁ is F. In certain embodiments R₂ is t-Bu. In specific embodiments, R₁ is methyl. In more specific embodiments, the compounds are selected from:

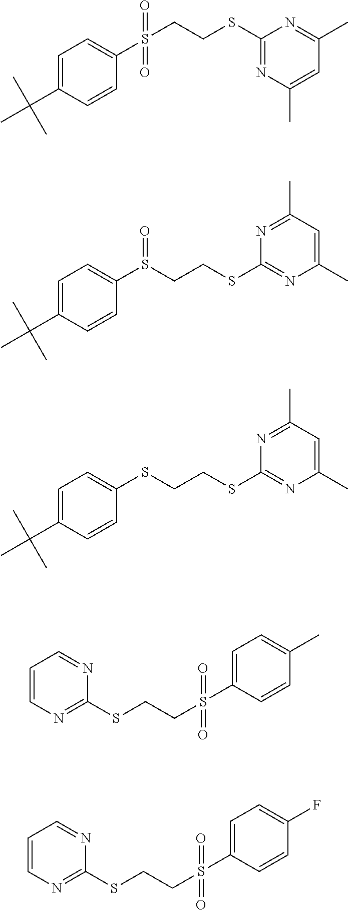

In a related embodiment, PDE7 inhibitors useful in the methods of the invention have the formula (30):

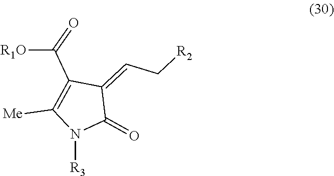

wherein
R₁ is alkyl,
R₂ is aryl or heteroaryl,
R₃ is alkyl, aryl, cycloakyl, or alkylaryl.
((30): Also referred to as 5-oxo-pyrrole-3-carboxylate derivatives.)

In specific embodiments, R₁ is methyl. In certain embodiments R₂ is furanyl or thiophenyl. In other specific embodiments, R₂ is substituted phenyl or benzyl. In preferred embodiments, R₃ is iso-butyl. In more specific embodiments, the compounds are selected from:

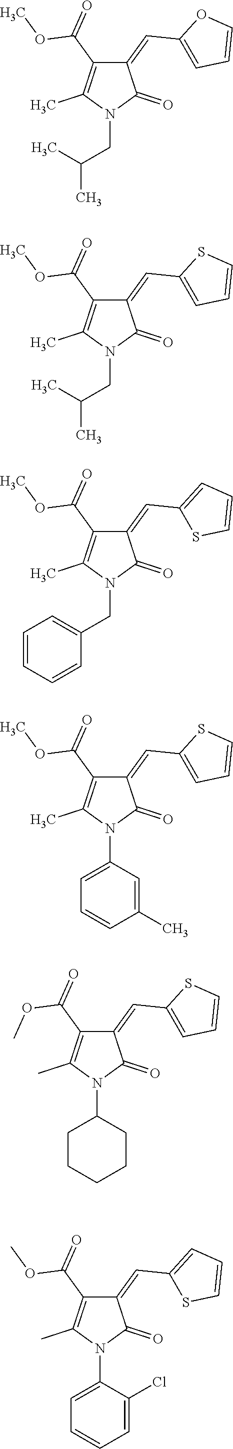

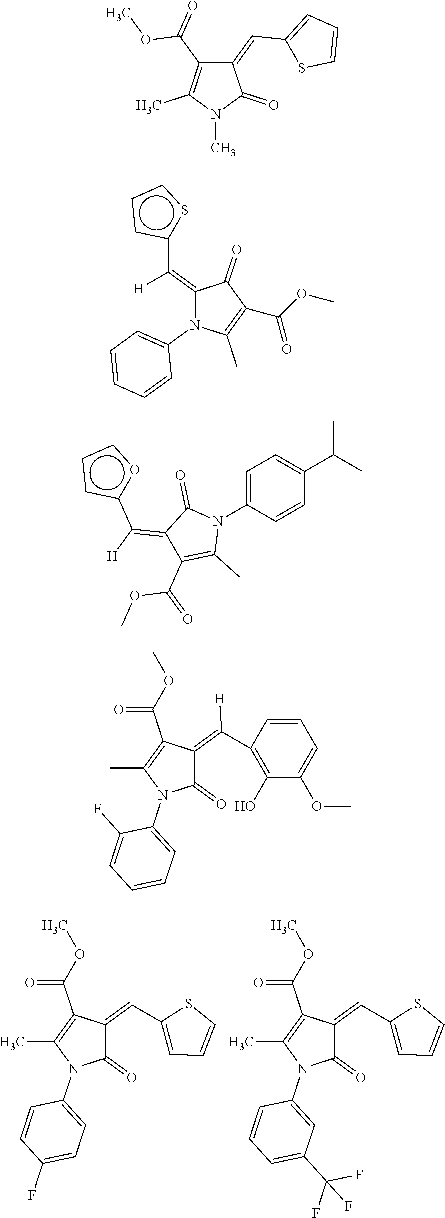

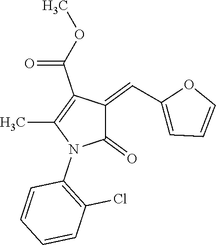 [SMILES: COC(=O)C1=C(C)N(c2ccccc2Cl)C(=O)/C1=C\c1ccco1]

In another related embodiment, PDE7 inhibitors useful in the methods of the invention have the formula (31):

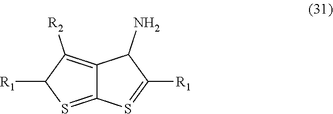

wherein
R₁ is nitrile, or alkylcarboxylate,
R₂ is alkyl, aryl, or heteroaryl.
((31): Also referred to as thieno[2,3-b]thiophen-3-amine derivatives.)

In specific embodiments, R₁ is nitrile or methylcarboxylate. In certain embodiments, R₂ is a five membered heteroaryl. In more specific embodiments, R₂ is furanyl, or thienyl. In other embodiments, R₂ is a six membered aryl. In more specific embodiments, R₂ is substituted phenyl.

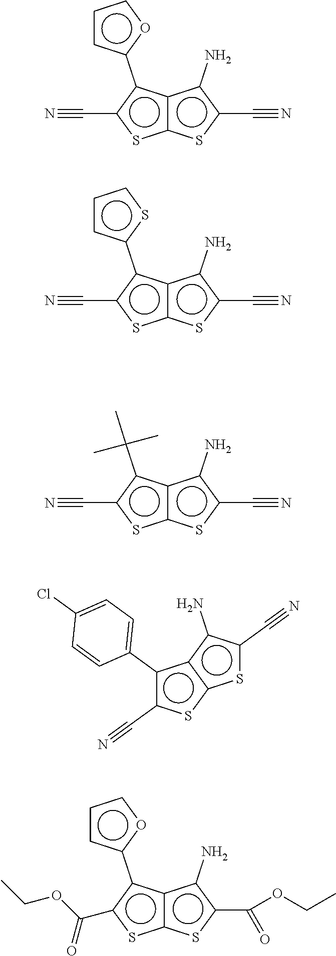

In another related embodiment, PDE7 inhibitors useful in the methods of the invention have the formula (32):

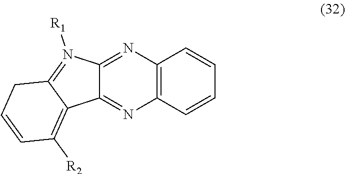

wherein
R₁ is alkyl, alkenyl, or alkylcarboxylic acid,
R₂ is halogen.
((32): Also referred to as indolo[2,3-b]quinoxaline derivatives.)

In certain embodiments R₁ is butyl. In other embodiments R₁ is terminal alkenyl.

In more specific embodiments R₁ is allyl, or vinyl. In other embodiments, R₁ is C₁₋₄alkyl. In specific embodiments R₁ is methylcarboxylic acid. In certain embodiments R₂ is Cl, or Br. In more specific embodiments, the compounds are selected from:

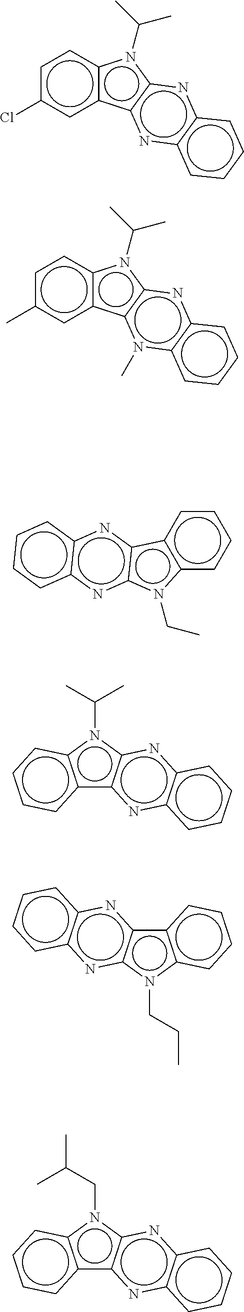

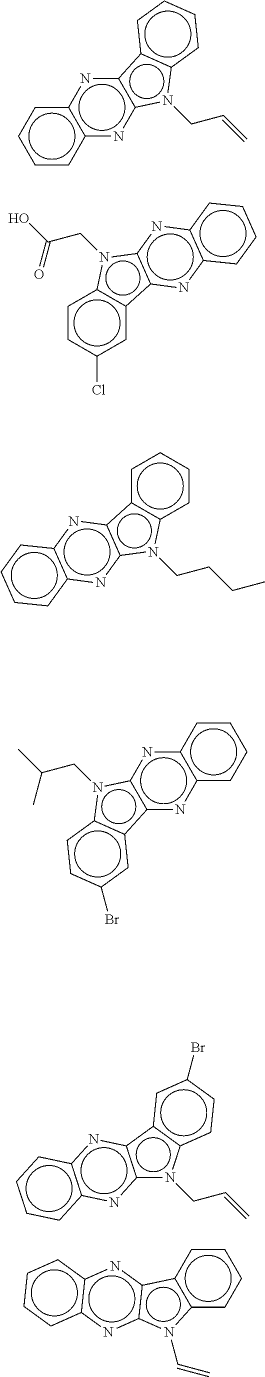

In other related embodiments, PDE7 inhibitors useful in the methods of the invention have the formula (33):

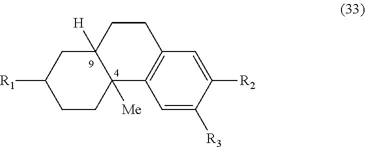

wherein
R₁ is CO, or alkylalcohol, R₂ is alkyl, R₃ is alkoxy, and the C₄ and C₉ stereocenters are independently (R) or(S).

In certain embodiments R₁ is carbonyl, or 2-methylpropan-1-ol. In specific embodiments R₂ is methyl.

In certain embodiments, R₃ is methoxy. In more specific embodiments the compounds are selected from:

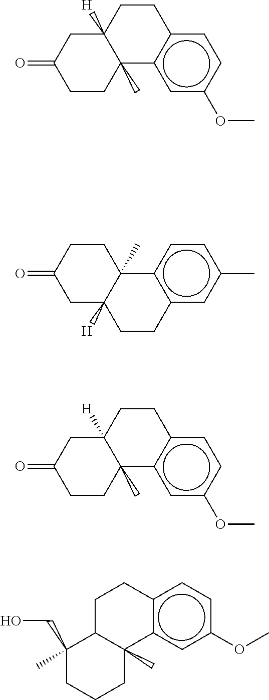

In another related embodiment, PDE7 inhibitors useful in the methods of the invention have the formula (34):

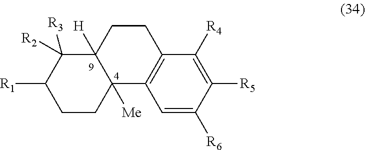

wherein
R₁ is hydrogen, hydroxyl, carbonyl, or alkylalcohol,
R₂ and R₃ are independently selected from hydrogen, alkyl, alkylcarboxylate, or carboxylic acid,
R₄ is hydrogen, or alkyl,
R₅ is hydrogen, alkyl, hydroxyl, or acetate,
R₆ is hydrogen, or alkoxy, and the C₄ and C₉ stereocenters are independently (R) or(S).
((33), (34): Also referred to as methylphenanthrene derivatives.)

In certain embodiments R1 is 2-methylpropan-1-ol. In specific embodiments R2 is methyl. In certain embodiments, R2 is methylcarboxylate. In specific embodiments R₂ and R3 are both methyl. In other embodiments, R2 is methyl, and R3 is methylcarboxylate. In specific embodiments R4 is iso-propyl. In specific embodiments, R5 is methyl. In certain embodiments, R6 is methoxy.

In more specific embodiments the compounds are selected from:

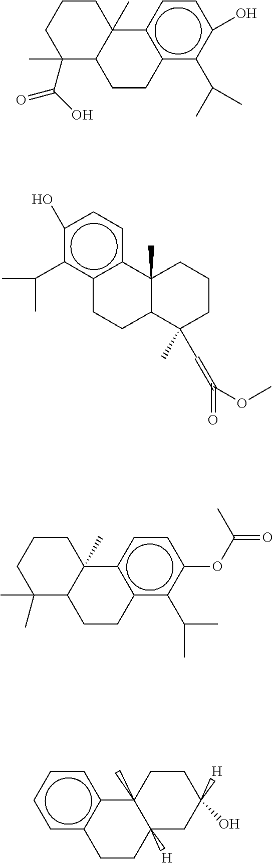

In regards to the above compounds, the terms “alkyl”, “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, adamantly, norbornane, and norbornee. This is also true of groups that include the prefix “alkyl-”, such as alkylcarboxylic acid, alkyl alcohol, alkylcarboxylate, alkylaryl, and the like. Examples of suitable alkylcarboxylic acid groups are methylcarboxylic acid, ethylcarboxylic acid, and the like. Examples of suitable alkylalcohols are methylalcohol, ethylalcohol, isopropylalcohol, 2-methylpropan-1-ol, and the like. Examples of suitable alkylcarboxylates are methylcarboxylate, ethylcarboxylate, and the like. Examples of suitable alkyl aryl groups are benzyl, phenylpropyl, and the like.

The term “aryl” as used herein includes carbocyclic aromatic rings or ring systems. Examples of aryl groups include phenyl, naphthyl, biphenyl, fluorenyl and indenyl. The term “heteroaryl” includes aromatic rings or ring systems that contain at least one ring hetero atom (e.g., O, S, N). Suitable heteroaryl groups include furyl, thienyl, pyridyl, quinolinyl, isoquinolinyl, indolyl, isoindolyl, thiazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

The aryl, and heteroaryl groups can be unsubstituted or substituted by one or more substituents independently selected from the group consisting of alkyl, alkoxy, methylenedioxy, ethylenedioxy, alkylthio, haloalkyl, haloalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonylthio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, arylcarbonylaminoalkyl, heteroarylcarbonylatnino, heteroarylalky carbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted,” then those groups can also be substituted by one or more of the above enumerated substituents.

In another embodiment, PDE7 inhibitors useful in the methods of the invention are selected from those compounds generally or specifically disclosed in WO 2008/142550 and US 2010/0216823, expressly incorporated herein by reference in its entirety. In one embodiment, PDE7 inhibitors useful in the methods of the invention have the formula (35):

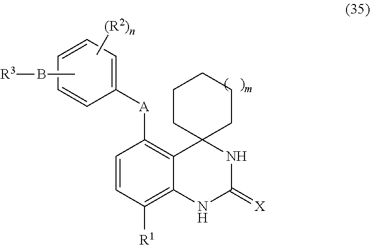

((35): Also referred to as spirocyclic and substituted quinazoline derivatives.)

The substituents for the above formula (35) are defined as follows:
m is 0, 1 or 2, n is 0, 1, 2 or 3,
X is O, S or N—CN,
R¹ is halogen or CN,
A is a single bond, CH₂, O or S,
B is a single bond, CH₂ or OCH₂, each R² is independently halogen, (C₁₋₆)alkyl (optionally substituted
by 1 to 3 fluorine atoms), OH, (C₁₋₆)alkylthio or CN,
R³ is selected from the following groups (i) to (x):

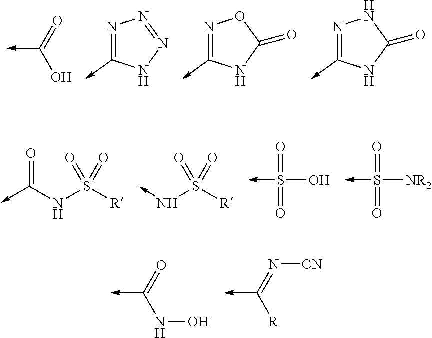

R is H or (C₁₋₆)alkyl (optionally substituted by 1 to 3 fluorine atoms), R′ is (C₁₋₆)alkyl (optionally substituted by 1 to 3 fluorine atoms), or a pharmaceutically acceptable salt, solvate, polymorph or prodrug thereof.

In regard to the above compounds, the term “alkyl” denotes a monovalent, straight or branched, saturated hydrocarbon chain containing 1 to 6 carbon atoms Examples of alkyl groups include methyl, ethyl, n-propyl, isopropyl, n-butyl, isobutyl, sec-butyl, tert-butyl, n-pentyl, 2-methylbutyl, 3-methylbutyl, neopentyl, n-hexyl, 2-methylpentyl, 3-methylpentyl, 4-methylpentyl, 2-ethylbutyl and 2,2-dimethylbutyl. Preferred alkyl groups are particularly methyl and ethyl, especially methyl. Where stated, alkyl groups may be substituted by 1 to 3 fluorine atoms. The substitution may be at any position on the alkyl chain. Preferably, such fluorinated alkyl groups have 1 to 4 carbon atoms, more preferably 1 or 2 carbon atoms. Mono-, di- and trifluoromethyl groups (especially trifluoromethyl), and mono-, di- and trifluoroethyl groups (especially 2,2,2-trifluoroethyl) are especially preferred.

The term “alkoxy” denotes “alkyl-O—”, wherein “alkyl” is as defined above, either in its broadest aspect or a preferred aspect. Preferred alkoxy groups are groups, particularly methoxy and ethoxy. The term “alkylthio” denotes “alkyl-S—”, wherein “alkyl” is as defined above, either in its broadest aspect or a preferred aspect. Preferred alkylthio groups are (C₁₋₄)alkylthio groups, particularly methylthio and ethylthio. The term “halogen” denotes fluoro, chloro, bromo or iodo. Preferred halogen groups are fluoro and chloro.

Preferably, m is 0 or 1, more preferably 1.

Preferably, n is 0 or 1, more preferably 0.

Preferably, X is O or N—CN, more preferably O.

Preferably, R¹ is F or Cl, more preferably C₁.

Preferably, A is a single bond or O, more preferably O.

When the group B is OCH₂, the oxygen atom is bonded to the benzene ring and the methylene group to the group R³.

Preferably, B is a single bond.

Preferably, R² is F or Cl, more preferably F.

Preferably, R³ is a group (i), (ii), (iii), (iv), (v) or (vi), more preferably a group (i) or (ii), and especially a group (ii).

In one embodiment, the group —B—R³ is present at the 2-position of the phenyl ring (the position of the group A being the 1-position). In other embodiments, the group —B—R³ is present at the 3-position In further embodiments, the group —B—R³ is present at the 4-position.

PDE7 inhibitors useful in the methods of the invention include those in which each variable in the above formula is selected from the suitable and/or preferred groups for each variable. Even more preferred PDE7 inhibitors useful in the methods of the invention include those where each variable in the above formula is selected from the more preferred or most preferred groups for each variable. In a related embodiment, the following PDE7 inhibitors are useful in the methods of the invention: 5-[(8′-chloro-2′-oxo-2′,3′-dihydro-1′H-spiro[cyclohexane-1,4′-quinazolin]-5′-yl)]-2-fluorobenzoic acid, 3-(8′-chloro-2-oxo-2′,3′-dihydro-1′H-spiro[cyclohexane-1,4′-quinazolin]-5′-ylbenzoic acid, 5-[(8′-chloro-2′-oxo-2′,3′-dihydro-1′H-spiro[cyclohexane-1,4′-quinazolin]-4′-yl)]-2-fluorobenzoic acid, 8-chloro-5′-[4-fluoro-3-(2H-tetrazol-5-yl)phenyl]-1′H-spiro[cyclohexane-1,4′-quinazolin]-2′(3′H)-one, [3-(8′-chloro-2′-oxo-2′,3′-dihydro-1′H-spiro[cyclohexane-1,4′-quinazolin]-5′-yl)phenoxy]acetic acid, 2-{(8′-chloro-2′-oxo-2,3′-dihydro-1′H-spiro[cyclohexane-1,4′-quinazolin]-5′-yl)oxy}-3-fluorobenzoic acid, 2-{(8′-chloro-2′-oxo-2′,3′-dihydro-1′H-spiro[cyclopentane-1,4′-quinazolin]-5′-oxy}-3-fluorobenzoic acid, 3-chloro-2-{(8′-chloro-2′-oxo-2′,3′-dihydro-1′H-spiro[cyclohexane-1,4′-quinazolin]-5′-yl)oxy}benzoic acid, 3-chloro-2-{(8′-fluoro-2′-oxo-2′,3′-dihydro-1′H-spiro[cyclohexane-1,4′-quinazolin]-5′-yl)oxy}benzoic acid, 8′-chloro-5′-[2-fluoro-6-(2H-tetrazol-5-yl)phenoxy]-1′H-spiro[cyclohexane-1,4′-quinazolin]-2′(3′H)-one, 8′-chloro-5′-[4-fluoro-2-(1H-tetrazol-5-yl)phenoxy]-1′H-spiro[cyclohexane-1,4′-quinazolin]-2′(3′H)-one, 8′-chloro-5′-[6-fluoro-2-(1H-tetrazol-5-yl)phenoxy]-1′H-spiro[cyclohexane-1,4′-quinazolin]-2′(3′H)-one, 8′-chloro-5′-[4-fluoro-2-(1H-tetrazol-5-yl)phenoxy]-1′H-spiro[cyclopentane-1,4′-quinazolin]-2′(3′H)-one, 8′-chloro-5′-[6-fluoro-2-(1H-tetrazol-5-yl)phenoxy]-1′H-spiro[cyclopentane-1,4′-quinazolin]-2′(3′H)-one, 8′-chloro-5′-[6-chloro-2-(1H-tetrazol-5-yl)phenoxy]-1′H-spiro[cyclopentane-1,4′-quinazolin]-2′(3′H)-one, 8′-chloro-5′-[2-(1H-tetrazol-5-yl)phenoxy]-1′H-spiro[cyclopentane-1,4′-quinazolin]-2′(3′H)-one, 8′-chloro-5′-[2-(1H-tetrazol-5-yl)phenoxy]-1′H-spiro[cyclohexane-1,4′-quinazolin]-2′(3′H)-one, 8′-chloro-5′-[2-fluoro-6-(5-oxo-4,5-dihydro-1,2,4-oxadiazol-3-yl)phenoxy]-1′H-spiro[cyclohexane-1,4′-quinazolin]-2′(3′H)-one, 8′-chloro-5′-[2-fluoro-6-(5-oxo-4,5-dihydro-1H-1,2,4-triazol-3-yl)phenoxy]-1′H-spiro[cyclohexane-1,4′-quinazolin]-2′(3′H)-one, 2-[(8′-chloro-2′-oxo-2′,3′-dihydro-1′H-spiro[cyclohexane-1,4′-quinazolin]-5′-yl)oxy]-3-fluoro-N-(methylsulfonyl)benzamide, N-{2-[(8′-chloro-2′-oxo-2′,3′-dihydro-1′H-spiro[cyclohexane-1,4′-quinazolin]-5′-yl)oxy]-3-fluorophenyl}-1,1,1-trifluoromethanesulfonamide, {2-[(8′-chloro-2′-oxo-2′,3′-dihydro-1′H-spiro[cyclohexane-1,4′-quinazolin]-5′-yl)oxy]-3-fluorophenyl}acetic acid, {2-[(8′-chloro-2′-oxo-2′,3′-dihydro-1′H-spiro[cyclohexane-1,4′-quinazolin]-5′-yl)oxy]phenoxy}acetic acid, {4-[(8′-chloro-2′-oxo-2′,3′-dihydro-1′H-spiro[cyclohexane-1,4′-quinazoline-5′-yl)oxy]phenoxy}acetic acid, methyl 2-[(8′-chloro-2′-oxo-2′,3′-dihydro-1′H-spiro[cyclohexane-1,4′-quinazolin]-5′-yl)oxy]-3-fluorobenzoate, and pharmaceutically acceptable salts, solvates and prodrugs thereof.

In another related embodiment, the following PDE7 inhibitors are useful in the methods of the invention: 8′-chloro-5′-[2-fluoro-6-(2H-tetrazol-5-yl)phenoxy]-1′H-spirocyclohexane-1,4′-quinazolin]-2′(3′H)-one, 8′-chloro-5′-[4-fluoro-2-(1H-tetrazol-5-yl)phenoxy]-1′H-spiro[cyclohexane-1,4′-quinazolin]-2′(3′H)-one, 8′-chloro-5′-[6-fluoro-2-(1H-tetrazol-5-yl)phenoxy]-1′H-spiro[cyclohexane-1,4′-quinazolin]-2′(3′H)-one, 8′-chloro-5′-[4-fluoro-2-(1H-tetrazol-5-yl)phenoxy]-1′H-spiro[cyclopentane-1,4′-quinazolin]-2′(3′H)-one, 8′-chloro-5′-[6-fluoro-2-(1H-tetrazol-5-yl)phenoxy]-1′H-spiro[cyclopentane-1,4′-quinazolin]-2′(3′H)-one, 8′-chloro-5′-[6-chloro-2-(1H-tetrazol-5-yl)phenoxy]-1′H-spiro[cyclopentane-1,4′-quinazolin]-2′(3′H)-one, 8′-chloro-5′-[2-(1H-tetrazol-5-yl)phenoxy]-1′H-spiro[cyclopentane-1,4′-quinazolin]-2′(3′H)-one, 8′-chloro-5′-[2-(1H-tetrazol-5-yl)phenoxy]-1′H-spiro[cyclohexane-1,4′-quinazolin]-2′(3′H)-one,
and pharmaceutically acceptable salts, solvates and prodrugs thereof.

The following compounds are most preferred: 8′-chloro-5′-[2-fluoro-6-(2H-tetrazol-5-yl)phenoxy]-1′H-spiro[cyclohexane-1,4′-quinazolin]-2′(3′H)-one, 8′-chloro-5′-[4-fluoro-2-(1H-tetrazol-5-yl)phenoxy]-1′H-spiro[cyclohexane-1,4′-quinazolin]-2′(3′H)-one, 8′-chloro-5′-[6-fluoro-2-(1H-tetrazol-5-yl)phenoxy]-1′H-spiro[cyclopentane-1,4′-quinazolin]-2′(3′H)-one, 8′-chloro-5′-[2-(1H-tetrazol-5-yl)phenoxy]-1′H-spiro[cyclohexane-1,4′-quinazolin]-2′(3′H)-one,
and pharmaceutically acceptable salts, solvates and prodrugs thereof.

In one embodiment, a PDE7 inhibitor useful in the methods of the invention has the formula:

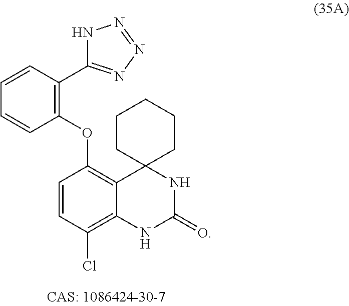 [SMILES: O=C1Nc2c(Cl)ccc(Oc3ccccc3-c3nnn[nH]3)c2C2(CCCCC2)N1]

The formula of 35A is also referred to in ISBN: 978-3-527-33219-9 (Liras and Bell, Phosphodiesterases and Their Inhibitors, Wiley-VCH).

The preparation of these compounds is described in WO 2008/142550 and US 2010/0216823.

In another embodiment, PDE7 inhibitors useful in the methods of the invention are selected from those compounds generally or specifically disclosed in U.S. Pat. No. 7,498,334, US 2005/0059686 and WO 2003/055882, each expressly incorporated herein by reference in its entirety. In one embodiment, PDE7 inhibitors useful in the methods of the invention have the formula (36):

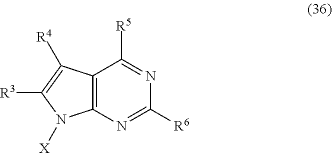

((36): Also referred to as pyrrolopyrimidine derivatives.)

The substituents for the above formula (36) are defined as follows:
X is phenyl or Het, each of which is unsubstituted or monosubstituted or polysubstituted by R₁ and/or R₂, R₁ and R₂ are each, independently of one another, A, OH, OA, SA, SOA, SO2A, SO2NH2, SO2NHA, SO2AA′, CN, NO2, NH2, NHA, NAA′, NHCOA, NHCOOA, COOH, COOA, CONH2, CONHA, CONAA′ or Hal, R′ and R₂ together are alternatively —OCH2O— or —OCH2CH2O—, R³ is A, OH, OA, SA, SOA, SO2A, SO2NH2, SO2NHA, SO2AA′, CN, NO2, NH2, NHA, NHB, NAA′, NHCOA, NHCOOA, NHCOB, NHCOOB, COOH, COOA, COOB, CONH2, CONHA, CONHB, CONAA′ or Hal, R⁴ is branched or unbranched alkyl or alkenyl having up to 10 carbon atoms, which may be substituted by from 1 to 5 F and/or Cl atoms and/or in which one or more CH2 groups may be replaced by O, S, SO, SO₂, NH, NA, NHCO, NACO, NHCOO or NACOO, or cycloalkyl or cycloalkenyl having from 3 to 7 carbon atoms, in which one or two CH2 groups may be replaced by O, S, SO, SO2, SO2NH, SO2NA, NH, NHA, NHCONH, NACONH, NACONA, NHCO, NACO, NHCOO or NACOO, R⁵ is OH, OA, SA, SOA, SO2A, SO2NH2, SO2NHA, SO2AA′, CN, NO2, NH2, NHA, NAA′, NHCOA, NHCOOA, COOH, COOA, CONH2, CONHA, CONAA′ or Hal, R⁶ is H, OH, OA, SA, SOA, SO2A, SO2NH2, SO2NHA, SO2AA′, CN, NO2, NH2, NHA, NAA′, NHCOA, NHCOOA, COOH, COOA, CONH2, CONHA, CONAA′ or Hal, A and A′ are each, independently of one another, branched or unbranched alkyl or alkenyl having up to 10 carbon atoms, which may be substituted by from 1 to 5 F and/or Cl atoms and/or in which one or more CH2 groups may be replaced by O, S, SO, SO2, NH, NR7, NHCO, NR7CO, NHCOO or NR7COO. A and A′ together are alternatively alkylene having from 3 to 7 carbon atoms, in which one or two CH2 groups may be replaced by CHR7, CHR7R8, O, S, SO, SO2, NH, NR7, NHCO, NR7CO, NHCOO or NR7COO. B is phenyl or Het, each of which is unsubstituted or monosubstituted or polysubstituted by R1 and/or R2, Het is an aromatic 5- or 6-membered heterocyclic ring having 1-3 N, O and/or S atoms which is unsubstituted or monosubstituted, disubstituted or trisubstituted by A″, Hal or CF3, R7 and R8 are each, independently of one another, branched or unbranched alkyl or alkenyl having up to 5 carbon atoms, which may be substituted by from 1 to 5 F and/or Cl atoms and/or in which one or more CH2 groups may be replaced by O, S, SO, SO2 or NH, A″ is alkyl having from 1 to 6 carbon atoms, and Hal is F, Cl, Br or I, and pharmaceutically usable derivatives, solvates and stereoisomers thereof, including mixtures thereof in all ratios.

In a related embodiment, PDE7 inhibitors useful in the methods of the invention include compounds of the above formula in which R⁵ is OH may also be in the form of their tautomers of the formula:

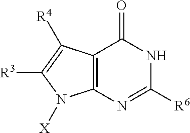

In regard to the above compounds, PDE7 inhibitors useful in methods of the invention include the optically active forms (stereo-isomers), the enantiomers, the racemates, the diastereomers and the hydrates and solvates of these compounds. The term solvates of the compounds is taken to mean adductions of inert solvent molecules onto the compounds which form owing to their mutual attractive force. Solvates are, for example, monohydrates, dihydrates or alcoholates.

In regards to the above compounds, the term pharmaceutically usable derivatives is taken to mean, for example, the salts of the above compounds and so-called prodrug compounds. The term prodrug derivatives is taken to mean, for example, the above compounds which have been modified, for example, with alkyl or acyl groups, sugars or oligopeptides and which are rapidly cleaved in the organism and thus release the active compounds. These also include biodegradable polymer derivatives of the above compounds, as described, for example, in Int. J. Pharm. 115, 61-67 (1995). In regard to the above compounds, the meanings of all radicals which occur more than once are in each case independent of one another.

A and A′ are preferably alkyl, furthermore preferably alkyl which is substituted by from 1 to 5 fluorine and/or chlorine atoms, furthermore preferably alkenyl.

In the above formulae, alkyl is preferably unbranched and has 1, 2, 3, 4, 5, 6, 7, 8, 9 or 10 carbon atoms, preferably 1, 2, 3, 4, 5 or 6 carbon atoms, and is preferably methyl, ethyl, trifluoromethyl, pentafluoroethyl or propyl, furthermore preferably isopropyl, butyl, isobutyl, sec-butyl or tert-butyl, but also n-pentyl, neopentyl, isopentyl or n-hexyl. Particular preference is given to methyl, ethyl, trifluoromethyl, propyl, isopropyl, butyl, n-pentyl, n-hexyl or n-decyl.

A″ is preferably alkyl having 1, 2, 3, 4, 5 or 6 carbon atoms, for example methyl, ethyl or propyl, furthermore preferably isopropyl, butyl, isobutyl, sec-butyl or tert-butyl, but also n-pentyl, neopentyl, isopentyl or n-hexyl. Particular preference is given to methyl, ethyl, propyl, isopropyl or butyl.

Cycloalkyl preferably has 3-7 carbon atoms and is preferably cyclopropyl or cyclobutyl, furthermore preferably cyclopentyl or cyclohexyl, furthermore also cycloheptyl; particular preference is given to cyclopentyl.

Alkenyl is preferably vinyl, allyl, 2- or 3-butenyl, isobutenyl or sec-butenyl; preference is furthermore given to 4-pentenyl, isopentenyl or 5-hexenyl.

Alkylene is preferably unbranched and is preferably methylene or ethylene, furthermore preferably propylene or butylene.

Hal is preferably F, Cl or Br, furthermore also I.

The radicals R₁ and R₂ may be identical or different and are preferably in the 2- or 4-position of the phenyl ring. They are, for example, independently of one another, A or Hal, or together are methylenedioxy.

However, they are preferably each methyl, ethyl, propyl, methoxy, ethoxy, propoxy, isopropoxy, benzyloxy, but also fluoro-, difluoro- or trifluoro-methoxy, or 1-fluoro-, 2-fluoro-, 1,2-difluoro-, 2,2-difluoro-, 1,2,2-trifluoro- or 2,2,2-trifluoroethoxy, furthermore fluorine or chlorine.
R₁ is particularly preferably fluorine, chlorine, methyl, ethyl or propyl.
R₂ is particularly preferably fluorine, chlorine, methyl, ethyl or propyl.
X is preferably a phenyl radical which is monosubstituted by R₁ or is unsubstituted Het.
X is particularly preferably 2-chlorophenyl, 2-fluorophenyl, 4-methyl-phenyl, 3-chlorophenyl or 4-chlorophenyl.
Het is preferably, for example, unsubstituted 2- or 3-furyl, 2- or 3-thienyl, 1-, 2- or 3-pyrrolyl, 1-, 2-, 4- or 5-imidazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, or 1,2,3-thia-diazol-4- or -5-yl.
R³ is preferably, for example, COOA″ or COOH.
R⁴ is preferably, for example, unbranched or branched alkyl having 1, 2, 3, 4, 5 or 6 carbon atoms, which may be substituted by 1-5 F or Cl atoms, preferably methyl, ethyl, trifluoromethyl, pentafluoroethyl or propyl, furthermore preferably isopropyl, butyl, isobutyl, sec-butyl or tert-butyl, but also n-pentyl, neopentyl, isopentyl or n-hexyl. Particular preference is given to methyl, ethyl, trifluoromethyl, propyl, isopropyl, butyl, n-pentyl, n-hexyl or n-decyl.
R⁵ is preferably Cl or OH.
R⁶ is preferably H.

In regard to the above compounds, at least one of the said radicals has one of the preferred meanings indicated above.

In a related embodiment, PDE7 inhibitors useful in the methods of the invention include the following compounds, wherein X is a phenyl radical which is monosubstituted by R₁, or is unsubstituted Het; R¹ is A or Hal; R³ is COOA″ or COOH; R⁴ is unbranched or branched alkyl having 1, 2, 3, 4, 5 or 6 carbon atoms, which may be substituted by 1-5 F or Cl atoms; R⁵ is Cl or OH; and R⁶ is H; In other related embodiments, PDE7 inhibitors useful in the methods of the invention include the following compounds, wherein X is a phenyl radical which is monosubstituted by R¹, or is unsubstituted Het, R¹ is A or Hal, R³ is COOA″ or COOH, R⁴ is unbranched or branched alkyl having 1, 2, 3, 4, 5 or 6 carbon atoms, which may be substituted by 1-5 F or Cl atoms, R⁵ is Cl or OH, R⁶ is H, Het is furyl, thienyl, pyrrolyl, imidazolyl, pyridyl or pyrimidinyl, A and A″ are each, independently of one another, unbranched or branched alkyl having 1, 2, 3, 4, 5 or 6 carbon atoms, which may be substituted by 1-5 F or Cl atoms, Hal is F, Cl or Br, and pharmaceutically usable derivatives, solvates and stereoisomers thereof, including mixtures thereof in all ratios.

The preparation of the above compounds and also the starting materials for their preparation are described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

In another related embodiment, PDE7 inhibitors useful in the methods of the invention include: ethyl 5-isopropyl-4-oxo-7-p-tolyl-4,7-dihydro-3H-pyrrolo[2,3-d]-pyrimidine-6-carboxylate, ethyl 5-methyl-4-oxo-7-(3-chlorophenyl)-4,7-dihydro-3H-pyrrolo[2,3-d]pyrimidine-6-carboxylate, ethyl 5-methyl-4-oxo-7-(2-chlorophenyl)-4,7-dihydro-3H-pyrrolo[2,3-d]pyrimidine-6-carboxylate, ethyl 5-methyl-4-oxo-7-(2-fluorophenyl)-4,7-dihydro-3H-pyrrolo[2,3-d]pyrimidine-6-carboxylate, ethyl 5-propyl-4-oxo-7-(2-chlorophenyl)-4,7-dihydro-3H-pyrrolo[2,3-d]pyrimidine-6-carboxylate, ethyl 5-methyl-4-oxo-7-(4-chlorophenyl)-4,7-dihydro-3H-pyrrolo[2,3-d]pyrimidine-6-carboxylate, ethyl 5-methyl-4-oxo-7-p-tolyl-4,7-dihydro-3H-pyrrolo[2,3-d]-pyrimidine-6-carboxylate, methyl 5-methyl-4-oxo-7-(2-chlorophenyl)-4,7-dihydro-3H-pyrrolo[2,3-d]pyrimidine-6-carboxylate, methyl 5-methyl-4-oxo-7-phenyl-4,7-dihydro-3H-pyrrolo[2,3-d]-pyrimidine-6-carboxylate, methyl 5-methyl-4-oxo-7-(2-thienyl)-4,7-dihydro-3H-pyrrolo[2,3-d]-pyrimidine-6-carboxylate, and pharmaceutically usable derivatives, solvates and stereoisomers thereof, including mixtures thereof in all ratios.

The preparation of the above compounds is described in U.S. Pat. No. 7,498,334 and WO 2003/055882.

In another embodiment, PDE7 inhibitors useful in the methods of the invention are selected from those compounds generally or specifically disclosed in U.S. Pat. No. 6,884,800 and WO 2001/036425, each expressly incorporated herein by reference in its entirety. In one embodiment, PDE7 inhibitors useful in the methods of the invention have the formula (37):

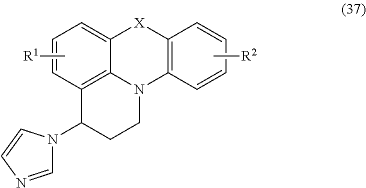

The substituents for the above formula (37) are defined as follows:
R¹ and R², independently of one another, each denote A1, OA1, SA1 or Hal, A1 denotes H, A, alkenyl, cycloalkyl or alkylenecycloalkyl, A denotes alkyl having 1-10 carbon atoms, Hal denotes F, Cl, Br or I, and x denotes O, S, SO or SO₂, and their physiologically acceptable salts and/or solvates.

In regard to the above compounds, A denotes alkyl having 1-10 carbon atoms and has 1, 2, 3, 4, 5, 6, 7, 8, 9 or 10 carbon atoms and preferably denotes methyl, ethyl or propyl, furthermore preferably isopropyl, butyl, isobutyl, sec-butyl or tert-butyl, but also n-pentyl, neopentyl, isopentyl or hexyl. In these radicals, 1-7H atoms may also be replaced by F and/or Cl. A therefore also denotes, for example, trifluoromethyl or pentafluoroethyl. Cycloalkyl has 3-9 carbon atoms and preferably denotes, for example, cyclopentyl or cyclohexyl. Alkenyl has 2-10 carbon atoms, is linear or branched and preferably denotes vinyl, propenyl or butenyl. Alkylenecycloalkyl has 4-10 carbon atoms and denotes, for example, methylenecyclopentyl, ethylenecyclopentyl, methylenecyclohexyl or ethylenecyclohexyl. R¹ and R² preferably denote, in each case independently of one another, H, fluorine, chlorine, methyl, ethyl, propyl, methoxy, ethoxy, propoxy, methylthio, cyclopentyl or cyclohexyl.

In a related embodiment, PDE7 inhibitors useful in the methods of the invention include the following compounds, wherein
X is S;
X is S, R¹ is H;
X is S, R¹ is F or Cl;
X is S, R² is H;
X is S, R² is F or Cl;
X is S, R¹ is H, R² is F or Cl;
X is S, R¹ is F or Cl, R² is H;
X is S; A1 is H or A, A is alkyl having 1, 2, 3 or 4 carbon atoms;
X is S, R¹ and R², independently of one another, each denote A1 or Hal, A1 is H or A, A is alkyl having 1, 2, 3 or 4 carbon atoms, Hal is F or Cl;
and their physiologically acceptable salts and solvates.

In another related embodiment, PDE7 inhibitors useful in the methods of the invention include the following compounds:
10-Chloro-3-imidazol-1-yl-2,3-dihydro-1H-pyrido[3,2,1-kl]phenothiazine, 4-chloro-3-imidazol-1-yl-2,3-dihydro-1H-pyrido[3,2,1-kl]phenothiazine, 10-methoxy-3-imidazol-1-yl-2,3-dihydro-1H-pyrido[3,2,1-kl]phenothiazine, 10-propoxy-3-imidazol-1-yl-2,3-dihydro-1H-pyrido[3,2,1-kl]phenothiazine, 10-methylthio-3-imidazol-1-yl-2,3-dihydro-1H-pyrido[3,2,1-kl]phenothiazine, 10-fluoro-3-imidazol-1-yl-2,3-dihydro-1H-pyrido[3,2,1-kl]phenothiazine, 4,10-dichloro-3-imidazol-1-yl-2,3-dihydro-1H-pyrido[3,2,1-kl]phenothiazine, 10-trifluoromethyl-3-imidazol-1-yl-2,3-dihydro-1H-pyrido[3,2,1-kl]phenothiazine, 4-cyclopentoxy-3-imidazol-1-yl-2,3-dihydro-1H-pyrido[3,2,1-kl]phenothiazine, 10-chloro-3-imidazol-1-yl-2,3-dihydro-1H-7-oxa-1 b-azabenzo[de]-anthracene, and 10-chloro-3-imidazol-1-yl-2,3-dihydro-1H-pyrido[3,2,1-kl]phenothiazine 7,7-dioxide.

The preparation of these compounds is described in U.S. Pat. No. 6,884,800 and WO 01/036425.

In another embodiment, PDE7 inhibitors useful in the methods of the invention are selected from those compounds generally or specifically disclosed in U.S. Pat. No. 6,531,498 and WO 2001/032175, each expressly incorporated herein by reference in its entirety.

In one embodiment, PDE7 inhibitors useful in the methods of the invention have the formula (38):

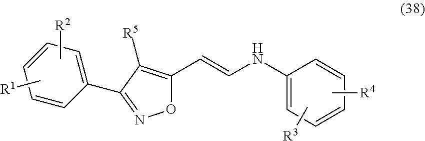

((38): Also referred to as isoxazole derivatives.)

The substituents of the above formula (38) are defined as follows:
R¹, R², R³, R⁴ are each, independently of one another, Ha1, OA1, SA1, A, H, COOA1, CN or CONA1A2,
R⁵ is COOA1, CN or CONA1A2,
A1, A2 are each, independently of one another, H, A, alkenyl, cycloalkyl or alkylenecycloalkyl,
A is alkyl having 1 to 10 C atoms,
Hal is F, Cl, Br or I,
and their physiologically acceptable salts and/or solvates.

In regard to the above compounds, A is alkyl having 1-10 C atoms and has 1, 2, 3, 4, 5, 6, 7, 8, 9 or 10 C atoms and is preferably methyl, ethyl or propyl, also preferably isopropyl, butyl, isobutyl, sec-butyl or tert-butyl, but is also n-pentyl, neopentyl, isopentyl or hexyl. It is also possible for 1-7H atoms in the radicals to be replaced by F and/or Cl. A is therefore also, for example, trifluoromethyl or pentafluoroethyl.

Cycloalkyl has 3-9 C atoms and is preferably, for example, cyclopentyl or cyclohexyl. Alkenyl has 2-10 C atoms, is linear or branched and is preferably vinyl, propenyl or butenyl.

Alkylenecycloalkyl has 4-10 C atoms and is, for example methylenecyclopentyl, ethylenecyclopentyl, methylenecyclohexyl or ethylenecyclohexyl.

In a related embodiment, PDE7 inhibitors useful in the methods of the invention include the compounds wherein
R¹ is H;
R¹ and R² are H;
R¹ is H and R² is F or Cl;
R¹, R² are each, independently of one another, H or Hal;
R¹, R² are each, independently of one another, H or Hal, A1, A2 are each, independently of one another, H or A;
A1, A2 are each, independently of one another, H or A;
R¹, R² are each, independently of one another, H or Hal, A1, A2 are each, independently of one another, H or A, A is alkyl having 1, 2, 3 or 4 C atoms, Hal is F or Cl.

In another related embodiment, PDE7 inhibitors useful in the methods of the invention include the compounds:
5-[2-(2-Fluoro-4-hydroxyphenylamino)vinyl]-4-cyano-3-phenylisoxazole, 5-[2-(2,4-Difluorophenylamino)vinyl]-4-cyano-3-phenylisoxazole, 5-[2-(3-Methylthiophenylamino)vinyl]-4-cyano-3-phenylisoxazole, 5-[2-(2,4-Dimethoxyphenylamino)vinyl]-4-cyano-3-(2-chlorophenyl)isoxazole, 5-(2-Amino-2-phenylvinyl)-4-methylaminocarbonyl-3-phenylisoxazole, 5-(2-Phenylaminovinyl)-4-methoxycarbonyl-3-phenylisoxazole, 5-[2-(4-Carboxyphenylamino)vinyl]-4-cyano-3-phenylisoxazole, 5-[2-(4-Carboxyphenylamino)vinyl]-4-methoxycarbonyl-3-phenylisoxazole, 5-[2-(5-Chloro-2-hydroxyphenylamino)vinyl]-4-cyano-3-phenylisoxazole, 5-[2-(3,4-Dimethylphenylamino)vinyl]-4-cyano-3-(2-chlorophenyl)isoxazole, 5-[2-(4-Chlorophenylamino)vinyl]-4-cyano-3-(2-chlorophenyl)isoxazole, 5-(2-Phenylaminovinyl)-4-cyano-3-(2-chlorophenyl)isoxazole, 5-[2-(4-Methoxyphenylamino)vinyl]-4-cyano-3-(2-chlorophenyl)isoxazole, 5-[2-(4-Carboxyphenylamino)vinyl]-4-cyano-3-(2-chlorophenyl)isoxazole, 5-[2-(2-Fluoro-4-hydroxyphenylamino)vinyl]-4-cyano-3-(2-chlorophenyl)isoxazole, S-[2-(4-Fluorophenylamino)vinyl]-4-cyano-3-(2-chlorophenyl)isoxazole, 5-[2-(3,5-Dichlorophenylamino)vinyl]-4-cyano-3-(2-chlorophenyl)isoxazole, S-[2-(3-Chlorophenylamino)vinyl]-4-cyano-3-(2-chlorophenyl)isoxazole, S-(2-Phenylaminovinyl)-4-cyano-3-(2,6-dichlorophenyl)isoxazole, S-[2-(4-Chlorophenylamino)vinyl]-4-cyano-3-(2,6-dichlorophenyl)isoxazole, S-(2-Phenylaminovinyl)-4-methoxycarbonyl-3-(2,6-dichlorophenyl)isoxazole, 5-[2-(4-Chlorophenylamino)vinyl]-4-methoxycarbonyl-3-(2,6-dichlorophenyl)isoxazole, S-[2-(4-Carboxyphenylamino)vinyl]-4-methoxycarbonyl-3-(2,6-dichlorophenyl)isoxazole, S-[2-(2,4-Difluorophenylamino)vinyl]-4-cyano-3-(2,6-dichlorophenyl)isoxazole, 5-[2-(2,4-Dichlorophenylamino)vinyl]-4-cyano-3-(2,6-dichlorophenyl)isoxazole, S-[2-(4-Carboxyphenylamino)vinyl]-4-cyano-3-(2,6-dichlorophenyl)isoxazole, S-[2-(3,5-Dichlorophenylamino)vinyl]-4-cyano-3-(2,6-dichlorophenyl)isoxazole, 5-[2-(4-Methoxyphenylamino)vinyl]-4-cyano-3-(2,6-dichlorophenyl)isoxazole, 5-[2-(2,4-Dimethoxyphenylamino)vinyl]-4-cyano-3-(2,6-dichlorophenyl)isoxazole, 5-[2-(2-Phenylphenylamino)vinyl]-4-cyano-3-(2,6-dichlorophenyl)isoxazole, 5-[2-(4-Methylphenylamino)vinyl]-4-cyano-3-(2,6-dichlorophenyl)isoxazole, 5-(2-Phenylaminovinyl)-4-cyano-3-(2-chloro-6-fluorophenyl)isoxazole, 5-[2-(4-Carboxyphenylamino)vinyl]-4-cyano-3-(2-chloro-6-fluorophenyl)isoxazole, 5-[2-(4-Chlorophenylamino)vinyl]-4-cyano-3-(2-chloro-6-fluorophenyl)isoxazole, 5-[2-(3-Methoxyphenylamino)vinyl]-4-cyano-3-(2-chloro-6-fluorophenyl)isoxazole, 5-[2-(4-Chlorophenylamino)vinyl]-4-methoxycarbonyl-3-(2-chloro-6-fluorophenyl)isoxazole, 5-(2-Phenylaminovinyl)-4-methoxycarbonyl-3-(2-chloro-6-fluorophenyl)isoxazole, 5-[2-(2,4-Dichlorophenylamino)vinyl]-4-methoxycarbonyl-3-(2-chloro-6-fluorophenyl)isoxazole, 5-(2-Phenylaminovinyl)-4-cyano-3-phenylisoxazole, 5-[2-(3-Trifluoromethoxyphenylamino)vinyl]-4-cyano-3-phenylisoxazole, 5-[2-(4-Methoxyphenylamino)vinyl]-4-cyano-3-phenylisoxazole, 5-[2-(4-Methoxyphenylamino)vinyl]-4-methoxycarbonyl-3-(2-chloro-6-fluorophenyl)isoxazole, 5-[2-(3-Methylthiophenylamino)vinyl]-4-cyano-3-phenylisoxazole, 5-[2-(2,4-Difluorophenylamino)vinyl]-4-cyano-3-phenylisoxazole, 5-[2-(2-Fluoro-4-hydroxyphenylamino)vinyl]-4-cyano-3-phenylisoxazole.

The preparation of these compounds is described in U.S. Pat. No. 6,531,498 and WO 01/032175.

In another embodiment, PDE7 inhibitors useful in the methods of the invention are selected from those compounds generally or specifically disclosed in U.S. Pat. No. 7,491,742 and WO 2001/029049, each expressly incorporated by reference in its entirety.

In one embodiment, PDE7 inhibitors useful in the methods of the invention have the formula (39):

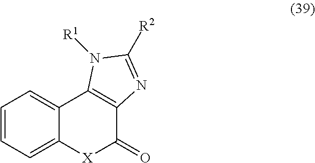

((37), (39): Also referred to as imidazole derivatives.)

The substituents of the above formula (39) are defined as follows:
R¹ is H, A, benzyl, indan-5-yl, 1,2,3,4-tetrahydronaphthalen-5-yl, dibenzothien-2-yl, or phenyl which is unsubstituted or mono-, di- or trisubstituted by Hal, A, A-CO—NH, benzyloxy, alkoxy, COOH or COOA,
R² is H or A, X is O or S, Hal is F, Cl, Br or I, A is alkyl with 1 to 6 C atoms, and the physiologically acceptable salts and/or solvates thereof.

In regard to the above compounds, A is alkyl with 1-6 C atoms and has 1, 2, 3, 4, 5 or 6 C atoms and is preferably methyl, ethyl or propyl, also preferably isopropyl, butyl, isobutyl, sec-butyl or tert-butyl, but also n-pentyl, neopentyl, isopentyl or hexyl. A is also cycloalkyl such as, for example, cyclohexyl. Alkoxy is preferably methoxy, ethoxy, propoxy or butoxy. Hal is preferably F or C₁. A-CO—NH is preferably acetamido.

In a related embodiment, PDE7 inhibitors useful in the methods of the invention are selected from the following compounds:
1-Phenyl-[1]benzopyrano[3,4-d]imidazol-4-(1H)-one, 1-Benzyl-[1]benzopyrano[3,4-d]imidazol-4-(1H)-one, 1-Cyclohexyl-[1]benzopyrano[3,4-d]imidazol-4-(1H)-one, 1-Cyclopentyl-[1]benzopyrano[3,4-d]imidazol-4-(1H)-one, 1-Butyl-[1]benzopyrano[3,4-d]imidazol-4-(1H)-one, 1-Isopropyl-[1]benzopyrano[3,4-d]imidazol-4-(1H)-one, 1-Propyl-[1]benzopyrano[3,4-d]imidazol-4-(1H)-one, 1-Ethyl-[1]benzopyrano[3,4-d]imidazol-4-(1H)-one, 1-Methyl-[1]benzopyrano[3,4-d]imidazol-4-(1H)-one, [1]Benzopyrano[3,4-d]imidazol-4-(1H)-one, 2-Methyl-[1]benzopyrano[3,4-d]imidazol-4-(1H)-one, 1-Phenyl-[1]benzothiopyrano[3,4-d]imidazol-4-(1H)-one, 1-Benzyl-[1]benzothiopyrano[3,4-d]imidazol-4-(1H)-one, 1-Cyclohexyl-[1]benzothiopyrano[3,4-d]imidazol-4-(1H)-one, 1-Cyclopentyl-[1]benzothiopyrano[3,4-d]imidazol-4-(1H)-one, 1-Butyl-[1]benzothiopyrano[3,4-d]imidazol-4-(1H)-one, 1-Isopropyl-[1]benzothiopyrano[3,4-d]imidazol-4-(1H)-one, 1-Propyl-[1]benzothiopyrano[3,4-d]imidazol-4-(1H)-one, 1-Ethyl-[1]benzothiopyrano[3,4-d]imidazol-4-(1H)-one, 1-Methyl-[1]benzothiopyrano[3,4-d]imidazol-4-(1H)-one, [1]Benzothiopyrano[3,4-d]imidazol-4-(1H)-one, 2-Methyl-[1]benzothiopyrano[3,4-d]imidazol-4-(1H)-one, 1-(2-Chlorophenyl-[1]benzopyrano[3,4-d]imidazol-4-(1H)-one, 1-(4-Methyl-phenyl)-[1]benzopyrano[3,4-d]imidazol-4-(1H)-one, 1-(4-Fluorophenyl)-[1]benzopyrano[3,4-d]imidazol-4-(1H)-one, 1-(2,4-Dimethyl-phenyl)-[1]benzopyrano[3,4-d]imidazol-4-(1H)-one, 1-(3-Chlorophenyl)-[1]benzopyrano[3,4-d]imidazol-4-(1H)-one, 1-(2,4-Dichlorophenyl)-[1]benzopyrano[3,4-d]imidazol-4-(1H)-one, 1-(2,5-Dichlorophenyl)-[1]benzopyrano[3,4-d]imidazol-4-(1H)-one, 1-(4-Acetamido-phenyl)-[1]benzopyrano[3,4-d]imidazol-4-(1H)-one, 1-(2-Fluorophenyl)-[1]benzopyrano[3,4-d]imidazol-4-(1H)-one, 1-(3-Fluorophenyl)-[1]benzopyrano[3,4-d]imidazol-4-(1H)-one, 1-(2-Benzyloxy-phenyl)-[1]benzopyrano[3,4-d]imidazol-4-(1H)-one, 1-(2,6-Dimethyl-phenyl)-[1]benzopyrano[3,4-d]imidazol-4-(1H)-one, 1-(Indan-5-yl)-[1]benzopyrano[3,4-d]imidazol-4-(1H)-one, 1-(2-Methoxy-phenyl)-[1]benzopyrano[3,4-d]imidazol-4-(1H)-one, 1-(2,3-Dimethyl-phenyl)-[1]benzopyrano[3,4-d]imidazol-(1H)-4-one, 1-(2,3-Dichlorophenyl)-[1]benzopyrano[3,4-d]imidazol-4-(1H)-one, 1-(3-Chloro-4-methyl-phenyl)-[1]benzopyrano[3,4-d]imidazol-4-(1H)-one, 1-(2,5-Dimethyl-phenyl)-[1]benzopyrano[3,4-d]imidazol-4-(1H)-one, 1-(4-Chlorophenyl)-[1]benzopyrano[3,4-d]imidazol-4-(1H)-one, 1-(1,2,3,4-Tetrahydronaphthalen-5-yl)-[1]benzopyrano-[3,4-d]imidazol-4-(1-H)-one, 1-(Dibenzothien-2-yl)-[1]benzopyrano[3,4-d]imidazol-4-(1H)-one, 1-(3-Methoxy-phenyl)-[1]benzopyrano[3,4-d]imidazol-4-(1H)-one, 1-(4-Carboxy-2-methyl-phenyl)-[1]benzopyrano[3,4-d]imidazol-4-(1H)-one, and their physiologically acceptable salts and/or solvates thereof.

In another embodiment, PDE7 inhibitors useful in the methods of the invention are selected from those compounds generally or specifically disclosed in U.S. Pat. No. 6,737,436 and WO 2001/032618, expressly incorporated herein by reference in its entirety.

In one embodiment, PDE7 inhibitors useful in the methods of the invention have the formula (40):

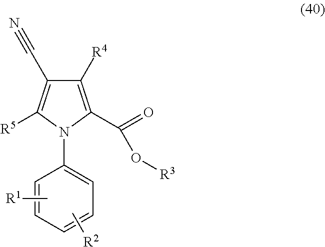

((40): Also referred to as 4-cyano-1H-pyrrole-2-carboxylate derivatives.)

The substituents for the above formula (40) are defined as follows:
R¹ and R², independently of one another, each denote H, A, OA, SA or Hal,
R³ denotes H or A,
R⁴ denotes A or NH₂,
R⁵ denotes H, NH₂, NHA or NA2,
A denotes alkyl having 1 to 10 carbon atoms, alkenyl, cycloalkyl or alkylenecycloalkyl,
Hal denotes F, Cl, Br or I,
and their physiologically acceptable salts and/or solvates.

In regard to the above compounds, A denotes alkyl having 1-10 carbon atoms and has 1, 2, 3, 4, 5, 6, 7, 8, 9 or 10 carbon atoms and preferably denotes methyl, ethyl or propyl, furthermore preferably isopropyl, butyl, isobutyl, sec-butyl or tert-butyl, but also n-pentyl, neopentyl, isopentyl or hexyl. In these radicals, 1-7H atoms may also be replaced by F and/or Cl. A therefore also denotes, for example, trifluoromethyl or pentafluoroethyl.
A also denotes cycloalkyl having 3-8 carbon atoms and preferably denotes, for example, cyclopentyl or cyclohexyl.
A also denotes alkenyl. Alkenyl has 2-10 carbon atoms, is linear or branched and denotes, for example, vinyl, propenyl or butenyl. A furthermore denotes alkylenecycloalkyl. Alkylenecycloalkyl has 4-10 carbon atoms and preferably denotes, for example, methylenecyclopentyl, ethylenecyclopentyl, methylenecyclohexyl or ethylenecyclohexyl.
R¹ and R² preferably each denote, independently of one another, H, methyl, ethyl, propyl, butyl, isopropyl, tert-butyl, methoxy, ethoxy, propoxy, isopropoxy, butoxy, S-methyl, S-ethyl, F or Cl.
R³ preferably denotes H, methyl or ethyl.
R⁴ preferably denotes methyl, ethyl, propyl, butyl or NH2.
R⁵ preferably denotes H, amino, methylamino, ethylamino, dimethylamino or diethylamino.

In a related embodiment, PDE7 inhibitors useful in the methods of the invention include compounds of the above formula wherein R¹ and R² are not both H and wherein when one of R¹ or R² is H, the other cannot be CH3, OCH3 or C₁.

In another related embodiment, PDE7 inhibitors useful in the methods of the invention include compounds wherein
R¹, R², R³ and R⁵ are H and R⁴ is methyl;
R¹ is 4-C₁, R² is H, R³ is ethyl, R⁴ is amino and R⁵ is H;
R¹ and R² are H, R³ is ethyl, R⁴ is methyl and R⁵ is amino;
R¹ and R² are H, R³ is ethyl, R⁴ is amino and R⁵ is H;
R¹ and R² are H, R³ is ethyl, R⁴ is H and R⁵ is amino;
R¹ is 3-C₁, R² is 4-O-methyl, R³ is ethyl, R⁴ is amino and R⁵ is H;
R¹ is 3-C₁, R² is 4-O-methyl, R³ is ethyl, R⁴ is methyl and R⁵ is amino;
R¹ is 4-OCF3, R² is H, R³ is ethyl, R⁴ is amino and R⁵ is H;
R¹ is 3-Cl, R² is 4-O-methyl, R³ is ethyl, R⁴ is amino and R⁵ is H;
R¹ is 3-Cl, R² is 4-O-methyl, R³ is ethyl, R⁴ is methyl and R⁵ is amino;
R¹ is 4-OCF3, R² is H, R³ is ethyl, and R⁴ is amino and R⁵ is H.

In another embodiment, PDE7 inhibitors useful in the methods of the invention are selected from those compounds generally or specifically disclosed in U.S. Pat. No. 6,613,778 and WO 2001/034601, expressly incorporated herein by reference in its entirety.

In one embodiment, PDE7 inhibitors useful in the methods of the invention have the formula (41):

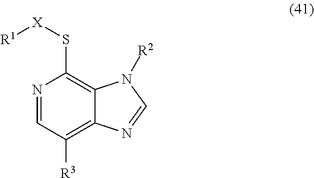

((41): Also referred to as imidazo[4,5-c]pyridine derivatives.)

The substituents for the above formula 41 are defined as follows:
R¹ denotes CONR4R5,
R² denotes H or A,
R4 and R5, independently of one another, each denote H or A1,
R³ denotes Hal,
Hal denotes F, Cl, Br or I,
A denotes alkyl having 1-4 carbon atoms,
A1 denotes alkyl having 1-10 carbon atoms,
X denotes alkylene having 1-4 carbon atoms, in which an ethylene group may also be replaced by a double or triple bond,
and their physiologically acceptable salts and/or solvates.

In regard to the above compounds, A denotes alkyl having 1-4 carbon atoms and has 1, 2, 3 or 4 carbon atoms and preferably denotes methyl, ethyl or propyl, furthermore preferably isopropyl, butyl, isobutyl, sec-butyl or tert-butyl. 1-7H atoms in the radicals may also be replaced by F and/or Cl. A therefore also denotes, for example, trifluoromethyl or pentafluoroethyl.

A1 denotes alkyl having 1-10 carbon atoms and has 1, 2, 3, 4, 5, 6, 7, 8, 9 or 10 carbon atoms and preferably denotes methyl, ethyl or propyl, furthermore preferably isopropyl, butyl, isobutyl, sec-butyl or tert-butyl, but also n-pentyl, neopentyl, isopentyl or hexyl. 1-7H atoms in the radicals may also be replaced by F and/or Cl. A1 therefore also denotes, for example, trifluoromethyl or pentafluoroethyl.

X denotes alkylene having 1-4 carbon atoms, preferably methylene, ethylene, propylene or butylene, in which one ethylene group may also be replaced by a double or triple bond. X therefore also denotes, for example, —CH2-CH═CH—H2- or —C≡C—.

In a related embodiment, PDE7 inhibitors useful in the methods of the invention include the following compounds: 2-(3-Butyl-7-chloro-3H-imidazo[4,5-c]pyridin-4-ylsulfanyl)-N,N-dimethylacetamide

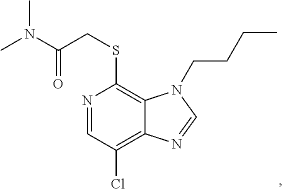 [SMILES: CCCCn1cnc2c(Cl)cnc(SCC(=O)N(C)C)c21]

### 2-(3-butyl-7-chloro-3H-imidazo[4,5-c]pyridin-4-ylsulfanyl) acetamide, 2-(3-butyl-7-chloro-3H-imidazo[4,5-c]pyridin-4-ylsulfanyl) propionamide, 2-(3-butyl-7-chloro-3H-imidazo[4,5-c]pyridin-4-ylsulfanyl) butyramide, 2-(3-butyl-7-chloro-3H-imidazo[4,5-c]pyridin-4-ylsulfanyl)-N-hexylacetamide, 2-(3-butyl-7-chloro-3H-imidazo[4,5-c]pyridin-4-ylsulfanyl)-N-octylacetamide, 4-(3-butyl-7-chloro-3H-imidazo[4,5-c]pyridin-4-ylsulfanyl)-but-2-enoic acid dimethylamide

In another related embodiment, PDE7 inhibitors useful in the methods of the invention include the following compounds according to formula (41), wherein
R³ is Cl;
R³ is Cl, and X is alkylene having 1-4 carbon atoms;
R³ is Cl, X is alkylene having 1, 2, 3 or 4 carbon atoms, and A1 is alkyl having 1, 2, 3 or 4 carbon atoms. The preparation of these compounds is described in U.S. Pat. No. 6,613,778 and WO 01/034601.

In another embodiment, PDE7 inhibitors useful in the methods of the invention are selected from those compounds generally or specifically disclosed in WO 2008/113881, US 2010/0152213, and ES 2308916, U.S. Pat. No. 9,192,610, WO 2010/133742, and EP-A-2433637 each expressly incorporated herein by reference in its entirety.

In one embodiment, PDE7 inhibitors useful in the methods of the invention have the formula (42):

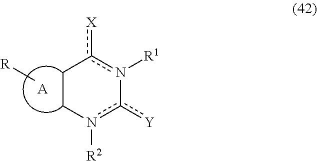

((42): Also referred to as quinazoline derivatives.)

The substituents for the above formula (42) are defined as follows:
A is fused carbocyclo or heterocyclo of 5, 6 or 7 members and may be saturated or unsaturated; the dashed lines represent, independently, a single or double bond; X and Y are chosen independently from the group consisting of alkyl, hydrogen, ═O, ═S, —N(alkyl), —N(aryl), aryl, O-alkyl, O-aryl, alkyl-S and —S-aryl; and R¹ and R² are chosen independently from the group consisting of hydrogen, halogen, alkyl, haloalkyl, aryl, cycloalkyl, (Z)ₙ-aryl, heteroaryl, —OR3; —C(O)OR3, —(Z)ₙ—C(O)OR3 and —S(O), or a pharmaceutically acceptable salt, derivative, prodrug, solvate or stereoisomer of the same. Exception: when A is unsubstituted benzene, X═O, Y═S, when A is unsubstituted benzene, X═O, Y═O, when A is unsubstituted benzene, X═O, Y═S-Me, when A is unsubstituted thiophene, X═O, Y═S, and when A is unsubstituted benzothiophene, X═O, Y═S.

In related embodiments, the above compounds constitute a useful pharmaceutical composition that includes a therapeutically effective amount of the above compounds, or mixtures of the same, a salt, derivative, prodrug, solvate or pharmaceutically acceptable stereoisomer of the same along with a carrier, adjuvant or pharmaceutically acceptable vehicle, for IV administration to patient.

In other related embodiments, the PDE7 inhibitors useful in the methods of the present invention include the following compound: 4-oxo-2-dioxo-1,2,3,4-tetrahydroquinazoline, and derivatives thereof selected from the following group:

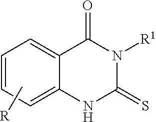

### 6-Bromo-2,3,4-tetrahydroquinazoline, 6-Bromo-(2,6-difluorophenyl)-4-oxo-2-dioxo-1,2,3,4-tetrahydroquinazoline, 6-Bromo-(2,3,4-trifluorophenyl)-4-oxo-2-dioxo-1,2,3,4-tetrahydroquinazoline, 6-Bromo-(2-bromophenyl)-4-oxo-2-dioxo-1,2,3,4-tetrahydroquinazoline, 3-(2,6-Difluorophenyl)-8-methyl-4-oxo-2-dioxo-1,2,3,4-tetrahydroquinazoline, 3-(2,3,4-Trifluorophenyl)-8-methyl-4-oxo-2-dioxo-1,2,3,4-tetrahydroquinazoline, and 3-(2-Bromophenyl)-8-methyl-4-oxo-2-dioxo-1,2,3,4-tetrahydroquinazoline

6-Bromo-(2,6-difluorophenyl)-4-oxo-2-thioxo-1,2,3,4-tetrahydroquinazoline, 6-Bromo-(2,3,4-trifluorophenyl)-4-oxo-2-thioxo-1,2,3,4-tetrahydroquinazoline, 6-Bromo-(2-bromophenyl)-4-oxo-2-thioxo-1,2,3,4-tetrahydroquinazoline, 3-(2,6-Difluorophenyl)-8-methyl-4-oxo-2-thioxo-1,2,3,4-tetrahydroquinazoline, 3-(2,3,4-Trifluorophenyl)-8-methyl-4-oxo-2-thioxo-1,2,3,4-tetrahydroquinazoline, and 3-(2-Bromophenyl)-8-methyl-4-oxo-2-thioxo-1,2,3,4-tetrahydroquinazoline, and 3-Phenyl-4-oxo-2-thioxo-1,2,3,4-tetrahydroquinazoline, 3-(2,6-Difluorophenyl)-4-oxo-2-thioxo-1,2,3,4-tetrahydroquinazoline, and 3-(2-Bromophenyl)-8-methyl-4-oxo-2-thioxo-1,2,3,4-tetrahydroquinazoline.

In a further related embodiment, the PDE7 inhibitors useful in the methods of the present invention include the following compound: 2-methylthio-4-oxo-3,4-dihydroquinazoline and derivatives thereof selected from the following group:

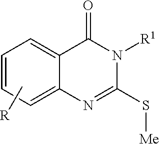

### 6-Bromo-(2,6-difluorophenyl)-2-methylthio-4-oxo-3,4-dihydroquinazoline, 6-Bromo-(2,3,4-trifluorophenyl)-2-methylthio-4-oxo-3,4-dihydroquinazoline, 6-Bromo-(2-bromophenyl)-2-methylthio-4-oxo-3,4-dihydroquinazoline, 3-Phenyl-8-methyl-2-methylthio-4-oxo-3,4-dihydroquinazoline, 3-(2,6-Difluorophenyl)-8-methyl-2-methylthio-4-oxo-3,4-dihydroquinazoline, 3-(2,3,4-Trifluorophenyl)-8-methyl-2-methylthio-4-oxo-3,4-dihydroquinazoline, and 3-(2-Bromophenyl)-8-methyl-2-methylthio-4-oxo-3,4-dihydroquinazoline

In another related embodiment, the PDE7 inhibitors useful in the methods of the present invention include the following compound: 2,4-dithioxo-1,2,3,4-tetrahydroquinazoline, and derivatives thereof selected from the following group:

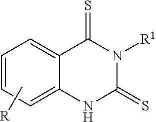

### 3-Phenyl-2,4-dithioxo-1,2,3,4-tetrahydroquinazoline, 3-(2,6-Difluorophenyl)-2,4-dithioxo-1,2,3,4-tetrahydroquinazoline, and 3-(2,3,4-Trifluorophenyl)-2,4-dithioxo-1,2,3,4-tetrahydroquinazoline

In a more specific embodiment the compound is the formula (42A) shown below:

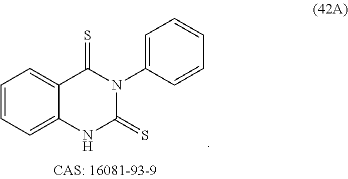 [SMILES: S=c1[nH]c2ccccc2c(=S)n1-c1ccccc1]

In another related embodiment, PDE7 inhibitors useful in the methods of the present invention include the following compound: (2-methylthio-4-thioxo-3,4-dihydroquinazoline) and derivatives thereof selected from the following group:

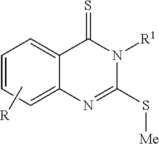

### 3-Phenyl-2-methylthio-4-thioxo-3,4-dihydroquinazoline, 3-(2,6-Difluorophenyl)-2-methylthio-4-thioxo-3,4-dihydroquinazoline, 3-(2,3,4-Trifluorophenyl)-2-methylthio-4-thioxo-3,4-dihydroquinazoline, and 3-(2-Bromophenyl)-2-methylthio-4-thioxo-3,4-dihydroquinazoline

The preparation of the above compounds is described in WO 2008/113881, US 2010/0152213, and ES 2308916.

In a related embodiment, PDE7 inhibitors useful in the methods of the invention are described in WO 2008/113881, US 2010/0152213, and ES 2308916, expressly incorporated by reference herein in its entirety.

In one embodiment, PDE7 inhibitors useful in the methods of the invention have the formulas (43A) and/or (43B):

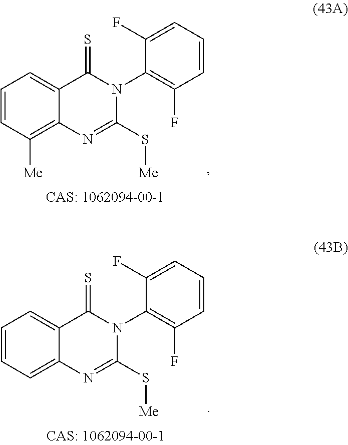 [SMILES: CSc1nc2c(C)cccc2c(=S)n1-c1c(F)cccc1F ; CSc1nc2ccccc2c(=S)n1-c1c(F)cccc1F]

In another embodiment, PDE7 inhibitors useful in the methods of the invention are selected from those compounds generally or specifically disclosed in U.S. Pat. No. 7,214,676, and U.S. 2007/0049558, each expressly incorporated herein by reference in its entirety.

In one embodiment, PDE7 inhibitors useful in the methods of the invention include the following compounds:
Spiro[cyclohexane-1-4′-(3′,4′-dihydro)quinazolin]-2′(1′H)-one, 6′-Methoxyspiro[cyclohexane-1-4′-(3′,4′-dihydro)quinazolin]-2′(1′H)-one, Spiro[cycloheptane-1-4′-(3′,4′-dihydro)quinazolin]-2′(1′H)-one, 7′-Methoxyspiro[cyclohexane-1-4′-(3′,4′-dihydro)quinazolin]-2′(1′H)-one, 6′-Phenylspiro[cycloheptane-1-4′-(3′,4′-dihydro)quinazolin]-2′(1′H)-one, 8′-Methoxyspiro[cyclohexane-1-4′-(3′,4′-dihydro)quinazolin]-2′(1′H)-one, 8′-Chlorospiro[cyclohexane-1-4′-(3′,4′-dihydro)quinazolin]-2′(1′H)-one, 7′-chlorospiro[cyclohexane-1-4′-(3′,4′-dihydro)quinazolin]-2′(1′H)-one, 5′-chlorospiro[cyclohexane-1-4′-(3′,4′-dihydro)quinazolin]-2′(1′H)-one, 8′-methylspiro[cyclohexane-1-4′-(3′,4′-dihydro)quinazolin]-2′(1′H)-one, 6′-chlorospiro[cyclohexane-1-4′-(3′,4′-dihydro)quinazolin]-2′(1′H)-one, 8′-bromospiro[cyclohexane-1-4′-(3′,4′-dihydro)quinazolin]-2′(1′H)-one, 8′-fluorospiro[cyclohexane-1-4′-(3′,4′-dihydro)quinazolin]-2′(1′H)-one, 6′-methylspiro[cyclohexane-1-4′-(3′,4′-dihydro)quinazolin]-2′(1′H)-one, 5′,8′-dichlorospiro[cyclohexane-1-4′-(3′,4′-dihydro)quinazolin]-2′(1′H)-one, 6′,7′-dichlorospiro[cyclohexane-1-4′-(3′,4′-dihydro)quinazolin]-2′(1′H)-one, 5′,6′-dichlorospiro[cyclohexane-1-4′-(3′,4′-dihydro)quinazolin]-2′(1′H)-one, 6′-phenylspiro[cyclohexane-1-4′-(3′,4′-dihydro)quinazolin]-2′(1′H)-one, 8′-iodospiro[cyclohexane-1-4′-(3′,4′-dihydro)quinazolin]-2′(1′H)-one, 8′-Bromospiro[cyclobutane-1-4′-(3′,4′-dihydro)quinazolin]-2′(1′H)-one, 8′-Bromospiro[cycloheptane-1-4′-(3′,4′-dihydro)quinazolin]-2′(1′H)-one, 8′-Bromo-4-methylspiro[cyclohexane-1-4′-(3′,4′-dihydro)quinazolin]-2′(1′H)-one, 8′-Bromospiro[bicyclo[3,2,1]octane-2-4′-(3′,4′-dihydro)quinazolin]-2′(1′H)-one, 6′,8′-dichlorospiro[cyclohexane-1-4′-(3′,4′-dihydro)quinazolin]-2′(1′H)-one, 8′-chloro-6′-iodospiro[cyclohexane-1-4′-(3′,4′-dihydro)quinazolin]-2′(1′H)-one, 8′-chloro-6′-methoxyspiro[cyclohexane-1-4′-(3′,4′-dihydro)quinazolin]-2′(1′H)-one, 8′-chloro-6′-phenylspiro[cycloheptane-1-4′-(3′,4′-dihydro)quinazolin]-2′(1′H)-one, 8′-chloro-6′-phenylspiro[cyclohexane-1-4′-(3′,4′-dihydro)quinazolin]-2′(1′H)-one, 8′-chloro-6′-methylspiro[cyclohexane-1-4′-(3′,4′-dihydro)quinazolin]-2′(1′H)-one, 8′-chloro-6′-(3-pyridyl)spiro[cyclohexane-1-4′-(3′,4′-dihydro)quinazolin]-2′(1′H)-one, 8′-chloro-6′-(4-pyridyl)spiro[cyclohexane-1-4′-(3′,4′-dihydro)quinazolin]-2′(1′H)-one, 6′-(4-carboxyphenyl)-8′-chlorospiro[cyclohexane-1-4′-(3′,4′-dihydro)quinazolin]-2′(1′H)-one, 6′-(3-carboxyphenyl)-8′-chlorospiro[cyclohexane-1-4′-(3′,4′-dihydro)-quinazolin]-2′(1′H)-one, 8′-chloro-6′-(1H-indol-5yl)spiro[cyclohexane-1-4′-(3′,4′-dihydro)quinazolin]-2′(1′H)-one, 8′-chloro-6′-(2-pyridyl)spiro[cyclohexane-1-4′-(3′,4′-dihydro)quinazolin]-2′(1′H)-one, 8′-chloro-6′-(3-dimethylamino-prop-1-ynyl)spiro[cyclohexane-1-4′-(3′,4′-dihydro)-quinazolin]-2′(1′H)-one, 8′-chloro-6′-(3-methylamino-prop-1-ynyl)spiro[cyclohexane-1-4′-(3′,4′-dihydro)quinazolin]-2′(1′H)-one, 8′-chloro-6′-[4-(4-methyl-piperazine-1-carbonyl)phenyl]spiro[cyclohexane-1-4′-(3′,4′-dihydro)quinazolin]-2′(1′H)-one, 8′-chloro-6′-[4-(3-N-dimethylamino-propylcarboxamide)phenyl]-spiro-[cyclohexane-1-4′-(3′,4′-dihydro)quinazolin]-2′(1′H)-one, 8′-chloro-6′-[4-(2-N-dimethylamino-ethylcarboxamide)phenyl]-spiro-[cyclohexane-1-4′-(3′,4′-dihydro)quinazolin]-2′(1′H)-one, 8′-chloro-6′-[3-(3-N-dimethylamino-propylcarboxamide)phenyl]-spiro-[cyclohexane-1-4′-(3′,4′-dihydro)quinazolin]-2′(1′H)-one, 8′-chloro-6′-[3-(4-methyl-piperazine-1-carbonyl)-phenyl]spiro-[cyclohexane-1-4′-(3′,4′-dihydro)quinazolin]-2′(1′H)-one, 8′-chloro-6′-[3-(2-N-dimethylamino-ethylcarboxamide)phenyl]spiro-[cyclohexane-1-4′-(3′,4′-dihydro)quinazolin]-2′(1′H)-one, 8′-Chlorospiro[cyclohexane-1-4′-(3′,4′-dihydro)quinazolin]-2′(1′H)-thione, 8′-Chloro-2′-cyanoiminospiro[cyclohexane-1-4′-(3′,4′-dihydro)quinazoline, 8′-chloro-6′-[4-(4-pyrimidin-2-yl-piperazine-1-carbonyl)phenyl]spiro[-cyclohexane-1-4′-(3′,4′-dihydro)quinazolin]-2′(1′H)-one, 8′-chloro-6′-[4-(4-(2-morpholin-4-yl-ethyl)-piperazine-1-carbonyl)-phenyl]spiro[-cyclohexane-1-4′-(3′,4′-dihydro)quinazolin]-2′(1′H)-one, 8′-chloro-6′-[4-(4-(2-morpholin-4-yl-2-oxo-ethyl)-piperazine-1-carbonyl)phenyl]spiro[-cyclohexane-1-4′-(3′,4′-dihydro)quinazolin]-2′(1′H)-one, 8′-chloro-6′-[4-(4-(2-hydroxy-ethoxy)-ethyl)-piperazine-1-carbonyl)-phenyl]spiro[-cyclohexane-1-4′-(3′,4′-dihydro)quinazolin]-2′(1′H)-one, Spiro[cyclohexane-1-9′-(8′,9′-dihydro)-pyrazolo[4′,3′-f]quinazolin]-7′(6′H)-one, 8′-Chloro-5′-methoxyspiro[cyclohexane-1-4′-(3′,4′-dihydro)quinazolin]-2′(1′H)-one, 5′,8′-difluorospiro[cyclohexane-1-4′-(3′,4′-dihydro)quinazolin]-2′(1′H)-one, 8′-Chloro-5′-methylspiro[cyclohexane-1-4′-(3′,4′-dihydro)quinazolin]-2′(1′H)-one, 8′-Chloro-6′-(morpholin-4-yl)methylspiro[cyclohexane-1-4′-(3′,4′-dihydro)quinazolin]-2′(1′H)-one, 8′-Chloro-5′-hydroxyspiro[cyclohexane-1-4′-(3′,4′-dihydro)quinazolin]-2′(1′H)-one, 8′-Chloro-5′-hydroxy-6′-iodo-spiro[cyclohexane-1-4′-(3′,4′-dihydro)quinazolin]-2′(1′H)-one, 8′-Chloro-6′-iodo-5′-methoxy-spiro[cyclohexane-1-4′-(3′,4′-dihydro)quinazolin]-2′(1′H)-one, 8′-Chloro-6′-cyano-5′-methoxy-spiro[cyclohexane-1-4′-(3′,4′-dihydro)quinazolin]-2′(1′H)-one, 8′-Chloro-5′-[2-(4-morpholino)ethoxy]spiro[cyclohexane-1-4′-(3′,4′-dihydro)quinazolin]-2′(1′H)-one, 8′-Chloro-5′-[2-dimethylaminoethoxy]spiro[cyclohexane-1-4′-(3′,4′-dihydro)quinazolin]-2′(1′H)-one, 8′-Chloro-5′-(2-aminoethoxy)-spiro[cyclohexane-1-4′-(3′,4′-dihydro)quinazolin]-2′(1′H)-one, 8′-Chloro-5′-[2-(methylamino)ethoxy]-spiro[cyclohexane-1-4′-(3′,4′-dihydro)quinazolin]-2′(1′H)-one, 8′-Chloro-5′-[2-(2-aminoethoxy)ethoxy]spiro[cyclohexane-1-4′-(3′,4′-dihydro)quinazolin]-2′(1′H)-one, 8′-Chloro-5′-[3-dimethylaminopropoxy]spiro[cyclohexane-1-4′-(3′,4′-dihydro)quinazolin]-2′(1′H)-one, 8′-Chloro-5′-ethoxycarbonylmethoxyspiro[cyclohexane-1-4′-(3′,4′-dihydro)quinazolin]-2′(1′H)-one, 5′-carboxymethoxy-8′-chloro-spiro[cyclohexane-1-4′-(3′,4′-dihydro)quinazolin]-2′(1′H)-one, 5′-carboxypropoxy-8′-chloro-spiro[cyclohexane-1-4′-(3′,4′-dihydro)quinazolin]-2′(1′H)-one, 8′-chloro-5′-(3-sulphopropoxy)-spiro[cyclohexane-1-4′-(3′,4′-dihydro)quinazolin]-2′(1′H)-one, 8′-Chloro-5′-[2-(tetrahydro-pyran-2-yloxy)-ethoxy]-spiro[cyclohexane-1-4′-(3′,4′-dihydro)quinazolin]-2′(1′H)-one, 8′-Chloro-5′-(2-hydroxy-ethoxy)-spiro[cyclohexane-1-4′-(3′,4′-dihydro)quinazolin]-2′(1′H)-one, 8′-Chloro-5′-(5-ethoxycarbonyl-furan-2-ylmethoxy)-spiro[cyclohexane-1-4′-(3′,4′-dihydro)quinazolin]-2′(1′H)-one, 8′-Chloro-5′-(5-carboxy-furan-2-ylmethoxy)-spiro[cyclohexane-1-4′-(3′,4′-dihydro)quinazolin]-2′(1′H)-one, 8′-Chloro-5′-cyanomethoxyspiro[cyclohexane-1-4′-(3′,4′-dihydro)quinazolin]-2′(1′H)-one, 8′-Chloro-5′-(1H-tetrazol-5-ylmethoxy)-spiro[cyclohexane-1-4′-(3′,4′-dihydro)quinazolin]-2′(1′H)-one, 8′-Chloro-5′-(5-hydroxy-[1,2,4]oxadiazol-3-ylmethoxy)-spiro[cyclohexane-1-4′-(3′,4′-dihydro)quinazolin]-2′(1′H)-one, 8′-Chloro-6′-iodo-5′-[2-dimethylamino-ethoxy]spiro[cyclohexane-1-4′-(3′,4′-dihydro)quinazolin]-2′(1′H)-one, 6′-(4-carboxyphenyl)-8′-chloro-5′-methoxyspiro[cyclohexane-1-4′-(3′,4′-dihydro)quinazolin]-2′(1′H)-one, 6′-(3-carboxyphenyl)-8′-chloro-5′-methoxyspiro[cyclohexane-1-4′-(3′,4′-dihydro)quinazolin]-2′(1′H)-one, 8′-chloro-6′-[2-(4-methyl-piperazine-1-carbonyl)phenyl]spiro[cyclohexane-1-4′-(3′,4′-dihydro)quinazolin]-2′(1′H)-one, 8′-chloro-6′-[2-methyl-4-(4-methyl-piperazine-1-carbonyl)phenyl]spiro[cyclohexane-1-4′-(3′,4′-dihydro)quinazolin]-2′(1′H)-one, 8′-chloro-6′-[4-(piperazine-1-carbonyl)phenyl]spiro[cyclohexane-1-4′-(3′,4′-dihydro)quinazolin]-2′(1′H)-one, 8′-chloro-6′-[4-carbamoyl-phenyl]spiro[cyclohexane-1-4′-(3′,4′-dihydro)quinazolin]-2′(1′H)-one, 8′-chloro-6′-[4-((1-methyl-piperidin-4-yl)-piperazine-1-carbonyl)phenyl]spiro[cyclohexane-1-4′-(3′,4′-dihydro)quinazolin]-2′(1′H)-one, 8′-chloro-5′-methoxy-6-[4-(4-methyl-piperazine-1-carbonyl)phenyl]spiro[cyclohexane-1-4′-(3′,4′-dihydro)quinazolin]-2′(1′H)-one, 8′-Trifluoromethylspiro[cyclohexane-1-4′-(3′,4′-dihydro)quinazolin]-2′(1′H)-one, 8′-Chloro-6′-cyanomethylspiro[cyclohexane-1-4′-(3′,4′-dihydro)quinazolin]-2′(1′H)-one, 8′-Chloro-5′-(3-dimethylamino-2-hydroxy-propoxy)-spiro[cyclohexane-1-4′-(3′,4′-dihydro)quinazolin]-2′(1′H)-one, 8′-Chloro-5′-(3-methylamino-2-hydroxy-propoxy)-spiro[cyclohexane-1-4′-(3′,4′-dihydro)quinazolin]-2′(1′H)-one, 8′-Chloro-5′-[2-(ethoxycarbonylmethyl-amino)-ethoxy]-spiro[cyclohexane-1-4′-(3′,4′-dihydro)quinazolin]-2′(1′H)-one, 8′-Chloro-5′-[2-(carboxymethyl-amino)-ethoxy]-spiro[cyclohexane-1-4′-(3′,4′-dihydro)quinazolin]-2′(1′H)-one hydrochloride, 8′-Chloro-5′-(2-methanesulfonylamino-2-oxo-ethoxy)-spiro[cyclohexane-1-4′-(3′,4′-dihydro)quinazolin]-2′(1′H)-one, 8′-Chloro-5′-(2-[(5-methyl-isoxazol-3-ylmethyl)-amino]ethoxy)-spiro[cyclohexane-1-4′-(3′,4′-dihydro)quinazolin]-2′(1′H)-one.

Preparation of these compounds is described in U.S. Pat. No. 7,087,614, US 2007/0049558 and WO 2002/074754.

In another embodiment, PDE7 inhibitors and dual PDE4/7 inhibitors useful in the methods of the invention are selected from those compounds generally or specifically disclosed in U.S. Pat. No. 7,087,614, US 2003/0162802 and WO 2002/102313, each expressly incorporated herein by reference in its entirety.

In one embodiment, PDE7 inhibitors useful in the methods of the invention have the formula (54):

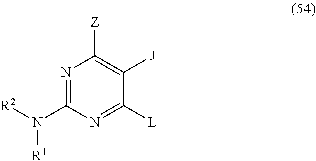

((42): Also referred to as 2-amino substituted pyrimidine derivatives.)

The PDE7 inhibitors useful in the methods of the invention include enantiomers, diastereomers, tautomers, and pharmaceutically acceptable salts, prodrugs, and solvates of the compounds of the above formula.

The substituents for the above formula (54) are defined as follows:
R¹ is H or alkyl;
R² is (a) heteroaryl, or heterocyclo, either of which may be optionally substituted with one to three groups T1, T2, T3; (b) aryl substituted with one to three groups T1, T2, T3 provided that at least one of T1, T2, T3 is other than H; or (c) aryl fused to a heteroaryl or heterocyclo ring wherein the combined ring system may be optionally substituted with one to three groups T1, T2, T3;
Z is (a) —OR4, —C(O)R4, —C(O)OR4, —SR4, —NR3R4, —C(O)NR3R4, —NR3SO₂R4c, halogen, nitro, haloalkyl; or (b) alkyl, aryl, heteroaryl, heterocyclo, or cycloalkyl any of which may be optionally substituted with one to three groups T1a, T2a T3a;
J is (a) hydrogen, halo, —OR4a, or (b) alkyl, alkenyl, or alkynyl any of which may be optionally substituted with one to three groups T1b, T2b or T3b;
L is (a) hydrogen, —OR4b, —C(O)R4b, —C(O)OR4b, —SR4b, —NR5R6, —C(O)NR5R6, —NR₅SO₂R4d, halogen, haloalkyl, nitro, or (b) alkyl, aryl, heteroaryl, heterocyclo, or cycloalkyl any of which may be optionally substituted with one to three groups T1c, T2c or T3c;
R3 and R4 are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally substituted with one to three groups T1a, T2a or T3a;
or R3 and R4 together with the nitrogen atom to which they are attached may combine to form a 4 to 8 membered heterocyclo ring optionally substituted with one to three groups T1a, T2a or T3a;
R4a is hydrogen, alkyl, alkenyl, aryl, heteroaryl, (aryl)alkyl, (heteroaryl)alkyl, heterocylo, (heterocyclo)alkyl, cycloalkyl or (cycloalkyl)alkyl any of which may be optionally substituted with one to three groups T1b, T2b or T3b;
R4b is hydrogen, alkyl, alkenyl, aryl, heteroaryl, (aryl)alkyl, (heteroaryl)alkyl, heterocylo, (heterocyclo)alkyl, cycloalkyl or (cycloalkyl)alkyl any of which may be optionally substituted with one to three groups T1c, T2c or T3c;
R4c and R4d are independently alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally substituted with one to three groups T1a, T2a or T3a;
R5 and R6 are independently H, alkyl, alkenyl, aryl, (aryl)alkyl, heteroaryl, (heteroaryl)alkyl, cycloalkyl, (cycloalkyl)alkyl, heterocylo or (heterocyclo)alkyl any of which may be optionally independently substituted where valance allows with one to three groups T1c, T2c or T3c;
or R5 and R6 together with the nitrogen atom to which they are attached may combine to form a 4 to 8-membered heterocyclo ring optionally substituted with one to three groups T1c, T2c or T3c;
T1-1c, T2-2c, and T3-3c are each independently (1) hydrogen or T6, where T6 is (i)alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl, or (heteroaryl)alkyl;
(ii) a group (i) which is itself substituted by one or more of the same or different groups (i); or (iii) a group (i) or (ii) which is independently substituted by one or more (preferably 1 to 3) of the following groups (2) to (13) of the definition of T1-1c, T2-2c and T3-3c (2) —OH or —OT6, (3) —SH or —ST6, (4) —C(O)ₜH, —C(O)ₜT6, or —O—C(O)T6, where t is 1 or 2; (5) —SO₃H, —S(O)T6, or S(O)ₜN(T9) T6, (6) halo, (7) cyano, (8) nitro, (9) -T4-NT7T8, (10) -T4-N(T9)-T5-NT7T8, (11) -T4-N(T10)-T5-T6, (12) -T4-N(T10)-T5-H, (13) oxo,
T4 and T5 are each independently (1) a single bond, (2) -T11-S(O)ₜ-T12-, (3) -T11-C(O)-T12-, (4) -T11-C(S)-T12-, (5) -T11-O-T12-, (6) -T11-S-T12-, (7) -T11-O—C(O)-T12-, (8) -T11-C(O)—O-T12-, (9) -T11-C(═NT9a)-T12-, or (10) -T11-C(O)—C(O)-T12,
T7, T8, T9, T9a and T10 (1) are each independently hydrogen or a group provided in the definition of T6, or (2) T7 and T8 may together be alkylene or alkenylene, completing a 3- to 8-membered saturated or unsaturated ring together with the atoms to which they are attached, which ring is unsubstituted or substituted with one or more groups listed in the description of T1-1c, T2-2c and T3-3c, or (3) T7 or T8, together with T9, may be alkylene or alkenylene completing a 3- to 8-membered saturated or unsaturated ring together with the nitrogen atoms to which they are attached, which ring is unsubstituted or substituted with one or more groups listed in the description of T1-1c, T2-2c and T3-3c, or (4) T7 and T8 or T9 and T10 together with the nitrogen atom to which they are attached may combine to form a group —N═CT13T14 where T13 and T14 are each independently H or a group provided in the definition of T6;
and T11 and T12 are each independently (1) a single bond, (2) alkylene, (3) alkenylene, or (4) alkynylene.

In a related embodiment, PDE7 inhibitors useful in the methods of the present invention include the above compounds, wherein:
Z is (a) halogen, alkoxy, haloalkyl, —NR3R4, —C(O)OR4, —C(O)NR3R4; (b) aryl or heteroaryl either of which may be optionally substituted with one or more T1a, T2a, T3a (especially cyano, optionally substituted alkyl, (hydroxy)alkyl, —OH, —OT6, —ST6, —SOₜT6, —COₜH, —COₜT6, -T4NT7T8, or -T4N(T10)-T5-T6); (c) optionally substituted alkyl (especially substituted with one or more —OH, —COₜH, —COₜT6, -T4-NT7T8, -T4-N(T10)-T5-H, or -T4-N(T10)-T5-T6);
J is (a) H, or (b) alkyl or alkenyl either of which may be optionally substituted (especially with one or more —OH, —OT6, —COₜH, or —COₜT6);
L is (a) H; (b) halogen, alkoxy, haloalkyl, —NR5R6, —C(O)OR_{4b}, —C(O)NR5R6; (c) aryl or heteroaryl either of which may be optionally substituted with one or more T1c, T2c, T3c (especially cyano, optionally substituted alkyl, (hydroxy)alkyl, —OH, —OT6, —ST6, —SOₜT6, —COₜH, —COₜT6, -T4NT7T8, or -T4N(T10)-T5-T6); or (d) optionally substituted alkyl (especially substituted with one or more —OH, —COₜH, —COₜT6, -T4-NT7T8, -T4-N(T10)-T5-H, or; -T4-N(T10)-T5-T6);
R¹ is H or alkyl;
R² is (a) heteroaryl (more preferably thiazolyl or oxazolyl) optionally substituted with one to three groups T1, T2, T3, preferably including H, alkyl, haloalkyl, halo, heteroaryl, cyano, C(O)ₜT6, OT6, -T4NT7T8; (b) aryl substituted with one to three groups T1, T2, T3 (preferably including heteroaryl (preferably, imidazolyl, oxazolyl, or thiazolyl any of which may be further optionally substituted), cyano, C(O)ₜT6, S(O)ₜN(T9) T6, halo alkyl, and haloalkyl); or (c) aryl fused to a heterocyclo ring (e.g., 2,3-dihydro-1H-indole bound through the aryl ring, quinolyl bound through the aryl ring (especially quinol-6-yl), quinazolinyl bound through the aryl ring (especially quinazolin-7-yl), cinnolinyl bound through the aryl ring (especially cinnolin-6-yl), isoqinolinyl bound through the aryl ring (especially isoquinol-6-yl), and phthalazinyl bound through the aryl ring (especially phthalazin-6-yl)) wherein the combined ring system may be optionally substituted with one to three groups T1, T2, T3 (especially halo, OH, OT6, alkyl, —COₜH, —COₜT6, or —C(O) NT7T8);
R3 is H or optionally substituted alkyl (especially substituted with one or more —OH, or —OT6);
R4 is (a) hydrogen; (b) (aryl)alkyl where the aryl group is optionally independently substituted with one or more groups T1a, T2a, T3a (especially optionally substituted alkyl, halo, cyano, nitro, (hydroxy)alkyl, —OH, —OT6, —ST6, —COₜH, —COₜT6, —SO3H, —SOₜT6, —SOₜN(T9)(T6), -T4NT7T8, -T4-N(T10)-T5-T6, heterocyclo, or heteroaryl); (c) (heteroaryl)alkyl where the heteroaryl group is optionally independently substituted with one or more groups T1a, T2a, T3a (especially optionally substituted alkyl, halo, cyano, nitro, (hydroxy)alkyl, —OH, —OT6, —ST6, —COₜH, —COₜT6, —SO₃H, —SOₜT6, —SOₜN(T9)(T6), -T4NT7T8, -T4-N(T10)-T5-T6, heterocyclo, or heteroaryl); (d) (heterocyclo)alkyl where the heterocyclo group is optionally independently substituted with one or more groups T1a, T2a, T3a (especially optionally substituted alkyl, halo, cyano, nitro, oxo, (hydroxy)alkyl, —OH, —OT6, —ST6, —COₜH, —COₜT6, —SO₃H, —SOₜT6, —SOₜN(T9)(T6), -T4NT7T8, -T4-N(T10)-T5-T6, heterocyclo, or heteroaryl); (e) alkyl optionally independently substituted with one or more groups T1a, T2a, T3a (especially —OH, —OT6, —COₜH, —COₜT6, -T4NTT8 or -T4-N(T10)-T5-T6); (f) heterocyclo optionally independently substituted with one or more groups T1a, T2a, T3a (especially optionally substituted alkyl, optionally substituted aryl, optionally substituted heteroaryl, optionally substituted aralkyl, optionally substituted heterocyclo, cyano, —OH, —OT6, —COₜH, —COₜT6, oxo, hydroxy(alkyl), (alkoxy)alkyl, -T4-N(T10)-T5-T6, or -T4-NT2T8);
or R3 and R4 together with the nitrogen atom to which they are attached combine to form a 4 to 8-membered heterocyclo ring (especially pyrrolidinyl, piperadinyl, piperazinyl, morpholinyl, diazapanyl or 1,4-dioxa-8-azaspiro[4.5]decan-8-yl) optionally substituted with one to three groups T1a, T2a, T3a (especially optionally substituted alkyl, optionally substituted aryl, optionally substituted heteroaryl, optionally substituted aralkyl, optionally substituted heterocyclo, cyano, —OH, —OT6, —COₜH, —COₜT6, oxo, hydroxy(alkyl), (alkoxy)alkyl, -T4-N(T10)-T5-T6, or -T4-NT7T8); R5 is hydrogen or alkyl;
R6 is (a) hydrogen; (b) (aryl)alkyl where the aryl group is optionally independently substituted with one or more groups T1c, T2c, T3c (especially optionally substituted alkyl, halo, cyano, nitro, (hydroxy)alkyl, —OH, —OT6, —ST6, —COₜH, —COₜT6, —SO₃H, —SOₜT6, —SOₜN(T9)(T6), -T4-N(T10)-T5-T6, heterocyclo, or heteroaryl); (c) (heteroaryl)alkyl where the heteroaryl group is optionally independently substituted with one or more groups T1c, T2c, T3c (especially optionally substituted alkyl, halo, cyano, nitro, (hydroxy)alkyl, —OH, —OT6, —ST6, —COₜH, —COₜT6, —SO₃H, —SOₜT6, —SOₜN(T9)(T6), -T4-N(T10)-T5-T6, heterocyclo, or heteroaryl); (d) (heterocyclo)alkyl where the heterocyclo group is optionally independently substituted with one or more groups T1c, T2c, T3c (especially optionally substituted alkyl, halo, cyano, nitro, oxo, (hydroxy)alkyl, —OH, —OT6, —ST6, —COₜH, —COₜT6, —SO₃H, —SOₜT6, —SOₜN(T9)(T6), -T4-N(T10)-T5-T6, heterocyclo, or heteroaryl); (e) alkyl optionally independently substituted with one or more groups T1c, T2c, T3c (especially —OH, —OT6, —COₜH, —COₜT6, -T4NT7T8 or -T4-N(T10)-T5-T6); (f) heterocyclo optionally independently substituted with one or more groups T1c, T2c, T3c (especially optionally substituted alkyl, optionally substituted aryl, optionally substituted heteroaryl, optionally substituted aralkyl, optionally substituted heterocyclo, cyano, —OH, —OT6, —COₜH, —COₜT6, oxo, hydroxy(alkyl), (alkoxy)alkyl, -T4-N(T10)-T5-T6, or -T4-NT7T8);
or R5 and R6 together with the nitrogen atom to which they are attached combine to form a 4 to 8-membered heterocyclo ring (especially pyrrolidinyl, piperadinyl, piperazinyl, morpholinyl, diazapanyl or 1,4-dioxa-8-azaspiro[4.5]decan-8-yl) optionally substituted with one to three groups T1c, T2c, T3c (especially optionally substituted alkyl, optionally substituted aryl, optionally substituted heteroaryl, optionally substituted aralkyl, optionally substituted heterocyclo, cyano, —OH, —OT6, —COtH, —COtT6, oxo, hydroxy(alkyl), (alkoxy)alkyl, -T4-N(T10)-T5-T6, or -T4-NT7T8).

In another related embodiment, PDE7 inhibitors useful in the methods of the present invention include the above compounds, wherein:
Z is (a) halogen, alkoxy, haloalkyl, —NR3R4, —C(O)OR4, —C(O)NR3R4; (b) aryl or heteroaryl either of which may be optionally substituted with one or more T1a, T2a, T3a selected from cyano, optionally substituted alkyl, (hydroxy)alkyl, —OH, —OT6, —ST6, —SOₜT6, —COₜH, —COₜT6, -T4NT7T8, or -T4N(T10)-T5-T6, where T4 is a bond or —C(O)—; T5 is —C(O)—, or —C(O)O—; T6 is alkyl or haloalkyl; T7 and T8 are independently H; alkyl optionally substituted with cycloalkyl, heteroaryl, hydroxy or -NT7T8 cycloalkyl; or aryl optionally substituted with halogen; or T7 and T8 together with the nitrogen atom to which they are attached combine to form a heterocyclo ring optionally substituted with (hydroxy)alkyl, COₜH or COₜT6, T10 is hydrogen; (c) alkyl optionally substituted with one or more —OH, —COₜH, —COₜT6, -T4-NT7T8, -T4-N(T10)-T5-H, or -T4-N(T10)-T5-T6 where T4 is —C(O)—; T5 is -alkylene-O—; T6 is alkyl; T7 and T8 are independently H, alkyl, cycloalkyl, aryl, (aryl)alkyl (optionally substituted as described in the definition of R4), or heterocyclo (optionally substituted as described in the definition of R3 and R4 combining to form a heterocyclo ring); and T10 is H;
J is (a) H, or (b) alkyl or alkenyl either of which may be optionally substituted with one or more —OH, —OT6, —COₜH, or —COₜT6, where T6 is alkyl;
L is (a) H; (b) halogen, alkoxy, haloalkyl, —NR5R6, —C(O)OR4b, —C(O)NR5R6; (c) aryl or heteroaryl either of which may be optionally substituted with one or more T1c, T2c, T3c selected from cyano, optionally substituted alkyl (especially substituted with COₜH or COₜT6), (hydroxy)alkyl, —OH, —OT6, —ST6, —SOₜT6, —COₜH, —COₜT6, -T4NT7T8, or -T4N(T10)-T5-T6, where T4 is a bond or —C(O)—; T5 is —C(O)—, or —C(O)O—; T6 is alkyl or haloalkyl; T7 and T8 are independently H; alkyl optionally substituted with cycloalkyl, heteroaryl, hydroxy or -NT7T8; cycloalkyl; or aryl optionally substituted with halogen; or T7 and T8 together with the nitrogen atom to which they are attached combine to form a heterocyclo ring optionally substituted with (hydroxy)alkyl, COₜH or COₜT6; T10 is hydrogen; (d) alkyl optionally substituted with one or more —OH, —COₜH, —COₜT6, -T4-NT7T8, -T4-N(T10)-T5-H, or -T4-N(T10)-T5-T6 where T4 is —C(O)—; T5 is -alkylene-O—; T6 is alkyl; T7 and T8 are independently H, alkyl, cycloalkyl, aryl, (aryl)alkyl (optionally substituted as described in the definition of R4), or heterocyclo (optionally substituted as described in the definition of R3 and R4 combining to form a heterocyclo ring); and T10 is H;
R¹ is H or alkyl;
R² is (a) heteroaryl (more preferably thiazolyl or oxazolyl) optionally substituted with one to three groups T1, T2, T3, preferably including H, alkyl, haloalkyl, halo, heteroaryl, cyano, C(O)ₜT6, OT6, -T4NT7T8; (b) aryl substituted with one to three groups T1, T2, T3 (preferably including heteroaryl (preferably, imidazolyl, oxazolyl, or thiazolyl any of which may be further optionally substituted), cyano, C(O)ₜT6, S(O)ₜN(T9) T6, halo alkyl, and haloalkyl); or (c) aryl fused to a heterocyclo ring (e.g., 2,3-dihydro-1H-indole bound through the aryl ring) wherein the combined ring system may be optionally substituted with one to three groups T1, T2, T3 (especially halo, —OH, —OT6, alkyl, —COₜH, —COₜT6, or —C(O) NT7T8);
R3 is H or optionally substituted alkyl (especially substituted with one or more —OH, or —OT6);
R4 is (a) hydrogen; (b) (aryl)alkyl where the aryl group is optionally independently substituted with one or more groups T1a, T2a, T3a selected from optionally substituted alkyl, halo, cyano, nitro, (hydroxy)alkyl, —OH, —OT6, —ST6, —COₜH, —COₜT6, —SO₃H, —SOₜT6, —SOₜN(T9)(T6), -T4NT7T8, -T4N(T10)-T5-T6, heterocyclo, or heteroaryl) where T4 is a bond, —SO₂—, or —C(O)—; T5 is —SO₂—, or -alkylene-O—; T6 is alkyl, or cycloalkyl; T7 and T8 are independently H or alkyl; and T9 and T10 are hydrogen; (c) (heteroaryl)alkyl where the heteroaryl group is optionally independently substituted with one or more groups T1a, T2a, T3a selected from optionally substituted alkyl, halo, cyano, nitro, oxo, (hydroxy)alkyl, —OH, —OT6, —ST6, —COₜH, —COₜT6, —SO3H, —SOₜT6, —SOₜN(T9)(T6), -T4NT7T8, -T4-N(T10)-T5-T6, heterocyclo, or heteroaryl) where T4 is a bond, —SO₂—, or —C(O)—; T5 is —SO₂—, or -alkylene-O—; T6 is alkyl, or cycloalkyl; T7 and T8 are independently H or alkyl; and T9 and T10 are hydrogen; (d) (heterocyclo)alkyl where the heterocyclo group is optionally independently substituted with one or more groups T1a, T2a, T3a selected from optionally substituted alkyl, halo, cyano, nitro, (hydroxy)alkyl, —OH, —OT6, —ST6, —CO; H, —COₜT6, —SO3H, —SOₜT6, -T4NT7T8, -T4-N(T10)-T5-T6, heterocyclo, or heteroaryl) where T4 is a bond, —SO₂—, or —C(O)—; T5 is —SO₂—, or -alkylene-O—; T6 is alkyl, or cycloalkyl; T7 and T8 are independently H or alkyl; and T9 and T10 are hydrogen; (e) alkyl optionally independently substituted with one or more groups T1a, T2a T3a selected from —OH, —OT6, —COₜH, —COₜT6, -T4NT7T8 or -T4-N(T10)-T5-T6) where T4 is a bond; T5 is —C(O)—; T6 is alkyl; T7 and T8 are independently H or alkyl; and T10 is hydrogen; heterocyclo optionally independently substituted with one or more groups T1a, T2a, T3a selected from optionally substituted alkyl (especially substituted with -T4NT7T8), optionally substituted aryl (especially substituted with halogen or haloalkyl), cyano, —OH, —OT6, —COₜH, —COₜT6, oxo, hydroxy(alkyl), (alkoxy)alkyl, -T4-N(T10)-T5-T6, or -T4-NT7T8) where T4 is a bond or —C(O)—; T5 is —C(O)—, —SO₂—, or -alkylene-C(O)O—; T6 is alkyl, alkoxy, or heteroaryl; T7 and T8 are independently H, alkyl, or cycloalkyl; or T7 and T8 together with the nitrogen atom to which they are attached combine to form an optionally substituted heterocyclo ring;
or R3 and R4 together with the nitrogen atom to which they are attached combine to form a heterocyclo ring selected from pyrrolidinyl, piperadinyl, piperazinyl, morpholinyl, diazapanyl or 1,4-dioxa-8-azaspiro[4.5]decan-8-yl), any of which are optionally independently substituted with one to three groups T1a, T2a, T3a selected from optionally substituted alkyl (especially substituted with -T4NT7T8), optionally substituted aryl (especially substituted with halogen or haloalkyl), cyano, —OH, —OT6, —COₜH, —COₜT6, oxo, hydroxy(alkyl), (alkoxy)alkyl, -T4-N(T10)-T5-T6, or -T4-NT7T8) where T4 is a bond or —C(O)—; T5 is —C(O)—, —SO₂—, or -alkylene-C(O)O—; T6 is alkyl, alkoxy, or heteroaryl; T7 and T8 are independently H, alkyl, or cycloalkyl; or T7 and T8 together with the nitrogen atom to which they are attached combine to form an optionally substituted heterocyclo ring;
R5 is hydrogen or alkyl;
R6 is (a) hydrogen; (b) (aryl)alkyl where the aryl group is optionally independently substituted with one or more groups T1c, T2c, T3c selected from optionally substituted alkyl, halo, cyano, nitro, (hydroxy)alkyl, —OH, —OT6, —ST6, —COₜH, —COₜT6, —SO3H, —SOₜT6, —SOₜN(T9)(T6), -TNT7T8, -T4-N(T10)-T5-T6, heterocyclo, or heteroaryl) where T4 is a bond, —SO2-, or —C(O)—; T5 is —SO2-, or -alkylene-O—; T6 is alkyl, or cycloalkyl; T7 and T8 are independently H or alkyl; and T9 and T10 are hydrogen; (c) (heteroaryl)alkyl where the heteroaryl group is optionally independently substituted with one or more groups T1c, T2c, T3c selected from optionally substituted alkyl, halo, cyano, nitro, oxo, (hydroxy)alkyl, —OH, —OT6, —ST6, —COₜH, −COₜT6, —SO3H, —SOₜT6, —SOₜN(T9)(T6), -T4NT7T8, -T4-N(T10)-T5-T6, heterocyclo, or heteroaryl) where T4 is a bond, —SO2-, or —C(O)—; T5 is —SO₂—, or -alkylene-O—; T6 is alkyl, or cycloalkyl; T7 and T8 are independently H or alkyl; and T9 and T10 are hydrogen; (d) (heterocyclo)alkyl where the heterocyclo group is optionally independently substituted with one or more groups T1c, T2c, T3c selected from optionally substituted alkyl, halo, cyano, nitro, (hydroxy)alkyl, —OH, —OT6, —ST6, —COₜH, —COₜT6, —SO3H, —SOₜT6, -T4NT7T8, -T4-N(T10)-T5-T6, heterocyclo, or heteroaryl) where T4 is a bond, —SO₂—, or —C(O)—; T5 is —SO₂—, or -alkylene-O—; T6 is alkyl, or cycloalkyl; T7 and T8 are independently H or alkyl; and T9 and T10 are hydrogen; (e) alkyl optionally independently substituted with one or more groups T1c, T2c, T3c selected from —OH, —OT6, —OCH, —COₜT6, -T4NT7T8 or -T4-N(T10)-T5-T6) where T4 is a bond; T5 is —C(O)—; T6 is alkyl; T7 and T8 are independently H or alkyl; and T10 is hydrogen; heterocyclo optionally independently substituted with one or more groups T1c, T2c, T3c selected from optionally substituted alkyl (especially substituted with -T4NT7T8), optionally substituted aryl (especially substituted with halogen or haloalkyl), cyano, —OH, —OT6, —COₜH, —COₜT6, oxo, hydroxy(alkyl), (alkoxy)alkyl, -T4-N(T10)-T5-T6, or -T4-NT7T8, where T4 is a bond or —C(O)—; T5 is —C(O)—, —SO₂—, or -alkylene-C(O)O—; T6 is alkyl, alkoxy, or heteroaryl; T7 and T8 are independently H, alkyl, or cycloalkyl; or T7 and T8 together with the nitrogen atom to which they are attached combine to form an optionally substituted heterocyclo ring;
or R5 and R6 together with the nitrogen atom to which they are attached combine to form a heterocyclo ring selected from pyrrolidinyl, piperadinyl, piperazinyl, morpholinyl, diazapanyl or 1,4-dioxa-8-azaspiro[4.5]decan-8-yl), any of which are optionally independently substituted with one to three groups T1a, T2a, T3a selected from optionally substituted alkyl (especially substituted with -T4NT7T8), optionally substituted aryl (especially substituted with halogen or haloalkyl), cyano, —OH, —OT6, —COₜH, —COₜT6, oxo, hydroxy(alkyl), (alkoxy)alkyl, -T4-N(T10)-T5-T6, or -T4-NT7T8 where T4 is a bond or —C(O)—; 5 is —C(O)—, —SO₂—, or -alkylene-C(O)O—; T6 is alkyl, alkoxy, or heteroaryl; T7 and T8 are independently H, alkyl, or cycloalkyl; or T7 and T8 together with the nitrogen atom to which they are attached combine to form a an optionally substituted heterocyclo ring.

In a further related embodiment, PDE7 inhibitors useful in the methods of the present invention include the following compounds:
2-[[4-[[[4-(Aminosulfonyl)phenyl]methy]amino]-6-(4-methyl-1-piperazinyl)-2-pyrimidinyl]amino]-4-methyl-5-thiazolecarboxylic acid ethyl ester; 2-[[4-[[(3,4-Dimethoxyphenyl)methyl]amino]-6-(1-piperazinyl)-2-pyrimidinyl]amino]-4-methyl-5-thiazolecarboxylic acid ethyl ester trifluoroacetate salt; 2-[[4-[[[4-(Aminosulfonyl)phenyl]methyl]amino]-6-(1-piperazinyl)-2-pyrimidinyl]amino]-4-methyl-5-thiazolecarboxylic acid ethyl ester; 4-Methyl-2-[[4-[[[4-(methylsulfonyl)phenyl]methyl]amino]-6-(1-piperazinyl)-2-pyrimidinyl]amino]-5-thiazolecarboxylic acid ethyl ester; 2-[[4-[[(4-Methoxyphenyl)methyl]amino]-6-(1-piperazinyl)-2-pyrimidinyl]amino]-4-methyl-5-thiazolecarboxylic acid ethyl ester; 2-[[4-[[(3-Methoxyphenyl)methyl]amino]-6-(1-piperazinyl)-2-pyrimidinyl]amino]-4-methyl-5-thiazolecarboxylic acid ethyl ester; 2-[[4-[[(2-Methoxyphenyl)methyl]amino]-6-(1-piperazinyl)-2-pyrimidinyl]amino]-4-methyl-5-thiazolecarboxylic acid ethyl ester; 4-Methyl-2-[[4-(1-piperazinyl)-6-[[(3,4,5-trimethoxyphenyl)methyl]amino]-2-pyrimidinyl]amino]-5-thiazolecarboxylic acid ethyl ester; 2-[[4-[[(2-Ethoxyphenyl)methyl]amino]-6-(1-piperazinyl)-2-pyrimidinyl]amino]-4-methyl-5-thiazolecarboxylic acid ethyl ester; 2-[[4-[[(2,5-Dimethoxyphenyl)methyl]amino]-6-(1-piperazinyl)-2-pyrimidinyl]amino]-4-methyl-5-thiazolecarboxylic acid ethyl ester; 2-[[4-[[(3,5-Dimethoxyphenyl)methyl]amino]-6-(1-piperazinyl)-2-pyrimidinyl]amino]-4-methyl-5-thiazolecarboxylic acid ethyl ester; 2-[[4-[[(2,6-Dimethylphenyl)methyl]amino]-6-(1-piperazinyl)-2-pyrimidinyl]amino]-4-methyl-5-thiazolecarboxylic acid ethyl ester; 2-[[4-[[[4-(Methoxycarbonyl)phenyl]methyl]amino]-6-(1-piperazinyl)-2-pyrimidinyl]amino]-4-methyl-5-thiazolecarboxylic acid ethyl ester; 2-[[4-[[(3-Bromophenyl)methyl]amino]-6-(1-piperazinyl)-2-pyrimidinyl]amino]-4-methyl-5-thiazolecarboxylic acid ethyl ester; 2-[[4-[(1,3-Benzodioxol-5-ylmethyl)amino]-6-(1-piperazinyl)-2-pyrimidinyl]amino]-4-methyl-5-thiazolecarboxylic acid ethyl ester; 4-Methyl-2-[[4-[methyl (3-pyridinylmethyl)amino]-6-(1-piperazinyl)-2-pyrimidinyl]amino]-5-thiazolecarboxylic acid ethyl ester; 4-Methyl-2-[[4-(1-piperazinyl)-6-[[[4-(1,2,3-thiadiazol-4-yl)phenyl]methyl]amino]-2-pyrimidinyl]amino]-5-thiazolecarboxylic acid ethyl ester; 2-[[4-[[(3-(Cyclopentyloxy)-4-methoxyphenyl]methyl]amino]-6-(1-piperazinyl)-2-pyrimidinyl]amino]-4-methyl-5-thiazolecarboxylic acid ethyl ester; 4-Methyl-2-[[4-[(phenylmethyl)amino]-6-(1-piperazinyl)-2-pyrimidinyl]amino]-5-thiazolecarboxylic acid ethyl ester; 4-Methyl-2-[[4-(4-methyl-1-piperazinyl)-6-[[(3,4,5-trimethoxyphenyl)methyl]amino]-2-pyrimidinyl]amino]-5-thiazolecarboxylic acid ethyl ester; 2-[[4-(4-Hydroxy-1-piperidinyl)-6-[[[4-(methylsulfonyl)phenyl]methyl]amino]-2-pyrimidinyl]amino]-4-methyl-5-thiazolecarboxylic acid ethyl ester; 4-Methyl-2-[[4-[[2-(1-methylethoxy)ethyl]amino]-6-[[[4-(methylsulfonyl)phenyl]methyl]amino]-2-pyrimidinyl]amino]-5-thiazolecarboxylic acid ethyl ester; 2-[[4-[3-(Aminocarbonyl)-1-piperidinyl]-6-[[[4-(methylsulfonyl)phenyl]methyl]amino]-2-pyrimidinyl]amino]-4-methyl-5-thiazolecarboxylic acid ethyl ester; 2-[[4-[[(2-(1H-imidazol-4-yl)ethyl]amino]-6-[[[4-(methylsulfonyl)phenyl]methyl]amino]-2-pyrimidinyl]amino]-4-methyl-5-thiazolecarboxylic acid ethyl ester; 4-Methyl-2-[[4-[[[4-(methylsulfonyl)phenyl]methyl]amino]-6-[[3-(4-morpholinyl) propyl]amino]-2-pyrimidinyl]amino]-5-thiazolecarboxylic acid ethyl ester; 2-[[4-[(2-Methoxy-1-methylethyl)amino]-6-[[[4-(methylsulfonyl)phenyl]methyl]amino]-2-pyrimidinyl]amino]-4-methyl-5-thiazolecarboxylic acid ethyl ester; 4-Methyl-2-[[4-[[[4-(methylsulfonyl)phenyl]methyl]amino]-6-[[(tetrahydro-2-furanyl)methyl]amino]-2-pyrimidinyl]amino]-5-thiazolecarboxylic acid ethyl ester; 2-[[4-[4-(2-Hydroxyethyl)-1-piperazinyl]-6-[[[4-(methylsulfonyl)phenyl]methyl]amino]-2-pyrimidinyl]amino]-4-methyl-5-thiazolecarboxylic acid ethyl ester; 2-[[4-[2-(Aminocarbonyl)-1-pyrrolidinyl]-6-[[[4-(methylsulfonyl)phenyl]methyl]amino]-2-pyrimidinyl]amino]-4-methyl-5-thiazolecarboxylic acid ethyl ester; 4-Methyl-2-[[4-[methyl (3-pyridinyl methyl)amino]-6-[[[4-(methylsulfonyl)phenyl]methyl]amino]-2-pyrimidinyl]amino]-5-thiazolecarboxylic acid ethyl ester; 2-[[4-[4-(Hydroxymethyl)-1-piperidinyl]-6-[[[4-(methylsulfonyl)phenyl]methyl]amino]-2-pyrimidinyl]amino]-4-methyl-5-thiazolecarboxylic acid ethyl ester; 2-[[4-[[2-(Diethylamino)ethyl]methylamino]-6-[[[4-(methylsulfonyl)phenyl]methyl]amino]-2-pyrimidinyl]amino]-4-methyl-5-thiazolecarboxylic acid ethyl ester; 4-Methyl-2-[[4-[[[4-(methylsulfonyl)phenyl]methyl]amino]-6-[[3-(2-oxo-1-pyrrolidinyl)propyl]amino]-2-pyrimidinyl]amino]-5-thiazolecarboxylic acid ethyl ester; 2-[[4-[3-(Hydroxymethyl)-1-piperidinyl]-6-[[[4-(methylsulfonyl)phenyl]methyl]amino]-2-pyrimidinyl]amino]-4-methyl-5-thiazolecarboxylic acid ethyl ester; 4-Methyl-2-[[4-(4-methyl-1-piperazinyl)-6-[[[(4-(methylsulfonyl)phenyl]methyl]amino]-2-pyrimidinyl]amino]-5-thiazolecarboxylic acid ethyl ester; 2-[4-[2-[(Acetylamino)ethyl]amino]-6-[[[(4-(methylsulfonyl)phenyl]methyl]amino]-2-pyrimidinyl]amino]-4-methyl-5-thiazolecarboxylic acid ethyl ester; 2-[[4-(4-Ethyl-1-piperazinyl)-6-[[[4-(methylsulfonyl)phenyl]methyl]amino]-2-pyrimidinyl]amino]-4-methyl-5-thiazolecarboxylic acid ethyl ester; 2-[[4-(4-Acetyl-1-piperazinyl)-6-[[[4-(methylsulfonyl)phenyl]methyl]amino]-2-pyrimidinyl]amino]-4-methyl-5-thiazolecarboxylic acid ethyl ester; 2-[[4-[[2-(Dimethylamino)ethyl]amino]-[[[4-(methylsulfonyl)phenyl]methyl]amino]-2-pyrimidinyl]amino]-4-methyl-5-thiazolecarboxylic acid ethyl ester; 2-[[4-[3-(Aminocarbonyl)-1-piperazinyl]-6-[[[4-(methylsulfonyl)phenyl]methyl]amino]-2-pyrimidinyl]amino]-4-methyl-5-thiazolecarboxylic acid ethyl ester; 2-[[4-(3-Hydroxy-1-pyrrolidinyl)-6-[[[4-(methylsulfonyl)phenyl]methyl]amino]-2-pyrimidinyl]amino]-4-methyl-5-thiazolecarboxylic acid ethyl ester; 2-[[4-[(4-Hydroxybutyl)amino]-6-[[[(4-(methylsulfonyl)phenyl]methyl]amino]-2-pyrimidinyl]amino]-4-methyl-5-thiazolecarboxylic acid ethyl ester; 2-[[4-[(2,3-Dihydroxypropyl)amino]-6-[[[4-(methylsulfonyl)phenyl]methyl]amino]-2-pyrimidinyl]amino]-4-methyl-5-thiazolecarboxylic acid ethyl ester; 2-[[4-[(4-Amino-1-piperidinyl)-6-[[4-(methylsulfonyl)phenyl]methyl]amino]-2-pyrimidinyl]amino]-4-methyl-5-thiazolecarboxylic acid ethyl ester; 2-[[4-[4-Hydroxy-3-(hydroxymethyl)-1-piperidinyl]-6-[[[4-(methylsulfonyl)phenyl]methyl]amino]-2-pyrimidinyl]amino]-4-methyl-5-thiazolecarboxylic acid ethyl ester; 2-[[4-(4-Dimethylamino-1-piperidinyl)-6-[[[4-(methylsulfonyl)phenyl]methyl]amino]-2-pyrimidinyl]amino]-4-methyl-5-thiazolecarboxylic acid ethyl ester; 2-[[4-[[[4-(Aminosulfonyl)phenyl]methyl]amino]-6-(methylamino)-2-pyrimidinyl]amino]-4-methyl-5-thiazolecarboxylic acid, ethyl ester; 2-[4,6-Bis-(4-methyl-piperazin-1-yl)-pyrimidin-2-ylamino]-4-methyl-thiazole-5-carboxylic acid ethyl ester; 2-[4-(4-Hydroxy-piperidin-1-yl)-6-(4-methyl-piperazin-1-yl)-pyrimidin-2-ylamino]-4-methyl-thiazole-5-carboxylic acid ethyl ester; 2-[4-(3-Hydroxymethyl-piperidin-1-yl)-6-(4-methyl-piperazin-1-yl)-pyrimidin-2-ylamino]-4-methyl-thiazole-5-carboxylic acid ethyl ester; 4-Methyl-2-[4-(4-methyl-piperazin-1-yl)-6-morpholin-4-yl-pyrimidin-2-ylamino]-thiazole-5-carboxylic acid ethyl ester; 2-[4-(4-Amino-piperidin-1-yl)-6-(4-methyl-piperazin-1-yl)-pyrimidin-2-ylamino]-4-methyl-thiazole-5-carboxylic acid ethyl ester; 2-[4,6-Bis-(4-hydroxy-piperidin-1-yl)-pyrimidin-2-ylamino]-4-methyl-thiazole-5-carboxylic acid ethyl ester; 2-[4-(4-oxo-piperidin-1-yl)-6-(4-methyl-piperazin-1-yl)-pyrimidin-2-ylamino]-4-methyl-thiazole-5-carboxylic acid ethyl ester; 2-[4-(4-methyl-4-hydroxy-piperidin-1-yl)-6-(4-methyl-piperazin-1-yl)-pyrimidin-2-ylamino]-4-methyl-thiazole-5-carboxylic acid ethyl ester; 2-[-(4-hydroxy-piperidin-1-yl)-6-(4-dimethylmethyl-piperazin-1-yl)-pyrimidin-2-ylamino]-4-methyl-thiazole-5-carboxylic acid ethyl ester; 2-[4-(4-hydroxymethyl-piperidin-1-yl)-6-(4-dimethylmethyl-piperazin-1-yl)-pyrimidin-2-ylamino]-4-methyl-thiazole-5-carboxylic acid ethyl ester; 2-[4-(3-hydroxymethyl-piperidin-1-yl)-6-(4-dimethylmethyl-piperazin-1-yl)-pyrimidin-2-ylamino]-4-methyl-thiazole-5-carboxylic acid ethyl ester; 2-[4-(4-hydroxymethyl-piperidin-1-yl)-6-(4-hydroxy-piperazin-1-yl)-pyrimidin-2-ylamino]-4-methyl-thiazole-5-carboxylic acid ethyl ester; 4-Methyl-2-[4-(4-hydroxy-piperazin-1-yl)-6-morpholin-4-yl-pyrimidin-2-ylamino]-thiazole-5-carboxylic acid ethyl ester; 2-[[(4-[[[4-(Methylsulfonyl)phenyl]methyl]amino]-6-chloro-2-pyrimidinyl]amino]-4-methyl-5-thiazolecarboxylic acid, ethyl ester; 2-[[4-[[[4-(Aminosulfonyl)phenyl]methyl]amino]-6-chloro-2-pyrimidinyl]amino]-4-methyl-5-thiazolecarboxylic acid, ethyl ester; 2-[[4-[4-(Dimethylamino)-1-piperidinyl]-6-[[(3,4,5-trimethoxyphenyl)methyl]amino]-2-pyrimidinyl]amino]-4-methyl-5-thiazolecarboxylic acid, ethyl ester; 2-[[4-[1-piperizinyl]-6-methyl-6-[[(3,4,5-trimethoxyphenyl)methyl]amino]-2-pyrimidinyl]amino]-4-methyl-5-thiazolecarboxylic acid, ethyl ester; 2-[[4-(4-Amino-1-piperidinyl)-6-[[[4-(methylsulfonyl)phenyl]methyl]amino]-2-pyrimidinyl]amino]-4-methyl-5-thiazolecarboxylic acid, ethyl ester; 2-[[4-[4-hydroxy-1-piperidinyl]-6-methyl-6-[[(3,4,5-trimethoxyphenyl)methyl]amino]-2-pyrimidinyl]amino]-4-methyl-5-thiazolecarboxylic acid, ethyl ester; 2-[[4-[4-(Hydroxymethyl)-1-piperidinyl]-6-methyl-6-[[(3,4,5-trimethoxyphenyl)methyl]amino]-2-pyrimidinyl]amino]-4-methyl-5-thiazolecarboxylic acid, ethyl ester; 2-[4-(4-Hydroxypiperidin-1-yl)-6-(3-oxo-piperazin-1-yl)-pyrimidin-2-ylamino]-4-methyl-thiazole-5-carboxylic acid ethyl ester; 2-[[4-[3-(Aminocarbonyl)-1-piperizinyl]-6-methyl-6-[[(3,4,5-trimethoxyphenyl)methyl]amino]-2-pyrimidinyl]amino]-4-methyl-5-thiazolecarboxylic acid, ethyl ester; 2-[[4-[1-morpholinyl]-6-methyl-6-[[(3,4,5-trimethoxyphenyl)methyl]amino]-2-pyrimidinyl]amino]-4-methyl-5-thiazolecarboxylic acid, ethyl ester; 2-[[4-[3-Oxo-1-piperizinyl]-6-[[(1,1-dioxido-3-oxo-1,2-benzisothiazol-2-(3H)-yl)methyl]amino]-2-pyrimidinyl]amino]-4-methyl-5-thiazolecarboxylic acid, ethyl ester; 2-[[4-[3-Oxo-1-piperizinyl]-6-[[(4-(ethylsulfonylamino)phenyl)methyl]amino]-2-pyrimidinyl]amino]-4-methyl-5-thiazolecarboxylic acid, ethyl ester; 2-[[4-[3-Oxo-1-piperizinyl]-6-[[(4-(hydroxysulfonyl)phenyl)methyl]amino]-2-pyrimidinyl]amino]-4-methyl-5-thiazolecarboxylic acid, ethyl ester; 2-[4-(4-Hydroxypiperidin-1-yl)-6-(4-methyl-3-oxo-piperazin-1-yl)-pyrimidin-2-ylamino]-4-methyl-thiazole-5-carboxylic acid ethyl ester; 2-[4-(4 (Dimethylamino)-piperizin-1-yl)-6-(4-((1-pyrrolidinyl) carbonylmethyl) piperazin-1-yl)-pyrimidin-2-ylamino]-4-methyl-thiazole-5-carboxylic acid ethyl ester; 2-[[4-[3-(Aminocarbonyl)-1-piperazinyl]-6-[[(3,4,5-trimethoxyphenyl)methyl]amino]-2-pyrimidinyl]amino]-4-methyl-5-thiazolecarboxylic acid, ethyl ester; 2-[[4-(4-Amino-1-piperidinyl)-6-[[(3,4,5-trimethoxyphenyl)methyl]amino]-2-pyrimidinyl]amino]-4-methyl-5-thiazolecarboxylic acid, ethyl ester; 2-[[4-[4-(Hydroxymethyl)-1-piperidinyl]-6-[4-[tetrazol-5-yl]-4-hydroxypiperidin-1-yl] 2-pyrimidinyl]amino]-4-methyl-5-thiazolecarboxylic acid, ethyl ester; 2-[[4-[4-methyl-1-piperazinyl]-6-[N-methyl-N-[(3,4,5-trimethoxyphenyl)methyl]amino]-2-pyrimidinyl]amino]-4-methyl-5-thiazolecarboxylic acid, ethyl ester; 2-[[4-[4-Hydroxy-1-piperidinyl]-6-[[(4-(hydroxysulfonyl)phenyl)methyl]amino]-2-pyrimidinyl]amino]-4-methyl-5-thiazolecarboxylic acid, ethyl ester; 2-[[4-[[(4-Cyanophenyl)methyl]amino]-6-(1-piperazinyl)-2-pyrimidinyl]amino]-4-methyl-5-thiazolecarboxylic acid, ethyl ester; trifluoroacetate (1:1); 2-[[4-[[[4-(Aminosulfonyl)phenyl]methyl]amino]-6-(4-morpholinyl)-2-pyrimidinyl]amino]-4-methyl-5-thiazolecarboxylic acid, ethyl ester; 2-[[4-[4-Hydroxy-1-piperidinyl]-6-[(1-oxa-3,8-diazaspiro[4.5]decan-2,4,dion-8-yl]-2-pyrimidinyl]amino]-4-methyl-5-thiazolecarboxylic acid, ethyl ester; 2-[4-(2-(Dimethylamino)ethyl)-piperazin-1-yl)-6-(4-methylpiperazin-1-yl)-pyrimidin-2-ylamino]-4-methyl-thiazole-5-carboxylic acid ethyl ester; 2-[[4-(4-Hydroxy-1-piperidinyl)-6-[methyl (3-pyridinylmethyl)amino]-2-pyrimidinyl]amino]-4-methyl-5-thiazolecarboxylic acid, ethyl ester; 2-[[4-[4-Hydroxy-3-hydroxymethylpiperidin-1-yl]-6-[[(3,4,5-trimethoxyphenyl)methyl]amino]-2-pyrimidinyl]amino]-4-methyl-5-thiazolecarboxylic acid, ethyl ester; 2-[[4-(3,4-Dihydro-6,7-dihydroxy-2(1H)-isoquinolinyl)-6-(4-methyl-1-piperazinyl)-2-pyrimidinyl]amino]-4-methyl-5-thiazolecarboxylic acid, ethyl ester, trifluoroacetate 1:1); 2-[[4-[4-[(Methoxyacetyl)amino]-1-piperidinyl]-6-[[4-(methylsulfonyl)phenyl]methyl]amino]-2-pyrimidinyl]amino]-4-methyl-5-thiazolecarboxylic acid, ethyl ester; 2-[[4-[[(3,4-Dimethoxyphenyl)methyl]amino]-6-[4-(dimethylamino)-1-piperidinyl]-2-pyrimidinyl]amino]-4-methyl-thiazolecarboxylic acid, ethyl ester; 2-[[4-[4-(Hydroxyethyl) piperidin-1-yl]-6-[4-(dimethylamino)-1-piperidinyl]-2-pyrimidinyl]amino]-4-methyl-5-thiazolecarboxylic acid, ethyl ester; 2-[[4-[4-(Dimethylamino)-1-piperidinyl]-6-[methyl (3-pyridinylmethyl)amino]-2-pyrimidinyl]amino]-4-methyl-5-thiazolecarboxylic acid, ethyl ester; 2-[[4-[4-(Hydroxy) piperidin-1-yl]-6-[4-(methoxycarbonyl)-1-piperidinyl]-2-pyrimidinyl]amino]-4-methyl-5-thiazolecarboxylic acid, ethyl ester; 2-[[4-[4-(Hydroxy) piperidin-1-yl]-6-[4-(methyl)-4-(hydroxy)-1-piperidinyl]-2-pyrimidinyl]amino]-4-methyl-5-thiazolecarboxylic acid, ethyl ester; 2-[4-(3-oxopiperazin-1-yl)-6-(4-methylpiperazin-1-yl)-pyrimidin-2-ylamino]-4-methyl-thiazole-5-carboxylic acid ethyl ester; 2-[[4-[[(4-Cyanophenyl)methyl]amino]-6-[4-dimethylamino)-1-piperidinyl]-2-pyrimidinyl]amino]-4-methyl-5-thiazolecarboxylic acid, ethyl ester; 4-Methyl-2-[[4-[[(3-nitrophenyl)methyl]amino]-6-(1-piperazinyl)-2-pyrimidinyl]amino]-5-thiazolecarboxylic acid, ethyl ester, trifluoroacetate (1:1); 2-[[4-(4-Hydroxy-1-piperidinyl)-6-[[(3,4,5-trimethoxyphenyl)methyl]amino]-2-pyrimidinyl]amino]-4-methyl-5-thiazolecarboxylic acid, ethyl ester; 2-[4-(Dimethylamino)-piperazin-1-yl)-6-(4-methyl piperazin-1-yl)-pyrimidin-2-2-ylamino]-4-methyl-thiazole-5-carboxylic acid ethyl ester; 2-[4-(Dimethylamino)-piperidin-1-yl)-6-(3-(aminocarbonyl)-1-piperazinyl)pyrimidin-2-ylamino]-4-methyl-thiazole-5-carboxylic acid ethyl ester; 2-[4-(2-Hydroxyethyl)-piperazin-1-yl)-6-(4-methyl-1-piperazinyl)-pyrimidin-2-ylamino]-4-methyl-thiazole-5-carboxylic acid ethyl ester; 2-[[4-[4-(Aminocarbonyl)-1-piperidinyl]-6-[[[4-(methylsulfonyl)phenyl]methyl]amino]-2-pyrimidinyl]amino]-4-methyl-5-thiazolecarboxylic acid, ethyl ester; 2-[[4-[4-(Hydroxymethyl)-1-piperidinyl]-6-[N-methyl-N-(3-pyridinylmethyl)amino]-2-pyrimidinyl]amino]-4-methy-5-thiazolecarboxylic acid, ethyl ester; 2-[[4-[4-Methylpiperazin-1-yl]-6-[[(3,4-dimethoxyphenyl)methyl]amino]-2-pyrimidinyl]amino]-4-methyl-5-thiazolecarboxylic acid, ethyl ester; 2-[[4-[piperazin-1-yl]-6-[[(4-carboxyphenyl)methyl]amino]-2-pyrimidinyl]amino]-4-methyl-5-thiazolecarboxylic acid, ethyl ester; 2-[[4-[3-Hydroxymethylpiperidin-1-yl]-6-[[N-[(3,4,5-trimethoxyphenyl)methyl]-N-(methyl)amino]-2-pyrimidinyl]amino]-4-methyl-5-thiazolecarboxylic acid, ethyl ester; 2-[4-(4-Hydroxypiperidin-1-yl)-6-(4-carboxypiperidin-1-yl)-pyrimidin-2-ylamino]-4-methyl-thiazole-5-carboxylic acid ethyl ester; 2-[[4-[Piperazin-1-yl]-6-[[(3,4-dimethoxyphenyl)methyl]amino]-2-pyrimidinyl]amino]-4-methyl-5-thiazolecarboxylic acid, ethyl ester; 2-[[4-(4-Formyl-1-piperazinyl)-6-[[[4-(methylsulfonyl)phenyl]methyl]amino]-2-pyrimidinyl]amino]-4-methyl-5-thiazolecarboxylic acid, ethyl ester; 2-[4-(4-Hydroxypiperidin-1-yl)-6-(4-(hydroxy)-4-(4-chlorophenyl) piperidin-1-yl)-pyrimidin-2-ylamino]-4-methyl-thiazole-5-carboxylic acid ethyl ester; 4-Methyl-2-[[4-[4-dimethylamino-1-piperidinyl]-6-[[(tetrahydro-2-furanyl)methyl]amino]-2-pyrimidinyl]amino]-5-thiazolecarboxylic acid ethyl ester; 2-[[4-[Piperazin-1-yl]-6-[[N-methyl-N-(5-tetrazolylmethyl]amino]-2-pyrimidinyl]amino]-4-methyl-5-thiazolecarboxylic acid, ethyl ester; 2-[[4-[4-Morpholinyl]-6-[4-[tetrazol-5-yl]-4-hydroxypiperidin-1-yl]-2-pyrimidinyl]amino]-4-methyl-5-thiazolecarboxylic acid, ethyl ester; 2-[[4-[4-Hydroxy-1-piperidinyl]-6-[[(1,1-dioxido-3-oxo-1,2-benzisothiazol-2-(3H)-yl)methyl]amino]-2-pyrimidinyl]amino]-4-methyl-5-thiazolecarboxylic acid, ethyl ester; 2-[4-(4-Hydroxypiperidin-1-yl)-6-(4-(1-methyl-1-hydroxyethyl) piperidin-1-yl)-pyrimidin-2-ylamino]-4-methyl-thiazole-5-carboxylic acid ethyl ester; 2-[[4-[3-(Aminocarbonyl)-1-piperidinyl]-6-[[N-methyl-N-(3-pyridinylmethyl)]amino]-2-pyrimidinyl]amino]-4-methyl-5-thiazolecarboxylic acid, ethyl ester; 2-[[4-[4-Hydroxymethyl-1-piperidinyl]-6-[[(4-(ethylsulfonylamino)phenyl)methyl]amino]-2-pyrimidinyl]amino]-4-methyl-5-thiazolecarboxylic acid, ethyl ester; 2-[[4-[4-Hydroxy-1-piperidinyl]-6-[4-[tetrazol-5-yl]-4-hydroxypiperidin-1-yl] 2-pyrimidinyl]amino]-4-methyl-5-thiazolecarboxylic acid, ethyl ester; 2-[[4-[4-tertButyloxycarbonylamino-1-piperidinyl]-6-[[N-[(3,4,5-trimethoxyphenyl)methyl]]-N-(methyl)amino]-2-pyrimidinyl]amino]-4-methyl-5-thiazolecarboxylic acid, ethyl ester; 2-[[4-[[(4-Cyanophenyl)methyl]amino]-6-(4-methyl-1-piperazinyl)-2-pyrimidinyl]amino]-4-methyl-5-thiazolecarboxylic acid, ethyl ester, trifluoroacetate (1:1); 2-[[4-[4-[[(2-Ethoxy-2-oxoethyl)amino]carbonyl]-1-piperazinyl]-6-[methyl (3-pyridinylmethy)amino]-2-pyrimidinyl]amino]-4-methyl-5-thiazolecarboxylic acid, ethyl ester, trifluoroacetate (1:1); 2-[4-(4-Hydroxypiperidin-1-yl)-6-(3-hydroxypiperidin-1-yl)-pyrimidin-2-ylamino]-4-methyl-thiazole-5-carboxylic acid ethyl ester; 2-[4-(4-Hydroxypiperidin-1-yl)-6-(4-hydroxy-4-phenyl-1-piperidinyl)-pyrimidin-2-ylamino]-4-methyl-thiazole-5-carboxylic acid ethyl ester; 4-Methyl-2-[[4-[4-morpholinyl]-6-[[(tetrahydro-2-furanyl)methyl]amino]-2-pyrimidinyl]amino]-5-thiazolecarboxylic acid ethyl ester; 2-[[4-[(Tetrahydro-2-furanyl)methyl]amino]-6-[[N-[(3,4,5-trimethoxyphenyl)methyl][-N-(methyl)amino]-2-pyrimidinyl]amino]-4-methyl-5-thiazolecarboxylic acid, ethyl ester; 2-[[4-[4-Morpholinyl]-6-[[(4-(hydroxysulfonyl)phenyl)methyl]amino]-2-pyrimidinyl]amino]-4-methyl-5-thiazolecarboxylic acid, ethyl ester; 2-[Bis-4,6-(4-Cyano-1-piperidinyl)-pyrimidin-2-ylamino]-4-methyl-thiazole-5-carboxylic acid ethyl ester; 2-[[4-[4-(Cyclopentylaminocarbonyl)-1-piperazinyl]-6-[N-methyl-N-(3-pyridinylmethyl)amino]-2-pyrimidinyl]amino]-4-methyl-5-thiazolecarboxylic acid, ethyl ester; 2-[4-(2-Methoxyethyl)-piperazin-1-yl)-6-(4-methyl-1-piperzinyl)-pyrimidin-2-ylamino]-4-methyl-thiazole-5-carboxylic acid ethyl ester; 2-[4-(4-Hydroxypiperidin-1-yl)-6-(3-carboxypiperidin-1-yl)-pyrimidin-2-ylamino]-4-methyl-thiazole-5-carboxylic acid ethyl ester; 2-[[4-[4-Methylpiperazin-1-yl]-6-[3-(acetylamino)-1-pyrrolidinyl]-2-pyrimidinyl]amino]-4-methyl-5-thiazolecarboxylic acid, ethyl ester; 2-[[4-[3-(Aminocarbonyl)-1-piperazinyl]-6-[[N-methyl-N-(3-pyridinylmethyl)]amino]-2-pyrimidinyl]amino]-4-methyl-5-thiazolecarboxylic acid, ethyl ester; 2-[[4-[2-Methyl-3-oxol-piperizinyl]-6-[4-methyl-1-piperazinyl]-2-pyrimidinyl]amino]-4-methyl-5-thiazolecarboxylic acid, ethyl ester; 2-[[4-[3-(Aminocarbonyl)-1-piperazinyl]-6-(4-methyl-1-piperazinyl)-2-pyrimidinyl]amino]-4-methyl-5-thiazolecarboxylic acid, ethyl ester; 2-[[4-[3-(Aminocarbonyl)-1-piperidinyl]-6-(4-dimethylamino-1-piperidinyl)-2-pyrimidinyl]amino]-4-methyl-5-thiazolecarboxylic acid, ethyl ester; 2-[[4-[1-piperazinyl]-6-[[N-methyl-N-(2-furylmethyl)]amino]-2-pyrimidinyl]amino]-4-methyl-5-thiazolecarboxylic acid, ethyl ester; 2-[[4-[[(4-Methoxycarbonylphenyl)methyl]amino]-6-(4-dimethyl-1-piperidinyl)-2-pyrimidinyl]amino]-4-methyl-5-thiazolecarboxylic acid, ethyl ester, trifluoroacetate (1:1); 2-[[4-[3-Oxo-1-piperazinyl]-6-[[(4-(methylsulfonylamino)phenyl)methyl]amino]-2-pyrimidinyl]amino]-4-methyl-5-thiazolecarboxylic acid, ethyl ester; 2-[[4-[3-Oxo-1-piperazinyl]-6-[[(4-(propylsulfonylamino)phenyl)methyl]amino]-2-pyrimidinyl]amino]-4-methyl-5-thiazolecarboxylic acid, ethyl ester; 2-[[4-[3-(Aminocarbonyl)-1-piperidinyl]-6-[[(3,4,5-trimethoxyphenyl)methyl]amino]-2-pyrimidinyl]amino]-4-methyl-5-thiazolecarboxylic acid, ethyl ester; 2-[Bis-4,6-(4-Hydroxy-4-methyl-1-piperidinyl)-pyrimidin-2-ylamino]-4-methyl-thiazole-5-carboxylic acid ethyl ester; 4-Methyl-2-[[4-[4-dimethylamino-1-piperidinyl]-6-[[(2-oxo-1-pyrrolidinyl)propyl]amino]-2-pyrimidinyl]amino]-5-thiazolecarboxylic acid ethyl ester; 2-[[4-[3-Oxo-1-piperazinyl]-6-[[(4-(iso-propylsulfonylamino)phenyl)methyl]amino]-2-pyrimidinyl]amino]-4-methyl-5-thiazolecarboxylic acid, ethyl ester; 2-[4-(4-Hydroxypiperidin-1-yl)-6-(3-hydroxymethyl-1-piperidinyl)-pyrimidin-2-ylamino]-4-methyl-thiazole-5-carboxylic acid ethyl ester; 4-Methyl-2-[[4-[4-hydroxy-1-piperidinyl]-6-[[(2-(4-morpholinyl)ethyl]amino]-2-pyrimidinyl]amino]-5-thiazolecarboxylic acid ethyl ester; 2-[[4-[[[4-(Ethylaminosulfonyl)phenyl]methyl]amino]-6-methoxy-2-pyrimidinyl]amino]-4-methyl-5-thiazolecarboxylic acid, methyl ester, trifluoroacetate (1:1); 2-[[4-[4-Morpholinyl]-6-[(1-oxa-3,8-diazaspiro[4.5]decan-2,4, dion-8-yl]-2-pyrimidinyl]amino]-4-methyl-5-thiazolecarboxylic acid, ethyl ester; 2-[[4-[4-Hydroxy-1-piperidinyl]-6-[[(4-(ethylsulfonylamino)phenyl)methyl]amino]-2-pyrimidinyl]amino]-4-methyl-5-thiazolecarboxylic acid, ethyl ester; 2-[[4-[tertButyloxycarbonyl-1-piperazinyl]-6-[[(3,4,5-trimethoxyphenyl)methyl]amino]-2-pyrimidinyl]amino]-4-methyl-5-thiazolecarboxylic acid, ethyl ester; 2-[[4-[3-(Aminocarbonyl)-1-piperidinyl]-6-[[(3,4-dimethoxyphenyl)methyl]amino]-2-pyrimidinyl]amino]-4-methyl-5-thiazolecarboxylic acid, ethyl ester; 2-[[4-[4-ethoxycarbonyl-1-piperazinyl]-6-[[N-methyl-N-(5-tetrazolylmethyl]amino]-2-pyrimidinyl]amino]-4-methyl-5-thiazolecarboxylic acid, ethyl ester; 2-[[4-[3-Oxo-1-piperizinyl]-6-[[(4-(cyclopropylsulfonylamino)phenyl)methyl]amino]-2-pyrimidinyl]amino]-4-methyl-5-thiazolecarboxylic acid, ethyl ester; 2-[[4-[4-Hydroxymethyl-1-piperidinyl]-6-[[(4-(methylsulfonylamino)phenyl)methyl]amino]-2-pyrimidinyl]amino]-4-methyl-5-thiazolecarboxylic acid, ethyl ester; 2-[4-(4-Dimethylamino-1-piperazinyl)-6-(4-tertbutyloxycarbonylamino-1-piperidinyl)-pyrimidin-2-ylamino]-4-methyl-thiazole-5-carboxylic acid ethyl ester; 2-[4-(4-Hydroxypiperidin-1-yl)-6-(4-methoxymethyl-1-piperidinyl)-pyrimidin-2-ylamino]-4-methyl-thiazole-5-carboxylic acid ethyl ester; 2-[4-(4-Hydroxypiperidin-1-yl)-6-(4-hydroxyethyl-1-piperidinyl)-pyrimidin-2-ylamino]-4-methyl-thiazole-5-carboxylic acid ethyl ester; 2-[4-(4-Hydroxypiperidin-1-yl)-6-(4-(hydroxy)-4-(3-trifluoromethylphenyl) piperidin-1-yl)-pyrimidin-2-ylamino]-4-methyl-thiazole-5-carboxylic acid ethyl ester; 2-[[4-[4-morpholinyl]-6-[4-[1-methyl-1-hydroxyethyl]-1-piperidinyl]-2-pyrimidinyl]amino]-4-methyl-5-thiazolecarboxylic acid, ethyl ester; 2-[[4-(3-Oxo-1-piperizinyl]-6-[[3-pyridyl]oxy]-2-pyrimidinyl]amino]-4-methyl-5-thiazolecarboxylic acid, ethyl ester; 2-[[4-[4-Methyl-1-piperazinyl]-6-[(1,4-dioxaspiro[4.5]decan-8-yl]-2-pyrimidinyl]amino]-4-methyl-5-thiazolecarboxylic acid, ethyl ester; 2-[[4-[4-Morpholinyl]-6-[[(4-(methylsulfonylamino)phenyl)methyl]amino]-2-pyrimidinyl]amino]-4-methyl-5-thiazolecarboxylic acid, ethyl ester; 2-[[4-[3-Oxo-1-piperazinyl]-6-[(1-oxa-3,8-diazospiro[4.5]decan-2,4, dion-8-yl]-2-pyrimidinyl]amino]-4-methyl-5-thiazolecarboxylic acid, ethyl ester; 2-[[4-[4-Hydroxy-1-piperidinyl]-6-[[(4-(carboxy)phenyl)methyl]amino]-2-pyrimidinyl]amino]-4-methyl-5-thiazolecarboxylic acid, ethyl ester; 2-[4-(4-Hydroxypiperidin-1-yl)-6-(4-(hydroxy)-4-(4-bromophenyl)piperidin-1-yl)-pyrimidin-2-ylamino]-4-methyl-thiazole-5-carboxylic acid ethyl ester; 2-[[4-[4-Morpholinyl]-6-[[[4-ethylsulfonylamino)phenyl)methyl]amino]-2-pyrimidinyl]amino]-4-methyl]-5-thiazolecarboxylic acid, ethyl ester; 2-[[4-[3-(Aminocarbonyl)-1-piperazinyl]-6-[[(3,4-dimethoxyphenyl)methyl]amino]-2-pyrimidinyl]amino]-4-methyl-5-thiazolecarboxylic acid, ethyl ester; 2-[[4-[4-Formyl-1-piperazinyl]-6-[[(3-(5-(1H)tetrazolyl)phenyl)methyl]amino]-2-pyrimidinyl]amino]-4-methyl-5-thiazolecarboxylic acid, ethyl ester; 2-[[4-[4-(Hydroxymethyl)-1-Piperidinyl]-6-[[N-methyl-N-(5-tetrazolylmethyl]amino]-2-pyrimidinyl]amino]-4-methyl-5-thiazolecarboxylic acid, ethyl ester; 2-[4-[4-Methyl-1-piperazinyl]-6-[[(2,5-dimethyl)phenyl)methyl)amino]-2-pyrimidinyl]amino]-4-methyl-5-thiazolecarboxylic acid, ethyl ester; 2-[[4-[3-(2-oxo-1-pyrrolidinyl)propyl]amino]-6-[N-methyl-N-(3-pyridinylmethyl)amino]-2-pyrimidinyl]amino]-4-methyl-5-thiazolecarboxylic acid, ethyl ester; 2-[[4-[(1-Morpholinyl)]-6-[[N-methyl-N-(5-tetrazolylmethyl]amino]-2-pyrimidinyl]amino]-4-methyl-5-thiazolecarboxylic acid, ethyl ester; 2-[[4-[4-methyl-1-piperazinyl]-6-[4-[methylsulfonylamino]-1-piperidinyl]-2-pyrimidinyl]amino]-4-methyl-5-thiazolecarboxylic acid, ethyl ester; 2-[[4-[4-hydroxy-1-piperidinyl]-6-[[(2,5-dimethyl)phenyl)methyl]amino]-2-pyrimidinyl]amino]-4-methyl-5-thiazolecarboxylic acid, ethyl ester; 4-Methyl-2-[[4-(4-morpholinyl)-6-[[(3,4,5-trimethoxyphenyl)methyl]amino-2-pyrimidinyl]amino]-5-thiazolecarboxylic acid, ethyl ester; 2-[4-(4-Hydroxypiperidin-1-yl)-6-(3-hydroxy-1-piperidinyl)-pyrimidin-2-ylamino]-4-methyl-thiazole-5-carboxylic acid ethyl ester; 4-Methyl-2-[[4-(4-methyl-1-piperazinyl)-6-[methyl (3-pyridinylmethyl)amino]-2-pyrimidinyl]amino]-5-thiazolecarboxylic acid, ethyl ester; 2-[[4-[3-Oxo-1-piperazinyl]-6-[[(2-(5-(1H)tetrazolyl)phenyl)methyl]amino]-2-pyrimidinyl]amino]-4-methyl-4-thiazolecarboxylic acid, ethyl ester; 2-[[4-[(2-Furanylmethyl)amino]-6-(1-piperazinyl)-2-pyrimidinyl]amino]-4-methyl-5-thiazolecarboxylic acid, ethyl ester, trifluoroacetate (1:1); 2-[[4-[[(3,4-Dimethoxyphenyl)methyl]amino]-6-(4-morpholinyl)-2-pyrimidinyl]amino]-4-methyl-5-thiazolecarboxylic acid, ethyl ester; 4-Methyl-2-[[4-[methyl (3-pyridinylmethyl)amino]-6-[[(tetrahydro-2-furanyl)methyl]amino]-2-pyrimidinyl]amino]-5-thiazolecarboxylic acid, ethyl ester; 2-[[4-[(4-hydroxy-1-piperidinyl)]-6-[[N-methyl-N-(5-tetrazolylmethyl]amino]-2-pyrimidinyl]amino]-4-methyl-5-thiazolecarboxylic acid, ethyl ester; 2-[4-(4-Hydroxypiperidin-1-yl)-6-[(4-(hydroxy)-4-(phenylmethyl) piperidin-1-yl)]-pyrimidin-2-ylamino]-4-methyl-thiazole-5-carboxylic acid ethyl ester; 2-[4-(4-Dimethylamino-1-piperazinyl)-6-[[2-(1-morpholinyl)ethyl]amino]pyrimidin-2-ylamino]-4-methyl-thiazole-5-carboxylic acid ethyl ester; 2-[[4-[4-hydroxy-1-piperidinyl]-6-[[(3-pyridinyl methyl)]oxy]-2-pyrimidinyl]amino]-4-methyl-5-thiazolecarboxylic acid, ethyl ester; 2-[[4-[3-(Aminocarbonyl)-1-piperidinyl]-6-[[(2,6-dimethylphenyl)methyl]amino]-2-pyrimidinyl]amino]-4-methyl-5-thiazolecarboxylic acid, ethyl ester; 2-[[4-[4-hydroxy-1-piperidinyl]-6-[[(4-(methylsulfonylamino)phenyl)methyl]amino]-2-pyrimidinyl]amino]-4-methyl-5-thiazolecarboxylic acid, ethyl ester; 2-[[4-[4-hydroxy-1-piperidinyl]-6-[[(4-(propylsulfonylamino)phenyl)methyl]amino]-2-pyrimidinyl]amino]-4-methyl-5-thiazolecarboxylic acid, ethyl ester; 2-[[4-[3-(Aminocarbonyl)-1-piperidinyl]-6-(4-methyl-1-piperazinyl)-2-pyrimidinyl]amino]-4-methyl-5-thiazolecarboxylic acid, ethyl ester; 2-[[4-(3,4-Dihydro-6,7-dimethoxy-2(1H)-isoquinolinyl)-6-(4-methyl-1-piperazinyl)-2-pyrimidinyl]amino]-4-methyl-5-thiazolecarboxylic acid, ethyl ester; 2-[[4-[4-Formyl-1-piperazinyl]-6-[[N-methyl-N-(5-tetrazolylmethyl]amino]-2-pyrimidinyl]amino]-4-methyl-5-thiazolecarboxylic acid, ethyl ester; 2-[[4-[[(4-Carboxyphenyl)methyl]amino]-6-[4-(hydroxymethyl)-1-piperidinyl]-2-pyrimidinyl]amino]-4-methyl-5-thiazolecarboxylic acid, ethyl ester; 2-[[4-[[(4-Carboxyphenyl)methyl]amino]-6-(4-methyl-1-piperazinyl)-2-pyrimidinyl]amino]-4-methyl-5-thiazolecarboxylic acid, ethyl ester, monohydrochloride; 4-Methyl-2-[[4-(4-methyl-1-piperazinyl)-6-[[(tetrahydro-2-furanyl)methyl]amino]-2-pyrimidinyl]amino]-5-thiazolecarboxylic acid, ethyl ester; 2-[[4-[[(4-Carboxyphenyl)methyl]amino]-6-[3-(hydroxymethyl)-1-piperidinyl]-2-pyrimidinyl]amino]-4-methyl-5-thiazolecarboxylic acid, ethyl ester; 2-[[4-[[[4-[[(2-Methoxyethyl)amino]carbonyl]phenyl]methyl]amino]-6-(4-methyl-1-piperazinyl)-2-pyrimidinyl]amino]-4-methyl-5-thiazolecarboxylic acid, ethyl ester, trifluoroacetate (1:1); 2-[4,6-Bis-(1-morpholinyl)-pyrimidin-2-ylamino]-4-methyl-thiazole-5-carboxylic acid ethyl ester; 2-[[4-[3-(Aminocarbonyl)-1-piperazinyl]-6-[[N-methyl-N-(5-tetrazolylmethyl]amino]-2-pyrimidinyl]amino]-4-methyl-5-thiazolecarboxylic acid ethyl ester; 4-Methyl-2-[[4-[-methyl (3-pyridinylmethyl)amino]-6-[4-morpholinyl]-2-pyridinylmethyl]amino]-5-thiazolecarboxylic acid, ethyl ester; 2-[[4-[3-(Aminocarbonyl)-1-piperazinyl]-6-[[[4-(methoxycarbonyl)phenyl]methyl]amino]-2-pyrimidinyl]amino]-4-methyl-5-thiazolecarboxylic acid, ethyl ester; 2-[[4-Chloro-6-[(1-oxa-3,8-diazaspiro[4.5]decan-2,4, dion-8-yl]-2-pyrimidinyl]amino]-4-methyl-5-thiazolecarboxylic acid, ethyl ester; 2-[[4-[4-(Hydroxymethyl)-1-piperidinyl]-6-[[(3,4,5-trimethoxyphenyl)methyl]amino]-2-pyrimidinyl]amino]-4-methyl-5-thiazolecarboxylic acid, ethyl ester; 2-[[4-[3-(Hydroxymethyl)-1-Piperidinyl]-6-[[N-methyl-N-(5-tetrazolylmethyl]amino]-2-pyrimidinyl]amino]-4-methyl-5-thiazolecarboxylic acid, ethyl ester; 2-[[4-[3-(Hydroxymethyl)-1-pyrrolidinyl]-6-[[N-methyl-N-(5-tetrazolylmethyl]amino]-2-pyrimidinyl]amino]-4-methyl-5-thiazolecarboxylic acid, ethyl ester; 4-Methyl-2-[[4-[methyl(phenylmethyl)amino]-6-(4-methyl-1-piperazinyl)-2-pyrimidinyl]amino]-5-thiazolecarboxylic acid, ethyl ester; 2-[[4-(Dimethylamino)-6-[[[4-(methylsulfonyl)phenyl]methyl]amino]-2-pyrimidinyl]amino]-4-methyl-5-thiazolecarboxylic acid, ethyl ester; 2-[[4-[4-Hydroxy-1-piperidinyl]-6-[[(3-(5-(1H)tetrazolyl)phenyl)methyl]amino]-2-pyrimidinyl]amino]-4-methyl-5-thiazolecarboxylic acid, ethyl ester; 2-[[4-[4-hydroxymethyl-1-piperidinyl]-6-[[(4-(propylsulfonylamino)phenyl)methyl]amino]-2-pyrimidinyl]amino]-4-methyl-5-thiazolecarboxylic acid, ethyl ester; 2-[[4-[4-hydroxymethyl-1-piperidinyl]-6-[[(4-(cyclopropylsulfonylamino)phenyl)methyl]amino]-2-pyrimidinyl]amino]-4-methyl-5-thiazolecarboxylic acid, ethyl ester; 2-[[4-[3-(Hydroxymethyl)-1-piperidinyl]-6-[[(3,4,5-trimethoxyphenyl)methyl]amino]-2-pyrimidinyl]amino]-4-methyl-5-thiazolecarboxylic acid, ethyl ester; 2-[[4-[4-tetrahydropyranyl]oxy-6-[[N-[(3,4,5-trimethoxyphenyl)methyl]]-N-(methyl)amino]-2-pyrimidinyl]amino]-4-methyl-5-thiazolecarboxylic acid, ethyl ester; 2-[[4-[4-Methyl-1-piperazinyl]-6-[(4-methoxyphenyl)oxy]-2-pyrimidinyl]amino]-4-methyl-5-thiazolecarboxylic acid, ethyl ester; 4-Methyl-2-[4-(4-methyl-piperazin-1-yl)-6-[[[4-(aminosulfonyl)phenyl]methyl]amino]pyrimidin-2-ylamino]-thiazole-5-carboxylic acid ethyl ester; 2-[4-Isopropyl-6-(4-sulfamoyl-benzylamino]-4-methyl-thiazole-5-carboxylic acid ethyl ester; 4-Methyl-2-[4-(4-sulfamoyl-benzylamino)-6-methyl-pyrimidin-2-ylamino]-thiazole-5-carboxylic acid ethyl ester; 4-Methyl-2-[4-(4-sulfamoyl-benzylamino)-6-hydroxymethyl-pyrimidin-2-ylamino]-thiazole-5-carboxylic acid ethyl ester; 4-Methyl-2-[4-(4-methyl-piperazin-1-yl)-6-[4-(1H-tetrazol-5-yl)-benzylamino]-pyrimidin-2-ylamino]-thiazole-5-carboxylic acid ethyl ester; 2-[4-(4-Hydroxy-piperidin-1-yl)-6-[4-(1H-tetrazol-5-yl)-benzylamino]-pyrimidin-2-ylamino]-4-methyl-thiazole-5-carboxylic acid ethyl ester; 4-Methyl-2-[4-[(tetrahydro-furan-2-ylmethyl)-amino]-6-[4-(1H-tetrazol-5-yl)-benzylamino]-pyrimidin-2-ylamino]-thiazole-5-carboxylic acid ethyl ester; 4-Methyl-2-[4-morpholin-4-yl-6-[4-(1H-tetrazol-5-yl)-benzylamino]pyrimidin-2-ylamino]-thiazole-5-carboxylic acid ethyl ester; 2-[4-(3-Carbamoyl-piperidin-1-yl)-6-[4-(1H-tetrazol-5-yl)-benzylamino]-pyrimidin-2-ylamino])-4-methyl-thiazole-5-carboxylic acid ethyl ester; 2-[4-(4-Hydroxymethylpiperidin-1-yl)-6-[4-(1H-tetrazol-5-yl)-benzylamino]-pyrimidin-2-ylamino]-4-methyl-thiazole-5-carboxylic acid ethyl ester; 2-[4-(2-Hydroxymethyl-1-pyrrolidinyl)-6-[4-(1H-tetrazol-5-yl)-benzylamino]-pyrimidin-2-ylamino]-4-methyl-thiazole-5-carboxylic acid ethyl ester; 2-[4-(3-N,N-Diethylcarbamoyl-1-piperidinyl)-6-[4-(1H-tetrazol-5-yl)-benzylamino]-pyrimidin-2-ylamino]-4-methyl-thiazole-5-carboxylic acid ethyl ester; 2-[4-(3-Hydroxy-1-pyrrolidinyl)-6-[4-(1H-tetrazol-5-yl)-benzylamino]-pyrimidin-2-ylamino]-4-methyl-thiazole-5-carboxylic acid ethyl ester; 4-Methyl-2-[[(2-[4-morpholin-4-yl]ethyl]amino-6-[4-(1H-tetrazol-5-yl)-benzylamino]pyrimidin-2-ylamino]-thiazole-5-carboxylic acid ethyl ester; 4-Methyl-2-[[[4-hydroxyl]butyl]amino-6-[4-(1H-tetrazol-5-yl)-benzylamino]-pyrimidin-2-ylamino]-thiazole-5-carboxylic acid ethyl ester; 2-[4-(4-Formyl-1-piperazinyl)-6-[4-(1H-tetrazol-5-yl)-benzylamino]-pyrimidin-2-ylamino]-4-methyl-thiazole-5-carboxylic acid ethyl ester; 2-[[4-[[(4-Chlorophenyl)methyl]amino]-6-(5-oxazolyl)-2-pyrimidinyl]amino]-4-methyl-5-thiazolecarboxylic acid ethyl ester; 2-[[4-[[(4-Aminosylfonylphenyl)methyl]amino]-6-(5-oxazolyl)-2-pyrimidinyl]amino]-4-methyl-5-thiazolecarboxylic acid ethyl ester; 2-[[4-Morpholino-6-(5-oxazolyl)-2-pyrimidinyl]amino]-4-methyl-5-thiazolecarboxylic acid ethyl ester; 2-[[4-[[(3,4-Dimethoxyphenyl)methyl]amino]-6-(5-oxazolyl)-2-pyrimidinyl]amino]-4-methyl-5-thiazolecarboxylic acid ethyl ester; 2-[[4-(1,4-Dioxa-8-aza-spiro[4.5]dec-8-yl)-6-(5-oxazoly)-2-pyrimidinyl]amino]-4-methyl-5-thiazolecarboxylic acid ethyl ester; 2-[[4-[4-Hydroxy-4-phenyl-piperidinyl]-6-(5-oxazolyl)-2-pyrimidinyl]amino]-4-methyl-5-thiazolecarboxylic acid ethyl ester; 2-[[4-[[(4-Methylsulfonylphenyl)methyl]amino]-6-(5-oxazolyl)-2-pyrimidinyl]amino]-4-methyl-5-thiazolecarboxylic acid ethyl ester; 2-[[4-[4-Hydroxy-piperidinyl]-6-(5-oxazolyl)-2-pyrimidinyl]amino]-4-methyl-5-thiazolecarboxylic acid ethyl ester; 2-[[4-[4-Ethoxycarbonyl-piperidinyl]-6-(5-oxazolyl)-2-pyrimidinyl]amino]-4-methyl-5-thiazolecarboxylic acid ethyl ester; 2-[[4-Piperidinyl-6-(5-oxazolyl)-2-pyrimidinyl]amino]-4-methyl-5-thiazolecarboxylic acid ethyl ester; 2-[[4-[N-Methylpiperazinyl-6-(5-oxazolyl)-2-pyrimidinyl]amino]-4-methyl-5-thiazolecarboxylic acid ethyl ester; 2-[[4-[N-(2-Furylcarbonyl) piperazinyl-6-(5-oxazolyl)-2-pyrimidinyl]amino]-4-methyl-5-thiazolecarboxylic acid ethyl ester; 2-[[4-[N-Acetyl-[1,4-diazepyl]-6-(5-oxazolyl)-2-pyrimidinyl]amino]-4-methyl-5-thiazolecarboxylic acid ethyl ester; 2-[[4-[N-Methyl-N—(N-methyl-4-piperidinyl)-amino]-6-(5-oxazolyl)-2-pyrimidinyl]amino]-4-methyl-5-thiazolecarboxylic acid ethyl ester; 2-[[4-[N-Methyl-[1,4]-diazepyl]-6-(5-oxazolyl)-2-pyrimidinyl]amino]-4-methyl-5-thiazolecarboxylic acid ethyl ester; 2-[[4-N,N-Dimethoxyethylamino-6-(5-oxazolyl)-2-pyrimidinyl]amino]-4-methyl-5-thiazolecarboxylic acid ethyl ester; 2-[[4-[(1′,4)-Bipiperidinyl]-6-(5-oxazolyl)-2-pyrimidinyl]amino]-4-methyl-5-thiazolecarboxylic acid ethyl ester; 2-[4-(4-Hydroxy-piperidin-1-yl)-6-(3,4,5-trimethoxy-phenyl)-pyrimidin-2-ylamino]-4-methylthiazole-5-carboxylic acid ethyl ester; 2-[(4-(4-Hydroxy-piperidin-1-yl)-6-[4-(1H-tetrazol-5-yl)-phenyl]-pyrimidin-2-ylamino]-4-methylthiazole-5-carboxylic acid ethyl ester; 2-[4-(4-Hydroxy-piperidin-1-yl)-6-pyridin-3-yl-pyrimidin-2-ylamino]-4-methylthiazole-5-carboxylic acid ethyl ester; 2-[4-(4-Methanesulfonyl-benzylamino)-6-pyridin-3-yl-pyrimidin-2-ylamino]-4-methylthiazole-5-carboxylic acid ethyl ester; 2-[4-(4-Hydroxy-piperidin-1-yl)-6-pyrimidin-4-yl-pyrimidin-2-ylamino]-4-methylthiazole-5-carboxylic acid ethyl ester; 2-[4-(4-Cyano-phenyl)-6-(4-hydroxy-piperidin-1-yl)-pyrimidin-2-ylamino]-4-methylthiazole-5-carboxylic acid ethyl ester; 2-[4-(4-Acetyl-phenyl)-6-(4-hydroxy-piperidin-1-yl)-pyrimidin-2-ylamino]-4-methylthiazole-5-carboxylic acid ethyl ester; 2-[4-(4-Hydroxymethyl-phenyl)-6-(4-hydroxy-piperidin-1-yl)-pyrimidin-2-ylamino]-4-methylthiazole-5-carboxylic acid ethyl ester; 2-[4-(4-Hydroxy-phenyl)-6-(4-hydroxy-piperidin-1-yl)-pyrimidin-2-ylamino]-4-methylthiazole-5-carboxylic acid ethyl ester; 2-[4-(4-Methanesulfonyl-benzylamino)-6-(3,4,5-trimethoxy-phenyl)-pyrimidin-2-ylamino]-4-methylthiazole-5-carboxylic acid ethyl ester; 2-[4-(4-Methanesulfinylphenyl)-6-(4-hydroxypiperidin-1-yl)-pyrimidin-2-ylamino]-4-methylthiazole-5-carboxylic acid ethyl ester; 2-[4-(4-(Amino)phenyl)-6-(4-hydroxy-piperidin-1-yl)-pyrimidin-2-ylamino]-4-methylthiazole-5-carboxylic acid ethyl ester; 2-[4-(4-Carboxymethyl-phenyl)-6-(4-hydroxy-piperidin-1-yl)-pyrimidin-2-ylamino]-4-methylthiazole-5-carboxylic acid ethyl ester; 2-[4-(4-(Trifluoromethylcarbonylamino)phenyl)-6-(4-hydroxy-piperidin-1-yl)-pyrimidin-2-ylamino]-4-methylthiazole-5-carboxylic acid ethyl ester; 2-[4-(4-(Ethoxycarbonylmethyl)phenyl)-6-(4-hydroxy-piperidin-1-yl)-pyrimidin-2-ylamino]-4-methylthiazole-5-carboxylic acid ethyl ester; 2-[4-(1,2,3,6-Tetrahydropyridin-4-yl)-6-(4-hydroxy-piperidin-1-yl)-pyrimidin-2-ylamino]-4-methylthiazole-5-carboxylic acid ethyl ester; 2-[4-(3-(cyano)phenyl)-6-(4-hydroxy-piperidin-1-yl)-pyrimidin-2-ylamino]-4-methylthiazole-5-carboxylic acid ethyl ester; 2-[4-(4-(Methoxycarbonyl)phenyl)-6-(4-hydroxy-piperidin-1-yl)-pyrimidin-2-ylamino]-4-methylthiazole-5-carboxylic acid ethyl ester; 2-[4-(2-(Methoxy)-5-pyridinyl)-6-(4-hydroxy-piperidin-1-yl)-pyrimidin-2-ylamino]-4-methylthiazole-5-carboxylic acid ethyl ester; 2-[4-(4-tertButyloxycarbonyl-1,2,3,6-Tetrahydropyridin-4-yl)-6-(4-hydroxy-piperidin-1-yl)-pyrimidin-2-ylamino]-4-methylthiazole-5-carboxylic acid ethyl ester; 2-[4-(1,4-Dioxaspiro[4.5]dec-7-en-8-yl)-6-(4-hydroxy-piperidin-1-yl)-pyrimidin-2-ylamino]-4-methylthiazole-5-carboxylic acid ethyl ester; 2-[4-(4-Methyl-1-piperazin-yl)-6-(3,4,5-trimethoxy-phenyl)-pyrimidin-2-ylamino]-4-methylthiazole-5-carboxylic acid ethyl ester; 2-[4-(4-Morpholinyl)-6-(3,4,5-trimethoxy-phenyl)-pyrimidin-2-ylamino]-4-methylthiazole-5-carboxylic acid ethyl ester; 2-[4-(4-Morpholinyl)-6-(3-pyridinyl)-pyrimidin-2-ylamino]-4-methylthiazole-5-carboxylic acid ethyl ester; 2-[4-(Piperidin-4-yl)-6-(4-hydroxy-piperidin-1-yl)-pyrimidin-2-ylamino]-4-methylthiazole-5-carboxylic acid ethyl ester; 2-[[4-[4-Hydroxy-piperidinyl]-6-(3,5-dimethyl-4-isoxazolyl)-2-pyrimidinyl]amino]-4-methyl-5-thiazole-carboxylic acid ethyl ester; 2-[4-(4-tert-Butoxycarbonylamino-phenyl)-6-(4-hydroxy-piperidin-1-yl)-pyrimidin-2-ylamino]-4-methyl-5-thiazolecarboxylic acid ethyl ester; 2-[4-(4-Cyano-phenyl)-6-(4-methanesulfonyl-benzylamino)-pyrimidin-2-ylamino]-4-methyl-5-thiazolecarboxylic acid ethyl ester; 2-[4-(4-Methanesulfonylphenyl)-6-(4-hydroxypiperidin-1-yl)-pyrimidin-2-ylamino]-4-methylthiazole-5-carboxylic acid ethyl ester; 2-[4-(4-Methanesulfanylphenyl)-6-(4-hydroxypiperidin-1-yl)-pyrimidin-2-ylamino]-4-methylthiazole-5-carboxylic acid ethyl ester; 2-[4-(4-Carboxy-phenyl)-6-(4-hydroxy-piperidin-1-yl)-pyrimidin-2-ylamino]-4-methyl-thiazole-5-carboxylic acid ethyl ester; 2-[4-(4-Carboxy-phenyl)-6-(3-oxo-piperazin-1-yl)-pyrimidin-2-ylamino]-4-methylthiazole-5-carboxylic acid ethyl ester; 2-[4-(4-Carboxy-phenyl)-6-(4-methyl-piperazin-1-yl)-pyrimidin-2-ylamino]-4-methylthiazole-5-carboxylic acid ethyl ester; 2-[4-(4-Carboxy-phenyl)-6-morpholin-4-yl-pyrimidin-2-ylamino]-4-methylthiazole-5-carboxylic acid ethyl ester; 2-[4-(4-Carboxy-phenyl)-6-(4-methyl-[1,4]diazepan-1-yl)-pyrimidin-2-ylamino]-4-methylthiazole-5-carboxylic acid ethyl ester; 2-[4-(4-Carboxy-phenyl)-6-(3-R-hydrox-piperidin-1-yl)-pyrimidin-2-ylamino]-4-methylthiazole-5-carboxylic acid ethyl ester; 2-[4-(4-Carboxy-phenyl)-6-(3-hydroxymethyl-piperidin-1-yl)-pyrimidin-2-ylamino]-4-methylthiazole-5-carboxylic acid ethyl ester; 2-[4-(4-Acetyl-[1,4]diazepan-1-yl)-6-(4-carboxy-phenyl)-pyrimidin-2-ylamino]-4-methylthiazole-5-carboxylic acid ethyl ester; 2-[4-(4-Carboxy-phenyl)-6-[N-methyl-N-(1-N-methyl-piperidin-4-yl)-amino]pyrimidin-2-ylamino]-4-methylthiazole-5-carboxylic acid ethyl ester; 2-[4-(4-Carboxy-phenyl)-6-piperazin-1-yl-pyrimidin-2-ylamino]-4-methylthiazole-5-carboxylic acid ethyl ester; 2-[4-(4-Carboxy-phenyl)-6-(4-sulfamoyl-benzylamino)-pyrimidin-2-ylamino]-4-methylthiazole-5-carboxylic acid ethyl ester; 2-[[4-[[5-Allyl[4-(aminosulfonyl)phenyl]methyl]amino]-6-chloro-2-pyrimidinyl]amino]-4-methyl-5-thiazolecarboxylic acid, ethyl ester; 2-[[4-[[[4-(Aminosulfonyl)phenyl]methyl]amino]-5-methyl-6-(1-piperazinyl)-2-pyrimidinyl]amino]-4-methyl-5-thiazolecarboxylic acid, ethyl ester, trifluoroacetate (1:3); 2-[[4-[[[4-(Aminosulfonyl)phenyl]methyl]amino]-5-methyl-6-(4-morpholinyl)-2-pyrimidinyl]amino]-4-methyl-5-thiazolecarboxylic acid, ethyl ester; 2-[[4-[[5-Allyl[4-(aminosulfonyl)phenyl]methyl]amino]-6-(4-methylpiperazinyl)-2-pyrimidinyl]amino]-4-methyl-5-thiazolecarboxylic acid, ethyl ester; 2-[[4-[[5-[2-[2-Methylprop-3-en][-4-[4-(aminosulfonyl)phenyl]methyl]amino]-6-(4-methylpiperazinyl)-2-pyrimidinyl]amino]-4-methyl-5-thiazolecarboxylic acid, ethyl ester; 2-[[4-[[[(3,4,5-(Trimethoxy)phenyl]methyl]amino]-5-methyl-6-(1-piperazinyl)-2-pyrimidinyl]amino]-4-methyl-5-thiazolecarboxylic acid, ethyl ester, trifluoroacetate; 2-[[4-[[5-[2,3-propandiol][4-(aminosulfonyl)phenyl]methyl]amino]-6-(4-methylpiperazinyl)-2-pyrimidinyl]amino]-4-methyl-5-thiazolecarboxylic acid, ethyl ester; 2-[[4-[[(3,4,5-(Trimethoxy)phenyl]methyl]amino]-5-methyl-6-(4-methyl-1-piperazinyl)-2-pyrimidinyl]amino]-4-methyl-5-thiazolecarboxylic acid, ethyl ester, trifluoroacetate; 2-[[4-[[5-[2-[2-Methylprop-3-en][-4-[4-(aminosulfonyl)phenyl]methyl]amino]-6-chloro-2-pyrimidinyl]amino]-4-methyl-5-thiazolecarboxylic acid, ethyl ester; 2-[[4-[[[4-(Aminosulfonyl)phenyl]methyl]amino]-5-methyl-6-(4-tertbutyloxycarbonyl-1-piperazinyl)-2-pyrimidinyl]amino]-4-methyl-5-thiazolecarboxylic acid, ethyl ester; 2-[[4-[N-[[3,4,5-(Trimethoxy)phenyl]methyl]-N-methylamino]-5-methyl-6-(4-methyl-1-piperazinyl)-2-pyrimidinyl]amino]-4-methyl-5-thiazolecarboxylic acid, ethyl ester; 2-[4,6-Bis-(4-hydroxy-piperidin-1-yl)-5-methylpyrimidin-2-ylamino]-4-methyl-thiazole-5-carboxylic acid ethyl ester; 2-[4,6-Bis-(3-oxo-piperazin-1-yl)-5-[ethoxycarbonylmethyl]pyrimidin-2-ylamino]-4-methyl-thiazole-5-carboxylic acid ethyl ester; 2-[4,6-Bis-(4-hydroxy-piperidin-1-yl)-5-methoxypyrimidin-2-ylamino]-4-methyl-thiazole-5-carboxylic acid ethyl ester; 2-[[4-[N-[[3,4,5-(Trimethoxy)phenyl]methyl]-N-methylamino]-5-methoxy-6-(4-methyl-1-piperazinyl)-2-pyrimidinyl]amino]-4-methyl-5-thiazolecarboxylic acid, ethyl ester; 2-[[4-[[3-pyridyl]methyloxy]-5-(2-propenyl-6-(4-morpholinyl)-2-pyrimidinyl]amino]-4-methyl-5-thiazolecarboxylic acid, ethyl ester; 2-[(4-Ethoxycarbonylmethyl-6-morpholin-4-yl-pyrimidin-2-yl)-amino]-4-methyl-5-thiazolecarboxylic acid ethyl ester; 2-[(4-Ethoxycarbonylmethyl-6-[3-oxo-1-piperazinyl]-pyrimidin-2-yl)-amino]-4-methyl-5-thiazolecarboxylic acid ethyl ester; 2-[(4-Carboxymethyl-6-morpholin-4-yl-pyrimidin-2-yl)-amino]-4-methyl-5-thiazolecarboxylic acid; 2-[4-Morpholin-4-yl-6-[(3,4,5-trimethoxy-phenylcarbamoyl)-methyl]-pyrimidin-2-ylamino]-4-methyl-5-thiazolecarboxylic acid ethyl ester; 2-[[4-[2-oxo-2-(3-oxo-piperazin-1-yl)-ethyl]-6-(4-sulfamoyl-benzylamino)-2-pyrimidinyl]amino]-4-methyl-5-thiazolecarboxylic acid ethyl ester; 2-[[4-(4-sulfamoyl-benzylamino)-6-[(4-sulfamoyl-benzylcarbamoyl)-methyl]-2-pyrimidinyl]amino]-4-methyl-5-thiazolecarboxylic acid ethyl ester; 2-[4-[2-(1,4-Dioxa-8-aza-spiro[4.5]dec-8-yl)-2-oxo-ethyl]-6-(4-sulfamoylbenzylamino)-2-pyrimidinyl]amino]-4-methyl-5-thiazolecarboxylic acid ethyl ester; 2-[[4-[[(4-Chloro-phenyl)-methyl-carbamoyl]-methyl]-6-(4-sulfamoyl-benzylamino) 2-pyrimidinyl]amino]-4-methyl-5-thiazolecarboxylic acid ethyl ester; 2-[[4-[2-(4-Hydroxy-piperidin-1-yl)-2-oxo-ethyl]-6-(4-sulfamoyl-benzylamino)-2-pyrimidinyl]amino]-4-methyl-5-thiazolecarboxylic acid ethyl ester; 2-[[4-[2-(4-Ethoxycarbonyl-piperidin-1-yl)-2-oxo-ethyl]-6-(4-sulfamoyl-benzylamino)-2-pyrimidinyl]amino]-4-methyl-5-thiazolecarboxylic acid ethyl ester; 2-[[4-(2-oxo-2-piperidin-1-yl-ethyl)-6-(4-sulfamoyl-benzylamino) 2-pyrimidinyl]amino]-4-methyl-5-thiazolecarboxylic acid ethyl ester; 2-[[4-[2-[4-(Furan-2-carbonyl)-piperazin-1-yl]-2-oxo-ethyl]-6-(4-sulfamoyl-benzylamino) 2-pyrimidinyl]amino]-4-methyl-5-thiazolecarboxylic acid ethyl ester; 2-[[4-[(Cyclohexyl-methyl-carbamoyl)-methyl]-6-(4-sulfamoyl-benzylamino) 2-pyrimidinyl]amino]-4-methyl-5-thiazolecarboxylic acid ethyl ester; 2-[[4-[2-(4-Acetyl-[1,4]diazepan-1-yl)-2-oxo-ethyl]-6-(4-sulfamoyl-benzylamino)-2-pyrimidinyl]amino]-4-methyl-5-thiazolecarboxylic acid ethyl ester; 2-[[4-[[Methyl-(1-methyl-piperidin-4-yl)-carbamoyl]-methyl]-6-(4-sulfamoyl-benzylamino)-2-pyrimidinyl]amino]-4-methyl-5-thiazolecarboxylic acid ethyl ester; 2-[[4-[2-(4-methyl-[1,4]diazepan-1-yl)-2-oxo-ethyl]-6-(4-sulfamoyl-benzylamino)-2-pyrimidinyl]amino]-4-methyl-5-thiazolecarboxylic acid ethyl ester; 2-[[4-[[Bis-(2-methoxy-ethyl)-carbamoyl]-methyl]-6-(4-sulfamoyl-benzylamino)-2-pyrimidinyl]amino]-4-methyl-5-thiazolecarboxylic acid ethyl ester; 2-[[4-(2-[1,4′]Bipiperidinyl-1′-yl-2-oxo-ethyl)-6-(4-sulfamoyl-benzylamino)-2-pyrimidinyl]amino]-4-methyl-5-thiazolecarboxylic acid ethyl ester; 2-[[4-[2-(4-Hydroxy-4-phenyl-piperidin-1-yl)-2-oxo-ethyl]-6-(4-sulfamoyl-benzylamino)-2-pyrimidinyl]amino]-4-methyl-5-thiazolecarboxylic acid ethyl ester; 2-[[4-Ethoxycarbonyl-6-(4-sulfamoyl-benzylamino)-2-pyrimidinyl]amino]-4-methyl-5-thiazolecarboxylic acid ethyl ester; 2-[[4-Carboxyl-6-(4-sulfamoyl-benzylamino)-2-pyrimidinyl]amino]-4-methyl-5-thiazolecarboxylic acid ethyl ester; 2-[[4-(Carboxymethyl-carbamoyl)-6-(4-sulfamoyl-benzylamino)-2-pyrimidinyl]amino]-4-methyl-5-thiazolecarboxylic acid ethyl ester; 2-[4-(4-Hydroxy-piperidin-1-yl)-6-(4-methylsulfanyl-benzyl)-pyrimidin-2-ylamino]-4-methyl-5-thiazolecarboxylic acid ethyl ester; 2-[4-(4-Hydroxy-piperidin-1-yl)-6-(4-methanesulfinyl-benzyl)-pyrimidin-2-ylamino]-4-methyl-5-thiazolecarboxylic acid ethyl ester; 2-[4-(4-Hydroxy-piperidin-1-yl)-6-(4-methanesulfonyl-benzyl)-2-pyrimidinyl]amino]-4-methyl-5-thiazolecarboxylic acid ethyl ester; 2-[[4-[4-methyl-1-piperazinyl]-6-[N-methyl-N-[(3,4,5-trimethoxyphenyl)methyl]amino]-2-pyrimidinyl]amino]-4-trifluoromethyl-5-thiazolecarboxylic acid, ethyl ester; 2-[[4-[4-Methylpiperazin-1-yl]-6-(N-methyl-N-[[(3,4,5-trimethoxyphenyl)methyl]amino]-2-pyrimidinyl]amino]-4-methyl-5-cyanothiazole; 2-[[4-[4-Methylpiperazin-1-yl]-6-methyl-6-[[(3,4,5-trimethoxyphenyl)methyl]amino]-2-pyrimidinyl]amino]-4-methyl-5-thiazolecarboxylic acid, 2-methoxyethyl ester; 2-[[4-[4-Hydroxy-piperidin-1-yl]-6-[N-methyl[[N-[(3,4,5-trimethoxyphenyl)methyl][-N-methyl]amino]-2-pyrimidinyl]amino]-4-methyl-5-thiazolecarboxylic acid, butyl ester; 2-[[4-[1-morpholinyl]-6-[[2-[1-morpholinyl]ethyl]amino]-2-pyrimidinyl]amino]-4-methyl-5-thiazolecarboxylic acid, butyl ester; 2-[[4-[4-methyl-1-piperazinyl]-6-[N-[(3,4,5-trimethoxyphenyl)methyl][-N-(methyl)amino]-2-pyrimidinyl]amino]-4-isopropyl-5-thiazolecarboxylic acid, ethyl ester; 2-[[4-[4-methyl-1-piperazinyl]-6-[[N-[(3,4,5-trimethoxyphenyl)methyl]]-N-(methyl)amino]-2-pyrimidinyl]amino]-4-methyl-5-thiazolecarboxylic acid, methyl amide; 2-[4-[4-(2-Diisopropylamino-ethylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(3-Dimethylamino-propylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(Cyclohexylmethylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(Pyridin-4-ylmethylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(Isobutylcarbomoyl)-phenyl]-6-(4-hydroxy-piperidin-1-yl)-pyrimidin-2-ylamino]-4-methyl-5-thiazolecarboxylic acid ethyl ester; 2-[4-[4-(N-Cyclohexyl-N-methylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(N-Cyclopropylmethyl-N-propylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(4-Ethoxycarbonylpyperidine-1-carbamoyl)-phenyl]-6-(4-hydroxy-piperidin-1-yl)-pyrimidin-2-ylamino]-4-methyl-5-thiazolecarboxylic acid ethyl ester; 2-[4-[4-(3-Hydroxymethyl-piperidine-1-carbonyl)-phenyl]-6-(4-hydroxy-piperidin-1-yl)-pyrimidin-2-ylamino]-4-methyl-5-thiazolecarboxylic acid ethyl ester; 2-[4-[4-(N-2-Hydroxyethyl-N-ethylcarbamoyl)-phenyl]-6-(4-hydroxy-piperidin-1-yl)-pyrimidin-2-ylamino]-4-methyl-5-thiazolecarboxylic acid ethyl ester; 2-[4-[4-(Thiomorpholine-1-carbonyl)-phenyl]-6-(4-hydroxy-piperidin-1-yl)pyrimidin-2-ylamino]-4-methyl-5-thiazolecarboxylic acid ethyl ester; 2-[4-[4-(Morpholine-1-carbonyl)-phenyl]-6-(4-hydroxy-piperidin-1-yl)-pyrimidin-2-ylamino]-4-methyl-5-thiazolecarboxylic acid ethyl ester; and 2-[4-[4-(4-Chloro-phenylcarbamoyl)-phenyl]-6-(4-hydroxy-piperidin-1-yl)-pyrimidin-2-ylamino]-4-methyl-5-thiazolecarboxylic acid ethyl ester; or a stereoisomer, a pharmaceutically acceptable salt, or a hydrate thereof.

In another related embodiment, PDE7 inhibitors useful in the methods of the present invention include the following compounds:

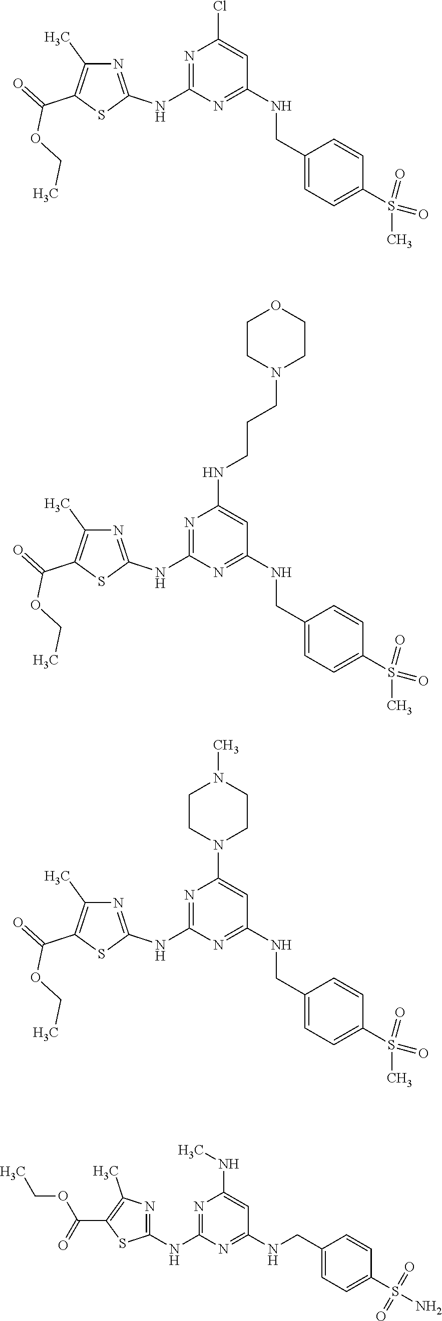 [SMILES: CCOC(=O)c1sc(Nc2nc(Cl)cc(NCc3ccc(S(C)(=O)=O)cc3)n2)nc1C ; CCOC(=O)c1sc(Nc2nc(NCCCN3CCOCC3)cc(NCc3ccc(S(C)(=O)=O)cc3)n2)nc1C ; CCOC(=O)c1sc(Nc2nc(NCc3ccc(S(C)(=O)=O)cc3)cc(N3CCN(C)CC3)n2)nc1C ; CCOC(=O)c1sc(Nc2nc(NC)cc(NCc3ccc(S(N)(=O)=O)cc3)n2)nc1C]

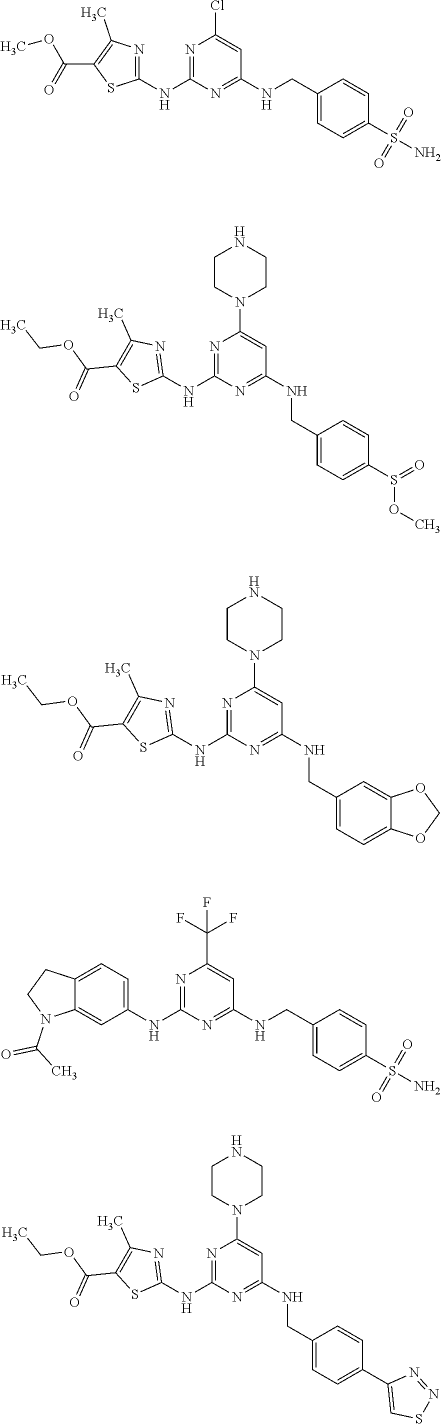

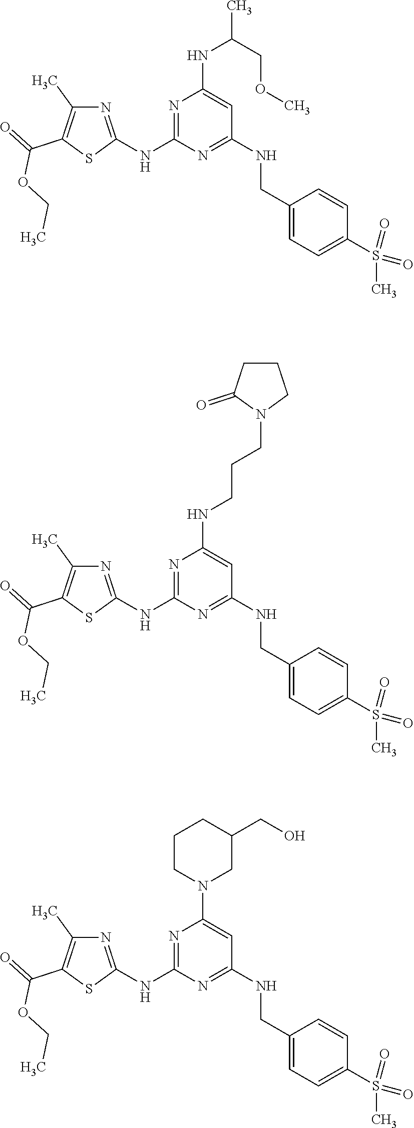 [SMILES: CCOC(=O)c1sc(Nc2nc(NCc3ccc(S(C)(=O)=O)cc3)cc(NC(C)COC)n2)nc1C ; CCOC(=O)c1sc(Nc2nc(NCCCN3CCCC3=O)cc(NCc3ccc(S(C)(=O)=O)cc3)n2)nc1C ; CCOC(=O)c1sc(Nc2nc(NCc3ccc(S(C)(=O)=O)cc3)cc(N3CCCC(CO)C3)n2)nc1C]

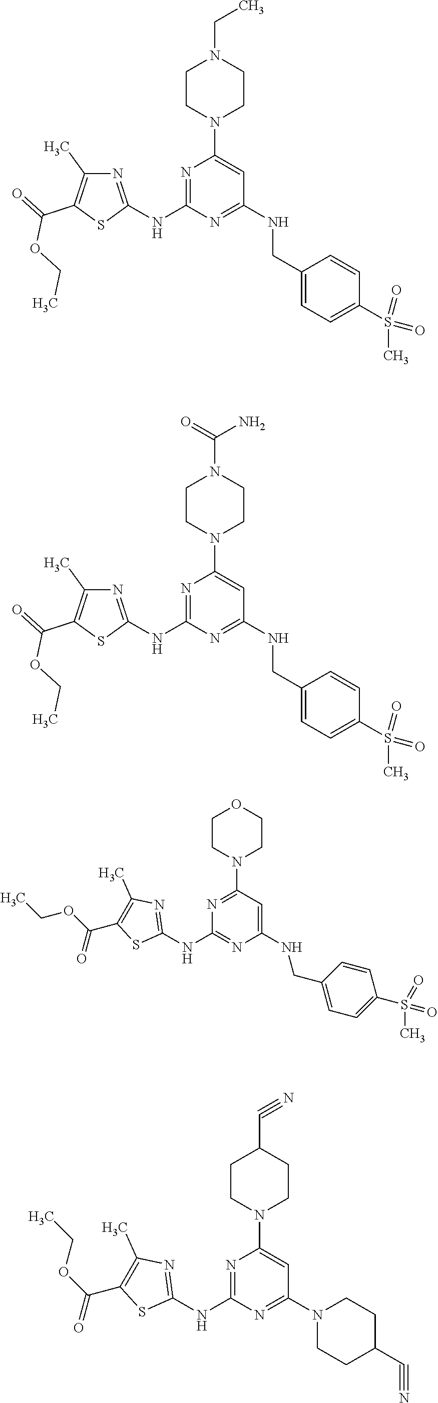 [SMILES: CCOC(=O)c1sc(Nc2nc(NCc3ccc(S(C)(=O)=O)cc3)cc(N3CCN(CC)CC3)n2)nc1C ; CCOC(=O)c1sc(Nc2nc(NCc3ccc(S(C)(=O)=O)cc3)cc(N3CCN(C(N)=O)CC3)n2)nc1C ; CCOC(=O)c1sc(Nc2nc(NCc3ccc(S(C)(=O)=O)cc3)cc(N3CCOCC3)n2)nc1C ; CCOC(=O)c1sc(Nc2nc(N3CCC(C#N)CC3)cc(N3CCC(C#N)CC3)n2)nc1C]

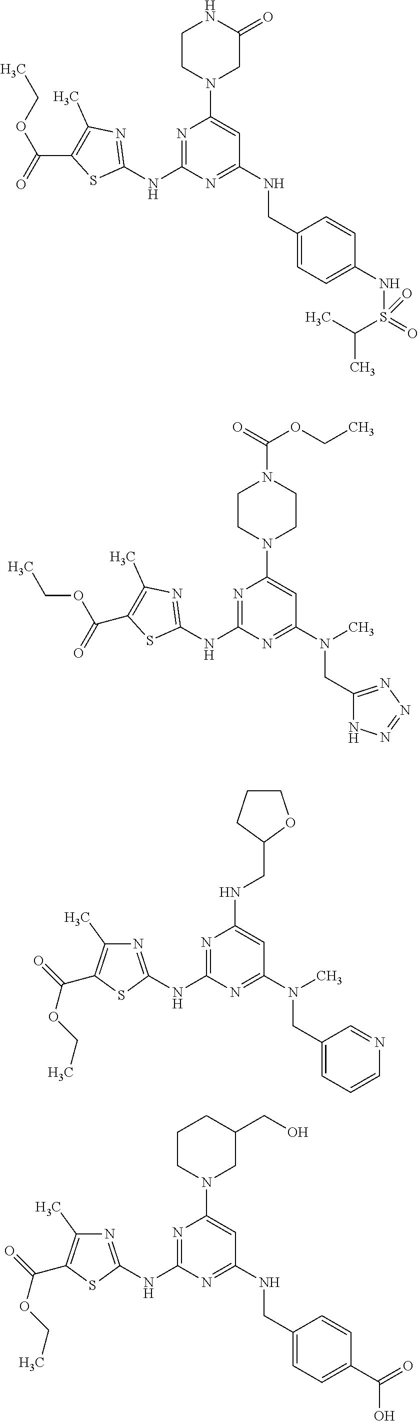 [SMILES: CCOC(=O)c1sc(Nc2nc(NCc3ccc(NS(=O)(=O)C(C)C)cc3)cc(N3CCNC(=O)C3)n2)nc1C ; CCOC(=O)c1sc(Nc2nc(N(C)Cc3nnn[nH]3)cc(N3CCN(C(=O)OCC)CC3)n2)nc1C ; CCOC(=O)c1sc(Nc2nc(NCC3CCCO3)cc(N(C)Cc3cccnc3)n2)nc1C ; CCOC(=O)c1sc(Nc2nc(NCc3ccc(C(=O)O)cc3)cc(N3CCCC(CO)C3)n2)nc1C]

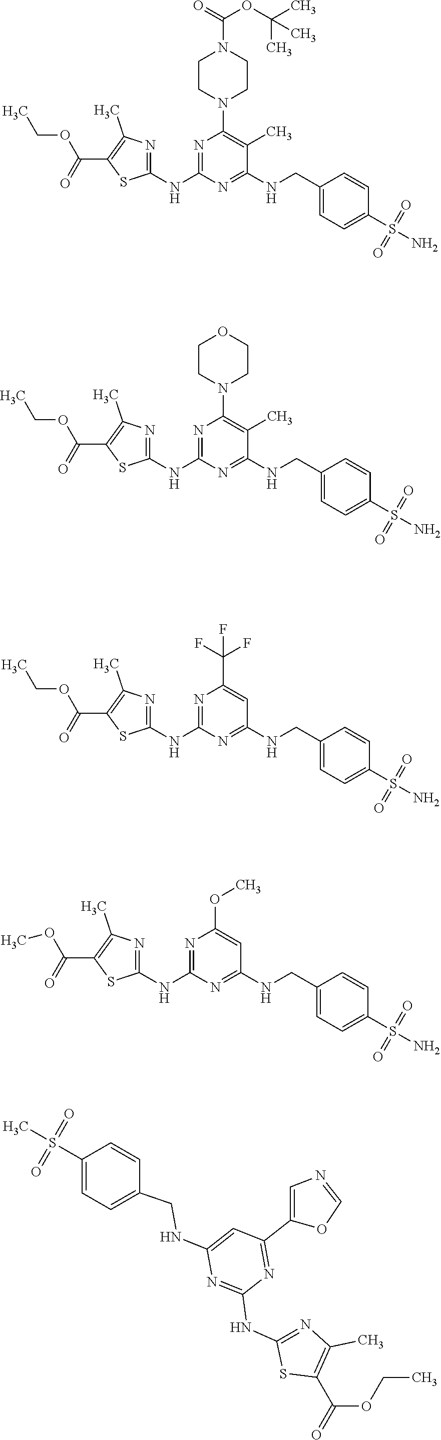 [SMILES: CCOC(=O)c1sc(Nc2nc(NCc3ccc(S(N)(=O)=O)cc3)c(C)c(N3CCN(C(=O)OC(C)(C)C)CC3)n2)nc1C ; CCOC(=O)c1sc(Nc2nc(NCc3ccc(S(N)(=O)=O)cc3)c(C)c(N3CCOCC3)n2)nc1C ; CCOC(=O)c1sc(Nc2nc(NCc3ccc(S(N)(=O)=O)cc3)cc(C(F)(F)F)n2)nc1C ; COC(=O)c1sc(Nc2nc(NCc3ccc(S(N)(=O)=O)cc3)cc(OC)n2)nc1C ; CCOC(=O)c1sc(Nc2nc(NCc3ccc(S(C)(=O)=O)cc3)cc(-c3cnco3)n2)nc1C]

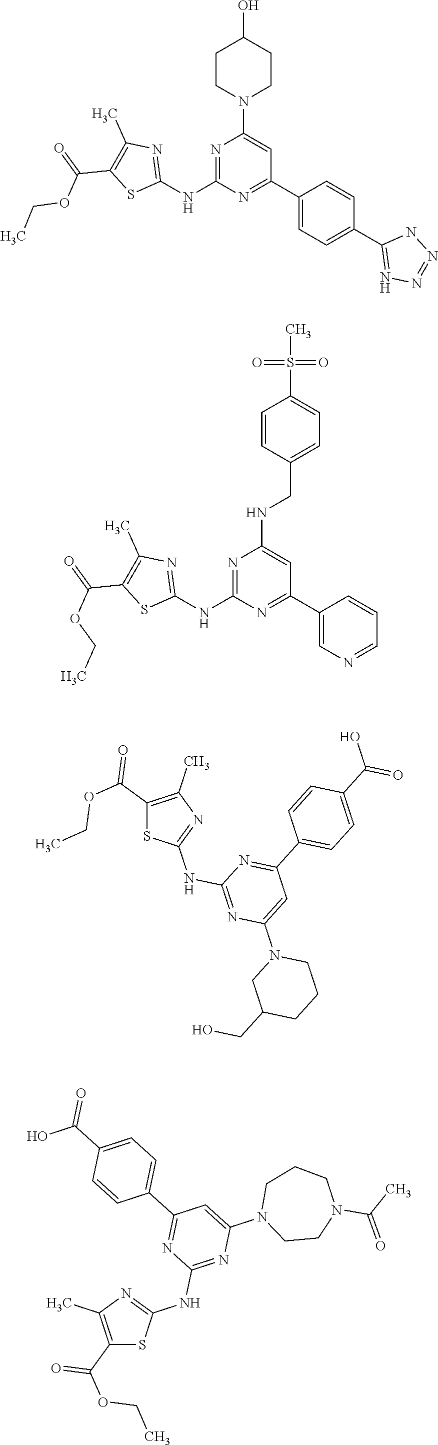 [SMILES: CCOC(=O)c1sc(Nc2nc(-c3ccc(-c4nnn[nH]4)cc3)cc(N3CCC(O)CC3)n2)nc1C ; CCOC(=O)c1sc(Nc2nc(NCc3ccc(S(C)(=O)=O)cc3)cc(-c3cccnc3)n2)nc1C ; CCOC(=O)c1sc(Nc2nc(-c3ccc(C(=O)O)cc3)cc(N3CCCC(CO)C3)n2)nc1C ; CCOC(=O)c1sc(Nc2nc(-c3ccc(C(=O)O)cc3)cc(N3CCCN(C(C)=O)CC3)n2)nc1C]

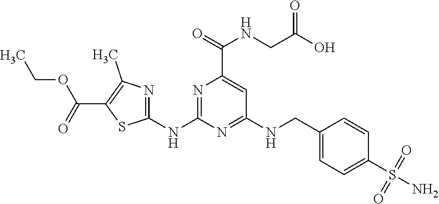 [SMILES: CCOC(=O)c1sc(Nc2nc(NCc3ccc(S(N)(=O)=O)cc3)cc(C(=O)NCC(=O)O)n2)nc1C]

or a stereoisomer, a pharmaceutically acceptable salt, or a hydrate thereof.

The preparation of these compounds is described in U.S. Pat. No. 7,087,614, U.S. 2003/0162802, and WO 2002/102313.

In another embodiment, PDE7 inhibitors useful in the methods of the invention are selected from those compounds generally or specifically disclosed in US 2007/0129388, U.S. Pat. No. 7,507,742, WO 2007/063391 and US 2009/0111837, each expressly incorporated by reference herein in its entirety.

In one embodiment, PDE7 inhibitors useful in the methods of the invention have the formula (44):

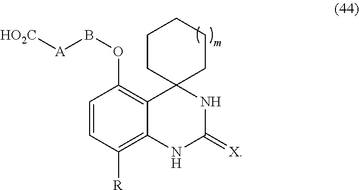

((44): Also referred to as spirocyclic and 5,8-substituted (1H,3H)-quinazoline derivatives.)

The substituents for the above formula (44) are defined as follows:
m is 0, 1 or 2; X is O, S or N—CN; R is F, Cl or CN; A is a C₃₋₆ cycloalkylene group optionally substituted with a C₁₋₄ alkyl group; and B is a single bond or a C₁₋₂ alkylene group; or a pharmaceutically acceptable salt, solvate, polymorph or prodrug thereof.

In regard to the above compounds, the term “alkylene” denotes a divalent saturated hydrocarbon chain having 1 or 2 carbon atoms. Examples of alkylene groups include methylene, ethylene and methylmethylene, of which methylene is preferred.

The term “cycloalkylene” denotes a divalent saturated carbocyclic ring having 3 to 6 carbon atoms. Examples of cycloalkylene groups include cyclopropylene (e.g., 1,1-cyclopropylene and cis- and trans-1,2-cyclopropylene), cyclobutylene (e.g., 1,1-cyclobutylene, cis and trans-1,2-cyclobutylene, and cis and trans-1,3-cyclobutylene), cyclopentylene (e.g., 1,1-cyclopentylene, cis and trans-1,2-cyclopentylene, and cis- and trans-1,3-cyclopentylene) and cyclohexylene (e.g., 1,1-cyclohexylene, cis- and trans-1,2-cyclohexylene, cis- and trans-1,3-cyclohexylene) and cis- and trans-1,4-cyclohexylene).

Preferred examples include cyclobutylene and cyclohexylene, more preferably cyclobutylene, even more preferably 1,3-cyclobutylene, and most preferably trans-1,3-cyclobutylene.

The term “alkyl” denotes a monovalent, straight or branched, saturated hydrocarbon chain containing 1 to 4 carbon atoms. Examples of alkyl groups include methyl, ethyl, n-propyl, isopropyl, n-butyl, isobutyl, sec-butyl and tert-butyl. Preferred examples include methyl and ethyl, especially methyl.

The cycloalkylene group is optionally substituted with a C₁₋₄ alkyl group. Preferably, the alkyl substituent, if present, is a methyl or ethyl group, more preferably a methyl group. The alkyl substituent, if present, may be present at any position on the ring, but is preferably present at the 1-position (i.e., the same position as the carboxylic acid group).

Preferably, m is 1 or 2, more preferably 1.

Preferably, X is O or N—CN, more preferably O.

Preferably, R is F or Cl, more preferably Cl.

Preferably, A is a cyclobutylene or cyclohexylene group optionally substituted with a methyl group. More preferably, A is a cyclobutylene group. Even more preferably, A is a 1,3-cyclobutylene group, especially a trans-1,3-cyclobutylene group.

Preferably, B is a single bond or a methylene group. More preferably, B is a single bond.

In another embodiment, a PDE7 inhibitor useful in the methods of the invention is selected from the following compounds:
cis-3-[(8′-Chloro-2′-oxo-2′,3′-dihydro-1′H-spiro[cyclohexane-1,4′-quinazolin]-5′-yl)oxy]cyclobutanecarboxylic acid; trans-3-[(8′-Chloro-2′-oxo-2′,3′-dihydro-1′H-spiro[cyclohexane-1,4′-quinazolin]-5′-yl)oxy]cyclobutanecarboxylic acid; 3-[(8′-fluoro-2′-oxo-2′,3′-dihydro-1′H-spiro[cyclohexane-1,4′-quinazolin]-5′-yl)oxymethyl]cyclobutanecarboxylic acid; trans-3-[(8′-cyano-2′-oxo-2′,3′-dihydro-1′H-spiro[cyclohexane-1,4′-quinazolin]-5′-yl)oxy]cyclobutanecarboxylic acid; 1-[(8′-fluoro-2′-oxo-2′,3′-dihydro-1′H-spiro[cyclohexane-1,4′-quinazolin]-5′-yl)oxymethyl]cyclobutanecarboxylic acid; trans-3-[(8′-chloro-2′-oxo-2′,3′-dihydro-1′H-spiro[cycloheptyl-1,4′-quinazolin]-5′-yl)oxy]cyclobutanecarboxylic acid; and trans-3-[(8′-chloro-2′-oxo-2′,3′-dihydro-1′H-spiro[cyclopentyl-1,4′-quinazolin]-5′-yl)oxy]cyclobutanecarboxylic acid; or a pharmaceutically acceptable salt, solvate, polymorph or prodrug thereof.

In other embodiments, PDE7 inhibitors useful in the methods of the invention have the formulas:

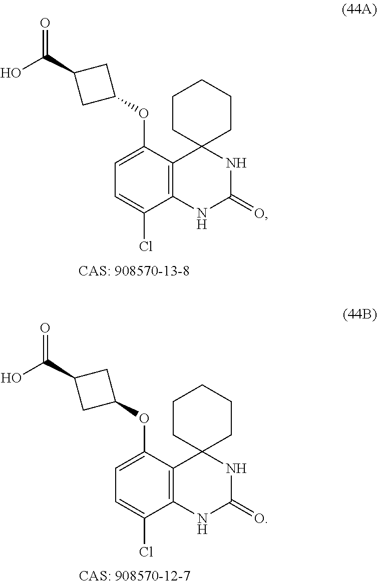 [SMILES: O=C1Nc2c(Cl)ccc(O[C@H]3C[C@H](C(=O)O)C3)c2C2(CCCCC2)N1 ; O=C1Nc2c(Cl)ccc(O[C@H]3C[C@@H](C(=O)O)C3)c2C2(CCCCC2)N1]

The formulas of 44A and 44B are also referred to in WO 2006/092691, US 2009/0111837, and WO 2006/092692, and ISBN: 978-3-527-33219-9 (Liras and Bell, Phosphodiesterases and Their Inhibitors, Wiley-VCH). The formula 44A is also referred to in https://db.idrblab.net/ttd/data/drug/details/d0pz4y.

The preparation of the above compounds is described in US 2007/0129388, U.S. Pat. No. 7,507,742 and WO 2007/063391.

In another embodiment, PDE7 inhibitors useful in the methods of the invention include the compound ASB16165 (1-Cyclohexyl-N-[6-(4-hydroxy-1-piperidinyl)-3-pyridinyl]-3-methyl-1H-thieno[2,3-c]pyrazole-5-carboxamide monohydrate) described in Kadoshima-Yamaoka, K. et al., “ASB16165, a novel inhibitor for phosphodiesterase 7A (PDE7A), suppresses IL-12-induced IFN-g production by mouse activated T lymphocytes,” Immunology Letters 122:193-197, 2009, expressly incorporated by reference herein.

In one embodiment, a PDE7 inhibitor useful in the methods of the invention has the formula (45):

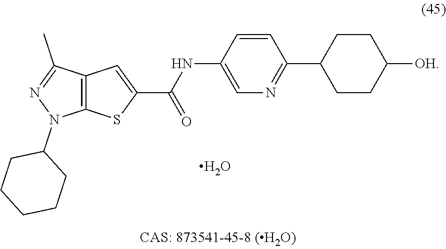 [SMILES: Cc1nn(C2CCCCC2)c2sc(C(=O)Nc3ccc(C4CCC(O)CC4)nc3)cc12 ; O]

Methods for preparing the above formula (45) are described in WO 2006/004040.

In another embodiment, PDE7 inhibitors useful in the methods of the invention include the compound YM-393059 ((+)—N-(4,6-dimethylpyrimidin-2-yl)-4-[2-(4-methoxy-3-methylphenyl)-5-(4-methylpiperazin-1-yl)-4,5,6,7-tetrahydro-1H-indol-1-yl]benzenesulfonamide difumarate) described in Yamamoto, S. et al., “The effects of a novel phosphodiesterase 7A and-4 dual inhibitor, YM-393059, on T-cell-related cytokine production in vitro and in vivo.” European Journal of Pharmacology 541:106-114, 2006, expressly incorporated by reference herein in its entirety.

In one embodiment, a PDE7 inhibitors useful in the methods of the invention has the formula (46):

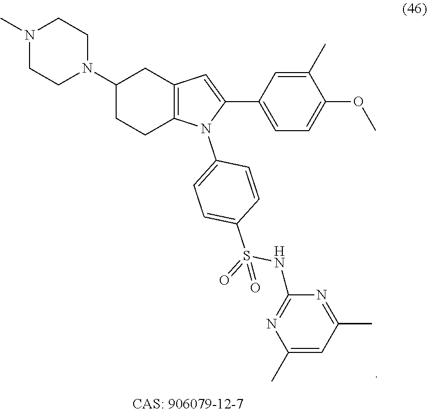

In another embodiment, PDE7 inhibitors useful in the methods of the invention are selected from those compounds generally or specifically disclosed in Martinez et al., “Benzyl derivatives of 2,1,3-benzo-and benzothieno 3,2-aathiadiazine 2,2-dioxides: first phosphodiesterase 7 inhibitors,” J. Med. Chem. 43:683-689, 2000, which is expressly incorporated herein by reference in its entirety.

In one embodiment, PDE7 inhibitors useful in the methods of the invention include the following compounds:
1-[(4-Methoxyphenyl) carbonylmethyl]benzothieno-[3,2-a]-1,2,6-thiadiazin-4-93H)-one 2,2-dioxide; and 1-[(3,4-dichlorophenyl)-methyl]-2,1,3-benzothiadiazin-4(3H)-one 2,2 dioxide.

The preparation of the above compounds is described in J. Med. Chem. 43:683-689, 2000.

In another embodiment, PDE7 inhibitors useful in the methods of the invention are selected from those compounds generally or specifically disclosed in CN 112574202.

In another embodiment, PDE7 inhibitors useful in the methods of the invention are selected from those compounds generally or specifically disclosed in Castro, A. et al., “CODES, a novel procedure for ligand-based virtual screening: PDE7 inhibitors as an application example,”. Eur. J. Med. Chem. 43:1349-1359, 2008, which is expressly incorporated herein by reference in its entirety.

In one embodiment, PDE7 inhibitors useful in the methods of the invention include the following compounds:

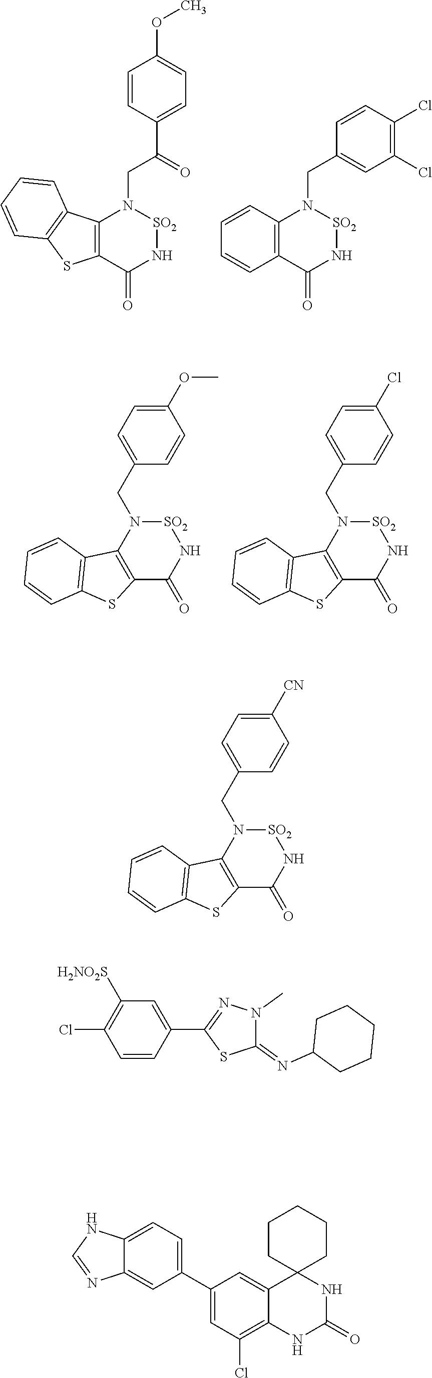

In another embodiment, PDE7 inhibitors useful in the methods of the invention have the formulas (47), (48) and/or (49):

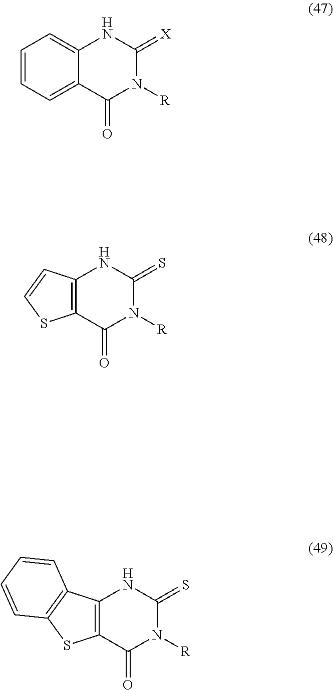

((47): Also referred to as 3-substituted 2,3-dihydro-2-thioxo-4(1H)-quinazolinone and 3-substituted 2,3-dihydro-2-oxo-4(1H)-quinazolinone derivatives.)
((48): Also referred to as 3-substituted 2,3-dihydro-2-thioxothieno[3,2-d]pyrimidin-4(1H)-one derivatives.)
((49): Also referred to as 3-substituted 2,3-dihydro-2-thioxo[1]benzothieno[3,2-d]pyrimidin-4(1H)-one derivatives.)

The substituents for the above formula (47), (48) or (49) are defined as follows:
X═O or S,
R═H, Ph (Phenyl), 4-OMePh, 2,6-diFPh, 2,3,4-triFPh, 2-BrPh, Bn, Naphthyl, or Me (Methyl).

In another embodiment, PDE7 inhibitors useful in the methods of the invention include the following compounds:
3-(2,3,4-Trifluorophenyl)-2-thioxo-(1H)-quinazolin-4-one;
3-Phenyl-2-thioxo-(1H)-thieno[3,2-d]pyrimidin-4-one;
3-(2,6-Difluorophenyl)-2-thioxo-(1H)-thieno[3,2-d]pyrimidin-4-one; and
3-(2,6-Difluorophenyl-2-thioxo-(1H)-benzo[4,5]-thieno[3,2-d]-pyrimidin-4-one.

The preparation of the above compounds is described in Eur. J. Med. Chem. 43:1349-1359, 2008. In another embodiment, PDE7 inhibitors useful in the methods of the invention include BMS-586353, as described in Yang, G. et al., “Phosphodiesterase 7A-deficient mice have functional T cells,” J. Immunol 171:6414-6420, 2003, which is expressly incorporated herein by reference in its entirety.

In another embodiment, PDE7 inhibitors useful in the methods of the invention are selected from those compounds generally or specifically disclosed in Pitts, W. J. et al., “Identification of purine inhibitors of phosphodiesterase 7 (PDE7),” Bioorg. Med. Chem. Lett. 14:2955-2958, 2004, and Kempson, J. et al., “Fused pyrimidine based inhibitors of phosphodiesterase 7 (PDE7): synthesis and initial structure-activity relationships,” Bioorg. Med. Chem. Lett. 15:1829-1833, 2005, each expressly incorporated herein by reference in its entirety. In one embodiment, PDE7 inhibitors useful in the methods of the invention have the formula (50):

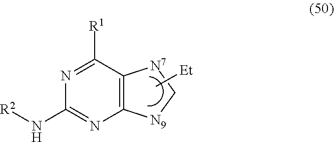

((50): Also referred to as ethyl-substituted purine-2-amine derivatives.)

The substituents for the above compounds are defined as follows:
R1 is

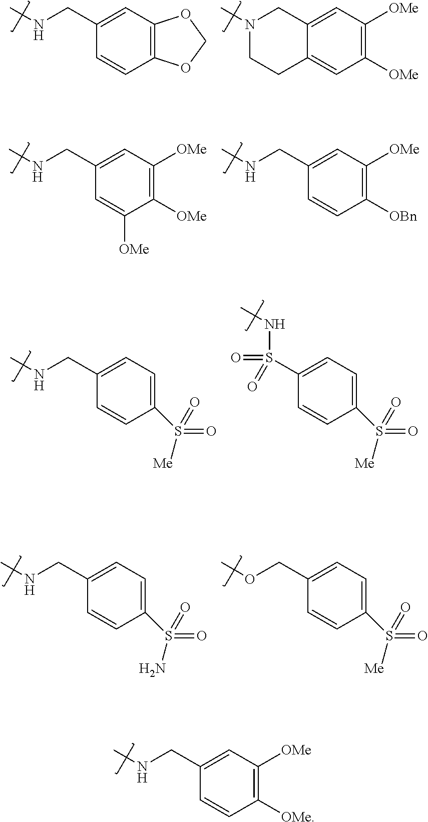

R2 is each

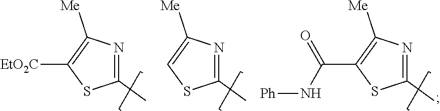

wherein the ethyl group may be attached to the 7 or 9 position.

In a related embodiment, PDE7 inhibitors useful in the methods of the invention have the formulas:

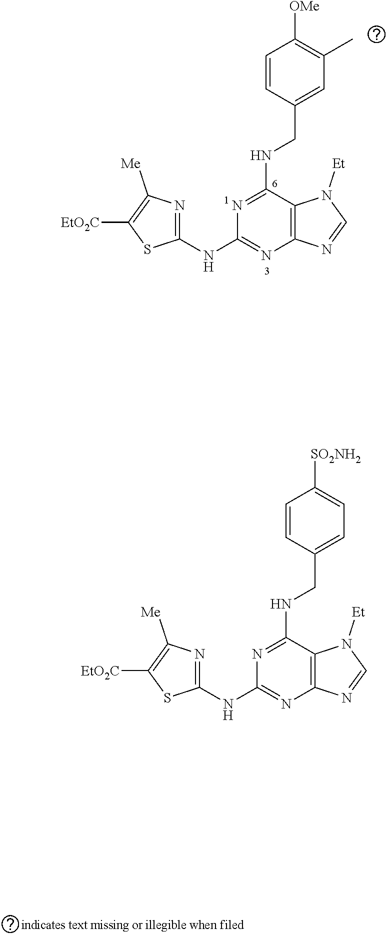 [SMILES: CCOC(=O)c1sc(Nc2nc(NCc3ccc(OC)c(C)c3)c3c(ncn3CC)n2)nc1C ; CCOC(=O)c1sc(Nc2nc(NCc3ccc(S(N)(=O)=O)cc3)c3c(ncn3CC)n2)nc1C]

In another related embodiment, PDE7 inhibitors useful in the methods of the invention have the formula:

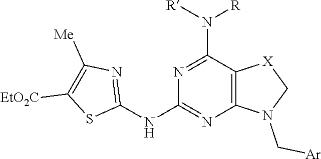

where X═CH2, CH2CH2 or OCH2;
Ar is

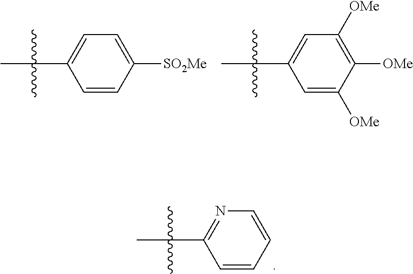 [SMILES: *c1ccc(S(C)(=O)=O)cc1 ; *c1cc(OC)c(OC)c(OC)c1 ; *c1ccccn1]

and NRR′ is

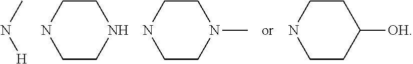

In another embodiment, PDE7 inhibitors useful in the methods of the invention are selected from those compounds generally or specifically disclosed in Kang, N. S. et al., “Docking and 3-D QSAR studies of dual PDE4-PDE7 inhibitors,” Molecular Simulation 33:1109-1117, 2007, expressly incorporated by reference herein in its entirety.

In one embodiment, PDE7 inhibitors useful in the methods of the invention include the following compounds (51), (52) and/or (53):

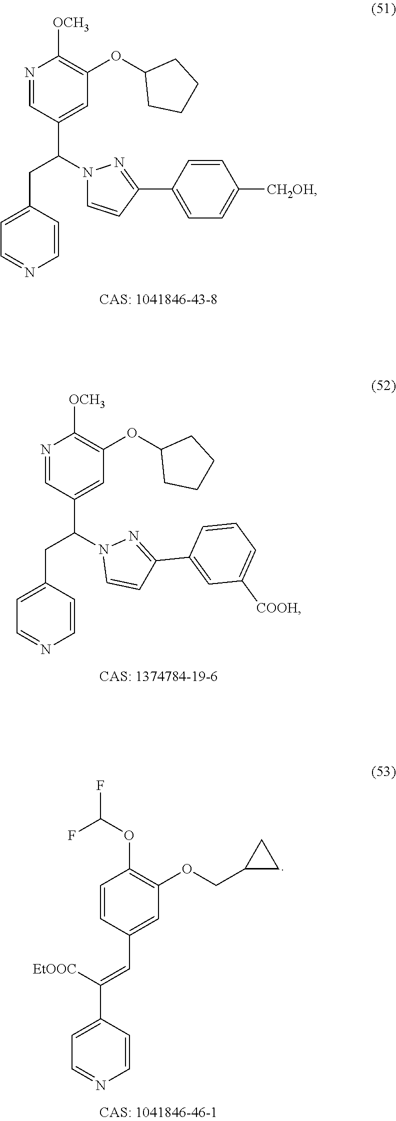 [SMILES: COc1ncc(C(Cc2ccncc2)n2ccc(-c3ccc(CO)cc3)n2)cc1OC1CCCC1 ; COc1ncc(C(Cc2ccncc2)n2ccc(-c3cccc(C(=O)O)c3)n2)cc1OC1CCCC1 ; CCOC(=O)/C(=C\c1ccc(OC(F)F)c(OCC2CC2)c1)c1ccncc1]

Methods for preparing the above compounds are described in Kang, N. S. et al., “Docking and 3-D QSAR studies of dual PDE4-PDE7 inhibitors, Molecular Simulation 33:1109-1117, 2007.

In another embodiment, PDE7 inhibitors useful in the methods of the invention are selected from those compounds generally or specifically disclosed in U.S. Pat. No. 8,242,126 and WO 2009/077680, U.S. Pat. No. 8,748,441 and WO 2010/109148, and U.S. Pat. No. 8,846,654 and WO 2010/116090 each expressly incorporated by reference herein in its entirety. In one embodiment, PDE7 inhibitors useful in the methods of the invention have the formula (55):
((55), (56): Also referred to as quinazolinedione derivatives.)

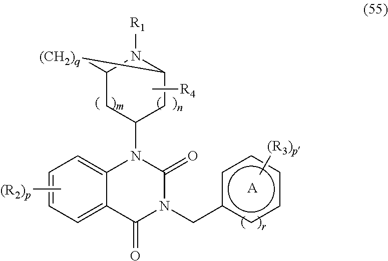

in which the substituents for the above formula (55) are defined as follows:
A represents an aryl group or a heteroaryl group;
R₁ represents: a hydrogen atom, —C(O)R in which R is a hydrogen atom, a (C₁-C₆)alkoxy group, an aryl group, a (C₃-C₆) cycloalkyl group or a (C₁-C₆) alkyl group, the said alkyl optionally being substituted by:
one to five hydroxyl group(s),
a benzyloxy group,
a (C₁-C₆)alkoxy group, optionally substituted by an aryl, or
a (C₃-C₆) cycloalkyl group,
(C₁-C₆) alkyl group;
R₂ represents:
a hydrogen atom, a halogen atom, a cyano group, a nitro group, a (C₁-C₆) alkyl group optionally substituted by an —NH₂ or else by an —NHC(O)Rb group, an —ORa group in which Ra represents: a hydrogen atom, a (C₁-C₆) alkyl group optionally substituted by one to 13 halogen atom(s), by one to five hydroxyl group(s), by an aryl group and/or by one to five cyano group(s), a (C₂-C₆) alkynyl group, an aryl group;
R₃ represents:
a hydrogen atom, a halogen atom, a hydroxyl group, a cyano group, an —SCF₃ group, a nitro group, an oxo group, an —S(O)₀₋₂-alkyl group, an —S(O)₀₋₂-heterocycloalkyl group, an —O—SO₂-aryl group optionally substituted by one to five halogen atom(s), an -alkylaminoalkyl or -cycloalkylaminoalkyl group -sulphonamide group, an aryl group or a heteroaryl group, the said group being monocyclic or polycyclic and in addition optionally being substituted by a (C₁-C₆) alkyl group, by one to five halogen atom(s) or by a (C₁-C₆)alkoxy group, a heterocycloalkyl group optionally substituted by a (C₁-C₆) alkyl group, a (C₁-C₆) alkyl group optionally substituted by:
one to five halogen atom(s),
an aryl group which can be substituted by one to five halogen atom(s) or by one to five hydroxyl group(s),
a heteroaryl group,
one to five hydroxyl group(s) which can be substituted by an aryl group itself optionally substituted by one to five halogen atom(s), or
a heterocycloalkyl group optionally substituted by a CO(O)Ra group or by a (C₁-C₆) alkyl group,
a —C(O)NRbRc group, a —C(O)ORc group or an —O—C(O)ORc group,
a (C₁-C₆)alkoxy group, optionally substituted by an aminoalkyl group, an aminocycloalkyl group, a cycloalkyl group, a heterocycloalkyl group, a monocyclic or polycyclic heteroaryl group, one to five hydroxyl group(s), one to five halogen atom(s), a (C₁-C₆)alkoxy group, a —C(O)ORc group, a —C(O)NRbRc group, an oxo group, and/or an aryl group, itself optionally substituted by one to five halogen atom(s), a cyano group, a (C₁-C₆)alkoxy group, an —O-haloalkyl group and/or a haloalkyl group,
an —O-cycloalkyl group, an —O-aryl group or an —O-heterocycloalkyl group, each optionally substituted by
an aryl group, itself optionally substituted by one to five halogen atom(s) or by a (C₁-C₆) alkyl group,
an oxo group,
one to five halogen atom(s), and/or
a (C₁-C₆) alkyl group, which can itself be substituted by an aryl group and/or an oxo group,
an —NH—CO—NH-aryl group, an —NH—CO—NH-heteroaryl group or an —NH—CO—NH—(C₁-C₆) alkyl group, each optionally being substituted by one to five halogen atom(s), by a cyano group, by a nitro group, by one to five hydroxyl group(s) or by a (C₁-C₆)alkoxy group,
an —NH—CO-aryl group or an —NH—CO-heteroaryl group, each optionally being substituted by one to five halogen atom(s);
or else R₃ forms, with A, a polycyclic heteroaryl group optionally substituted by a (C₁-C₆)alkoxy group or a (C₁-C₆) alkyl group optionally substituted by an aryl group which can itself be substituted by one to five halogen atom(s);
R₄ represents a hydrogen atom, an oxo group or a (C₁-C₆) alkyl group;
Rb represents:
a hydrogen atom,
a (C₁-C₆) alkyl group optionally substituted by one to five halogen atom(s), by one to five hydroxyl, cyano, amino, heterocycloalkyl or (C₁-C₆)alkoxy group(s) or by an aryl group optionally substituted by one to five halogen atom(s),
a (C₃-C₆) cycloalkyl group, a (C₂-C₆) alkynyl group, a (C₁-C₆)alkoxy group, an aryl group optionally substituted by one to five halogen atom(s);
Rc represents a hydrogen atom or a (C₁-C₆) alkyl group optionally substituted by one to five halogen atom(s);
or then Rb and Rc form, together with the nitrogen atom to which they are attached, a polycyclic heteroaryl group or a heterocycloalkyl group;
m and n represent, independently of one another, the value 0, 1 or 2, it being understood that m+n=2;
p and p′ represent, independently of one another, the value 1, 2 or 3, it being understood that, when p is greater than or equal to 2, then the R₂ groups are on separate carbon atoms and can be different from one another and, when p′ is greater than or equal to 2, then the R₃ groups are on separate carbon atoms and can be different from one another;
q represents the value 0, it being understood that, when q=0, then the nitrogenous heterocyclic group attached to the nitrogen situated in the 1 position of the 2,4-dioxo-1,2,3,4-tetrahydroquinazoline ring system is no longer bridged and is of the type:

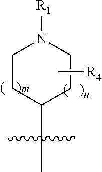

with R₁, R₄, m and n as defined above, and r represents the value 0 or 1; in the form of the base or of an addition salt with an acid.

The compound of general formula 55, characterized in that A represents a phenyl group or a pyridyl group.

The compound of general formula 55, characterized in that q=0, and m and n=1.

The compound of general formula 55, characterized in that R₂ represents a (C₁-C₆) alkyl group, or a methyl substituted by an —NH—CO—Rb group.

The compound of general formula 55, characterized in that R₂ represents an —ORa group.

The compound of general formula 55, characterized in that R₂ is a halogen atom or a cyano or a hydrogen or a hydroxyl or a (C₁-C₆) alkyl optionally substituted by an —NH₂ or else by an —NHC(O)Rb group.

The compound of general formula 55, characterized in that A is a phenyl, R₁ is a —C(O)R group in which R represents a hydrogen atom, q is equal to 0, n and m have the value 1 and R₂ is —ORa. The compound of general formula 55, characterized in that A is a phenyl, R₁ is a —C(O)R group in which R represents a hydrogen atom, q is equal to 0, n and m have the value 1 and R₂ is a methyl substituted by the —NH—CO—Rb group.

The compound of general formula 55, characterized in that A is a phenyl, R₁ is a —C(O)R group in which R represents a hydrogen atom, q is equal to 0, n and m have the value 1, p is equal to 2, one of the R₂ groups is —ORa, and the other of the R₂ groups is a halogen atom.

The compound of general formula 55, characterized in that the R₂ group is in the 6 position of the 2,4-dioxo-1,2,3,4-tetrahydroquinazoline ring system and in that there may additionally be an identical or different R₂ group in the 7 position of the 2,4-dioxo-1,2,3,4-tetrahydroquinazoline ring system.

A pharmaceutical composition, characterized in that it comprises at least one compound of formula 55 or an addition salt with a pharmaceutically acceptable acid thereof, and also at least one pharmaceutically acceptable excipient.

In another embodiment, a PDE7 inhibitor useful in the methods of the invention is selected from the following compounds:
2-{[3-(3,4-dimethoxybenzyl)-1-(1-formylpiperidin-4-yl)-2,4-dioxo-1,2,3,4-tetrahydroquinazolin-6-yl]oxy}propanenitrile;
1-(1-acetylpiperidin-4-yl)-3-3,4-dimethoxybenzyl)-6-hydroxyquinazoline-2,4(1H,3H)-dione; {[1-(1-acetylbenzyl)-4-yl)-3-(3,4-dimethoxybenzyl) 2,4-dioxo-1,2,3,4-tetrahydroquinazolin-6-yl]oxy}acetonitrile;
2-{[1-(1-acetylpiperidin-4-yl)-3-(3,4-dimethoxybenzyl)-2,4-dioxo-1,2,3,4-tetrahydroquinazolin-6-yl]oxy}propanenitrile;
{[3-(3,4-dimethoxybenzyl)-1-(1-formylpiperidin-4-yl)-2,4-dioxo-1,2,3,4-tetrahydroquinazolin-6-yl]oxy}acetonitrile;
4-[3-(3,4-dimethoxybenzyl)-6-[2-fluoro-1-(fluoromethyl)ethoxy]-2,4-dioxo-3,4-dihydroquinazolin-1(2H)-yl]piperidine-1-carbaldehyde;
1-(1-acetylpiperidin-4-yl)-3-(3,4-dimethoxybenzyl)-6-[2-fluoro-1-(fluoromethyl)ethoxy]quinazoline-2,4(1H,3H)-dione;
4-[3-(3,4-dimethoxybenzyl)-2,4-dioxo-6-(2,2,2-trifluoroethoxy)-3,4-dihydroquinazolin-1(2H)-yl]piperidine-1-carbaldehyde;
1-(1-acetylpiperidin-4-yl)-6-(2,2-difluoroethoxy)-3-(3,4-dimethoxybenzyl)quinazoline-2,4(1H,3H)-dione;
4-[6-(2,2-difluoroethoxy)-3-(3,4-dimethoxybenzyl)-2,4-dioxo-3,4-dihydroquinazolin-1(2H)-yl]piperidine-1-carbaldehyde;
N-{[3-(3,4-dimethoxybenzyl)-1-(1-formylpiperidin-4-yl)-2,4-dioxo-1,2,3,4-tetrahydroquinazolin-6-yl]methyl}acetamide;
1-(1-acetylpiperidin-4-yl)-6-(aminomethyl)-3-(3,4-dimethoxybenzyl)quinazoline-2,4(1H,3H)-dione hydrochloride;
N-{[3-(3,4-dimethoxybenzyl)-1-(1-formylpiperidin-4-yl)-2,4-dioxo-1,2,3,4-tetrahydroquinazolin-6-yl]methyl}formamide;
N-{[1-(1-acetylbenzyl-4-yl)-3-(3,4-dimethoxybenzyl)-2,4-dioxo-1,2,3,4-tetrahydroquinazolin-6-yl]methyl}formamide;
N-{[1-(1-acetylpiperidin-4-yl)-3-(3,4-dimethoxybenzyl)-2,4-dioxo-1,2,3,4-tetrahydroquinazolin-6-yl]methyl}acetamide;
4-[3-(3,4-dichlorobenzyl)-6-(2,2-difluoroethoxy)-2,4-dioxo-3,4-dihydroquinazolin-1(2H)-yl]piperidine-1-carbaldehyde;
4-[3-(4-chlorobenzyl)-6-(2,2-difluoroethoxy)-2,4-dioxo-3,4-dihydroquinazolin-1(2H)-yl]piperidine-1-carbaldehyde;
methyl 4-{[6-(2,2-difluoroethoxy)-1-(1-formylpiperidin-4-yl)-2,4-dioxo-1,4-dihydroquinazolin-3 (2H)-yl]methyl}benzoate;
4-{[6-(2,2-difluoroethoxy)-1-(1-formylpiperidin-4-yl)-2,4-dioxo-1,4-dihydroquinazolin-3 (2H)-yl]methyl}benzoic acid;
4-{[6-(2,2-difluoroethoxy)-1-(1-formylpiperidin-4-yl)-2,4-dioxo-1,4-dihydroquinazolin-3 (2H)-yl]methyl}-N-(2-methoxyethyl)benzamide;
4-[3-(3,4-dimethoxybenzyl)-6-methyl-2,4-dioxo-3,4-dihydroquinazolin-1(2H)-yl]piperidine-1-carbaldehyde;
4-[6-(2,2-difluoroethoxy)-3-(3-hydroxy-4-methoxybenzyl)-2,4-dioxo-3,4-dihydroquinazolin-1(2H)-yl]piperidine-1-carbaldehyde;
4-[6-(2,2-difluoroethoxy)-3-[3-(2-hydroxyethoxy)-4-methoxybenzyl]-2,4-dioxo-3,4-dihydroquinazolin-1(2H)-yl]piperidine-1-carbaldehyde;
4-[6-(2,2-difluoroethoxy)-3-(3-ethoxy-4-methoxybenzyl)-2,4-dioxo-3,4-dihydroquinazolin-1(2H)-yl]piperidine-1-carbaldehyde;
4-[6-(2,2-difluoroethoxy)-3-[4-methoxy-3-(2-methoxyethoxy)benzyl]-2,4-dioxo-3,4-dihydroquinazolin-1(2H)-yl]piperidine-1-carbaldehyde;
4-[6-(2,2-difluoroethoxy)-3-[3-(3-hydroxypropoxy)-4-methoxybenzyl]-2,4-dioxo-3,4-dihydroquinazolin-1(2H)-yl]piperidine-1-carbaldehyde;
4-[5-chloro-3-(3,4-dimethoxybenzyl)-2,4-dioxo-3,4-dihydroquinazolin-1(2H)-yl]piperidine-1-carbaldehyde;
4-{3-[3-(cyclopentyloxy)-4-methoxybenzyl]-6-(2,2-difluoroethoxy)-2,4-dioxo-3,4-dihydroquinazolin-1(2H)-yl}piperidine-1-carbaldehyde;
2-(5-{[6-(2,2-difluoroethoxy)-1-(1-formylpiperidin-4-yl)-2,4-dioxo-1,4-dihydroquinazolin-3 (2H)-yl]methyl}-2-methoxyphenoxy) acetamide;
4-[6-(2,2-difluoroethoxy)-3-(3,4-dimethoxybenzyl)-2,4-dioxo-3,4-dihydroquinazolin-1(2H)-yl]-3-methylpiperidine-1-carbaldehyde;
4-{3-[4-(cyclopentyloxy)-3-methoxybenzyl]-6-[2-fluoro-1-(fluoromethyl)ethoxy]-2,4-dioxo-3,4-dihydroquinazolin-1(2H)-yl}piperidine-1-carbaldehyde;
4-[3-(3-chlorobenzyl)-6-[2-fluoro-1-(fluoromethyl)ethoxy]-2,4-dioxo-3,4-dihydroquinazolin-1(2H)-yl]piperidine-1-carbaldehyde;
4-[3-(4-chlorobenzyl)-6-[2-fluoro-1-(fluoromethyl)ethoxy]-2,4-dioxo-3,4-dihydroquinazolin-1(2H)-yl]piperidine-1-carbaldehyde;
4-{3-[3-(cyclopentyloxy)-4-methoxybenzyl]-6-[2-fluoro-1-(fluoromethyl)ethoxy]-2,4-dioxo-3,4-dihydroquinazolin-1(2H)-yl}piperidine-1-carbaldehyde;
4-[3-(3,4-dimethoxybenzyl)-6-(2-hydroxyethoxy)-2,4-dioxo-3,4-dihydroquinazolin-1(2H)-yl]piperidine-1-carbaldehyde;
4-[3-(3,4-dichlorobenzyl)-6-[2-fluoro-1-(fluoromethyl)ethoxy]-2,4-dioxo-3,4-dihydroquinazolin-1(2H)-yl]piperidine-1-carbaldehyde;
4-{3-[(6-chloropyridin-3-yl)methyl]-6-[2-fluoro-1-(fluoromethyl)ethoxy]-2,4-dioxo-3,4-dihydroquinazolin-1(2H)-yl}piperidine-1-carbaldehyde;
4-[3-(3-chloro-4-methoxybenzyl)-6-[2-fluoro-1-(fluoromethyl)ethoxy]-2,4-dioxo-3,4-dihydroquinazolin-1(2H)-yl]piperidine-1-carbaldehyde;
4-[3-(3,4-dimethoxybenzyl)-6-(2-fluoroethoxy)-2,4-dioxo-3,4-dihydroquinazolin-1(2H)-yl]piperidine-1-carbaldehyde;
2-[5-({6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenoxy]acetamide;
4-{6-[2-fluoro-1-(fluoromethyl)ethoxy]-3-(3-hydroxy-4-methoxybenzyl)-2,4-dioxo-3,4-dihydroquinazolin-1(2H)-yl}piperidine-1-carbaldehyde;
4-[3-(3,4-dimethoxybenzyl)-6-ethoxy-2,4-dioxo-3,4-dihydroquinazolin-1(2H)-yl]piperidine-1-carbaldehyde;
4-[5,7-dichloro-3-(3,4-dimethoxybenzyl)-2,4-dioxo-3,4-dihydroquinazolin-1(2H)-yl]piperidine-1-carbaldehyde;
4-[7-chloro-3-(3,4-dimethoxybenzyl)-2,4-dioxo-3,4-dihydroquinazolin-1(2H)-yl]piperidine-1-carbaldehyde;
4-{6-[2-fluoro-1-(fluoromethyl)ethoxy]-3-(3-fluoro-4-methoxybenzyl)-2,4-dioxo-3,4-dihydroquinazolin-1(2H)-yl}piperidine-1-carbaldehyde;
4-[6-(difluoromethoxy)-3-(3,4-dimethoxybenzyl)-2,4-dioxo-3,4-dihydroquinazolin-1(2H)-yl]piperidine-1-carbaldehyde;
4-[3-(3,4-dimethoxybenzyl)-6-(1-methylethoxy)-2,4-dioxo-3,4-dihydroquinazolin-1(2H)-yl]piperidine-1-carbaldehyde;
4-{6-[2-fluoro-1-(fluoromethyl)ethoxy]-3-[4-methoxy-3-(1-methylethoxy)benzyl]-2,4-dioxo-3,4-dihydroquinazolin-1(2H)-yl}piperidine-1-carbaldehyde;
4-{6-[2-fluoro-1-(fluoromethyl)ethoxy]-3-(3-methoxybenzyl)-2,4-dioxo-3,4-dihydroquinazolin-1(2H)-yl}piperidine-1-carbaldehyde;
4-{3-[3,5-bis(trifluoromethyl)benzyl]-6-[2-fluoro-1-(fluoromethyl)ethoxy]-2,4-dioxo-3,4-dihydroquinazolin-1(2H)-yl}piperidine-1-carbaldehyde;
4-[3-(3-ethoxybenzyl)-6-[2-fluoro-1-(fluoromethyl)ethoxy]-2,4-dioxo-3,4-dihydroquinazolin-1(2H)-yl]piperidine-1-carbaldehyde;
4-{3-[3-chloro-4-(2-methoxyethoxy)benzyl]-6-[2-fluoro-1-(fluoromethyl)ethoxy]-2,4-dioxo-3,4-dihydroquinazolin-1(2H)-yl}piperidine-1-carbaldehyde;
4-[3-(3,4-diethoxybenzyl)-6-[2-fluoro-1-(fluoromethyl)ethoxy]-2,4-dioxo-3,4-dihydroquinazolin-1(2H)-yl]piperidine-1-carbaldehyde;
4-[3-(4-ethoxy-3-methoxybenzyl)-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-3-(4-methoxy-3-methylbenzyl)-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-[4-(trifluoromethyl)benzyl]-3,4-dihydroquinazolin-1(2H)-yl}piperidine-1-carbaldehyde;
4-{6-[2-fluoro-1-(fluoromethyl)ethoxy]-2,4-dioxo-3-[4-(trifluoromethyl)benzyl]-3,4-dihydroquinazolin-1(2H)-yl}piperidine-1-carbaldehyde;
4-{3-[4-(benzyloxy)-3-methoxybenzyl]-6-[2-fluoro-1-(fluoromethyl)ethoxy]-2,4-dioxo-3,4-dihydroquinazolin-1(2H)-yl}piperidine-1-carbaldehyde;
4-{6-[2-fluoro-1-(fluoromethyl)ethoxy]-3-(3-methoxy-4-nitrobenzyl)-2,4-dioxo-3,4-dihydroquinazolin-1(2H)-yl}piperidine-1-carbaldehyde;
4-[3-(4-ethoxybenzyl)-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-3-[4-(morpholin-4-ylmethyl)benzyl]-2,4-dioxo-3,4-dihydroquinazolin-1(2H)-yl}piperidine-1-carbaldehyde;
4-{6-[2-fluoro-1-(fluoromethyl)ethoxy]-3-(4-morpholin-4-ylbenzyl)-2,4-dioxo-3,4-dihydroquinazolin-1(2H)-yl}piperidine-1-carbaldehyde;
4-[3-(biphenyl-4-ylmethyl)-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-3-[4-(methylsulphanyl)benzyl]-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-(4-(pyridin-3-yl)benzyl)-3,4-dihydroquinazolin-1(2H)-yl}piperidine-1-carbaldehyde;
4-{6-[2-fluoro-1-(fluoromethyl)ethoxy]-3-(3-methoxy-4-methylbenzyl)-2,4-dioxo-3,4-dihydroquinazolin-1(2H)-yl}piperidine-1-carbaldehyde;
2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]acetamide;
4-{6-[2-fluoro-1-(fluoromethyl)ethoxy]-3-(3-methoxy-4-propoxybenzyl)-2,4-dioxo-3,4-dihydroquinazolin-1(2H)-yl}piperidine-1-carbaldehyde;
2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]-N-methylacetamide;
2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]-N,N-dimethylacetamide;
2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]-N-methoxy-N-methylacetamide;
4-{3-[4-(cyclopentyloxy)-3-ethoxybenzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(cyclopentyloxy)-3-(1-methylethoxy)benzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(cyclopentyloxy)-3-propoxybenzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(cyclopentyloxy)-3-hydroxybenzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(difluoromethoxy)-3-methoxybenzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(difluoromethoxy)-3-ethoxybenzyl]-6-[2-fluoro-1-(fluoromethyl)ethoxy]-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-(4-(thiophen-3-yl)-benzyl)-3,4-dihydroquinazolin-1(2H)-yl}piperidine-1-carbaldehyde;
4-{6-[2-fluoro-1-(fluoromethyl)ethoxy]-2,4-dioxo-3-(4-(pyridin-4-yl)benzyl)-3,4-dihydroquinazolin-1(2H)-yl}piperidine-1-carbaldehyde;
4-{6-[2-fluoro-1-(fluoromethyl)ethoxy]-3-[(1-methyl-1H-indol-6-yl)methyl]-2,4-dioxo-3,4-dihydroquinazolin-1(2H)-yl}piperidine-1-carbaldehyde;
4-{3-[4-(cyclopropylmethoxy)-3-methoxybenzyl]-6-[2-fluoro-1-(fluoromethyl)ethoxy]-2,4-dioxo-3,4-dihydroquinazolin-1(2H)-yl}piperidine-1-carbaldehyde;
2-[4-({6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenoxy]-N-methylacetamide;
4-{6-[2-fluoro-1-(fluoromethyl)ethoxy]-2,4-dioxo-3-[4-(1H-pyrazol-1-yl)benzyl]-3,4-dihydroquinazolin-1(2H)-yl}piperidine-1-carbaldehyde;
4-{6-[2-fluoro-1-(fluoromethyl)ethoxy]-2,4-dioxo-3-(4-(pyridin-2-yl)benzyl)-3,4-dihydroquinazolin-1(2H)-yl}piperidine-1-carbaldehyde;
4-{6-[2-fluoro-1-(fluoromethyl)ethoxy]-2,4-dioxo-3-(4-(thiophen-2-yl)-benzyl)-3,4-dihydroquinazolin-1(2H)-yl}piperidine-1-carbaldehyde;
4-{6-[2-fluoro-1-(fluoromethyl)ethoxy]-2,4-dioxo-3-(quinolin-7-ylmethyl)-3,4-dihydroquinazolin-1(2H)-yl}piperidine-1-carbaldehyde;
4-{6-[2-fluoro-1-(fluoromethyl)ethoxy]-3-[(6-methoxynaphthalen-2-yl)methyl]-2,4-dioxo-3,4-dihydroquinazolin-1(2H)-yl}piperidine-1-carbaldehyde;
4-{3-[4-(1H-benzimidazol-1-yl)benzyl]-6-[2-fluoro-1-(fluoromethyl)ethoxy]-2,4-dioxo-3,4-dihydroquinazolin-1(2H)-yl}piperidine-1-carbaldehyde;
4-{6-[2-fluoro-1-(fluoromethyl)ethoxy]-3-[3-methoxy-4-(2-methylpropoxy)benzyl]-2,4-dioxo-3,4-dihydroquinazolin-1(2H)-yl}piperidine-1-carbaldehyde;
4-{6-[2-fluoro-1-(fluoromethyl)ethoxy]-3-[3-methoxy-4-(tetrahydrofuran-3-yloxy)benzyl]-2,4-dioxo-3,4-dihydroquinazolin-1(2H)-yl}piperidine-1-carbaldehyde;
4-[3-{4-[(1-benzylpyrrolidin-3-yl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-(1-benzothiophen-5-ylmethyl)-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-3-[3-methoxy-4-(1-methylethoxy)benzyl]-2,4-dioxo-3,4-dihydroquinazolin-1(2H)-yl}piperidine-1-carbaldehyde;
4-[3-(3,4-dimethoxybenzyl)-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(1-acetylpyrrolidin-3-yl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{4-[(4-fluorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{4-[(4-chlorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{-4-[(3-chlorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-2,4-dioxo-3-(3-(thiophen-3-yl)benzyl)-3,4-dihydroquinazolin-1(2H)-yl}piperidine-1-carbaldehyde;
4-[3-(4-ethoxy-3-methoxybenzyl)-6-(2-hydroxyethoxy)-2,4-dioxo-3,4-dihydroquinazolin-1(2H)-yl]piperidine-1-carbaldehyde;
4-[3-{4-[2-(2,3-dihydro-1H-indol-1-yl)-2-oxoethoxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{-4-[(3,4-dichlorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-{6-[2-fluoro-1-(fluoromethyl)ethoxy]-3-[3-methoxy-4-(2-oxo-2-(piperidin-1-yl)ethoxy)benzyl]-2,4-dioxo-3,4-dihydroquinazolin-1(2H)-yl}piperidine-1-carbaldehyde;
4-[3-[3-ethoxy-4-(thiophen-2-ylmethoxy)benzyl]-6-[2-fluoro-1-(fluoromethyl)ethoxy]-2,4-dioxo-3,4-dihydroquinazolin-1(2H)-yl]piperidine-1-carbaldehyde;
4-[3-(3,4-dimethoxybenzyl)-6-[2-fluoro-1-(hydroxymethyl)ethoxy]-2,4-dioxo-3,4-dihydroquinazolin-1(2H)-yl]piperidine-1-carbaldehyde;
(2R)-2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]propanoic acid;
4-{6-[2-fluoro-1-(fluoromethyl)ethoxy]-3-[(1-methyl-3-(thiophen-2-yl)-1H-pyrazol-5-yl)methyl]-2 4-dioxo-3,4-dihydroquinazolin-1(2H)-yl}piperidine-1-carbaldehyde;
4-{6-[2-fluoro-1-(fluoromethyl)ethoxy]-3-[4-(5-methyl-1,2,4-oxadiazol-3-yl)benzyl]-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-(4-(pyrimidin-5-yl)benzyl)-3,4-dihydroquinazolin-1(2H)-yl}piperidine-1-carbaldehyde;
4-{6-[2-fluoro-1-(fluoromethyl)ethoxy]-3-[(1-methyl-3-phenyl-1H-pyrazol-5-yl)methyl]-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-{[6-(1H-pyrazol-1-yl) pyridin-3-yl]methyl}-3,4-dihydroquinazolin-1(2H)-yl}piperidine-1-carbaldehyde;
4-{6-[2-fluoro-1-(fluoromethyl)ethoxy]-2,4-dioxo-3-[(2-(thiophen-2-yl)pyrimidin-5-yl)methyl]-3,4-dihydroquinazolin-1(2H)-yl}piperidine-1-carbaldehyde;
4-{6-[2-fluoro-1-(fluoromethyl)ethoxy]-3-[4-(1-methyl-1H-pyrazol-3-yl)benzyl]-2,4-dioxo-3,4-dihydroquinazolin-1(2H)-yl}piperidine-1-carbaldehyde;
4-{6-[2-fluoro-1-(fluoromethyl)ethoxy]-3-[4-(3-methyl-1,2,4-oxadiazol-5-yl)benzyl]-2,4-dioxo-3,4-dihydroquinazolin-1(2H)-yl}piperidine-1-carbaldehyde;
[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]acetic acid;
4-{6-[2-fluoro-1-(fluoromethyl)ethoxy]-2,4-dioxo-3-(thieno[2,3-b]pyridin-2-ylmethyl)-3,4-dihydroquinazolin-1(2H)-yl}piperidine-1-carbaldehyde;
4-{6-[2-fluoro-1-(fluoromethyl)ethoxy]-2,4-dioxo-3-[(6-phenylpyridin-3-yl)methyl]-3,4-dihydroquinazolin-1(2H)-yl}piperidine-1-carbaldehyde;
4-{6-[2-fluoro-1-(fluoromethyl)ethoxy]-3-[(6-(morpholin-4-yl) pyridin-3-yl)methyl]-2,4-dioxo-3,4-dihydroquinazolin-1(2H)-yl}piperidine-1-carbaldehyde;
4-{6-[2-fluoro-1-(fluoromethyl)ethoxy]-2,4-dioxo-3-[(6-(thiophen-2-yl) pyridin-3-yl)methyl]-3,4-dihydroquinazolin-1(2H)-yl}piperidine-1-carbaldehyde;
4-{6-[2-fluoro-1-(fluoromethyl)ethoxy]-3-[(1-methyl-5-phenyl-1H-pyrazol-3-yl)methyl]-2,4-dioxo-3,4-dihydroquinazolin-1(2H)-yl}piperidine-1-carbaldehyde;
4-({6-[2-fluoro-1-(fluoromethyl)ethoxy]-1-(1-formylpiperidin-4-yl)-2,4-dioxo-1,4-dihydroquinazolin-3 (2H)-yl}methyl) biphenyl-2-carbonitrile;
(2R)-2-[2-(cyclopentyloxy)-5-({6-[2-fluoro-(fluoromethyl)ethoxy]-1-(1-formylpiperidin-4-yl)-2,4-dioxo-1,4-dihydroquinazolin-3 (2H)-yl}methyl)phenoxy]-N-methylpropanamide;
4-{7-fluoro-6-[2-fluoro-1-(fluoromethyl)ethoxy]-2,4-dioxo-3-(4-(thiophen-2-yl)benzyl)-3,4-dihydroquinazolin-1(2H)-yl}piperidine-1-carbaldehyde;
4-{6-[2-fluoro-1-(fluoromethyl)ethoxy]-3-[3-methoxy-4-(morpholin-4-ylmethyl)benzyl]-2,4-dioxo-3,4-dihydroquinazolin-1(2H)-yl}piperidine-1-carbaldehyde;
4-{6-[2-fluoro-1-(fluoromethyl)ethoxy]-3-[3-methoxy-4-(piperidin-1-ylmethyl)benzyl]-2,4-dioxo-3,4-dihydroquinazolin-1(2H)-yl}piperidine-1-carbaldehyde;
4-[3-{4-[(3,4-dichlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
2-[2-(cyclopentyloxy)-5-({6-[2-fluoromethyl)ethoxy]-1-(1-formylpiperidin-4-yl)-2,4-dioxo-1,4-dihydroquinazolin-3 (2H)-yl}methyl)phenoxy]-N-ethylacetamide;
(2S)-2-[2-(cyclopentyloxy)-5-({6-[2-fluoro-1-(fluoromethyl)ethoxy]-1-(1-formylpiperidin-4-yl)-2,4-dioxo-1,4-dihydroquinazolin-3 (2H)-yl}methyl)phenoxy]propanoic acid;
4-{6-[2-fluoro-1-(fluoromethyl)ethoxy]-3-(3-methoxy-4-{[(3R)-2-oxo-1-phenylpyrrolidin-3-yl]oxy}benzyl)-2,4-dioxo-3,4-dihydroquinazolin-1(2H)-yl}piperidine-1-carbaldehyde;
4-{3-[4-(cyclobutylmethoxy)-3-methoxybenzyl]-6-[2-fluoro-1-(fluoromethyl)ethoxy]-2,4-dioxo-3,4-dihydroquinazolin-1(2H)-yl}piperidine-1-carbaldehyde;
4-{3-[4-(benzyloxy)-3-methoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-{7-fluoro-6-[2-fluoro-1-(fluoromethyl)ethoxy]-3-(4-hydroxy-3-methoxybenzyl)-2,4-dioxo-3,4-dihydroquinazolin-1(2H)-yl}piperidine-1-carbaldehyde;
4-{3-[4-(cyclopropylmethoxy)-3-methoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-{7-fluoro-6-[2-fluoro-1-(fluoromethyl)ethoxy]-3-[3-methoxy-4-(2-methylpropoxy)benzyl]-2,4-dioxo-3,4-dihydroquinazolin-1(2H)-yl}piperidine-1-carbaldehyde;
4-{7-fluoro-6-[2-fluoro-1-(fluoromethyl)ethoxy]-3-[3-methoxy-4-(1-methylethoxy)benzyl]-2,4-dioxo-3,4-dihydroquinazolin-1(2H)-yl}piperidine-1-carbaldehyde;
4-[3-(4-ethoxy-3-methoxybenzyl)-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-3-{[6-(3-methoxyphenyl)pyridin-3-yl]methyl}-2,4-dioxo-3,4-dihydroquinazolin-1(2H)-yl}piperidine-1-carbaldehyde;
4-{7-fluoro-6-[2-fluoro-1-(fluoromethyl)ethoxy]-3-{[6-(2-fluorophenyl)pyridin-3-yl]methyl}-2,4-dioxo-3,4-dihydroquinazolin-1(2H)-yl}piperidine-1-carbaldehyde;
4-{7-fluoro-6-[2-fluoro-1-(fluoromethyl)ethoxy]-3-{[6-(4-fluorophenyl)pyridin-3-yl]methyl}-2,4-dioxo-3,4-dihydroquinazolin-1(2H)-yl}piperidine-1-carbaldehyde;
4-{7-fluoro-6-[2-fluoro-1-(fluoromethyl)ethoxy]-3-{[6-(4-methoxyphenyl)pyridin-3-yl]methyl}-2,4-dioxo-3,4-dihydroquinazolin-1(2H)-yl}piperidine-1-carbaldehyde;
4-{7-fluoro-6-[2-fluoro-1-(fluoromethyl)ethoxy]-2,4-dioxo-3-[(6-(thiophen-2-yl) pyridin-3-yl)methyl]-3,4-dihydroquinazolin-1(2H)-yl}piperidine-1-carbaldehyde;
4-{3-[3-ethoxy-4-(thiophen-2-ylmethoxy)benzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-{7-fluoro-6-[2-fluoro-1-(fluoromethyl)ethoxy]-3-[4-(1-methyl-1H-pyrazol-3-yl)benzyl]-2,4-dioxo-3,4-dihydroquinazolin-1(2H)-yl}piperidine-1-carbaldehyde;
4-{7-fluoro-6-[2-fluoro-1-(fluoromethyl)ethoxy]-2,4-dioxo-3-(4-(pyrimidin-5-yl)benzyl)-3,4-dihydroquinazolin-1(2H)-yl}piperidine-1-carbaldehyde;
4-{7-fluoro-6-[2-fluoro-1-(fluoromethyl)ethoxy]-3-[(1-methyl-3-(thiophen-2-yl)-1H-pyrazol-5-yl)methyl]-2,4-dioxo-3,4-dihydroquinazolin-1(2H)-yl}piperidine-1-carbaldehyde;
4-{7-fluoro-6-[2-fluoro-1-(fluoromethyl)ethoxy]-3-[3-methoxy-4-(2-oxo-2-(piperidin-1-yl)ethoxy)benzyl]-2,4-dioxo-3,4-dihydroquinazolin-1(2H)-yl}piperidine-1-carbaldehyde;
4-[3-{4-[2-(2,3-dihydro-1H-indol-1-yl)-2-oxoethoxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-3-[4-(5-methyl-1,2,4-oxadiazol-3-yl)benzyl]-2,4-dioxo-3,4-dihydroquinazolin-1(2H)-yl}piperidine-1-carbaldehyde;
4-[3-{4-[(3-chlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{[6-(3,5-dichlorophenyl)pyridin-3-yl]methyl}-6-[2-fluoro-1-(fluoromethyl)ethoxy]-2,4-dioxo-3,4-dihydroquinazolin-1(2H)-yl]piperidine-1-carbaldehyde;
4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl) biphenyl-2-carbonitrile;
4-{7-fluoro-6-[2-fluoro-1-(fluoromethyl)ethoxy]-2,4-dioxo-3-[4-(1H-pyrazol-1-yl)benzyl]-3,4-dihydroquinazolin-1(2H)-yl}piperidine-1-carbaldehyde;
4-{7-fluoro-6-[2-fluoro-1-(fluoromethyl)ethoxy]-3-{[6-(3-fluorophenyl)pyridin-3-yl]methyl}-2,4-dioxo-3,4-dihydroquinazolin-1(2H)-yl}piperidine-1-carbaldehyde;
3-[5-({7-fluoro-6-[2-fluoro-1-(fluoromethyl)ethoxy]-1-(1-formylpiperidin-4-yl)-2,4-dioxo-1,4-dihydroquinazolin-3 (2H)-yl]methyl)pyridin-2-yl}benzonitrile;
4-[3-(3,4-diethoxybenzyl)-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(4-chlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-3-[4-(morpholin-4-ylmethyl)benzyl]-2,4-dioxo-3,4-dihydroquinazolin-1(2H)-yl}piperidine-1-carbaldehyde;
4-{7-fluoro-6-[2-fluoro-1-(fluoromethyl)ethoxy]-2,4-dioxo-3-{[6-(1H-pyrazol-1-yl) pyridin-3-yl]methyl}-3,4-dihydroquinazolin-1(2H)-yl}piperidine-1-carbaldehyde;
4-{7-fluoro-6-[2-fluoro-1-(fluoromethyl)ethoxy]-3-(4-(morpholin-4-yl)-benzyl)-2,4-dioxo-3,4-dihydroquinazolin-1(2H)-yl}piperidine-1-carbaldehyde;
4-{7-fluoro-6-[2-fluoro-1-(fluoromethyl)ethoxy]-3-(3-methoxy-4-propoxybenzyl)-2,4-dioxo-3,4-dihydroquinazolin-1(2H)-yl}piperidine-1-carbaldehyde;
4-{3-[4-(1H-benzimidazol-1-yl)benzyl]-7-fluoro-6-[2-fluoro-1-(fluoromethyl)ethoxy]-2,4-dioxo-3,4-dihydroquinazolin-1(2H)-yl}piperidine-1-carbaldehyde;
5-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxybenzonitrile;
3-(3,4-dimethoxybenzyl)-6-[2-fluoro-1-(fluoromethyl)ethoxy]-1-(1-formylpiperidin-4-yl)-2,4-dioxo-1,2,3,4-tetrahydroquinazoline-7-carbonitrile;
4-[3-(4-bromobenzyl)-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(3,4-dichlorobenzyl)oxy]-3-(2-methoxyethoxy)benzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-{3-[4-(benzyloxy)benzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-[3-{4-[(3,4-dichlorobenzyl)oxy]-3-ethoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-[3-{4-[(3,4-dichlorobenzyl)oxy]-3-(2-fluoroethoxy)benzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-[3-{4-[(2-chloro-4-fluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(2,4-dichlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(2-chloro-6-fluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(2,6-dichlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(2-chlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[7-fluoro-3-{4-[(2-fluorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
2-[(3,4-dichlorobenzyl)oxy]-5-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)benzonitrile;
4-[3-{-4-[(3,4-dichlorophenoxy)methyl]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-2,4-dioxo-3-[4-(2-phenylethyl)benzyl]-3,4-dihydroquinazolin-1(2H)-yl}piperidine-1-carbaldehyde;
4-[3-{-4-[(4,5-dichloro-2-fluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(4-chlorophenoxy)methyl]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{3-chloro-4-[(4-chlorobenzyl)oxy]-5-ethoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{3-chloro-4-[(2,4-dichlorobenzyl)oxy]-5-ethoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-[7-fluoro-3-{-4-[(4-fluorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{-4-[(3,5-dichlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-(4-{[4-chloro-3-(trifluoromethyl)benzyl]oxy}-3-methoxybenzyl)-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{-4-[(3-chlorophenoxy)methyl]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[(3,5-difluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{3-[4-(benzyloxy)-3-methoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-[3-{4-[(3-chloro-5-fluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-3-(3-methoxy-4-{[4-(trifluoromethyl)benzyl]oxy}benzyl)-2,4-dioxo-3,4-dihydroquinazolin-1(2H)-yl}piperidine-1-carbaldehyde;
4-[3-{4-[(2,5-dichlorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{[4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenoxy]methyl}benzonitrile;
3-{[4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenoxy]methyl}benzonitrile;
4-[3-{-4-[(4-chloro-2-fluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-[3-{4-[1-(3,4-dichlorophenyl)ethoxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{7-fluoro-6-[2-fluoro-1-(fluoromethyl)ethoxy]-3-{4-[(3-hydroxybenzyl)oxy]-3-methoxybenzyl}-2,4-dioxo-3,4-dihydroquinazolin-1(2H)-yl}piperidine-1-carbaldehyde;
4-[7-fluoro-3-{4-[(3-fluorobenzyl)oxy]-3-methoxybenzyl}-6-[2-fluoro-1-(fluoromethyl)ethoxy]-2,4-dioxo-3,4-dihydroquinazolin-1(2H)-yl]piperidine-1-carbaldehyde;
4-[3-{-4-[(3,4-difluorobenzyl)oxy]-3-methoxybenzyl}-7-fluoro-6-[2-fluoro-1-(fluoromethyl)ethoxy]-2,4-dioxo-3,4-dihydroquinazolin-1(2H)-yl]piperidine-1-carbaldehyde;
4-{3-[4-(5,6-dichloro-1H-benzimidazol-1-yl)-3-methoxybenzyl]-7-fluoro-6-[2-fluoro-1-(fluoromethyl)ethoxy]-2,4-dioxo-3,4-dihydroquinazolin-1(2H)-yl}piperidine-1-carbaldehyde;
4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)phenyl 3,4-dichlorobenzenesulphonate;
4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenyl 3,4-dichlorobenzene-sulphonate;
3,4-dichloro-N-[4-({7-fluoro-6-[2-fluoro-1-(fluoromethyl)ethoxy]-1-(1-formylpiperidin-4-yl)-2,4-dioxo-1,4-dihydroquinazolin-3 (2H)-yl}methyl)-2-methoxyphenyl]benzamide.

A pharmaceutical composition, comprising at least one of the compounds listed above or an addition salt with a pharmaceutically acceptable acid thereof, and also at least one pharmaceutically acceptable excipient.

In more specific embodiments the compounds are selected from formulas (55A), (55B), (55C), (55D), (55E), (55F), (55G), (55H), (55I), (55J):

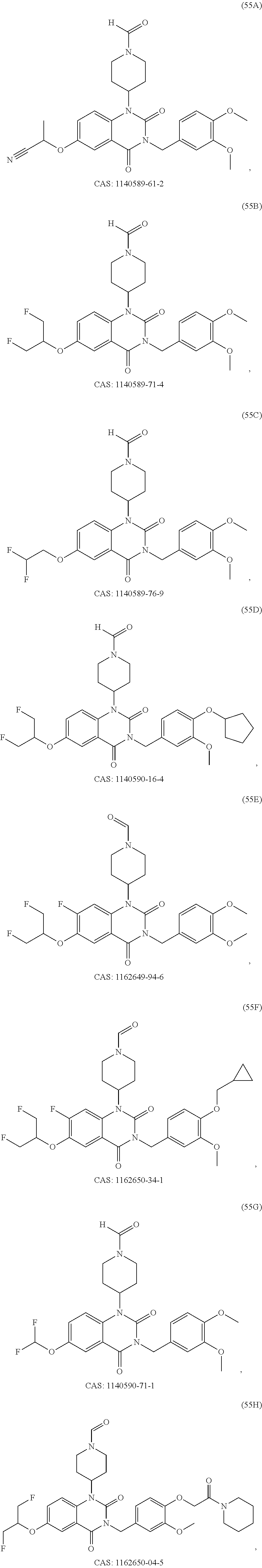

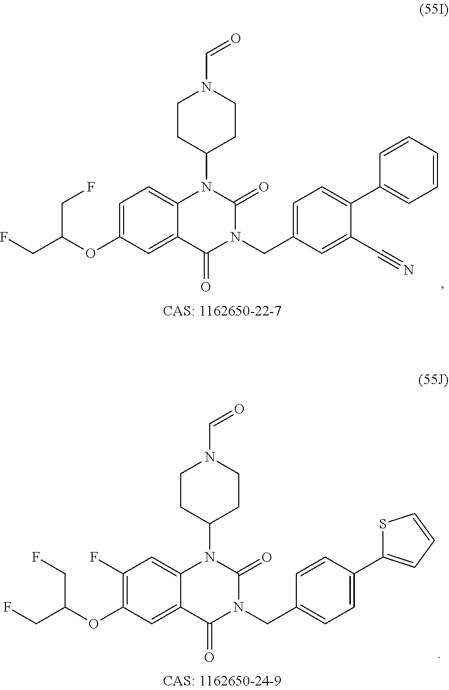 [SMILES: N#Cc1cc(Cn2c(=O)c3cc(OC(CF)CF)ccc3n(C3CCN(C=O)CC3)c2=O)ccc1-c1ccccc1 ; O=CN1CCC(n2c(=O)n(Cc3ccc(-c4cccs4)cc3)c(=O)c3cc(OC(CF)CF)c(F)cc32)CC1]

The preparation of the above compounds is described in U.S. Pat. No. 8,242,126 and WO 2009/077680.

In another embodiment, PDE7 inhibitors useful in the methods of the invention are selected from those compounds generally or specifically disclosed in U.S. Pat. No. 8,722,659 and WO 2010/116088 each expressly incorporated by reference herein in its entirety.

In one embodiment, PDE7 inhibitors useful in the methods of the invention have the formula (56):

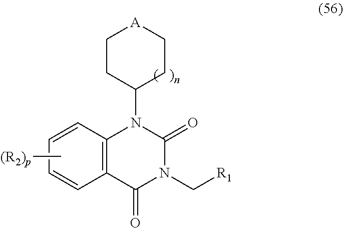

in which the substituents for the above formula (56) are defined as follows:

A represents either an oxygen or sulfur atom, or a sulfoxide function (SO function) or a sulfone function (SO₂ function);
n represents the value 1;
R₂ represents an atom or a group chosen from:
a hydrogen atom, a halogen atom, a cyano group, a nitro group, an aryl, arylalkyl or heteroaryl group, a group (C₁-C₅)alkyl, optionally substituted with a function —NH₂, with one or more halogen atoms, with one or more hydroxyl groups, with an alkynyl group, with an alkenyl group, with a group —NHC(O)Rb and/or with a group —NHC(O)—NRbRc, Rb and Rc being defined below,
a group —ORa, Ra being defined below,
a group NRbRc, Rb and Rc being defined below,
a group C(O)(C₁-C₆)alkyl,
an alkenyl group or an alkynyl group, the said groups being optionally substituted with at least one hydroxyl or with at least one halogen atom;
a group alkyl-S—, alkyl-S(O)—, and alkyl-S(O)₂—,
p represents the value 1, 2 or 3, it being understood that when p is equal to 2 or 3, then the atoms R₂ or the groups R₂ may be, respectively, identical or different;
R₁ represents an aryl, arylalkyl or heteroaryl group, the said groups being optionally substituted with (i) an atom R₃ or a group R₃, or with (ii) 2 or 3 atoms and/or groups R₃, the said atoms or groups R₃ being, respectively, identical or different,
given that R³ represents:
a hydrogen atom, a halogen atom, a hydroxyl group, a cyano group, a group —SCF₃, a nitro group, a group —S(O)₀₋₂-alkyl, a group —S(O)₀₋₂-heterocycloalkyl, a group —O—SO₂-aryl or O—SO₂-arylalkyl optionally substituted with one or more halogen atoms;
an alkyl-amino-alkyl group or a -cycloalkyl-amino-alkyl group, the said groups being optionally substituted on the terminal alkyl,
an optionally substituted sulfonamide group,
an aryl, arylalkyl or heteroaryl group, the said group being monocyclic or polycyclic and moreover being optionally substituted with a group (C₁-C₆)alkyl, with one or more hydroxyl groups, with one or more halogen atoms, with one or more cyano groups and/or with one or more groups (C₁-C₆)alkoxy, a heterocycloalkyl group optionally substituted with a group (C₁-C₆)alkyl,
a group (C₁-C₆)alkyl optionally substituted with:
one or more halogen atoms,
an aryl or arylalkyl group that may be substituted with one or more halogen atoms, with one or more groups (C₁-C₆)alkoxy, with one or more groups (C₁-C₆)alkyl, with one or more cyano groups and/or with one or more hydroxyl groups,
a heteroaryl group,
one or more hydroxyl groups that may be substituted with an aryl or arylalkyl group, which is itself optionally substituted with one or more halogen atoms, or
a heterocycloalkyl group optionally substituted with a group CO(O)Ra, or with a group (C₁-C₆)alkyl, Ra being defined below,
a group —C(O)NRbRc, Rb and Rc being defined below,
a group —C(O)ORc, or a group —O—C(O)ORc, Rc being defined below,
a group (C₁-C₆)alkoxy, optionally substituted with:
an amino-alkyl group, an amino-cycloalkyl group,
a cycloalkyl group,
a heterocycloalkyl group,
a monocyclic or polycyclic heteroaryl group,
one or more hydroxyl groups,
one or more halogen atoms,
a group (C₁-C₆)alkoxy,
a group —C(O)ORc, Rc being defined below,
a group —C(O)NRbRc, Rb and Rc being defined below, and/or
an aryl or arylalkyl group, which is itself optionally substituted with at least one atom and/or at least one group, the said atoms and groups being chosen from halogen atoms, a cyano group, groups (C₁-C₆)alkoxy, —O-haloalkyl groups and haloalkyl groups,
a group —O-cycloalkyl, —O-aryl or —O-arylalkyl, or a group —O-heterocycloalkyl, the said groups being optionally substituted with:
an aryl or arylalkyl group, which is itself optionally substituted with one or more halogen atoms, or with a group (C₁-C₆)alkyl,
one or more halogen atoms, and/or
a group (C₁-C₆)alkyl, which may itself be substituted with an aryl or arylalkyl group,
a group —NH—CO—NH-aryl, a group —NH—CO—NH-arylalkyl, a group —NH—CO—NH-heteroaryl, or a group —NH—CO—NH—(C₁-C₆)alkyl, the said aryl, arylalkyl, heteroaryl and alkyl being optionally substituted with at least one atom and/or at least one group, the said atoms and groups being chosen from halogen atoms, a cyano group, a nitro group, a hydroxyl group and groups (C₁-C₆)alkoxy,
a group —N—(C₁-C₆)alkyl, the group (C₁-C₆)alkyl possibly being substituted with at least one aryl or arylalkyl group optionally substituted with at least one halogen atom and/or with at least one group SO₂, or
a group —NH—C(O)-aryl, a group —NH—C(O)-aralkyl or a group —NH—C(O)-heteroaryl, the said groups being optionally substituted with at least one halogen atom;
Ra represents:
a hydrogen atom, a group (C₁-C₆)alkyl or a group (C₁-C₆) cycloalkyl, the said groups being optionally substituted with one or more halogen atoms, with one or more hydroxyl groups, with an aryl or arylalkyl group, with one or more cyano groups and/or with a group —C(O)NRbRc, Rb and Rc being defined below, a group (C₂-C₆) alkynyl, an aryl or arylalkyl group,
Rb represents:
a hydrogen atom, a group (C₁-C₆)alkyl optionally substituted with one or more halogen atoms, with one or more hydroxyl, cyano, amino, heterocycloalkyl or (C₁-C₆)alkoxy groups, or with an aryl or arylalkyl group optionally substituted with one or more halogen atoms, a group (C₃-C₆) cycloalkyl, a group (C₂-C₆) alkenyl or alkynyl, a group (C₁-C₆)alkoxy, an aryl or arylalkyl group optionally substituted with one or more halogen atoms;
Rc represents a hydrogen atom, or a group (C₁-C₆)alkyl optionally substituted with one or more halogen atoms;
given that in the groups-NRbRc, Rb and Rc may form with the nitrogen atom a heteroaryl or a heterocycloalkyl, the latter groups being optionally substituted;
in the form of the base or of an acid-addition salt.

A pharmaceutical composition, comprising at least one compound of formula (56) or an addition salt with a pharmaceutically acceptable acid thereof, and also at least one pharmaceutically acceptable excipient.

In another embodiment, a PDE7 inhibitor useful in the methods of the invention is selected from the following compounds:
3-(3,4-dimethoxybenzyl)-6-hydroxyl-(tetrahydro-2H-pyran-4-yl)quinazoline-2,4(1H,3H)-dione;
{[3-(3,4-dimethoxybenzyl)-2,4-dioxo-1-(tetrahydro-2H-pyran-4-yl) 1,2,3,4-tetrahydroquinazolin-6-yl]oxy}acetonitrile;
3-(3,4-dimethoxybenzyl)-6-pyridin-4-yl1-(tetrahydro-2H-pyran-4-yl)-quinazoline-2,4(1H,3H)-dione hydrochloride;
3[(6-methoxypyridin-3-yl)methyl]-6-pyridin-4-yl 1-(tetrahydro-2H-pyran-4-yl)-quinazoline-2,4(1H,3H)-dione;
6-bromo-3 [(6-methoxypyridin-3-yl)methyl]1-(tetrahydro-2H-pyran-4-yl)-quinazoline-2,4(1H,3H)-dione;
6-bromo-3-(3,4-dimethoxybenzyl) 1-(tetrahydro-2H-pyran-4-yl)quinazoline-2,4(1H,3H)-dione;
2-({3 [(6-methoxypyridin-3-yl)methyl]-2,4-dioxo1-(tetrahydro-2H-pyran-4-yl)-1,2,3,4-tetrahydroquinazolin-6-yl}oxy) propanenitrile;
2-{[3-3,4-dimethoxybenzyl)-2,4-dioxo-1-(tetrahydro-2H-pyran-4-yl) 1,2,3,4-tetrahydroquinazolin-6-yl]oxy}propanenitrile;
({3-[(6-methoxypyridin-3-yl)methyl]-2,4-dioxo-1-(tetrahydro-2H-pyran-4-yl)-1,2,3,4-tetrahydroquinazolin-6-yl}oxy) acetonitrile;
3-(3,4-dimethoxybenzyl)-6-hydroxy-1-(tetrahydro-2H-thiopyran-4-yl)-quinazoline-2,4(1H,3H)-dione;
{[3-(3,4-dimethoxybenzyl)-2,4-dioxo-1-(tetrahydro-2H-thiopyran-4-yl)-1,2,3,4-tetrahydroquinazolin-6-yl]oxy}acetonitrile;
2{[3-(3,4-dimethoxybenzyl)-1-(1,1-dioxidotetrahydro-2H-thiopyran-4-yl)-2,4-dioxo-1,2,3,4-tetrahydroquinazolin-6-yl]oxy}propanenitrile;
3-(3,4-dimethoxybenzyl)-6-hydroxy-1-(1-oxidotetrahydro-2H-thiopyran-4-yl)-quinazoline-2,4(1H,3H)-dione;
{[3-(3,4-dimethoxybenzyl)-1-(1,1-dioxidotetrahydro-2H-thiopyran-4-yl)-2,4-dioxo-1,2,3,4-tetrahydroquinazolin-6-yl]oxy}acetonitrile;
2{[3-(3,4-dimethoxybenzyl)-1-(1-oxidotetrahydro-2H-thiopyran-4-yl)-2,4-dioxo-1,2,3,4-tetrahydroquinazolin-6-yl]oxy}propanenitrile (isomer 1);
2{[3-(3,4-dimethoxybenzyl)-1-(1-oxidotetrahydro-2H-thiopyran-4-yl)-2,4-dioxo-1,2,3,4-tetrahydroquinazolin-6-yl]oxy}propanenitrile (isomer 2);
{[3-(3,4-dimethoxybenzyl)-1-(1-oxidotetrahydro-2H-thiopyran-4-yl)-2,4-dioxo-1,2,3,4-tetrahydroquinazolin-6-yl]oxy}acetonitrile (isomer 1);
{[3-(3,4-dimethoxybenzyl)-1-(1-oxidotetrahydro-2H-thiopyran-4-yl)-2,4-dioxo-1,2,3,4-tetrahydroquinazolin-6-yl]oxy}acetonitrile (isomer 2);
2-{[3-(3,4-dimethoxybenzyl)-2,4-dioxo-1-(tetrahydro-2H-pyran-4-yl)-1,2,3,4-tetrahydroquinazolin-6-yl]oxy}propanenitrilemethane (1:1) (enantiomer 1);
2-{[3-(3,4-dimethoxybenzyl)-2,4-dioxo-1-(tetrahydro-2H-pyran-4-yl)-1,2,3,4-tetrahydroquinazolin-6-yl]oxy}propanenitrilemethane (1:1) (enantiomer 2);
3-(3,4-dimethoxybenzyl)-6-(prop-2-yn-1-yloxy)-1-(tetrahydro-2H-pyran-4-yl)-quinazoline-2,4(1H,3H)-dione;
2{[3-(3,4-dimethoxybenzyl)-2,4-dioxo-1-(tetrahydro-2H-pyran-4-yl)-1,2,3,4-tetrahydroquinazolin-6-yl]oxy}-2-methylpropanenitrile;
2-{[3-(3,4-dimethoxybenzyl)-2,4-dioxo-1-(tetrahydro-2H-pyran-4-yl)-1,2,3,4-tetrahydroquinazolin-6-yl]oxy}-2-methylpropanamide;
3-(3,4-dimethoxybenzyl)-6-(2-hydroxyethoxy)-1-(tetrahydro-2H-pyran-4-yl)-quinazoline-2,4(1H,3H)-dione;
6-(cyclopropylmethoxy)-3-(3,4-dimethoxybenzyl)-1-(tetrahydro-2H-pyran-4-yl)quinazoline-2,4(1H,3H)-dione;
3-(3,4-dimethoxybenzyl)-6-[(1-methylprop-2-yn-1-yl)oxy]-1-(tetrahydro-2H-pyran-4-yl)quinazoline-2,4(1H,3H)-dione;
3-(3,4-dimethoxybenzyl)-6-(3-hydroxyazetidin-1-yl)-1-(tetrahydro-2H-pyran-4-yl)quinazoline-2,4(1H,3H)-dione;
3-(3,4-dimethoxybenzyl)-6-(prop-2-yn-1-ylamino)-1-(tetrahydro-2H-pyran-4-yl)quinazoline-2,4(1H,3H)-dione;
3-(3,4-dimethoxybenzyl)-2,4-dioxo-1-(tetrahydro-2H-pyran-4-yl)-1,2,3,4-tetrahydroquinazoline-6-carbonitrile;
N{[3-(3,4-dimethoxybenzyl)-2,4-dioxo-1-(tetrahydro-2H-pyran-4-yl)-1,2,3,4-tetrahydroquinazolin-6-yl]methyl}acetamide;
1-{[3-(3,4-dimethoxybenzyl)-2,4-dioxo-1-(tetrahydro-2H-pyran-4-yl)-1,2,3,4-tetrahydroquinazolin-6-yl]methyl}urea;
3-(3,4-dimethoxybenzyl)-6-[(1-methylprop-2-yn-1-oxy]-1-(tetrahydro-2H-pyran-4-yl)quinazoline-2,4(1H,3H)-dione (enantiomer 1);
3-(3,4-dimethoxybenzyl)-6-[(1-methylprop-2-yn-1-yl)oxy]-1-(tetrahydro-2H-pyran-4-yl)quinazoline-2,4(1H,3H)-dione (enantiomer 2);
3-(3,4-dimethoxybenzyl)-6-iodo-1-(tetrahydro-2H-pyran-4-yl)quinazoline-2,4(1H,3H)-dione;
3-(3,4-dimethoxybenzyl)-6-[(1-methylprop-2-yn-1-yl)amino]-1-(tetrahydro-2H-pyran-4-yl)quinazoline-2,4(1H,3H)-dione;
3-(3,4-dimethoxybenzyl)-6-propoxy-1-(tetrahydro-2H-pyran-4-yl)quinazoline-2,4(1H,3H)-dione;
3-(3,4-dimethoxybenzyl)-6-(2-methylpropoxy)-1-(tetrahydro-2H-pyran-4-yl)-quinazoline-2,4(1H,3H)-dione;
3-(3,4-dimethoxybenzyl)-6-(1-methylethoxy)-1-(tetrahydro-2H-pyran-4-yl)-quinazoline-2,4(1H,3H)-dione;
3-(3,4-dimethoxybenzyl)-6-(1-hydroxyethyl)-1-(tetrahydro-2H-pyran-4-yl)-quinazoline-2,4(1H,3H)-dione;
6-acetyl-3-(3,4-dimethoxybenzyl)-1-(tetrahydro-2H-pyran-4-yl)quinazoline-2,4(1H,3H)-dione;
6-(2,3-dihydroxypropoxy)-3-(3,4-dimethoxybenzyl)-1-(tetrahydro-2H-pyran-4-yl)quinazoline-2,4(1H,3H)-dione;
3-(3,4-dimethoxybenzyl)-6-(2-hydroxypropoxy)-1-(tetrahydro-2H-pyran-4-yl)-quinazoline-2,4(1H,3H)-dione;
3-(3,4-dimethoxybenzyl)-6-(1-hydroxy-1-methylethyl)-1-(tetrahydro-2H-pyran-4-yl)quinazoline-2,4(1H,3H)-dione;
3-(3,4-dimethoxybenzyl)-6-ethenyl-1-(tetrahydro-2H-pyran-4yl)quinazoline-2,4(1H,3H)dione;
3 (3,4-dimethoxybenzyl)-6-(hydroxymethyl)-1-(tetrahydro-2H-pyran-4-yl)-quinazoline-2,4(1H,3H)dione;
3-(3,4-dimethoxybenzyl)-6-(1-hydroxy-1-methylbut-3-yn-1-yl) 1-(tetrahydro-2H-pyran-4-yl)quinazoline-2,4(1H,3H)-dione;
3-(3,4-dimethoxybenzyl)-6-(2-hydroxyethyl)-1 (tetrahydro-2H-pyran-4-yl)-quinazoline-2,4(1H,3H)dione;
3 (3,4-dimethoxybenzyl)-6-[(1R)-2-hydroxy-1-methylethoxy]1 (tetrahydro-2H-pyran-4-yl)quinazoline-2,4(1H,3H)-dione;
3 (3,4-dimethoxybenzyl)-6-ethoxy-1-(tetrahydro-2H-pyran-4-yl)quinazoline-2,4(1H,3H)-dione;
3-(3,4-dimethoxybenzyl)-6-(2-fluoroethoxy)-1-(tetrahydro-2H-pyran4-yl)-quinazoline-2,4(1H,3H)-dione;
3-(3,4-dimethoxybenzyl)-1-(tetrahydro-2H-pyran-4-yl)-6-(2,2,2-trifluoroethoxy)quinazoline-2,4(1H,3H)-dione;
3-(3,4-dimethoxybenzyl)-6-[(1S)-2-hydroxy-1-methylethoxy]-1-(tetrahydro-2H-pyran-4-yl) quinazoline-2,4(1H,3H)-dione;
6-(2,2difluoroethoxy)-3(3,4-dimethoxybenzyl) 1 (tetrahydro-2H-pyran-4yl)-quinazoline-2,4(1H,3H)-dione;
3-(3,4-dimethoxybenzyl)-1 (tetrahydro-2H-pyran-4-yl)-6-(3,3,3-trifluoropropoxy)quinazoline-2,4(1H,3H)-dione;
3-(3,4-dimethoxybenzyl)-6-{[(1R)-1-methylpropyl]oxy}-1-(tetrahydro-2H-pyran-4-yl)quinazoline-2,4(1H,3H)-dione;
3-(3,4-dimethoxybenzyl)-6-{[(1S)-1-methylpropyl]oxy}-1 (tetrahydro-2H-pyran-4yl)quinazoline-2,4(1H,3H)dione;
3 (3,4-dimethoxybenzyl)-6-[2-fluoro-1 (fluoromethyl)ethoxy]-1-(tetrahydro-2H-pyran-4-yl)quinazoline-2,4(1H,3H)-dione;
3-(3,4-dimethoxybenzyl)-6-[(1S)-2-fluoro-1-methylethoxy]-1-(tetrahydro-2H-pyran-4-yl)quinazoline-2,4(1H,3H)-dione;
6 (2,2-difluoroethenyl)-3-(3,4-dimethoxybenzyl) 1 (tetrahydro-2H-pyran-4-yl)-quinazoline-2,4(1H,3H)-dione;
3-(3,4dimethoxybenzyl)-6-(fluoromethyl)-1-(tetrahydro-2H-pyran-4-yl)-quinazoline-2,4(1H,3H)-dione;
3-(3,4-dimethoxybenzyl)-7-fluoro-6-hydroxyl(tetrahydro-2H-pyran-4-yl)-quinazoline-2,4(1H,3H)-dione;
6-(benzyloxy)-3-(3,4-dimethoxybenzyl)-7fluoro-1-(tetrahydro-2H-pyran-4-yl)quinazoline-2,4(1H,3H)-dione;
6-(2,2-difluoroethoxy)-3-(3,4-dimethoxybenzyl)-7-fluoro-1-(tetrahydro-2H-pyran-4-yl)quinazoline-2,4(1H,3H)-dione;
3-[4-(benzyloxy)-3-methoxybenzyl]-6-[(1,3-difluoropropan-2-yl)oxy]-7-fluoro-1-(tetrahydro-2H-pyran-4-yl)quinazoline-2,4(1H,3H)-dione;
3-{4 [(3,4-dichlorobenzyl)oxy]-3-methoxybenzyl}-6-[(1,3-difluoropropan-2-yl)oxy]-7-fluoro-1-(tetrahydro-2H-pyran-4-yl)quinazoline-2,4(1H,3H)-dione;
3-(3,4-dimethoxybenzyl)-6-nitro-1-(tetrahydro-2H-pyran-4-yl)quinazoline-2,4(1H,3H)-dione;
6-amino-3-(3,4-dimethoxybenzyl)-1-(tetrahydro-2H-pyran-4-yl)quinazoline-2,4(1H,3H)-dione;
6 [(1,3-difluoropropan-2-yl)oxy]-7-fluoro-3-(4-hydroxy-3-methoxybenzyl)-1-(tetrahydro-2H-pyran-4-yl)quinazoline-2,4(1H,3H)-dione;
   in the form of the base or of an acid-addition salt.

A pharmaceutical composition, comprising at least one of the compounds listed above or an addition salt with a pharmaceutically acceptable acid thereof, and also at least one pharmaceutically acceptable excipient.

In more specific embodiments the compounds are selected from (56B), (56C), (56D), (56 E), (56F), (56G), (56H):

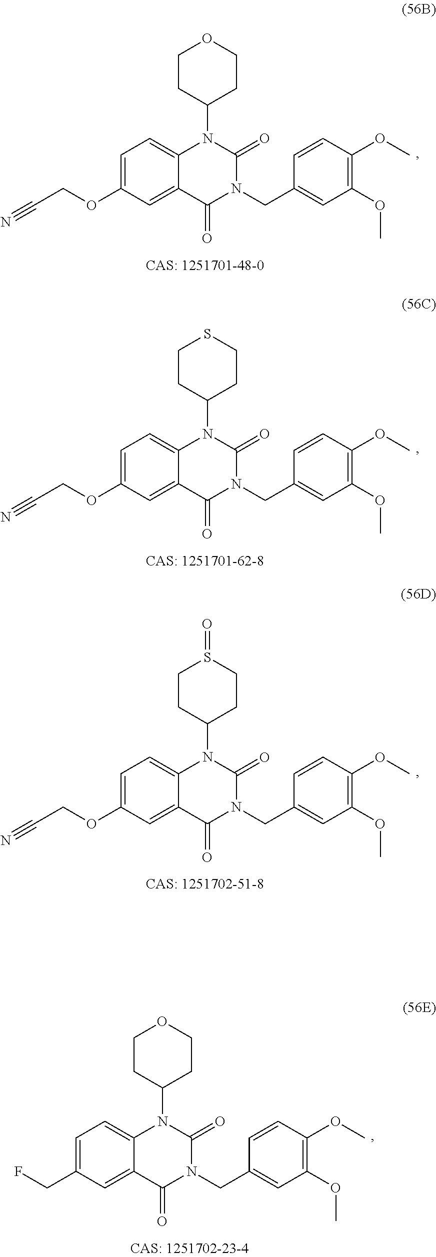 [SMILES: COc1ccc(Cn2c(=O)c3cc(OCC#N)ccc3n(C3CCOCC3)c2=O)cc1OC ; COc1ccc(Cn2c(=O)c3cc(OCC#N)ccc3n(C3CCSCC3)c2=O)cc1OC ; COc1ccc(Cn2c(=O)c3cc(OCC#N)ccc3n(C3CCS(=O)CC3)c2=O)cc1OC ; COc1ccc(Cn2c(=O)c3cc(CF)ccc3n(C3CCOCC3)c2=O)cc1OC]

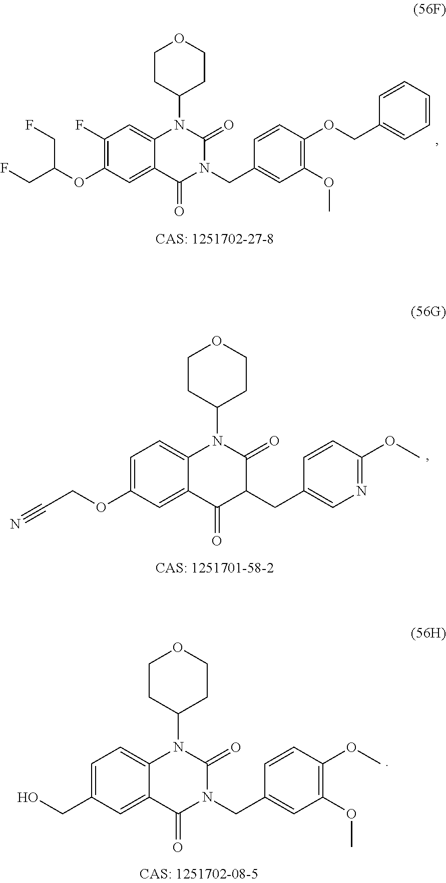 [SMILES: COc1cc(Cn2c(=O)c3cc(OC(CF)CF)c(F)cc3n(C3CCOCC3)c2=O)ccc1OCc1ccccc1 ; COc1ccc(CC2C(=O)c3cc(OCC#N)ccc3N(C3CCOCC3)C2=O)cn1 ; COc1ccc(Cn2c(=O)c3cc(CO)ccc3n(C3CCOCC3)c2=O)cc1OC]

The preparation of the above formulas (56B), (56C), (56D), (56 E), (56F), (56G), (56H) is described in U.S. Pat. No. 8,722,659 and WO 2010/116088.

In another embodiment, PDE7 inhibitors useful in the methods of the invention are selected from those compounds generally or specifically disclosed in US20190185479 and WO 2018/038265 each expressly incorporated by reference herein in its entirety.

In one embodiment, PDE7 inhibitors useful in the methods of the invention have the formula (57):

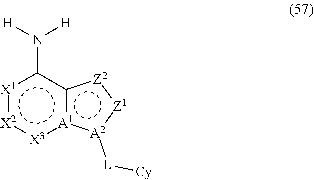

((57), (57A), (57B), (57C), (57D), (57E): Referred to as bicyclic nitrogen-containing heterocyclic compound derivatives containing an amino group.)
Wherein
the partial structure represented by formula 57A

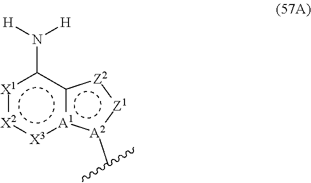

represents a partial structure selected from the group consisting of formula 57B

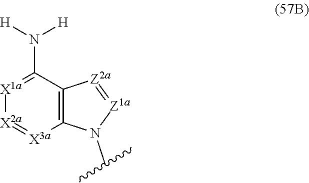

wherein X^{1a} is CR^{X1a} or N; X^{2a} is CR^{X2a} or N; X^{3a} is CR^{X3a} or N; one or two of X^{1a}, X^{2a}, and X^{3a} is/are N; Z^{1a} is CR^{Z1a} or N; and Z^{2a} is CR^{Z2a} or N;

### Formula 57C

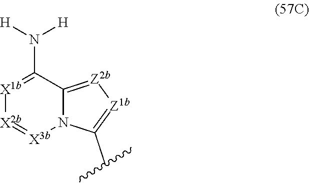

wherein X^{1b} is CR^{X1b} or N; X² is CR^{X2b} or N; X^{3b} is CR^{X3b} or N; zero, one, or two of X^{1b}, X^{2b}, and X^{3b} is/are N; Z^{1b} is CR^{Z1b} or N; and Z^{2b} is CR^{Z2b} or N;

### Formula 57D

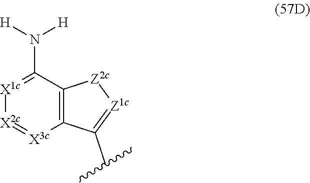

wherein X^{1c} is CR^{X1c} or N; X^{2c} is CR^{X2c} or N; X^{3c} is CR^{X3c} or N; one or two of X^{1c}, X^{2c}, and X^{3c} is/are N; Z^{1c} is CR^{Z1c} or N; and Z^{2c} is NR^{Z2c} or O;

### and Formula 57E

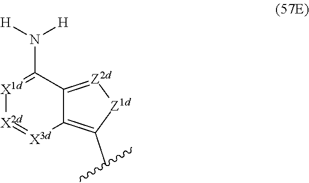

wherein X^{1d} is CR^{X1d} or N; X^{2d} is CR^{X2d} or N; X^{3d} is CR^{X3d} or N; one or two of X^{1d}, X^{2d}, and X^{3d} is/are N; Z^{1d} is NR^{Z1d} or O; and Z^{2d} is CR^{Z2d} or N;
R^{X1a}, R^{X1b}, R^{X1c}, and R^{X1d} each independently represent a hydrogen atom, an optionally substituted alkyl group, or a halogen atom;
R^{X2a}, R^{X2b}, R^{X2c}, and R^{X2d} each independently represent a hydrogen atom, an optionally substituted alkyl group, an optionally substituted alkoxy group, or an optionally substituted alkylthio group;
R^{X3a}, R^{X3b}, R^{X3c}, and R^{X3d} each independently represent a hydrogen atom, an optionally substituted alkyl group, an optionally substituted cycloalkyl group, a halogen atom, a cyano group, or an optionally substituted aryl group;
R^{Z1a}, R^{Z1b}, and R^{21c} each independently represent a hydrogen atom, a hydroxy group, or an optionally substituted alkyl group;
R^{Z1d} represents a hydrogen atom or an optionally substituted alkyl group;
R^{Z2a}, R^{Z2b}, and R^{Z2d} each independently represent a hydrogen atom, an optionally substituted alkyl group, an optionally substituted cycloalkyl group, or a halogen atom;
R^{Z2c} represents a hydrogen atom or an optionally substituted alkyl group;
L represents a single bond or CR^{L1}R^{L2};
R^{L1} and R^{L2} each independently represent a hydrogen atom or an optionally substituted alkyl group, or
R^{L1} and R^{L2} each independently represent an alkylene group and are combined with each other together with the carbon atom to which they are attached to form an optionally substituted monocyclic saturated hydrocarbon group; and
Cy represents
(i) an aryl group optionally substituted with the same or different 1 to 5 substituent(s) selected from the group consisting of
an optionally substituted alkyl group; an optionally substituted alkoxy group; a halogen atom; and an optionally substituted carboxamide group;
(ii) a heteroaryl group optionally substituted with the same or different 1 to 5 substituent(s) selected from the group consisting of an optionally substituted alkyl group and a halogen atom;
(iii) an alicyclic hydrocarbon group optionally substituted with the same or different 1 to 5 substituent(s) selected from the group consisting of
an optionally substituted alkyl group; an optionally substituted alkenyl group; an optionally substituted alkylidene group; an optionally substituted alkoxy group; a hydroxy group; a halogen atom; an oxo group; an optionally substituted aryl group; and an optionally substituted heteroaryl group; or
(iv) a nonaromatic heterocyclic group optionally substituted with the same or different 1 to 5 substituent(s) selected from the group consisting of
an optionally substituted alkyl group; an optionally substituted cycloalkyl group; an optionally substituted alkoxy group; a hydroxy group; a halogen atom; an oxo group; an optionally substituted aryl group; an optionally substituted heteroaryl group; an optionally substituted alkylcarbonyl group; a formyl group; an optionally substituted alkoxycarbonyl group; and an optionally substituted arylcarbonyl group
or a pharmaceutically acceptable salt thereof as an active ingredient.

In another embodiment, a PDE7 inhibitor useful in the methods of the invention is selected from the above compounds wherein

R^{X1a}, R^{X1b}, R^{X1c}, and R^{X1d} each independently represent a hydrogen atom, an alkyl group optionally substituted with the same or different 1 to 7 halogen atom(s), or a halogen atom;
R^{X2a}, R^{X2b}, R^{X2c}, and R^{X2d} each independently represent a hydrogen atom, an alkyl group optionally substituted with the same or different 1 to 7 halogen atom(s), an alkoxy group optionally substituted with the same or different 1 to 7 halogen atom(s), or an alkylthio group optionally substituted with the same or different 1 to 7 halogen atom(s);
R^{X3a}, R^{X3b}, R^{X3c}, and R^{X3d} each independently represent a hydrogen atom, an alkyl group optionally substituted with the same or different 1 to 7 halogen atom(s), a cycloalkyl group optionally substituted with the same or different 1 to 5 halogen atom(s), a halogen atom, a cyano group, or an aryl group optionally substituted with the same or different 1 to 5 halogen atom(s);
R^{Z1a}, R^{Z1b}, and R^{Z1c} each independently represent a hydrogen atom, a hydroxy group, or an alkyl group optionally substituted with the same or different 1 to 7 halogen atom(s);
R^{Z1d} represents a hydrogen atom or an alkyl group optionally substituted with the same or different 1 to 5 halogen atom(s);
R^{Z2a}, R^{Z2b}, and R^{Z2d} each independently represent a hydrogen atom, an alkyl group optionally substituted with the same or different 1 to 7 halogen atom(s), a cycloalkyl group optionally substituted with the same or different 1 to 5 halogen atom(s), or a halogen atom;
R^{Z2c} represents a hydrogen atom or an alkyl group optionally substituted with the same or different 1 to 5 halogen atom(s);
L represents a single bond or CR^{L1}R^{L2};
R^{L1} and R^{L2} each independently represent a hydrogen atom or an alkyl group optionally substituted with the same or different 1 to 7 halogen atom(s), or R^{L1} and R^{L2} each independently represent a straight alkylene group and are combined with each other together with the carbon atom to which they are attached to form a monocyclic saturated hydrocarbon group optionally substituted with the same or different 1 to 6 halogen atom(s); and
Cy represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 substituent(s) selected from the group consisting of a halogen atom and an aryl group; a halogen atom; and
a carboxamide group optionally substituted with the same or different 1 or 2 alkyl group(s) optionally substituted with the same or different 1, 2, or 3 aryl group(s);
(ii) a heteroaryl group optionally substituted with the same or different 1 to 5 substituent(s) selected from the group consisting of an alkyl group optionally substituted with the same or different 1 to 7 halogen atom(s) and a halogen atom;
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, an arylalkyloxy group,
and an aryl group optionally substituted with the same or different 1, 2, or 3 substituent(s) selected from the group consisting of an alkyl group optionally substituted with the same or different 1 to 7 halogen atom(s) and a halogen atom;
an alkenyl group optionally substituted with the same or different 1 to 5 halogen atom(s);
an alkylidene group optionally substituted with the same or different 1 to 6 halogen atom(s);
an alkoxy group optionally substituted with the same or different 1 to 7 halogen atom(s);
a hydroxy group; a halogen atom; an oxo group; an aryl group optionally substituted with the same or different 1 to 5 halogen atom(s); and
a heteroaryl group optionally substituted with the same or different 1, 2, or 3 substituent(s) selected from the group consisting of an alkyl group optionally substituted with the same or different 1 to 7 halogen atom(s) and a halogen atom; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of an alkoxy group optionally substituted with the same or different 1 to 7 halogen atom(s), a halogen atom, and an aryl group optionally substituted with the same or different 1, 2, or 3 substituent(s) selected from the group consisting of an alkyl group optionally substituted with the same or different 1 to 7 halogen atom(s) and a halogen atom;
a cycloalkyl group optionally substituted with the same or different 1 to 5 halogen atom(s);
an alkoxy group optionally substituted with the same or different 1 to 7 halogen atom(s);
a hydroxy group; a halogen atom; an oxo group;
an aryl group optionally substituted with the same or different 1 to 5 halogen atom(s);
a heteroaryl group optionally substituted with the same or different 1 to 5 halogen atom(s);
an alkylcarbonyl group optionally substituted with the same or different 1, 2, or 3 aryl group(s);
a formyl group; an alkoxycarbonyl group optionally substituted with the same or different 1 to 7 halogen atom(s); and an arylcarbonyl group optionally substituted with the same or different 1 to 5 halogen atom(s).

In another embodiment, a PDE7 inhibitor useful in the methods of the invention is selected from the compounds wherein
Cy represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and
a carboxamide group optionally substituted with the same or different 1 or 2 alkyl group(s) optionally substituted with the same or different 1, 2, or 3 aryl group(s);
(ii) a heteroaryl group optionally substituted with the same or different 1 to 5 halogen atom(s);
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and a heteroaryl group optionally substituted with the same or different 1, 2, or 3 alkyl group(s); or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group.

In another embodiment, a PDE7 inhibitor useful in the methods of the invention is selected from the compounds wherein
Cy represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and
a carboxamide group optionally substituted with the same or different 1 or 2 alkyl group(s) optionally substituted with the same or different 1, 2, or 3 aryl group(s),
wherein said aryl group is a 6 to 11 membered monocyclic or bicyclic aromatic hydrocarbon group;
(ii) a heteroaryl group optionally substituted with the same or different 1 to 5 halogen atom(s), wherein said heteroaryl group is a 5 to 11 membered monocyclic or bicyclic aromatic heterocyclic group comprising 1 to 4 heteroatom(s) selected from the group consisting of an oxygen atom, a sulfur atom, and a nitrogen atom other than carbon atom(s);
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and
a heteroaryl group optionally substituted with the same or different 1, 2, or 3 alkyl group(s), wherein said alicyclic hydrocarbon group is a C₃-C₈ cycloalkyl group, a C₆-C₁₂ bicycloalkyl group, a C₆-C₁₂ bicycloalkenyl group, a C₆-C₁₂ spiroalkyl group, or a C₁₀-C₁₄ tricyclic tricycloalkyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group,
wherein said nonaromatic heterocyclic group is a 4 to 8 membered monocyclic nonaromatic heterocyclic group or a 6 to 12 membered bicyclic nonaromatic heterocyclic group.

In another embodiment, a PDE7 inhibitor useful in the methods of the invention is selected from the above compounds wherein
X^{1a}, X^{1b}, X^{1c}, and X^{1d} each represent N.
Z^{1a}, Z^{1b}, and Z^{1c} each represent N; and Z^{1d} represents NR^{Z1d}.
Z^{2a}, Z^{2b}, and Z^{2d} each represent N; and Z^{2c} represents NR^{Z2c}.
X^{3a}, X^{3b}, X^{3c}, and X^{3d} each represent N.

In another embodiment, a PDE7 inhibitor useful in the methods of the invention is selected from formula 58, compound or pharmaceutically acceptable salt thereof:

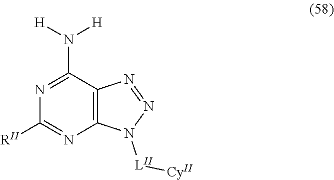

((58): Referred to as 3,5-substituted 1,2,3-triazolo[4,5-d]pyrimidin-7-amine derivatives.)
Wherein
R^{II} represents a hydrogen atom, an alkyl group optionally substituted with the same or different 1 to 7 halogen atom(s), an alkoxy group optionally substituted with the same or different 1 to 7 halogen atom(s), or an alkylthio group optionally substituted with the same or different 1 to 7 halogen atom(s);
L_{II} represents a single bond or CR^{LII-1}R^{LII-2};
R^{LII-1} and R^{LII-2} each independently represent a hydrogen atom or an alkyl group optionally substituted with the same or different 1 to 7 halogen atom(s), or R^{LII-1} and R^{LII-2} each independently represent an alkylene group and are combined with each other together with the carbon atom to which they are attached to form a monocyclic saturated hydrocarbon group optionally substituted with the same or different 1 to 6 halogen atom(s); and
Cy^{II} represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 substituent(s) selected from the group consisting of a halogen atom and an aryl group; a halogen atom; and
a carboxamide group optionally substituted with the same or different 1 or 2 alkyl group(s) optionally substituted with the same or different 1, 2, or 3 aryl group(s),
provided that said aryl group is not a phenyl group;
(ii) a heteroaryl group optionally substituted with the same or different 1 to 5 substituent(s) selected from the group consisting of an alkyl group optionally substituted with the same or different 1 to 7 halogen atom(s) and a halogen atom, provided that said heteroaryl group is not a furyl group;
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, an arylalkyloxy group, and an aryl group optionally substituted with the same or different 1, 2, or 3 substituent(s) selected from the group consisting of an alkyl group optionally substituted with the same or different 1 to 7 halogen atom(s) and a halogen atom;
an alkenyl group optionally substituted with the same or different 1 to 5 halogen atom(s);
an alkylidene group optionally substituted with the same or different 1 to 6 halogen atom(s);
an alkoxy group optionally substituted with the same or different 1 to 7 halogen atom(s);
a hydroxy group; a halogen atom; an oxo group; an aryl group optionally substituted with the same or different 1 to 5 halogen atom(s); and a heteroaryl group optionally substituted with the same or different 1, 2, or 3 substituent(s) selected from the group consisting of an alkyl group optionally substituted with the same or different 1 to 7 halogen atom(s) and a halogen atom, provided that said alicyclic hydrocarbon group is not a cyclobutyl group, a cyclopentyl group, a cyclopentenyl group, or a 2-cyclohexenyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of an alkoxy group optionally substituted with the same or different 1 to 7 halogen atom(s), a halogen atom, and an aryl group optionally substituted with the same or different 1, 2, or 3 substituent(s) selected from the group consisting of an alkyl group optionally substituted with the same or different 1 to 7 halogen atom(s) and a halogen atom;
a cycloalkyl group optionally substituted with the same or different 1 to 5 halogen atom(s);
an alkoxy group optionally substituted with the same or different 1 to 7 halogen atom(s);
a hydroxy group; a halogen atom; an oxo group; an aryl group optionally substituted with the same or different 1 to 5 halogen atom(s); a heteroaryl group optionally substituted with the same or different 1 to 5 halogen atom(s); an alkylcarbonyl group optionally substituted with the same or different 1, 2, or 3 aryl group(s); a formyl group; an alkoxycarbonyl group optionally substituted with the same or different 1 to 7 halogen atom(s); and an arylcarbonyl group optionally substituted with the same or different 1 to 5 halogen atom(s), provided that said nonaromatic heterocyclic group is not a tetrahydrofuryl group, a dihydrofuran-2-yl group, a tetrahydropyran-2-yl group, a pyrrolidin-3-yl group, a morpholin-2-yl group, or a thiolan-2-yl group,
provided that
(a) Cy^{II} is not a cyclopropyl group or a 2,2-dimethyl-1,3-dioxolanyl group; and
(b) the compound is not 3-cyclohexyl-3H-[1,2,3]triazolo[4,5-d]pyrimidin-7-amine, 2-[(7-amino-3H-1,2,3-triazolo[4,5-d]pyrimidin-3-yl)methyl]-1-azabicyclo[2.2.2]octan-3-one, 2-(7-amino-3H-1,2,3-triazolo[4,5-d]pyrimidin-3-yl)cyclohexanemethanol, or 4-(7-amino-3H-1,2,3-triazolo[4,5-d]pyrimidin-3-yl)-2-hydroxy-bicyclo[3.1.0]hexane-1-methanol),
or a pharmaceutically acceptable salt thereof.

In another embodiment, a PDE7 inhibitor useful in the methods of the invention is selected from formula 58, compound or pharmaceutically acceptable salt thereof, wherein
R^{II} represents a hydrogen atom, an alkyl group optionally substituted with the same or different 1 to 7 halogen atom(s), an alkoxy group, or an alkylthio group;
L^{II} represents a single bond or CR^{LII-1}R^{LII-2};
R^{LII-1} and R^{LII-2} each independently represent a hydrogen atom or an alkyl group, or R^{LII-1} and R^{LII-2} each independently represent a straight alkylene group and are combined with each other together with the carbon atom to which they are attached to form a monocyclic saturated hydrocarbon group; and
Cy^{II} represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and a carboxamide group optionally substituted with the same or different 1 or 2 alkyl group(s) optionally substituted with the same or different 1, 2, or 3 aryl group(s), wherein said aryl group is a 6 to 11 membered monocyclic or bicyclic aromatic hydrocarbon group;
(ii) a heteroaryl group optionally substituted with the same or different 1 to 5 halogen atom(s), wherein said heteroaryl group is a 5 to 11 membered monocyclic or bicyclic aromatic heterocyclic group comprising 1 to 4 heteroatom(s) selected from the group consisting of an oxygen atom, a sulfur atom, and a nitrogen atom other than carbon atom(s);
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and
a heteroaryl group optionally substituted with the same or different 1, 2, or 3 alkyl group(s), wherein said alicyclic hydrocarbon group is a C₃-C₈ cycloalkyl group, a C₆-C₁₂ bicycloalkyl group, a C₆-C₁₂ bicycloalkenyl group, a C₆-C₁₂ spiroalkyl group, or a C₁₀-C₁₄ tricyclic tricycloalkyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group,
wherein said nonaromatic heterocyclic group is a 4 to 8 membered monocyclic nonaromatic heterocyclic group or a 6 to 12 membered bicyclic nonaromatic heterocyclic group.

In another embodiment, a PDE7 inhibitor useful in the methods of the invention is selected from formula 58, compound or pharmaceutically acceptable salt thereof, wherein
L^{II} represents a single bond; and
Cy^{II} represents
(i) a naphthyl group or a tetrahydronaphthyl group, each of which is optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and
a carboxamide group optionally substituted with the same or different 1 or 2 alkyl group(s) optionally substituted with the same or different 1, 2, or 3 aryl group(s);
(ii) a tetrahydroindazolyl group;
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and
a heteroaryl group optionally substituted with the same or different 1, 2, or 3 alkyl group(s), wherein said alicyclic hydrocarbon group is a cyclohexyl group, a cycloheptyl group, a bicyclo[3.1.0]hexyl group, a bicyclo[3.1.0]hexenyl group, a bicyclo[2.2.1]heptyl group, a bicyclo[4.1.0]heptyl group, a spiro[2.3]hexyl group, a spiro[2.5]octyl group, or an adamantyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group,
wherein said nonaromatic heterocyclic group is a piperidinyl group, a piperidino group, a perhydroazepinyl group, a perhydroazocinyl group, a tetrahydropyranyl group, an azabicyclo[3.1.0]hexyl group, an azabicyclo[2.2.1]heptyl group, an azabicyclo[3.2.1]octyl group, an azabicyclo[2.2.2]octyl group, an azaspiro[2.5]octyl group, or an azaspiro[4.5]decyl group.

In another embodiment, a PDE7 inhibitor useful in the methods of the invention is selected from formula 59, compound or pharmaceutically acceptable salt thereof:

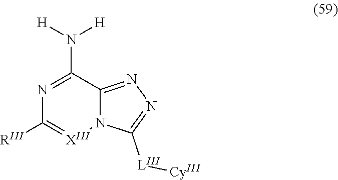

((59): Referred to as 3-substituted 1,2,4-triazolo derivatives.)
Wherein
X^{III} is CR^{XIII} or N;
R^{III} represents a hydrogen atom, an alkyl group optionally substituted with the same or different 1 to 7 halogen atom(s), an alkoxy group optionally substituted with the same or different 1 to 7 halogen atom(s), or an alkylthio group optionally substituted with the same or different 1 to 7 halogen atom(s);
R^{XIII} represents a hydrogen atom, an alkyl group optionally substituted with the same or different 1 to 7 halogen atom(s), a cycloalkyl group optionally substituted with the same or different 1 to 5 halogen atom(s), a halogen atom, a cyano group, or an aryl group optionally substituted with the same or different 1 to 5 halogen atom(s);
L^{III} represents a single bond or CR^{LIIII-1}R^{LIII-2};
R^{LIII-1} and R^{LII-2} each independently represent a hydrogen atom or an alkyl group optionally substituted with the same or different 1 to 7 halogen atom(s), or R^{LIIII-1} and R^{LIIII-2} each independently represent an alkylene group and are combined with each other together with the carbon atom to which they are attached to form a monocyclic saturated hydrocarbon group optionally substituted with the same or different 1 to 6 halogen atom(s); and
Cy^{III} represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 substituent(s) selected from the group consisting of a halogen atom and an aryl group; a halogen atom; and
a carboxamide group optionally substituted with the same or different 1 or 2 alkyl group(s) optionally substituted with the same or different 1, 2, or 3 aryl group(s);
(ii) a heteroaryl group optionally substituted with the same or different 1 to 5 substituent(s) selected from the group consisting of an alkyl group optionally substituted with the same or different 1 to 7 halogen atom(s) and a halogen atom;
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, an arylalkyloxy group, and an aryl group optionally substituted with the same or different 1, 2, or 3 substituent(s) selected from the group consisting of an alkyl group optionally substituted with the same or different 1 to 7 halogen atom(s) and a halogen atom;
an alkenyl group optionally substituted with the same or different 1 to 5 halogen atom(s);
an alkylidene group optionally substituted with the same or different 1 to 6 halogen atom(s);
an alkoxy group optionally substituted with the same or different 1 to 7 halogen atom(s);
a hydroxy group; a halogen atom; an oxo group; an aryl group optionally substituted with the same or different 1 to 5 halogen atom(s); and a heteroaryl group optionally substituted with the same or different 1, 2, or 3 substituent(s) selected from the group consisting of an alkyl group optionally substituted with the same or different 1 to 7 halogen atom(s) and a halogen atom; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of an alkoxy group optionally substituted with the same or different 1 to 7 halogen atom(s), a halogen atom, and an aryl group optionally substituted with the same or different 1, 2, or 3 substituent(s) selected from the group consisting of an alkyl group optionally substituted with the same or different 1 to 7 halogen atom(s) and a halogen atom;
a cycloalkyl group optionally substituted with the same or different 1 to 5 halogen atom(s);
an alkoxy group optionally substituted with the same or different 1 to 7 halogen atom(s);
a hydroxy group; a halogen atom; an oxo group; an aryl group optionally substituted with the same or different 1 to 5 halogen atom(s);
a heteroaryl group optionally substituted with the same or different 1 to 5 halogen atom(s);
an alkylcarbonyl group optionally substituted with the same or different 1, 2, or 3 aryl group(s);
a formyl group;
an alkoxycarbonyl group optionally substituted with the same or different 1 to 7 halogen atom(s); and
an arylcarbonyl group optionally substituted with the same or different 1 to 5 halogen atom(s), provided that said nonaromatic heterocyclic group is not a tetrahydrofuryl group,
provided that
(a) when X^{III} is CH, and Cy^{III} is a phenyl group optionally substituted with the same or different 1 or 2 halogen atom(s), then R^{III} is not a hydrogen atom; and
(b) the compound is not 3-cyclopropyl[1,2,4]triazolo[4,3-a]pyrazin-8-amine)],
or a pharmaceutically acceptable salt thereof.

In another embodiment, a PDE7 inhibitor useful in the methods of the invention is selected from formula 59, compound or pharmaceutically acceptable salt thereof, wherein
R^{III} represents a hydrogen atom, an alkyl group optionally substituted with the same or different 1 to 7 halogen atom(s), an alkoxy group, or an alkylthio group;
R^{XIII} represents a hydrogen atom, an alkyl group optionally substituted with the same or different 1 to 7 halogen atom(s), a cycloalkyl group, a halogen atom, a cyano group, or an aryl group;
L^{III} represents a single bond or CR^{LIIII-1}R^{LIII-2};
R^{LIII-1} and R^{LIII-2} each independently represent a hydrogen atom or an alkyl group, or R^{LIIII-1} and R^{LIIII-2} each independently represent a straight alkylene group and are combined with each other together with the carbon atom to which they are attached to form a monocyclic saturated hydrocarbon group; and
Cy^{III} represents
(i) an aryl group optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and
a carboxamide group optionally substituted with the same or different 1 or 2 alkyl group(s) optionally substituted with the same or different 1, 2, or 3 aryl group(s),
wherein said aryl group is a 6 to 11 membered monocyclic or bicyclic aromatic hydrocarbon group;
(ii) a heteroaryl group optionally substituted with the same or different 1 to 5 halogen atom(s), wherein said heteroaryl group is a 5 to 11 membered monocyclic or bicyclic aromatic heterocyclic group comprising 1 to 4 heteroatom(s) selected from the group consisting of an oxygen atom, a sulfur atom, and a nitrogen atom other than carbon atom(s);
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and a heteroaryl group optionally substituted with the same or different 1, 2, or 3 alkyl group(s), wherein said alicyclic hydrocarbon group is a C₃-C₈ cycloalkyl group, a C₆-C₁₂ bicycloalkyl group, a C₆-C₁₂ bicycloalkenyl group, a C₆-C₁₂ spiroalkyl group, or a C₁₀-C₁₄ tricyclic tricycloalkyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group,
wherein said nonaromatic heterocyclic group is a 4 to 8 membered monocyclic nonaromatic heterocyclic group or a 6 to 12 membered bicyclic nonaromatic heterocyclic group.

In another embodiment, a PDE7 inhibitor useful in the methods of the invention is selected from formula 59, compound or pharmaceutically acceptable salt thereof, wherein
L^{III} represents a single bond; and
Cy^{III} represents
(i) a phenyl group, a naphthyl group, or a tetrahydronaphthyl group, each of which is optionally substituted with the same or different 1 to 5 substituent(s) selected from the group consisting of
an alkyl group optionally substituted with the same or different 1 to 7 halogen atom(s);
an alkoxy group optionally substituted with the same or different 1, 2, or 3 aryl group(s);
a halogen atom; and
a carboxamide group optionally substituted with the same or different 1 or 2 alkyl group(s) optionally substituted with the same or different 1, 2, or 3 aryl group(s);
(ii) a tetrahydroindazolyl group;
(iii) an alicyclic hydrocarbon group optionally substituted with the same or different 1 to 5 substituent(s) selected from the group consisting of
an alkyl group optionally substituted with the same or different 1, 2, or 3 substituent(s) selected from the group consisting of a halogen atom, a hydroxy group, an aryloxy group, and an arylalkyloxy group; an alkenyl group; an alkylidene group; an alkoxy group; a hydroxy group; a halogen atom; and a heteroaryl group optionally substituted with the same or different 1, 2, or 3 alkyl group(s), wherein said alicyclic hydrocarbon group is a cyclobutyl group, a cyclopentyl group, a cyclohexyl group, a cycloheptyl group, a bicyclo[3.1.0]hexyl group, a bicyclo[3.1.0]hexenyl group, a bicyclo[2.2.1]heptyl group, a bicyclo[4.1.0]heptyl group, a spiro[2.3]hexyl group, a spiro[2.5]octyl group, or an adamantyl group; or
(iv) a nonaromatic heterocyclic group optionally substituted with the same or different 1 to 5 substituent(s) selected from the group consisting of
an alkyl group optionally substituted with the same or different 1, 2, or 3 substituent(s) selected from the group consisting of a halogen atom and an aryl group; a halogen atom; an aryl group; a heteroaryl group; and an alkoxycarbonyl group,
wherein said nonaromatic heterocyclic group is a pyrrolidinyl group, a piperidinyl group, a piperidino group, a perhydroazepinyl group, a perhydroazocinyl group, a morpholinyl group, a morpholino group, a tetrahydropyranyl group, an azabicyclo[3.1.0]hexyl group, an azabicyclo[2.2.1]heptyl group, an azabicyclo[3.2.1]octyl group, an azabicyclo[2.2.2]octyl group, an azaspiro[2.5]octyl group, or an azaspiro[4.5]decyl group.

In another embodiment, a PDE7 inhibitor useful in the methods of the invention is selected from formula 59, compound or pharmaceutically acceptable salt thereof, wherein X^{III} represents CR^{XIII}.

In another embodiment, a PDE7 inhibitor useful in the methods of the invention is selected from formula 59, compound or pharmaceutically acceptable salt thereof, wherein X^{III} represents N.

In another embodiment, a pharmaceutical composition of a PDE7 inhibitor useful in the methods of the invention is comprising: the compound or pharmaceutically acceptable salt of formula 58 as an active ingredient, and a pharmaceutically acceptable carrier.

In another embodiment, a pharmaceutical composition of a PDE7 inhibitor useful in the methods of the invention is comprising: the compound or pharmaceutically acceptable salt of formula 59 as an active ingredient, and a pharmaceutically acceptable carrier.

In another embodiment, a PDE7 inhibitor useful in the methods of the invention is selected from the following compounds:
3-(cis-2-methylcyclohexyl)-3H-[1,2,3]triazolo[4,5-d]pyrimidin-7-amine;
3-(trans-2-methylcyclohexyl)-3H-[1,2,3]triazolo[4,5-d]pyrimidin-7-amine;
3-(cis-2-fluorocyclohexyl)-3H-[1,2,3]triazolo[4,5-d]pyrimidin-7-amine;
3-(2,2-difluorocyclohexyl)-3H-[1,2,3]triazolo[4,5-d]pyrimidin-7-amine;
3-(cis-3-methylcyclohexyl)-3H-[1,2,3]triazolo[4,5-d]pyrimidin-7-amine;
3-(trans-3-methylcyclohexyl)-3H-[1,2,3]triazolo[4,5-d]pyrimidin-7-amine;
3-(3,3-dimethylcyclohexyl)-3H-[1,2,3]triazolo[4,5-d]pyrimidin-7-amine;
3-[cis-3-(trifluoromethyl)cyclohexyl]-3H-[1,2,3]triazolo[4,5-d]pyrimidin-7-amine;
3-(cis-4-methylcyclohexyl)-3H-[1,2,3]triazolo[4,5-d]pyrimidin-7-amine;
3-(trans-4-methylcyclohexyl)-3H-[1,2,3]triazolo[4,5-d]pyrimidin-7-amine;
3-(4,4-dimethylcyclohexyl)-3H-[1,2,3]triazolo[4,5-d]pyrimidin-7-amine;
3-(trans-3,3,5-trimethylcyclohexyl)-3H-[1,2,3]triazolo[4,5-d]pyrimidin-7-amine;
3-cycloheptyl-3H-[1,2,3]triazolo[4,5-d]pyrimidin-7-amine;
3-cyclohexyl[1,2,4]triazolo[4,3-a]pyrazin-8-amine;
3-(1-fluorocyclohexyl)[1,2,4]triazolo[4,3-a]pyrazin-8-amine;
3-(cis-3-methylcyclohexyl)[1,2,4]triazolo[4,3-a]pyrazin-8-amine;
3-(trans-3-methylcyclohexyl)[1,2,4]triazolo[4,3-a]pyrazin-8-amine;
3-(3,3-dimethylcyclohexyl)[1,2,4]triazolo[4,3-a]pyrazin-8-amine;
3-(spiro[2,5]oct-5-yl)[1,2,4]triazolo[4,3-a]pyrazin-8-amine;
3-[cis-3-(trifluoromethyl)cyclohexyl][1,2,4]triazolo[4,3-a]pyrazin-8-amine;
3-(3,3-difluorocyclohexyl)[1,2,4]triazolo[4,3-a]pyrazin-8-amine;
3-(trans-4-methylcyclohexyl)[1,2,4]triazolo[4,3-a]pyrazin-8-amine;
3-[2-methyl-5-(trifluoromethyl)cyclohexyl][1,2,4]triazolo[4,3-a]pyrazin-8-amine;
3-(cis-5,5-difluoro-2-methylcyclohexyl)[1,2,4]triazolo[4,3-a]pyrazin-8-amine;
3-(trans-3,3-difluoro-5-methylcyclohexyl[1,2,4]triazolo[4,3-a]pyrazin-8-amine;
3-(3,3-difluoro-5,5-dimethylcyclohexyl)[1,2,4]triazolo[4,3-a]pyrazin-8-amine;
3-[cis-2,2-difluoro-5-(trifluoromethyl)cyclohexyl][1,2,4]triazolo[4,3-a]pyrazin-8-amine;
3-(bicyclo[4.1.0]hept-3-yl)[1 1,2,4]triazolo[4,3-a]pyrazin-8-amine;
3-[(1R,6S,7r)-bicyclo[4.1.0]hept-7-yl][1,2,4]triazolo[4,3-a]pyrazin-8-amine;
3-(2-methylpiperidin-1-yl)[1,2,4]triazolo[4,3-a]pyrazin-8-amine;
3-(2-ethylpiperidin-1-yl)[1,2,4]triazolo[4,3-a]pyrazin-8-amine;
3-(3,3-dimethylpiperidin-1-yl)[1,2,4]triazolo[4,3-a]pyrazin-8-amine;
3-(3,3-dimethylpiperidin-1-yl)-5-methyl[1,2,4]triazolo[4,3-a]pyrazin-8-amine;
3-(3,3-dimethylpiperidin-1-yl)-5-ethyl[1,2,4]triazolo[4,3-a]pyrazin-8-amine;
5-cyclopropyl-3-(3,3-dimethylpiperidin-1-yl)[1,2,4]triazolo[4,3-a]pyrazin-8-amine;
3-(3,3-dimethylpiperidin-1-yl)-5-(trifluoromethyl)[1,2,4]triazolo[4,3-a]pyrazin-8-amine;
5-chloro-3-(3,3-dimethylpiperidin-1-yl)[1,2,4]triazolo[4,3-a]pyrazin-8-amine;
8-amino-3-(3,3-dimethylpiperidin-1-yl)[1,2,4]triazolo[4,3-a]pyrazine-5-carbonitrile;
3-[trans-3,5-dimethylpiperidin-1-yl][1,2,4]triazolo[4,3-a]pyrazin-8-amine;
8-amino-3-(3,5-dimethylpiperidin-1-yl)[1,2,4]triazolo[4,3-a]pyrazine-5-carbonitrile;
3-(3,4-dimethylpiperidin-1-yl)[1,2,4]triazolo[4,3-a]pyrazin-8-amine;
3-(2,3-dimethylpiperidin-1-yl)[1,2,4]triazolo[4,3-a]pyrazin-8-amine;
3-(2,5-dimethylpiperidin-1-yl)[1,2,4]triazolo[4,3-a]pyrazin-8-amine;
8-amino-3-(2,5-dimethylpiperidin-1-yl)[1,2,4]triazolo[4,3-a]pyrazine-5-carbonitrile;
3-(2,4-dimethylpiperidin-1-yl)[1,2,4]triazolo[4,3-a]pyrazin-8-amine;
3-(2,5,5-trimethylpiperidin-1-yl)[1,2,4]triazolo[4,3-a]pyrazin-8-amine;
3-cyclohexyl[1,2,4]triazolo[3,4-f][1,2,4]triazin-8-amine;
3-(cis-2-methylcyclohexyl)[1,2,4]triazolo[3,4-f][1,2,4]triazin-8-amine;
3-(trans-2-methylcyclohexyl)[1,2,4]triazolo[3,4-f][1,2,4]triazin-8-amine;
3-(cis-3-methylcyclohexyl)[1,2,4]triazolo[3,4-f][1,2,4]triazin-8-amine;
3-(trans-3-methylcyclohexyl)[1,2,4]triazolo[3,4-f][1,2,4]triazin-8-amine;
3-(3,3-dimethylcyclohexyl)[1,2,4]triazolo[3,4-f][1,2,4]triazin-8-amine;
3-[cis-3-(trifluoromethyl)cyclohexyl][1,2,4]triazolo[3,4-f][1,2,4]triazin-8-amine;
3-[trans-3-(trifluoromethyl)cyclohexyl][1,2,4]triazolo[3,4-f][1,2,4]triazin-8-amine;
3-(3,3-difluorocyclohexyl)[1,2,4]triazolo[3,4-f][1,2,4]triazin-8-amine;
3-(cis-5,5-difluoro-2-methylcyclohexyl)[1,2,4]triazolo[3,4-f][1,2,4]triazin-8-amine; and
3-[2-methyl-5-(trifluoromethyl)cyclohexyl][1,2,4]triazolo[3,4-f][1,2,4]triazin-8-amine.

A pharmaceutical composition comprising: the compound or pharmaceutically acceptable salt of at least one of the compounds listed above as an active ingredient, and a pharmaceutically acceptable carrier.

The preparation of the above compounds is described in US20190185479 and WO 2018/038265.

In another embodiment, PDE7 inhibitors useful in the methods of the invention are selected from those compounds generally or specifically disclosed in WO 2010/029299 each expressly incorporated by reference herein in its entirety.

In one embodiment, PDE7 inhibitors useful in the methods of the invention have the formula (60) or pharmaceutically acceptable salt thereof:

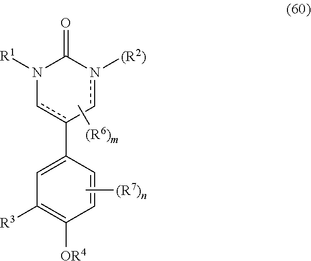

((60), (61): Also referred to as 2-pyrimindinone derivatives.)
Wherein
the dotted lines each independently represent an optional bond (and when the dotted line between the carbon and nitrogen is present, then R² is absent, and when the dotted line between the carbon and nitrogen is absent, then R² is present);
m represents 0, 1, 2, 3, 4 or 5;
n represents 0, 1, 2 or 3;
at least one of R¹ and, if present, R² represents -A¹-T²-B¹ and the other (if present) represents R⁵;
R³ represents hydrogen, —OR^{4a}, C₁₋₁₂ alkyl (optionally substituted by one or more substituents selected from =0 and X¹) or —B²;
R⁴ and R^{4a} independently represent hydrogen, d_₁₂ alkyl (optionally substituted by one or more substituents selected from =0 and X²) or —B³;
R⁵ represents hydrogen, C₁₋₁₂ alkyl (optionally substituted by one or more substituents selected from =0 and X³) or —B^{3a};
each R⁶ and each R⁷ independently represent X⁴, C₁₋₁₂ alkyl (optionally substituted by one or more substituents selected from ═O and X⁵) or —B⁴; or any two R⁶ groups may be linked together to form a further ring, which is formed either by the two relevant groups being linked together by a direct bond or C₁₋₅ alkylene;
A¹ represents C_{L12} alkylene (optionally substituted by one or more substituents selected from =0 and X⁶);
T² represents a direct bond, —N(R^{w1})— or —C(O)N(R^{w2})—;
R^{w1} and R^{w2} independently represent hydrogen, C_{M2} alkyl (optionally substituted by one or more substituents selected from X⁷) or —B⁵;
B¹ represents:
1) a monocyclic 5-membered heteroaryl group; 2) a polycyclic heteroaryl group;
3) a polycyclic aryl group; or
4) a heterocycloalkyl group, all four of which are optionally and independently substituted with one or more substituents selected from X⁸ and, in the case of heterocycloalkyl or any non-aromatic rings of a polycyclic aryl or heteroaryl group, ═O;
B², B³ and B³a independently represent aryl (optionally substituted by one or more substituents selected from X⁹), heterocycloalkyl (optionally substituted by one or more substituents selected from =0 and X¹⁰) or heteroaryl (optionally substituted by one or more substituents selected from X₁₁);
B⁴ and B⁵ independently represent heterocycloalkyl (optionally substituted by one or more substituents selected from =0 and X¹²);
X¹, X², X³, X⁴, X⁵, X⁶, X⁷, X⁸, X⁹, X¹⁰X¹¹ and X¹² independently represent B⁶, halo, —CN, —NO₂, —Si(R^{8a})₃, —OR^{9a}, —OC(O)—R^{9b}, —N(R^{9c})R^{9d}, —C(O)R^{9e}, —C(O)OR^{9f}, —C(O)N(R^{9g})R^{9h}, —N(R⁹ⁱ)C(O)OR^{8b}, —N(R^{9j}) C(O)R^{8c}, —N(R^{9k})S(O),R^{8d}, —S(O)ₜOR^{8e}, —S(O)ₚR^{8f}, —S(O)ₜN(R^{9m})R⁹ⁿ, —N(R^{9p})C(O)N(R^{9q})R^{9r}, —N(R^{9s}) S(O)ₜOR^{8g}, —OC(O)N(R^{9t})R^{9u} and/or —OS(O)ₜR^{8h};
R^{8a}, R^{8b}, R^{8d}, R^{8f}, R^{8g} and R^{8h} independently represent C₁₋₁₂ alkyl optionally substituted by one or more substituents selected from =0 and E¹;
R^{8c}, R^{8e}, R^{9a}, R^{9b}, R^{9c}, R^{9d}, R^{9e}, R^{9f}, R^{9g}, R^{9h}, R⁹ⁱ, R^{9j}, R^{9k}, R^{9m}, R⁹ⁿ, R^{9p}, R^{9q}, R^{9r}, R^{9s}, R^{9t} and R^{9u} independently represent hydrogen or C₁₋₁₂ alkyl optionally substituted by one or more substituents selected from =0 and E²; or any pair of R^{9c} and R^{9d}, R^{9g} and R^{9h}, R^{9m} and R⁹ⁿ, R^{9q} and R^{9r}, and R^{9t} and R^{9u} may be linked together with the nitrogen atom to which they are attached to form a 3- to 8-membered ring, optionally containing one or more unsaturations, optionally containing one or two further heteroatoms, and which ring is optionally substituted by one or more substituents selected from =0, halo and C₁₋₆ alkyl optionally substituted by one or more halo atoms;
B⁶ represents C₁₋₁₂ alkyl, heterocycloalkyl (which latter two groups are optionally substituted by one or substituents selected from =0 and E³), aryl or heteroaryl (which latter two groups are optionally substituted by one or substituents selected from E⁴);
t represents, at each occurrence when used herein, 1 or 2;
p represents 0, 1 or 2;
E¹, E², E³ and E⁴ independently represent halo, —CN, —NO₂, —OR^{10a}, —OC(O)—R^{10b}, —N(R^{10c}) R^{10d}, —C(O)R^{10e}, —C(O)OR^{10f}, —C(O)N(R^{10g})R^{10h}, —N(R¹⁰ⁱ)C(O)OR^{11a}, —N(R^{10j})C(O)R^{11b}, —N(R^{10k})S(O)ₜ₁R^{11c}, —S(O)ₜ₁OR^{11d}, —S(O)ₚ₁R^{11e}, —S(O)ₜ₁N(R^{10m})R¹⁰ⁿ, —N(R^{10p})C(O)N(R^{10q})R^{10r}, —N(R^{10s})S(O)ₜ₁OR^{11f}, —OC(O)N(R^{10t})R^{10u}, —OS(O)ₙR^{11g} and/or —Si(R^{11h})₃;
R^{10a}, R^{10b}, R^{10c}, R^{10d}, R^{10e}, R^{10f}, R^{10g}, R^{10h}, R¹⁰ⁱ, R^{10j}, R^{10k}, R^{10m}, R¹⁰ⁿ, R^{10p}, R^{10q}, R^{10r}, R^{10s}, R^{10t}, R^{10u}, R^{11b} and R^{11b} independently represent hydrogen or C₁₋₃ alkyl optionally substituted by one or more halo atoms;
R^{11a}, R^{11c}, R^{11e}, R^{11f}, R^{11g} and R^{11h} independently represent C₁₋₃ alkyl optionally substituted by one or more halo atoms;
t1 represents, at each occurrence when used herein, 1 or 2;
p1 represents 0, 1 or 2,
or a pharmaceutically acceptable salt thereof.

In another embodiment, a PDE7 inhibitor useful in the methods of the invention is selected from the above formula (60) derivatives, compound or pharmaceutically acceptable salt thereof,
wherein R¹ represents -A¹-T²-B¹,
wherein R¹ represents -A¹-T²-B¹ and/or R³ represents —OR^{4a},
wherein additionally R⁴ and R^{4a} independently represent C₁₋₁₂ alkyl (optionally substituted by one or more substituents selected from =0 and X²) or —B³,
wherein additionally each R⁵ represents C₁₋₆ alkyl (optionally substituted by one or more substituents selected from =0 and X³) or hydrogen,
wherein additionally each R⁶ and each R⁷ independently represent X⁴ or C₁₋₆ alkyl (optionally substituted by one or more substituents selected from =0 and X⁵),
wherein additionally A¹ represents unsubstituted C₁₋₆ alkylene,
wherein additionally R^{w1} and R^{w2} independently represent hydrogen,
wherein additionally B¹ represents a 5-membered heteroaryl group or a bicyclic heteroaryl group optionally substituted with one or more substituents selected from X⁸,
wherein additionally B², B³, B^{3a} independently represent phenyl (optionally substituted by one or more substituents selected from X⁹), a 5- or 6-membered heterocycloalkyl group (optionally substituted by one or more substituents selected from =0 and X¹⁰) or a 5- or 6-membered heteroaryl group (optionally substituted by one or more substituents selected from X¹¹),
wherein additionally B⁴ and B⁵ independently represent a 5- or 6-membered heterocycloalkyl group (optionally substituted by one or more substituents selected from =0 and X¹²),
and wherein additionally X¹, X², X³, X⁴, X⁵, X⁶, X⁷, X⁸, X⁹, X¹⁰, X¹¹ and X¹² independently represent B⁶, —C(O)OR^{9f}, —S(O)ₜN(R^{9m})R⁹ⁿ, —N(R^{9k})S(O)ₜR^{8d}, —CN, —NO₂, halo, —OR^{9a}, —N(R^{9c}) R^{9d}, —C(O)N(R^{9g})R^{9h} and/or —N(R^{9j})C(O)R^{8c}; R^{8a}, R^{8b}, R^{8d}, R^{8e}, R^{8f}, R^{8g} and R^{8h} independently represent C₁₋₆ alkyl optionally substituted by one or more substituents selected from E¹; R^{8c}, R^{9a}, R^{9b}, R^{9c}, R^{9d}, R^{9e}, R^{9f}, R^{9g}, R^{9h}, R⁹ⁱ, R^{9j}, R^{9k}, R^{9m}, R⁹ⁿ, R^{9p}, R^{9q}, R^{9r}, R^{9s}, R^{9t} and R^{9u} independently represent hydrogen or C₁₋₆ alkyl optionally substituted by one or more substituents selected from E²; B^{s} represents C₃₋₈ alkyl, 5- or 6-membered heterocycloalkyl (both of which are optionally substituted by one or more E³ substituents), heteroaryl or, aryl, which latter two groups are optionally substituted by one or more E⁴ substituents; E¹, E², E³ and E⁴ independently represent-N(R^{10k})S(O)ₜiR^{11c}, —S(O)ₜ₁N(R^{10m})R¹⁰ⁿ, —NO₂, —C(O)OR^{10f}, halo, —CN, —OR^{10a}, —N(R^{10c})R^{10d}, —C(O)N(R^{10g})R^{10h} and/or —N(R^{10j}) C(O)R^{11b}; R^{10a}, R^{10b}, R^{10c}, R^{10d}, R^{10e}, R^{10f}, R^{10g}, R^{10h}, R¹⁰ⁱ, R^{10j}, R^{10k}, R^{10m}, R¹⁰ⁿ, R^{10p}, R^{10q}, R^{10r}, R^{10s}, R^{10t}, R^{10u} and R^{11b} independently represent hydrogen, —CH₃ or —CF₃; and/or R^{11a}, R^{11c}, R^{11d}, R^{11e}, R^{11f}, R^{11g} and R^{11h} independently represent-CH₃ or —CF₃. The preparation of the above compounds is described in WO 2010/029299.

In another embodiment, PDE7 inhibitors useful in the methods of the invention are selected from those compounds generally or specifically disclosed in WO 2011/114103 each expressly incorporated by reference herein in its entirety.

In one embodiment, PDE7 inhibitors useful in the methods of the invention have the formula (61) or pharmaceutically acceptable salt thereof,

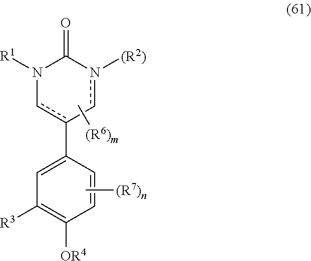

wherein
at least one of the dotted lines represents a bond (thereby forming a double bond), and the other represents an optional bond (and therefore denotes the presence of an optional double bond); when the dotted line between the carbon and nitrogen is present, then R² is absent, and when the dotted line between the carbon and nitrogen is absent, then R² is present;
m represents 0, 1, 2, 3 or 4;
n represents 0, 1, 2 or 3;
at least one of R¹ and, if present, R² represents -A¹-T²-B¹ and the other (if present) represents R⁵;
R³ represents hydrogen, —OR^{4a}, C₁₋₁₂ alkyl (optionally substituted by one or more substituents selected from ═O and X¹) or —B²;
R⁴ and R⁴³ independently represent hydrogen, C₁₋₁₂ alkyl (optionally substituted by one or more substituents selected from ═O and X²) or —B³;
R⁵ represents hydrogen, C₁₋₁₂ alkyl (optionally substituted by one or more substituents selected from ═O and X³) or —B^{3a};
each R⁶ and each R⁷ independently represent X⁴, C₁₋₁₂ alkyl (optionally substituted by one or more substituents selected from ═O and X⁵) or —B⁴; or
any two R⁶ groups may be linked together to form a further ring, which is formed either by the two relevant groups being linked together by a direct bond or C₁₋₅ alkylene;
A¹ represents C₁₋₁₂ alkylene (optionally substituted by one or more substituents selected from ═O and X⁶);
T² represents a direct bond, —N(R^{w1})— or —O(O)N(R^{w2})—;
R^{w1} and R^{w2} independently represent hydrogen, C₁₋₁₂ alkyl (optionally substituted by one or more substituents selected from X⁷) or —B⁵;
B¹ represents:
1) phenyl; or
2) a monocyclic 6-membered heteroaryl group,
both of which are optionally and independently substituted with one or more substituents selected from X⁸;
B², B³ and B^{3a} independently represent aryl (optionally substituted by one or more substituents selected from X⁹), heterocycloalkyi (optionally substituted by one or more substituents selected from ═O and X¹⁰) or heteroaryl (optionally substituted by one or more substituents selected from X¹¹); B⁴ and B⁵ independently represent heterocycloalkyi (optionally substituted by one or more substituents selected from ═O and X¹²);
X¹, X², X³, X⁴, X⁵, X⁶, X⁷, X⁸, X⁹, X¹⁰X¹¹ and X¹² independently represent B⁶, halo, —CN, —NO₂, —Si(R^{8a})₃, —OR^{9a}, —OC(O)—R^{9b}, —N(R^{9c})R^{9d}, —C(O)R^{9e}, —C(O)OR^{9f}, —C(O)N(R^{9g})R^{9h}, —N(R⁹ⁱ) C(O)OR^{8b}, —N(R^{9j}) C(O)R^{8c}, —N(R^{9k})S(O)ₜR^{8d}, —S(O)ₜOR^{8e}, —S(O)ₚR^{8f}, —S(O)ₜN(R^{9m})R⁹ⁿ, —N(R^{9p})C(O)N(R^{9q})R^{9r}, —N(R^{9s})S(O)ₜOR^{8g}, —OC(O)N(R^{9t})R^{9u} and/or —OS(O)ₜR^{8h};
R^{8a}, R^{8b}, R^{8d}, R^{8g} and R^{8h} independently represent C₁₋₁₂ alkyl optionally substituted by one or more substituents selected from ═O and E¹;
R^{8c}, R^{8e}, R^{8f}, R^{9a}, R^{9b}, R^{9c}, R^{9d}, R^{9e}, R⁹¹, R^{9g}, R^{9h}, R⁹ⁱ, R^{9j}, R^{9k}, R^{9m}, R⁹ⁿ, R^{9p}, R^{9q}, R^{9r}, R^{9s}, R^{9t} and R^{9u} independently represent hydrogen or C₁₋₁₂ alkyl optionally substituted by one or more substituents selected from ═O and E²; or
any pair of R^{9c} and R^{9d}, R^{9g} and R^{9h}, R^{9m} and R⁹ⁿ, R^{9q} and R^{9r}, and R^{9t} and R^{9u} may be linked together with the nitrogen atom to which they are attached to form a 3- to 8-membered ring, optionally containing one or more unsaturations, optionally containing one or two further heteroatoms, and which ring is optionally substituted by one or more substituents selected from ═O, halo and C₁₋₆ alkyl optionally substituted by one or more halo atoms;
B⁶ represents C₁₋₁₂ alkyl, heterocycloalkyl (which latter two groups are optionally substituted by one or substituents selected from ═O and E³), aryl or heteroaryl (which latter two groups are optionally substituted by one or substituents selected from E⁴);
t represents, at each occurrence when used herein, 1 or 2;
p represents 0, 1 or 2;
E¹, E², E³ and E⁴ independently represent halo, —CN, —NO₂, —OR^{10a}, —OC(O)—R^{10b}, —N(R^{10c})R^{10d}, —C(O)R^{10e}, —C(O)OR^{10f}, —C(O)N(R^{10g})R^{10h}, —N(R¹⁰ⁱ)C(O)OR^{11a}, —N(R^{10j})C(O)R^{11b}, —N(R^{10k})S(O)ₜ₁R^{11c}, —S(O)ₜ₁OR^{11d}, —S(O)ₚ₁R^{11e}, —S(O)ₜ₁N(R^{10m})R¹⁰ⁿ, —N(R^{10p})C(O)N(R^{10q})R^{10r}, —N(R^{10s})S(O)ₜ₁OR^{11f}, —OC(O)N(R¹⁰)R^{10u}, —OS(O)ₜ₁R^{11g} and/or —Si(R^{11h})₃; R^{10a}, R^{10b}, R^{10c}, R^{10d}, R^{10e}, R^{10f}, R^{10g}, R^{10h}, R¹⁰ⁱ, R^{10j}, R^{10k}, R^{10m}, R¹⁰ⁿ, R^{10p}, R^{10q}, R^{10r}, R^{10s}, R^{10t}, R^{10u}, R^{10b}, R^{10d} an d R^{11e} independently represent hydrogen or C₁₋₃ alkyl optionally substituted by one or more halo atoms;
R^{11a}, R^{11c}, R^{11f}, R^{11g} and R^{11h} independently represent C₁₋₃ alkyl optionally substituted by one or more halo atoms;
t1 represents, at each occurrence when used herein, 1 or 2;
p1 represents 0, 1 or 2,
or a pharmaceutically acceptable salt thereof.

In another embodiment, a PDE7 inhibitor useful in the methods of the invention is selected from the above formula 61 derivatives, compound or pharmaceutically acceptable salt thereof,
wherein R¹ represents -A¹-T²-B¹,
wherein R¹ represents -A¹-T²-B¹ and/or R³ represents —OR^{4a},
wherein additionally R⁴ and R^{4a} independently represent C₁₋₁₂ alkyl (optionally substituted by one or more substituents selected from ═O and X²) or —B³,
wherein additionally each R⁵ represents C₁₋₆ alkyl (optionally substituted by one or more substituents selected from ═O and X³) or hydrogen,
wherein additionally each R⁶ and each R⁷ independently represent X⁴ or C₁₋₆ alkyl (optionally substituted by one or more substituents selected from ═O and X⁵),
wherein additionally A¹ represents unsubstituted C₁₋₆ alkylene,
wherein additionally R^{w1} and R^{w2} independently represent hydrogen,
wherein additionally B¹ represents phenyl optionally substituted with one or more substituents selected from X⁸,
wherein additionally B², B³, B^{3a} independently represent phenyl (optionally substituted by one or more substituents selected from X⁹), a 5- or 6-membered heterocycloalkyi group (optionally substituted by one or more substituents selected from ═O and X¹⁰) or a 5- or 6-membered heteroaryl group (optionally substituted by one or more substituents selected from X¹¹),
wherein additionally B⁴ and B⁵ independently represent a 5- or 6-membered heterocycloalkyi group (optionally substituted by one or more substituents selected from ═O and X¹²),
wherein additionally X¹, X², X³, X⁴, X⁵, X⁶, X⁷, X⁸, X⁹, X¹⁰, X¹¹ and X¹² independently represent B⁶, —C(O)OR_{9f}, —S(O)ₜN(R^{9m})R⁹ⁿ, —N(R^{9k})S(O)ₜR^{8d}, —CN, —NO₂, halo, —OR^{9a}, —N(R^{9c})R^{9d}, —C(O)N(R^{9g})R^{9h} and/or —N(R^{9j})C(O)R^{8c}; R^{8a}, R^{8b}, R^{8d}, R^{8e}, R^{8f}, R^{8g} and R^{8h} independently represent C₁₋₆ alkyl optionally substituted by one or more substituents selected from E¹; R^{8c}C, R^{9a}, R^{9b}, R^{9c}, R^{9d}, R^{9e}, R^{9f}, R^{9g}, R^{9h}, R⁹ⁱ, R^{9j}, R^{9k}, R^{9m}, R⁹ⁿ, R^{9p}, R^{9q}, R^{9r}, R^{9s}, R^{9t} and R^{9u} independently represent hydrogen or C₁₋₆ alkyl optionally substituted by one or more substituents selected from E²; B⁶ represents C₃₋₈ alkyl, 5- or 6-membered heterocycloalkyl (both of which are optionally substituted by one or more E³ substituents), heteroaryl or, aryl, which latter two groups are optionally substituted by one or more E⁴ substituents; E¹, E², E³ and E⁴ independently represent-N(R^{10k})S(O)ₜ₁R^{11c}, —S(O)ₜ₁N(R^{10m}) R¹⁰ⁿ, —NO₂, —C(O)OR_{10f}, halo, —CN, —OR₁₀ₐ, —N(R^{10c}) R^{10d}, —C(O)N(R^{10g}) R^{10h} and/or —N(R^{10j}) C(O)R^{11b}; R^{10a}, R^{10b}, R^{10c}, R^{10d}, R^{10e}, R^{10f}, R^{10g}, R^{10h}, R¹⁰ⁱ, R^{10j}, R^{10k}, R^{10m}, R¹⁰ⁿ, R^{10p}, R^{10q}, R^{10r}, R^{10s}, R^{10t}, R^{10u} and R^{11b} independently represent hydrogen, —CH₃ or —CF₃; and/or R^{11a}, R^{11c}, R^{11d}, R^{11e}, R^{11f}, R^{11g} and R^{11h} independently represent-CH₃ or —CF₃.

The preparation of the above compounds is described in WO 2011/114103.

In another embodiment, PDE7 inhibitors useful in the methods of the invention are selected from those compounds generally or specifically disclosed in WO 2010/076564 each expressly incorporated by reference herein in its entirety.

In one embodiment, PDE7 inhibitors useful in the methods of the invention have the formula (62) or pharmaceutically acceptable salt thereof:

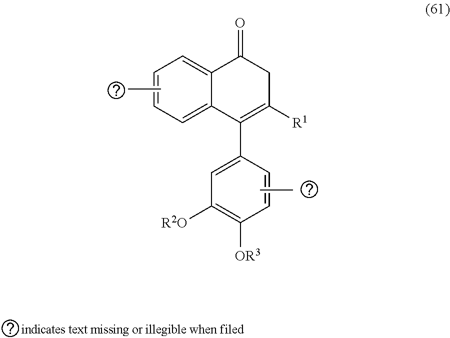

((62): Also referred to as isochromenone derivatives.)
Wherein
n represents 0, 1, 2, 3 or 4; m represents 0, 1, 2 or 3;
R¹ represents hydrogen, —C(OR^{4a})(R^{4b})₂—C(R^{4b})₃ or —Si(R^{6p})₃;
R² and R³ independently represent hydrogen, C₁₋₁₂ alkyl (optionally substituted by one or more substituents selected from A¹), aryl (optionally substituted by one or more substituents selected from A²), heteroaryl (optionally substituted by one or more substituents selected from A³), heterocycloalkyl (optionally substituted by one or more substituents selected from A⁴), —C(O)OR^{5a}, —C(O)N(R^{5b})R^{5c}, —S(O)₂R^{5d} or —C(O)R^{5e};
each R^{4a} and R^{4b} independently represent, on each occasion when used herein, hydrogen or C₁₋₁₂ alkyl optionally substituted by one or more substituents selected from A⁵; or any two R^{4b} substituents when attached to a common carbon atom may be linked together to form a 3- to 12-membered ring, optionally containing one to three heteroatoms, one to three unsaturations and which ring is optionally substituted with one or more substituents selected from A⁶;
each R^{x} and R^{y} independently represents, on each occasion when used herein, A⁷ and/or C₁₋₁₂ alkyl optionally substituted by one or more substituents selected from A⁸;
A¹, A², A³, A⁴, A⁵, A⁶, A⁷ and A⁸ independently represent aryl (optionally substituted by one or more substituents selected from B¹), heteroaryl (optionally substituted by one or more substituents selected from B²) heterocycloalkyl (optionally substituted by one or more substituents selected from B³), halo, —CN₁—NO₂, —C(O)OR^{6a}, —C(O)N(R^{7a})R^{8a}, —O—R^{6b}, —OC(O)R^{6c}, —N(R^{7b})R^{8b}, —C(O)R^{6d}, —OS(O)₂R^{6e}, —OC(O)N(R^{7c})R^{8c}, —N(R^{6f})C(O)OR^{6g}, —N(R^{6h})C(O)R⁶ⁱ, —N(R⁶ⁱ) S(O),R^{6k}, —S(O),OR^{6m}, —S(O)ₚR⁶ⁿ, —S(O)ₜN(R^{7d})R^{8d}, —Si(R^{6p})₃, —N(R^{6q})C(O)N(R^{7e})R^{8e} and/or —OC(O)OR^{6r}; and/or A¹, A⁴, A⁵, A⁶ and A⁸ may alternatively and independently represent=0;
t represents, on each occasion when used herein, 1 or 2;
p represents, on each occasion when used herein, 0, 1 or 2;
R^{5b}, R^{5c}, R^{5e}, R^{6a}, R^{6b}, R^{6d}, R^{6f}, R^{6h}, R⁶ⁱ, R^{6j}, R^{6m}, R⁶ⁿ, R^{6q}, R^{7a}, R^{7b}, R^{7c}, R^{7d}, R^{7e}, R^{8a}, R^{8b}, R^{8c}, R^{8d} and R^{8e} independently represent, on each occasion when used herein, hydrogen, C₁₋₁₂ alkyl (optionally substituted by one or more substituents selected from B⁴), aryl (optionally substituted by one or more substituents selected from B⁵), heteroaryl (optionally substituted by one or more substituents selected from B⁶) or heterocycloalkyl (optionally substituted by one or more substituents selected from B⁷); or any pair of R^{5b} and R^{5c}, R^{7a} and R^{8a}, R^{7b} and R^{8b}, R^{7c} and R^{8c}, R^{7d} and R^{8d} and R^{7e} and R^{8e} may be linked together to form, together with the nitrogen atom to which they are necessarily attached, a 3- to 12-membered ring, optionally containing a further one or two heteroatoms, one or two unsaturations, and which ring is optionally substituted by one or more substituents selected from B⁸;
R^{5a}, R^{5d}, R^{6c}, R^{6e}, R^{6g}, R^{6k}, R^{6p} and R^{6r} independently represent, on each occasion when used herein, C₁₋₁₂ alkyl (optionally substituted by one or more substituents selected from B⁴), aryl (optionally substituted by one or more substituents selected from B⁵), heteroaryl (optionally substituted by one or more substituents selected from B⁶) or heterocycloalkyl (optionally substituted by one or more substituents selected from B⁷);
B¹, B², B³, B⁴, B⁵, B⁶, B⁷ and B⁸ independently represent halo, C₁₋₆ alkyl (optionally substituted by one or more halo atoms), —OR^{9a}, —N(R^{9b})R^{10b}, —NO₂, —CN, aryl (optionally substituted by one or more substituents selected from E¹), heteroaryl (optionally substituted by one or more substituents selected from E²) and/or heterocycloalkyl (optionally substituted by one or more substituents selected from E³); and/or B³, B⁴, B⁷ and B⁸ may alternatively and independently represent=0;
R^{9a}, R^{9b} and R^{10b} independently represent hydrogen or C₁₋₆ alkyl optionally substituted by one or substituents selected from E⁴;
E¹, E², E³ and E⁴ independently represent halo, —CN, —OH, —OC₁₋₆ alkyl (optionally substituted by one or more fluoro atoms) and/or C₁₋₆ alkyl (optionally substituted by one or more halo atoms), or a pharmaceutically acceptable salt thereof.

In another embodiment, a PDE7 inhibitor useful in the methods of the invention is selected from the above formula 62 derivatives, compound or pharmaceutically acceptable salt thereof,
wherein R¹ represents hydrogen or —C(OR^{4a}XR^{4b}),
wherein R¹ represents R¹ represents hydrogen or —C(OR^{4a}XR^{4b}), and/or R² represents hydrogen or C₁₋₁₂ alkyl (optionally substituted by one or more substituents selected from A¹),
wherein additionally R^{4a} represents C₁₋₃ alkyl or hydrogen,
wherein additionally each R^{4b} independently represents hydrogen or C₁₋₆ alkyl optionally substituted by one or more substituents selected from A⁵; or two R^{4b} groups when attached to a common carbon atom are linked together to form a 3- to 8-membered ring, optionally containing one heteroatom, which ring may be substituted by one or more A⁶ substituents,
wherein additionally R³ represents hydrogen, C₁₋₁₂ alkyl (optionally substituted by one or more substituents selected from A¹) or —S(O)₂R^{5d},
wherein additionally A¹ to A⁸ independently represent aryl (optionally substituted by one or more B¹ substituents), heteroaryl (optionally substituted by one or more substituents selected from B²), heterocycloalkyl (optionally substituted by one or more substituents selected from B³), halo, —CN, —NO₂, —C(O)OR^{6a}, —O—R^{6b}, —N(R^{7b}) R^{8b} or —C(O)N(R^{7a})R^{8a},
wherein additionally R^{5b}, R^{5c}, R^{6a}, R^{6b}, R^{6d}, R^{6f}, R^{6h}, R⁶ⁱ, R^{6j}, R^{6m}, R⁶ⁿ, R^{6q}, R^{7a}, R^{7b}, R^{7c}, R^{7d}, R^{7e}, R^{8a}, R^{8b}, R^{8c}, R^{8d} and R^{8e} independently represent hydrogen, C₁₋₆ alkyl (optionally substituted by one or more substituents selected from B⁴) or aryl (optionally substituted by one or more substituents selected from B⁵); or any pair of R^{5b} and R^{5c}, R^{7a} and R^{8a}, R^{7b} and R^{8b}, R^{7c} and R^{8c}, R^{7d} and R^{8d} and R^{7e} and R^{8e} may be linked together to form, together with the nitrogen atom to which they are necessarily attached, a 3- to 8-membered ring, optionally containing a further heteroatom, which ring is optionally substituted by one or two substituent(s) selected from B⁸,
wherein additionally R^{5a}, R^{5d}, R^{6c}, R^{6e}, R^{6g}, R^{6k} and R^{6r} independently represent C₁₋₆ alkyl (optionally substituted by one or more substituents selected from B⁴) or aryl (optionally substituted by one or more substituents selected from B⁵),
wherein additionally B¹, B², B³, B⁴, B⁵, B⁶, B⁷ and B⁸ independently represent halo, C₁₋₆ alkyl (optionally substituted by one or more halo atoms) and/or aryl (optionally substituted by one or more substituents selected from E¹); and/or B³, B⁴, B⁷ and B⁸ may alternatively and independently represent=0,
wherein additionally R^{9a}, R^{9b} and R^{10b} independently represent hydrogen or C₁₋₃ (e.g. C₁₋₂) alkyl optionally substituted by one or substituents selected from E⁴,
wherein additionally E¹, E², E³ and E⁴ independently represent halo or C₁₋₂ alkyl.

The preparation of the above compounds is described in WO 2010/076564.

In another embodiment, PDE7 inhibitors useful in the methods of the invention are selected from those compounds generally or specifically disclosed in WO 2011/039403, U.S. Pat. No. 9,604,947, and EP-A-2484670 each expressly incorporated by reference herein in its entirety.

In one embodiment, PDE7 inhibitors useful in the methods of the invention have the formula (63):

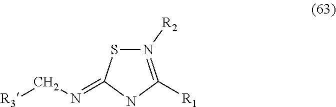

((63): Also referred to as 5-imino-1,2,4-thiadiazole derivatives.)
Wherein
R₁ is a group X₁—R₁′ wherein X₁ is a single bond;
R₁′ is C₁-C₆ alkyl, aryl, R₁′ being optionally substituted with one or more groups X₁′—R₈ which may be identical or different;
X₁′ is a single bond or a group selected from C₁-C₆ alkylene, X₁′ being optionally substituted with at least one or more groups which may be identical or different and are selected from F, Cl, Br, I;
R₈ is H, —OH, ═O, —NO₂, CN, F, Cl, Br, I, C₁-C₄ alkyl, —CO₂R₆ₐ, —C(═O)R₆ₐ, C(═S)R₆ₐ, SO₂R₆ₐ, SOR₆ₐ, SO₃R₆ₐ, SR₆ₐ, OR₆ₐ, C(═O)NR₆ₐ, R₇ₐ, C(═S)NR₆ₐR₇ₐ, C(═N—CN)NR₆ₐR₇ₐ, C(═N—SO₂NH₂)NR₆ₐR₇ₐ, C(═CH—NO₂)NR₆ₐR₇ₐ, SO₂NR₆ₐR₇ₐ, C(═NR₆ₐ) NHR₇ₐ, C(═NR₆ₐ)R₇ₐ or NR₆ₐR₇ₐ, R₆ₐ and R₇ₐ being independently selected from R₄ and R₅;
R₂ is aryl, R₂ being optionally substituted with at least one or more groups which may be identical or different and are selected from —NO₂, OR_{6b}, R_{6b} being independently selected from R₄ and R₅;
R₃′ is selected from pyridyl, —(CH₂)ₙ—(C₅-C₇-heterocycloalkyl), (CH₂)ₙ—OR₁₂ or —(CH₂)ₙ—C(O)OR₁₂, n is a number between 0 and 20;
R₁₂ is independently selected from the groups defined for R₁₀;
R₄ and R₅ are independently selected from: C₁-C₆ alkyl; and
R₁₀ is independently selected from H and C₁-C₆ alkyl;
or a pharmaceutically acceptable salt thereof. Such salt can be selected from the group consisting of hydrobromide and hydrochloride salts thereof.

In another embodiment, a PDE7 inhibitor useful in the methods of the invention is selected from the compounds derived from formula 63 wherein
R₁ is phenyl or naphthyl each of which is optionally substituted by one or more substituents independently selected from the group consisting of F, Cl, Br, I, OH, CN, CF₃, NO₂, C₁₋₆-alkyl and C₁₋₆-alkoxy;
R₂ is phenyl or naphthyl each of which is optionally substituted by one or more substituents independently selected from the group consisting of F, Cl, Br, I, OH, CN, CF₃, NO₂, C₁₋₆-alkyl and C₁₋₆-alkoxy;
R₃′ is selected from pyridyl, —(CH₂)ₙ—(C₅-C₇-heterocycloalkyl), (CH₂)ₙ—(C₃-C₁₀ heterocycloalkyl), —(CH₂)ₙ—OR₁₂ or —(CH₂)ₙ—C(O)OR₁₂;
n is 0-6; and
R₁₂ is H or C₁₋₆-alkyl;
or a pharmaceutically acceptable salt thereof.

In more specific embodiments the compounds are selected from:
2,3-Diphenyl-5-(3-pyridylmethylimino)-2,5-dihydro-1,2,4-thiadiazole;
3-(4-Methoxyphenyl)-2-phenyl-5-(3-pyridylmethylimino)-2,5-dihydro-1,2,4-thiadiazole;
2-(4-Methoxyphenyl)-3-phenyl-5-(3-pyridylmethylimino)-2,5-dihydro-1,2,4-thiadiazole;
2-(4-Nitrophenyl)-3-phenyl-5-(3-pyridylmethylimino)-2,5-dihydro-1,2,4-thiadiazole;
2-Phenyl-5-(3-pyridylmethylimino)-3-(4-trifluoromethylphenyl)-2,5-dihydro-1,2,4-thiadiazole;
2-(1-Naphthyl)-3-phenyl-5-(3-pyridylmethylimino)-2,5-dihydro-1,2,4-thiadiazole;
3-(1-Naphthyl)-2-phenyl-5-(3-pyridylmethylimino)-2,5-dihydro-1,2,4-thiadiazole;
3-Methyl-2-phenyl-5-(3-pyridylmethylimino)-2,5-dihydro-1,2,4-thiadiazole;
5-Ethoxycarbonylmethylimino-2,3-diphenyl-2,5-dihydro-1,2,4-thiadiazole;
5-Ethoxycarbonylmethylimino-3-(4-methoxyphenyl)-2-phenyl-2,5-dihydro-1,2,4-thiadiazole;
5-Ethoxycarbonylmethylimino-2-(4-methoxyphenyl)-3-phenyl-2,5-dihydro-1,2,4-thiadiazole;
5-Ethoxycarboxymethylimino-2-(4-nitrophenyl)-3-phenyl-2,5-dihydro-1,2,4-thiadiazole;
5-(2-Hydroxyethylimino)-2,3-diphenyl-2,5-dihydro-1,2,4-thiadiazole;
5-(2-Hydroxyethylimino)-3-(4-methoxyphenyl)-2-phenyl-2,5-dihydro-1,2,4-thiadiazole;
5-(2-Hydroxyethylimino)-2-(4-methoxyphenyl)-3-phenyl-2,5-dihydro-1,2,4-thiadiazole;
5-(2-Hydroxyethylimino)-2-(1-naphthyl)-3-phenyl-2,5-dihydro-1,2,4-thiadiazole;
5-(2-Hydroxyethylimino)-3-(1-naphthyl)-2-phenyl-2,5-dihydro-1,2,4-thiadiazole; and
5-(2-Morpholinethylimino)-2,3-diphenyl-2,5-dihydro-1,2,4-thiadiazole.

In more specific embodiments, a pharmaceutical composition comprising a pharmaceutically acceptable salt selected from the group consisting of hydrobromide and hydrochloride salts of at least one of the specific compounds listed above as an active ingredient, and a pharmaceutically acceptable carrier.

In a more specific embodiment the substance is:

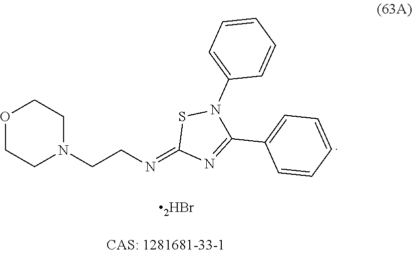 [SMILES: c1ccc(-c2n/c(=N/CCN3CCOCC3)sn2-c2ccccc2)cc1]

The preparation of the above compounds is described in WO 2011/039403, U.S. Pat. No. 9,604,947, and EP-A-2484670.

In another embodiment, PDE7 inhibitors useful in the methods of the invention are selected from those compounds generally or specifically disclosed in WO 2010/046791 each expressly incorporated by reference herein in its entirety.

In one embodiment, PDE7 inhibitors useful in the methods of the invention have the formula (64) and its pharmaceutically acceptable salts, pharmaceutically acceptable solvates, stereoisomers, tautomers, racemates, regioisomers, geometric isomers, prodrugs, metabolites, polymorphs and N-oxides:

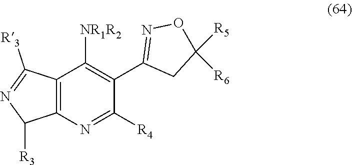

((64): Also referred to as 4,5-dihydroisoxazole-substituted pyrazolopyrimidine derivatives.) Wherein R₁ and R₂ independently are hydrogen, aryl, heteroaryl, —COR₇, —S(O)ₘR₇ (wherein R₇ is hydrogen, alkyl, cycloalkyl, aryl, aralkyl, heteroaryl or heterocyclyl and m is an integer from 0-2), or

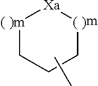

(wherein m is an integer from 0-2 and X is —O—, S(O)ₘ (wherein m is an integer from 0-2), NR₈ {wherein R₈ is hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ (wherein R₇ and R′₇ are hydrogen, alkyl, cycloalkyl, aryl, aralkyl, heteroaryl or heterocyclyl and m is the same as defined above)}, C(═O), C═NOH or CR_{f}R_{q} (wherein R_{f} and R_{q} independently are hydrogen, halogen, hydroxy, cyano, NR₈R′₈ [wherein R₈ and R′₈ are hydrogen, alkyl, alkenyl, alkynyl, cycloalkyl, cycloalkylalkyl, aryl, heteroaryl, heterocyclyl, —COR₇, —S(O)ₘR₇, —COOR₇ or —CONR₇R′₇ {wherein m, R₇ and R′₇ are the same as defined above}], —CONR₇R₇′, —COONR₇R₇′ or —COOR₇ (wherein R₇ and R′₇ are the same as defined above)), R₃ is alkyl, aryl, cycloalkyl, heterocyclyl or heteroaryl, R′₃ is hydrogen, alkyl, aryl, cycloalkyl, heteroaryl, heterocyclyl or (un) substituted amine, R₄ is alkyl, aryl, cycloalkyl, halogen, cyano, heteroaryl, heterocyclyl, or (un) substituted amine, R₅ and R₆ independently are alkyl, —CN, heterocyclyl, —(CH₂)ₘC(═O)NRⱼR′ⱼ {wherein m is an integer from 0-2 and Rⱼ and R′ⱼ independently are hydrogen, alkyl, alkenyl, cycloalkyl, aryl, aralkyl, heterocyclyl, heteroaryl, heterocyclylalkyl, heteroarylalkyl or Rⱼ and R′ⱼ taken together with the nitrogen atom to which they are attached can form a optionally substituted heterocyclyl ring}, —(CH₂)ₘC(═O)ORⱼ {wherein m and R₁ are the same as defined above}, —(CH₂)ₘ₁ORⱼ {wherein m1 is an integer from 0-3 and Rⱼ is the same as defined above} or —(CH₂)ₘC(═O) heterocyclyl {wherein m is the same as defined above}, or R₅ and R₆ together can form a 3-7 membered saturated, partially saturated or unsaturated ring containing carbon atoms wherein one or more carbon atoms optionally can be replaced by heteroatoms selected from O, S(O)ₘ {wherein m is an integer from 0-2} or NR₈ {wherein R₈ is the same as defined above}, or one or more carbon atoms optionally can be substituted with oxo, spiro-attached heterocyclyl, cyano, alkyl, heteroaryl, heteroarylalkyl, —(CH₂)ₘhalogen, —(CH₂)ₘNR₇R′₇, —(CH₂)ₘOR₇, —(CH₂)ₘCONR₇R′₇, —(CH₂)ₘNR₇COR₇ or —(CH₂)ₘCOOR₇ (wherein m, R₇ and R′₇ are the same as defined above).

The preparation of the above compounds is described in U.S. Pat. No. 8,242,126, and WO 2009/077680.

### Proteins, Polypeptides or Peptide Inhibitors:

In some embodiments, the PDE7 inhibitory agent comprises isolated PDE7 polypeptide or peptide inhibitors, including isolated natural peptide inhibitors and synthetic peptide inhibitors that inhibit PDE7 activity. As used herein, the term “isolated PDE7 polypeptide or peptide inhibitors” refers to polypeptides or peptides that inhibit PDE7 dependent cleavage of cAMP by binding to PDE7, competing with PDE7 for binding to a substrate, and/or directly interacting with PDE7 to inhibit PDE7-dependent cleavage of cAMP, that are substantially pure and are essentially free of other substances with which they may be found in nature to an extent practical and appropriate for their intended use.

Peptide inhibitors have been used successfully in vivo to interfere with protein-protein interactions and catalytic sites. For example, peptide inhibitors to adhesion molecules structurally related to LFA-1 have been approved for clinical use in coagulopathies. Short linear peptides (<30 amino acids) have been described that prevent or interfere with integrin-dependent adhesion. Longer peptides, ranging in length from 25 to 200 amino acid residues, have also been used successfully to block integrin-dependent adhesion. In general, longer peptide inhibitors have higher affinities and/or slower off-rates than short peptides and may therefore be more potent inhibitors. Cyclic peptide inhibitors have also been shown to be effective inhibitors of integrins in vivo for the treatment of human inflammatory disease. One method of producing cyclic peptides involves the synthesis of peptides in which the terminal amino acids of the peptide are cysteines, thereby allowing the peptide to exist in a cyclic form by disulfide bonding between the terminal amino acids, which has been shown to improve affinity and half-life in vivo for the treatment of hematopoietic neoplasms.

PDE7-binding proteins, antibodies, nanobodies or functionally related proteins or protein fragments or fusion proteins like single-chain variable fragments or DARPins (Designed Ankyrin Repeat Proteins) or lipocalins/anticalins or other such molecules, that enter cells or are modified/conjugated with other moieties so that they can enter cells, also inhibit PDE7.

These PDE7-bindung peptides or proteins can for example be made from the respective mRNA, or mRNA in a lipid nanoparticle formulation or plasmids.

### Synthetic PDE7 Peptide Inhibitors

PDE7 inhibitory peptides useful in the methods of the invention are exemplified by amino acid sequences that mimic the target regions important for PDE7 enzyme activity, such as the catalytic domain of PDE7. PDE7A and PDE7B have an identity of 70% in the catalytic domain. The catalytic domain of PDE7A1 is from amino acid residue 185 to 456. The catalytic domain of PDE7A2 is from amino acid residue 211 to 424. The catalytic domain of PDE7B is from amino acid residue 172 to 420. The inhibitory peptides useful in the practice of the methods of the invention range in size from about 5 amino acids to about 250 amino acids. One may also use molecular modeling and rational molecular design to generate and screen for peptides that mimic the molecular structure of the PDE7 catalytic regions and inhibit the enzyme activity of PDE7. The molecular structures used for modeling include the CDR regions of anti-PDE7 monoclonal antibodies. Methods for identifying peptides that bind to a particular target are well known in the art. For example, molecular imprinting may be used for the de novo construction of macromolecular structures such as peptides that bind to a particular molecule. See, for example, Shea, K. J., “Molecular Imprinting of Synthetic Network Polymers: The De Novo Synthesis of Macromolecular Binding and Catalytic Sites,” Trends in Polymer Science 2(5):166-173 (1994).

As an illustrative example, one method of preparing mimics of PDE7 binding peptides is as follows. Functional monomers of a binding region of an anti-PDE7 antibody that exhibits PDE7 inhibition (the template) are polymerized. The template is then removed, followed by polymerization of a second class of monomers in the void left by the template, to provide a new molecule that exhibits one or more desired properties that are similar to the template. In addition to preparing peptides in this manner, other PDE7 binding molecules that are PDE7 inhibitory agents, such as polysaccharides, nucleosides, drugs, nucleoproteins, lipoproteins, carbohydrates, glycoproteins, steroids, lipids, and other biologically active materials, can also be prepared. This method is useful for designing a wide variety of biological mimics that are more stable than their natural counterparts because they are typically prepared by free radical polymerization of functional monomers, resulting in a compound with a nonbiodegradable backbone.

The PDE7 inhibitory peptides can be prepared using techniques well known in the art, such as the solid-phase synthetic technique initially described by Merrifield in J. Amer. Chem. Soc. 85:2149-2154, 1963. Automated synthesis may be achieved, for example, using Applied Biosystems 431A Peptide Synthesizer (Foster City, Calif.) in accordance with the instructions provided by the manufacturer. Other techniques may be found, for example, in Bodanszky, M., et al., Peptide Synthesis, second edition, John Wiley & Sons, 1976, as well as in other reference works known to those skilled in the art. The peptides can also be prepared using standard genetic engineering techniques known to those skilled in the art.

A candidate PDE7 inhibitory peptide may be tested for the ability to function as a PDE7 inhibitory agent in one of several assays, including, for example, a PDE7 phosphodiesterase assay.

### Expression Inhibitors of PDE7, Nucleic Acids

In some embodiments of the methods of the invention, the PDE7 inhibitory agent is a PDE7 expression inhibitor capable of inhibiting PDE7-dependent cAMP cleavage (PDE7A, PDE7B, or both). In the practice of this embodiment of the invention, representative PDE7 expression inhibitors include PDE7 antisense nucleic acid molecules (such as antisense mRNA, antisense RNA, antisense DNA, or antisense oligonucleotides), PDE7 RNAi molecules, PDE7 ribozymes, and PDE7 DNAzymes. Anti-sense RNA and DNA molecules act to directly block the translation of PDE7 mRNA by hybridizing to PDE7 mRNA and preventing translation of PDE7 protein. An antisense nucleic acid molecule may be constructed in a number of different ways provided that it is capable of interfering with the expression of PDE7. For example, an antisense nucleic acid molecule can be constructed by inverting the coding region (or a portion thereof) of PDE7A1 cDNA, PDE7A2 cDNA, PDE7A3 cDNA, PDE7B1 cDNA, PDE7B2 cDNA or PDE7B3 cDNA relative to its normal orientation for transcription to allow for the transcription of its complement. Methods for designing and administering antisense oligonucleotides are well known in the art and are described, e.g., in Mautino et al., Hum Gene Ther 13:1027-37, 2002; and Pachori et al., Hypertension 39:969-75, 2002, each of which is hereby incorporated by reference.

The antisense nucleic acid molecule is usually substantially identical to at least a portion of the target gene or genes. The nucleic acid, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 8 or so nucleotides may be used as the antisense nucleic acid molecule, although a longer sequence is preferable. In the present invention, a representative example of a useful inhibitory agent of PDE7 is an antisense PDE7 nucleic acid molecule that is at least ninety percent identical to the complement of a portion of the PDE7A1 cDNA. Another representative example of a useful inhibitory agent of PDE7 is an antisense PDE7 nucleic acid molecule which is at least ninety percent identical to the complement of a portion of the PDE7A2 cDNA. Another representative example of a useful inhibitory agent of PDE7 is an antisense PDE7 nucleic acid molecule which is at least ninety percent identical to the complement of a portion of the PDE7A3 cDNA. Another representative example of a useful inhibitory agent of PDE7 is an antisense PDE7 nucleic acid molecule which is at least ninety percent identical to the complement of a portion of the PDE7B1 cDNA. Another representative example of a useful inhibitory agent of PDE7 is an antisense PDE7 nucleic acid molecule which is at least ninety percent identical to the complement of a portion of the PDE7B2 cDNA. Another representative example of a useful inhibitory agent of PDE7 is an antisense PDE7 nucleic acid molecule which is at least ninety percent identical to the complement of a portion of the PDE7B3 cDNA.

The targeting of antisense oligonucleotides to bind PDE7 mRNA is another mechanism that may be used to reduce the level of PDE7 protein synthesis. For example, U.S. Pat. No. 7,579,455 to Paolo or the synthesis of polygalacturonase and the muscarine type 2 acetylcholine receptor is inhibited by antisense oligonucleotides directed to their respective mRNA sequences (U.S. Pat. No. 5,739,119 to Galli, and U.S. Pat. No. 5,759,829 to Shewmaker). Furthermore, examples of antisense inhibition have been demonstrated with the nuclear protein cyclin, the multiple drug resistance gene (MDG1), ICAM-1, E-selectin, STK-1, striatal GABA, receptor and human EGF (see, e.g., U.S. Pat. No. 5,801,154 to Baracchini; U.S. Pat. No. 5,789,573 to Baker; U.S. Pat. No. 5,718,709 to Considine; and U.S. Pat. No. 5,610,288 to Rubenstein).

A system has been described that allows one of ordinary skill to determine which oligonucleotides are useful in the invention, which involves probing for suitable sites in the target mRNA using Rnase H cleavage as an indicator for accessibility of sequences within the transcripts. Scherr, M., et al., Nucleic Acids Res. 26:5079-5085, 1998; Lloyd, et al., Nucleic Acids Res. 29:3665-3673, 2001. A mixture of antisense oligonucleotides that are complementary to certain regions of the PDE7 transcript is added to cell extracts expressing PDE7 and hybridized in order to create an RNAseH vulnerable site. This method can be combined with computer-assisted sequence selection that can predict optimal sequence selection for antisense compositions based upon their relative ability to form dimers, hairpins, or other secondary structures that would reduce or prohibit specific binding to the target mRNA in a host cell. These secondary structure analysis and target site selection considerations may be performed using the OLIGO primer analysis software (Rychlik, I., 1997) and the BLASTN 2.0.5 algorithm software (Altschul, S. F., et al., Nucl. Acids Res. 25:3389-3402, 1997). The antisense compounds directed towards the target sequence preferably comprise from about 8 to about 50 nucleotides in length. Antisense oligonucleotides comprising from about 9 to about 35 or so nucleotides are particularly preferred. The inventors contemplate all oligonucleotide compositions in the range of 9 to 35 nucleotides (i.e., those of 9, 10, 11, 12, 13, 14, 15, 16, 17, 18, 19, 20, 21, 22, 23, 24, 25, 26, 27, 28, 29, 30, 31, 32, 33, 34, or 35 or so bases in length) are highly preferred for the practice of antisense oligonucleotide-based methods of the invention. Highly preferred target regions of the PDE7 mRNA are those that are at or near the AUG translation initiation codon, and those sequences that are substantially complementary to 5′ regions of the mRNA, e.g., between the 0 and +10 regions of the PDE7 gene nucleotide sequence.

The term “oligonucleotide” as used herein refers to an oligomer or polymer of ribonucleic acid (RNA) or deoxyribonucleic acid (DNA) or mimetics thereof. This term also covers those oligonucleobases composed of naturally occurring nucleotides, sugars and covalent internucleoside (backbone) linkages as well as oligonucleotides having non-naturally occurring modifications. These modifications allow one to introduce certain desirable properties that are not offered through naturally occurring oligonucleotides, such as reduced toxic properties, increased stability against nuclease degradation and enhanced cellular uptake. In illustrative embodiments, the antisense compounds of the invention differ from native DNA by the modification of the phosphodiester backbone to extend the life of the antisense oligonucleotide in which the phosphate substituents are replaced by phosphorothioates. Likewise, one or both ends of the oligonucleotide may be substituted by one or more acridine derivatives that intercalate between adjacent basepairs within a strand of nucleic acid.

Another alternative to antisense is the use of “RNA interference” (RNAi). Double-stranded RNAs (dsRNAs) can provoke gene silencing in mammals in vivo. The natural function of RNAi and co-suppression appears to be protection of the genome against invasion by mobile genetic elements such as retrotransposons and viruses that produce aberrant RNA or dsRNA in the host cell when they become active (see, e.g., Jensen, J., et al., Nat. Genet. 21:209-12, 1999). The double-stranded RNA molecule may be prepared by synthesizing two RNA strands capable of forming a double-stranded RNA molecule, each having a length from about 19 to 25 (e.g., 19-23 nucleotides). For example, a dsRNA molecule useful in the methods of the invention may comprise the RNA corresponding to a portion of at least one of human PDE7A or PDE7B and its complement. Preferably, at least one strand of RNA has a 3′ overhang from 1-5 nucleotides. The synthesized RNA strands are combined under conditions that form a double-stranded molecule. The RNA sequence may comprise at least an 8 nucleotide portion of of human PDE7A or PDE7B with a total length of 25 nucleotides or less. The design of siRNA sequences for a given target is within the ordinary skill of one in the art. Commercial services are available that design siRNA sequence and guarantee at least 70% knockdown of expression (Qiagen, Valencia, Calif.). Exemplary PDE7 shRNAs and siRNAs are commercially available from Sigma-Aldrich Company (product #SHDNA_-NM_002603; SASI_Hs01_00183420 to SASI_Hs01_00010490).

The dsRNA may be administered as a pharmaceutical composition and carried out by known methods, wherein a nucleic acid is introduced into a desired target cell. Commonly used gene transfer methods include calcium phosphate, DEAE-dextran, electroporation, microinjection and viral methods. Such methods are taught in Ausubel et al., Current Protocols in Molecular Biology, John Wiley & Sons, Inc., 1993.

### Ribozymes and DNAzymes

In some embodiments, a PDE7 inhibitory agent is a ribozyme or a DNAzyme that specifically cleaves the mRNA of a target PDE7, such as PDE7A, PDE7B or both, or a PDE7 itself. Ribozymes that target PDE7 may be utilized as PDE7 inhibitory agents to decrease the amount and/or biological activity of PDE7. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs. Ribozymes useful in the practice of the invention typically comprise a hybridizing region of at least about nine nucleotides, which is complementary in nucleotide sequence to at least part of the target PDE7 mRNA, and a catalytic region that is adapted to cleave the target PDE7 mRNA (see generally, European Patent No. 0 321 201; WO 88/04300; Haseloff, J., et al., Nature 334:585-591, 1988; Fedor, M. J., et al., Proc. Natl. Acad. Sci. USA 87:1668-1672, 1990; Cech, T. R., et al., Ann. Rev. Biochem. 55:599-629, 1986).

Ribozymes can either be targeted directly to cells in the form of RNA oligonucleotides incorporating ribozyme sequences, or introduced into the cell as an expression vector encoding the desired ribozymal RNA. Ribozymes may be used and applied in much the same way as described for antisense polynucleotides.

Anti-sense RNA and DNA, RNAi molecules, ribozymes, and DNAzymes useful in the methods of the invention may be prepared by any method known in the art for the synthesis of DNA and RNA molecules. These include techniques for chemically synthesizing oligodeoxyribonucleotides and oligoribonucleotides well known in the art, such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well known modifications of the DNA molecules may be introduced as a means of increasing stability and half-life. Useful modifications include, but are not limited to, the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Protein Degraders

Protein degradation technologies also decrease or inhibit PDE7 activity. Many technologies are in use via the proteasome or lysosome or other mechanisms like proteolysis targeting chimeras (PROTACs), which are bispecific molecules containing a (here PDE7) target protein binder and an ubiquitin ligase binder connected by a linker (PDEδ-Targeting PROTACs by Winzker, M., et al., Angew Chem Int Ed Engl 59(14):5595-5601, 2020), or other degradation technologies comprising SNIPERs, HaloPROTACs, HyTs, LYTACs, AUTACs, ATTECs, RIBOTECs, monomeric degraders, double-mechanism degrader, SARD, TF-PROTAC, dual-PROTAC, SERD, bispecific aptamer chimera, AbTAC, GlueTAC, AUTOTAC, CMA-based degrader, MADTAC, ATAC, molecular glue degraders (for example Li, H. et al., J Hematol Oncol 14(1):138, 2021), biodegraders, mRNA for the biodegrader protein, mRNA for the biodegrader protein in a lipid nanoparticle formulation (for example Chang et al., Cell Chem Biol 29(11):1601-1615, 2022 Nov. 17), all directed at PDE7.

### Gene Therapies

Modification, complementation, replacement, or deletion of the PDE7 genes in selected tissues also result in inhibiting the respective PDE7 activity.

The invention provides a method of treating diseases associated with chronic fatigue (DACF) by administering it to a patient in need thereof an amount of a PDE7 inhibitory agent effective to inhibit the enzymatic activity of PDE7, wherein such inhibition of PDE7 enzymatic activity is the principal therapeutic mode of action of the PDE7 inhibitor in the treatment of DACF.

For each of the PDE7 inhibitory chemical compounds useful in the method of the present invention, all possible stereoisomers and geometric isomers are included. The compounds include not only racemic compounds, but also the optically active isomers. When a PDE7 inhibitory agent is desired as a single enantiomer, it can be obtained either by resolution of the final product or by stereospecific synthesis from either isomerically pure starting material or use of a chiral auxiliary reagent, for example, see Ma, Z., et al., Tetrahedron: Asymmetry 8(6):883-888, 1997. Resolution of the final product, an intermediate, or a starting material can be achieved by any suitable method known in the art. Additionally, in situations where tautomers of the compounds are possible, the present invention is intended to include all tautomeric forms of the compounds.

The PDE7 inhibitory agents that contain acidic moieties can form pharmaceutically acceptable salts with suitable cations. Suitable pharmaceutically acceptable cations include alkali metal (e.g., sodium or potassium) and alkaline earth metal (e.g., calcium or magnesium) cations. The pharmaceutically acceptable salts of the PDE7 inhibitory agents, which contain a basic center, are acid addition salts formed with pharmaceutically acceptable acids. Examples include the hydrochloride, hydro bromide, sulfate or bisulfate, phosphate or hydrogen phosphate, acetate, benzoate, succinate, fumarate, maleate, lactate, citrate, tartarate, gluconate, methanefulgonate, bezenesulphonate, and p-toluenesulphonate salts. In light of the foregoing, any reference to compounds useful in the method of the invention appearing herein is intended to include PDE7 inhibitory agents, as well as pharmaceutically acceptable salts and solvates thereof.

The compounds of the present invention can be therapeutically administered as the neat chemical, but it is preferable to administer the PDE7 inhibitory agents as a pharmaceutical composition or formulation, by way of example, as a tablet or capsule. Accordingly, the present invention further provides for pharmaceutical compositions or formulations comprising a PDE7 inhibitory agent, or pharmaceutically acceptable salts thereof, together with one or more pharmaceutically acceptable carriers and, optionally, other therapeutic and/or prophylactic ingredients. Suitable carriers are compatible with the other ingredients of the formulation and not deleterious to the recipient thereof. Compounds of the present invention may also be carried in a delivery system to provide for sustained release or enhanced uptake or activity of the compound, such as a liposomal or hydrogel system for injection, a microparticle, nanopartical, or micelle system for oral or parenteral delivery, or a staged capsule system for oral delivery.

### EXAMPLES

The following examples describe the use of some compounds in accordance with the invention. These examples are not limiting and serve only to illustrate the present invention.

### Example 1-PDE7 Enzymatic Assay and IC₅₀ Determination

The ability of compounds to inhibit PDE7 may be measured using the following assay protocal: Assay methods for IC₅₀ determination of PDE7 and variants involve as enzyme source human recombinant insect Sf9 cells. The substrate is 0.1 μM FAM-cAMP and the vehicle is 1.0% DMSO or 1.0% PEG 400. Pre-incubation time and temperature are 15 minutes at 25° C. and incubation time and temperature are 30 minutes at 25° C. The incubation buffer consists of 10 mM Tris-HCl, PH 7.2, 10 mM MgCl₂, 0.05% NaN₃, and 0.1% phosphate-free BSA. The quantitation method used is spectrofluorimetric quantitation of Fluorescein-AMP-IMAP. IBMX reference standards are run as an integral part of each assay to ensure the validity of the results obtained. The IC₅₀ is the concentration of the compound (inhibitor) tested in the assay which makes it possible to reduce the enzymatic activity of PDE7 by 50%. IC₅₀ values are finally determined by a non-linear, least squares regression analysis using MathIQ™ (ID Business Solutions Ltd., UK).

The compounds to be tested as PDE7 inhibitors were initially dissolved in vehicle at a concentration of 30 mM. These solutions were tested as the highest test concentration and then further diluted in cascade always in a ratio of 1:6 to obtain solutions with the desired lower test concentrations. Each assay was carried out in duplicate.

As illustrative and nonlimiting examples, the following compounds in accordance with the invention inhibit PDE7 with the IC₅₀ values indicated below and show their advantage as therapeutically active substances:

| | | IC₅₀ | IC₅₀ | IC₅₀ |
|---|---|---|---|---|
| | | PDE7A | PDE7B | PDE7 |
| Formula | CAS number | (nM) | (nM) | (nM) |
| No. 6A | 873540-36-4 | | | 20 |
| No. 6B | 873537-95-2 | 11.1 | 53 | 33 |
| No. 6C | 873538-20-6 | | | 14 |
| No. 6D | 873538-81-9 | | | 37 |
| No. 6E | 873541-44-7 | | | 24 |
| No. 6F | 873541-70-9 | | | 19 |
| No. 6G | 873541-14-1 | | | 20 |
| No. 6H | 873541-04-9 | | | 34 |
| No. 8A | 433695-36-4 | 150 | 12,100 | |
| No. 35A | 1086424-30-7 | 0.3 | 0.9 | |
| No. 42A | 16081-93-9 | 1,040 | | |
| No. 44A | 908570-13-8 | 26 | 72 | |
| No. 55E | 1162649-94-6 | 14.2 | 83 | |
| No. 55J | 1162650-24-9 | | | <100 |
| Dipyridamole | 58-32-2 | 42,000 | | |
| IBMX | 28822-58-4 | 10,000 | | |

### Example 2-Ex Vivo Model on More Residual Muscle Force (Less Fatigue) Via Na⁺/K⁺ATPase Activation

### Introduction of the Model

The claim of this patent is that PDE7 inhibitors can improve disturbed body function and symptoms of ME/CFS, as an example of diseases associated with chronic fatigue (DACF). Muscular fatigue and loss of force (e.g., loss of handgrip strength) is a hallmark in ME/CFS patients. A main target of PDE7 inhibition is the skeletal muscle whose energetic function is deeply disturbed in ME/CFS and which is presumed to be very important in the symptomatology of ME/CFS. Insufficient stimulation of the Na⁺/K⁺ATPase plays a key role in the development of the disturbances by a rise in intramuscular sodium that finally leads to calcium overload. The latter causes a deep functional energetic disturbance or damage. The isolated rat muscle either soleus muscle or extensor digitorum longus muscle is a well-characterized and well-validated model for the study of the function of the Na⁺/K⁺ATPase. The isolated rat soleus muscle is particularly appropriate for experiments ex vivo as it is very thin so that there is no need for blood supply. It has been demonstrated under different experimental conditions that cAMP stimulates the Na⁺/K⁺ATPase via protein kinase A (PKA) to reduce the decline in contractile force that occurs after measures such as carbachol application or high KCl concentration (12.5 mM) or by worsening of the energetic situation during electrical stimulation. In the experimental setting used, the high KCl concentration buffer causes partial depolarization that leads to sodium influx to disturb and to decrease excitability to subsequently reduce muscle force. Stimulation of Na⁺/K⁺ATPase lowers intracellular sodium levels towards physiological levels to restore excitability and muscular force during electrical stimulation. Prevention or diminution of a loss of muscular force in this procedure indicates effective stimulation of Na⁺/K⁺ATPase by a test compound with the described mode of action of PDE7-inhibition.

The decline in force in the soleus muscle can be considered as muscular fatigue and related to the loss of hand grip muscular strength in ME/CFS patients. As the phosphodiesterase isoenzyme subtype 7 (PDE7) is the predominant PDE isoenzyme in skeletal muscle to raise cAMP this ex vivo model is suited for the testing the effect of PDE7 inhibitors and in particular their effect on force generation disturbed by mechanisms that worsen excitability or the energetic situation. Since the isoenzyme variant PDE7A shows a much higher expression in skeletal muscle than that of the variant PDE7B, higher inhibitory activity of the PDE7-inhibitor on PDE7A compared with PDE7B is desirable. High KCl in a buffer (12.5 mM) was used to cause a decline in contractile force. Electrical stimulations after adding the high KCl buffer show a decline in contractile force to values close to 13% of the initial force which is considered as muscular fatigue. High level activity of Na⁺/K⁺ATPase stimulated by cAMP can counteract the decrease in excitability and the loss of force. Drugs like salbutamol preserve contractile force and muscular fatigue when compared with the control situation. As PDE7 is the predominant phosphodiesterase isoenzyme in skeletal muscle, PDE7 inhibition is expected to strongly enhance the effect of salbutamol by inhibiting the degradation of cAMP, whose production is stimulated by salbutamol via adenylyl cyclase. The experiments with the PDE7 inhibitors were performed in the presence of the β2-adrenergic agonist salbutamol to generate a certain level of cAMP whose degradation was then inhibited by the PDE7 inhibitor to still considerably raise its levels.

### Method

Muscle preparation and incubation: Female Sprague Dawley rats of 5 weeks of age were used. After isoflurane anesthesia and cervical dislocation the intact soleus muscles of the animals were dissected. The soleus muscles were mounted at resting length in a vertical position between two field stimulation electrodes (thick copper wires) in organ bathes containing 15 mL incubation medium. The lower end of the muscle was fixed to a clip fixed to the bottom of the organ bath, the upper end of the muscle was fixed to an isometric force transducer (Force transducer K300 Hugo Sachs) by a clip allowing measurement of isometric contractions, and a tension of 100 mN was applied.

Force measurement: The experimental set-up used for force measurements allowed for the simultaneous recordings from 6 muscles in separate incubation chambers in parallel. Force was recorded and stored with a Notocord HEM data acquisition system. The standard incubation medium was a Krebs-Ringer (KR) bicarbonate buffer (pH 7.4) containing (in mM): 120.1 NaCl, 25.1 NaHCO₃, 4.7 KCl, 1.2 KH₂PO₄, 1.2 MgSO₄, 1.3 CaCl₂) and 5 glucose. Incubation took place at 30° C. The buffer was continuously gassed with a mixture of 95% O₂ and 5% CO₂. Muscles were equilibrated with a mixture of 95% O₂ and 5% CO₂ in the standard medium for 30 minutes (min) before stimulation. Electrical stimulation protocol: After an equilibration period of 30 min muscles were field-stimulated to deliver optimal force at 83 Hz, 2-3 ms pulses, 12 V (biphasic), 0.2-0.5 s train width with a Hugo Sachs stimulator (HSE). After optimizing for the stimulation protocol the muscles were stimulated every 10 min for 30 min in the buffer indicated above to obtain the control values of force before weakening the contractile force by the depolarizing effect of KCl at 12.5 mM in the buffer. Investigation of test compounds: At time zero (TO) the buffer described above was exchanged against a buffer containing KCl 12.5 mM. Stimulations were then performed every 5 min for 30 min. The high KCl buffer caused a strong loss of force in controls. The test compounds were added in a volume of 0.1 mL to the organ baths containing 15 mL buffer solution already 10 min before exchanging (10 min before TO) the initial buffer for the new buffer with high KCl of 12.5 mM and they were newly added again to the new high KCl buffer after the exchange. Two control groups were included. A group with high KCl 12.5 mM buffer only and a group with high KCl 12.5 mM plus salbutamol 1 μM. PDE7 inhibitors were always tested in the presence of salbutamol 1 μM.

### Evaluation

The depolarizing effect of high KCl 12.5 mM caused a strong loss of force in controls. An effective drug would show a lower loss of contractile force, thus preserve contractile force. The residual contractile force after 30 min in high KCl buffer was chosen as the efficacy parameter and related to the last value of stimulated force in the initial buffer in the same organ bath. The percentages of residual force were compared between the different groups and used for statistical calculation. PDE7 inhibitor drugs were tested in the presence of salbutamol 1 μM and the effects were compared with those of the salbutamol 1 μM control group.

### Results

As illustrative and nonlimiting examples, the following compounds in accordance with the invention were tested according to the above protocol and delayed the rat soleus muscle force decline as indicated below, thus, showing their advantage as therapeutically active substances:

| | | | | Mean residual contractile force in % |
|---|---|---|---|---|
| Formula | CAS number/name | Concentration | | (SEM in %); number (n); p-value |
| Control group | Salbutamol (alone) | 1 | μM | 26.5 (3.9); n = 30 |
| No. 6B | 873537-95-2 | 0.99 | μM | 50.7 (3.6); n = 6; p < 0.05 |
| No. 8A | 433695-36-4 | 4.5 | μM | 80.2 (6.0); n = 15; p < 0.001 |
| No. 42A | 16081-93-9 | 3.5 | μM | 48.5 (8.6); n = 7; p < 0.05 |
| No. 44A | 908570-13-8 | 26 | nM | 63.5 (7.8); n = 6; p < 0.01 |
| No. 55E | 1162649-94-6 | 29 | nM | 69.3 (4.1); n = 5; p < 0.01 |
| Dipyridamole | 58-32-2 | 30 | μM | 46.3 (6.3); n = 15; p < 0.01 |

Soleus muscle force strongly declined under the depolarizing effect of a high extracellular potassium concentration (12.5 mM) to 13.5±2.0% (mean±SEM, n=28) mean residual contractile force compared with baseline force at normal potassium ion concentration.

Results for control values (salbutamol 1 μM) and compounds (in the presence of salbutamol 1 μM) are shown in the table above as means with standard error of mean (SEM). Stimulation by salbutamol (1 μM) moderately diminished the loss of contractile force. The effect of salbutamol 10 μM alone leaving a mean of 33.6% (SEM: 7.3%) of residual contractile force at n=9 was only moderately stronger than that of salbutamol 1 μM.

PDE7 inhibitors were given in the presence of salbutamol 1 μM to generate cAMP whose concentration could then be augmented by the inhibition of its hydrolyzation by PDE7 inhibition. The PDE7 inhibitors were therefore evaluated against the effect of salbutamol 1 μM and tested for statistical significance. PDE inhibition by the pan-PDE inhibitor dipyridamole (high concentration) plus moderate dose of salbutamol (1 μM) shows an even numerically stronger effect than salbutamol alone at the high dose of 10 UM and so did the effective PDE7 inhibitors. The PDE7 inhibitors were evaluated against the effect of salbutamol 1 μM and tested for statistical significance. The specific PDE7 inhibitor with formula 8A (433695-36-4) is even more active than dipyridamole and 42A (16081-93-9) and shows strong, statistically significant effects over salbutamol alone. The same is true for the PDE7 inhibitors with formulas 6B (873537-95-2), 44A (908570-13-8), and 55E (1162649-94-6).

### CONCLUSION

PDE7 inhibitors, in particular PDE7A inhibitors, reduce muscular fatigue as shown by a preservation of contractile force under conditions of reduced excitability by sodium overload caused by the depolarizing effect of high KCl buffer. The mechanism is via restoring excitability by activation of the Na⁺/K⁺ATPase. Based on these actions PDE7 inhibitors can treat muscular fatigue and loss of force. Moreover, due to the finding that stimulation of the Na⁺/K⁺ATPase lowers intracellular sodium—high intramuscular sodium causes calcium overload via the reverse mode of the sodium-calcium-exchanger (NCX) to cause muscular damage and mitochondrial dysfunction—PDE7 inhibitors can treat and prevent mitochondrial dysfunction and the deleterious secondary consequences of energy deprivation. This deprivation otherwise results in the physiological stimulation of the production of vasoactive mediators from skeletal muscle, which are normally physiologically meant to raise muscular blood flow. These mediators are then excessively produced due to the very poor energetic situation and therefore released into the systemic blood stream (spillover). Thus, these mediators with algesic, hyperalgesic, spasmogenic and microvascular leakage-inducing properties can reach every organ to cause a variety of symptoms of diseases associated with chronic fatigue. These include pain, edema and spasms, which are prevented by treatment involving PDE7 inhibition in the form of an indirect effect.

1. Substance with phosphodiesterase-7 inhibitory activity (PDE7 inhibitors) for use as active ingredient in a therapeutic agent with phosphodiesterase-7 inhibitory activity for the treatment and prevention of chronic fatigue, exhaustion and/or exertional intolerance and for the treatment and prevention of diseases that are associated with chronic fatigue, exhaustion and/or exertional intolerance.

2. Substance as claimed in claim 1, wherein diseases that are associated with chronic fatigue, exhaustion and/or exertional intolerance are diseases diagnosed as Myalgic Encephalomyelitis/Chronic Fatigue Syndrome (ME/CFS), systemic exertional intolerance, exertional intolerance, Long COVID complaints, post-Covid-19 syndrome (PCS), post-acute Covid syndrome (PACS), Post-Acute Sequelae of SARS-CoV-2 Infection (PASC), post-vaccination syndrome (post-vac-syndrome) after COVID-19 vaccinations (Long Vax) and vaccinations against other germs, virus and pathogenic agents, and postinfectious fatigue after viral, bacterial, or fungal infections, in particular ME/CFS of non-infectious, non-inflammatory and non-immunological cause.

3. Substance as claimed in claim 1, wherein chronic fatigue and exhaustion are symptoms of or are associated with cancer (cancer-related fatigue), fibromyalgia, Ehlers-Danlos syndrome, Marfan syndrome, Gulf War illness, the autoimmune diseases Rheumatoid Arthritis, ANCA vasculitis and Sjögren's syndrome, and other autoimmune diseases with fatigue and exhaustion as debilitating symptoms.

4. The substance as claimed in one or more of claims 1 to 3, wherein the PDE7 inhibitory material is a material that inhibits the enzymatic activity of PDE7 (PDE7A, PDE7B, or PDE7A and PDE7B) at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

5. The substance as claimed in one or more of claims 1 to 3, wherein the PDE7 inhibitory material is a material that inhibits the enzymatic activity of PDE7A, preferably the PDE7 inhibitory material is a material that inhibits the enzymatic activity of PDE7A at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

6. The substance as claimed in one or more of claims 1, 2, 3, 4, and 5, wherein the PDE7 inhibitory agent stimulates the Na⁺/K⁺-ATPase and the NCLX in skeletal muscle and improves blood flow to skeletal muscles and brain by raising cAMP.

7. The substance as claimed in one or more of claims 1 to 6, wherein the PDE7 inhibitory agent is present in a pharmaceutical preparation, preferably in the form of an oral drug, intravenous, subcutaneous, intramuscular, pharyngeal, and nasal administration.

8. The substance as claimed in one or more of claims 1 to 7, wherein the PDE7 inhibitory agent is a chemical compound, a protein or polypeptide, a nucleic acid molecule, ribozyme, DNAzyme, a protein degrader, or a material for gene therapy.

9. The substance as claimed in claim 8, wherein the chemical compound is a small molecule inhibitor including natural and synthetic substances that have a low molecular weight, such as, a peptide, a peptidomimetic and a non-peptide inhibitor such as a classical small molecule chemical compound, preferably the small molecule inhibitor having a molecular weight of less than about 750 g/mol and an IC₅₀ for inhibiting PDE7 (PDE7A, PDE7B, or PDE7A and PDE7B) at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

10. The substance as claimed in claim 8, wherein the chemical compound is a small molecule inhibitor including natural and synthetic substances that have a low molecular weight, such as, a peptide, a peptidomimetic and a non-peptide inhibitor such as a classical small molecule chemical compound, preferably the small molecule inhibitor having a molecular weight of less than about 750 g/mol and wherein the PDE7 inhibitory material is a material that inhibits the enzymatic activity of PDE7A, preferably the PDE7 inhibitory material is a material that inhibits the enzymatic activity of PDE7A at an IC₅₀<50 μM, preferably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.

11. The substance as claimed in claim 8, wherein the PDE7 inhibitory agent is a protein or polypeptide including a PDE7-binding antibody, nanobody, or functionally related protein or protein fragment or fusion protein, including a single-chain variable fragment or a designed ankyrin repeat protein or lipocalin/anticalin.

12. The substance as claimed in claim 8, wherein the PDE7 inhibitory agent inhibits PDE7 expression and is an antisense nucleic acid molecule such as antisense oligonucleotide, antisense RNA, antisense mRNA, antisense DNA, or a PDE7 RNAi molecule, siRNA, microRNA or other such molecule, or a PDE7 altering ribozyme, or a PDE7 altering DNAzyme.

13. The substance as claimed in claim 8, wherein the PDE7 inhibitory agent is a protein degrader, like a PROTAC, SNIPER, HaloPROTAC, HyT, LYTAC, AUTAC, ATTEC, RIBOTEC, monomeric degrader, double-mechanism degrader, SARD, TF-PROTAC, dual-PROTAC, SERD, bispecific aptamer chimera, AbTAC, GlueTAC, AUTOTAC, CMA-based degrader, MADTAC, ATAC, molecular glue, biodegraders, mRNA for the biodegrader protein, mRNA for the biodegrader protein in a lipid nanoparticle formulation, all targeted at PDE7.

14. The substance as claimed in claim 8, wherein the PDE7 inhibitory agent is a gene therapy modifying, completing, replacing, or deleting PDE7 genes.

15. The substance as claimed in one or more of claims 1 to 14, wherein the PDE7 inhibitory agent is a member selected from the group consisting of substances containing the structural units pyrazolopyrimidinone, spirocycle, spirocyclic quinazoline, 8′-chloro-2′,3′-dihydro-2′-oxospiro[cyclohexane-1,4′(1′H)-quinazolin]-5′-yl, thiadiazole and oxadiazole, pyridinylpyrazolopyrimidinone, arylindenopyridine, thienopyrazole, 3-methyl-1H-thieno[2,3-c]pyrazole-5-carboxamide, 3-methyl-1-(tetrahydro-2H-pyran-4-yl)-1H-thieno[2,3-c]pyrazole-5-carboxamide, imidazotriazinone, benzenesulfonamide, thiazol-2-yl-imine, (4,2-disubstituted-thiazol-5-yl)amine, amine-substituted thiopheno[2,3-d]pyrimidine, nitrogen-containing bicycles, 4-substituted fused heteropyrimidine and fused hetero-4-pyrimidone, 2-amine-substituted quinazoline and pyrido[2,3-d]pyrimidine, purine, amine-substituted pyrimidine, amine-substituted purine, fused heterocycles containing a 2-amine-substituted pyrimidine, phthalazinone, 3,4-dihydroisoquinoline and isoquinoline, 1-phenyl-derivative-substituted 3,4-dihydroisoquinoline, sulphonamide, heterobiaryl sulphonamide, naphthalenyl of dihydropurin-6-one, spirocondensed quinazolin-2-one, spirocyclic and substituted quinazoline, imidazotriazine, spirocondensed 4-amino-butanecarboxylic adic, 4-amine-substituted thieno[2,3-d]pyrimidine-6-carbonitrile, pyrimidin-2-yl sulfide, 5-oxo-pyrrole-3-carboxylate, thieno[2,3-b]thiophen-3-amine, indolo[2,3-b]quinoxaline, methylphenanthrene, spirocyclic and 5,8-substituted (1H,3H)-quinazoline, pyrrolopyrimidine, imidazole, isoxazole, 4-cyano-1H-pyrrole-2-carboxylate, imidazo[4,5-c]pyridine, quinazoline, 3-substituted 2,3-dihydro-2-thioxo-4(1H)-quinazolinone and 3-substituted 2,3-dihydro-2-oxo-4(1H)-quinazolinone, 3-substituted 2,3-dihydro-2-thioxothieno[3,2-d]pyrimidin-4(1H)-one, 3-substituted 2,3-dihydro-2-thioxo[1]benzothieno[3,2-d]pyrimidin-4(1H)-one, ethyl-substituted purine-2-amine, quinazolinedione, bicyclic nitrogen-containing heterocycles substituted with an amino group, 3,5-substituted 1,2,3-triazolo[4,5-d]pyrimidin-7-amine, bicyclic 3-substituted 1,2,4-triazole, 2-pyrimindinone, isochromenone, 5-imino-1,2,4-thiadiazole, 4,5-dihydroisoxazole-substituted pyrazolopyrimidine, imidazopyridine, dihydropurine, pyrrole, benzothiopyranoimidazolone, guanine, arylindenopyrimidine, sulfonylbenzene, trans-aconitic acid, thienopyrimidine/thienopyrimidinone, 2-(isopropylamino)thieno[3,2-d]pyrimidin-4(3H)-one, isothiazole and isoxazole fused pyrimidone, imidazopyridazinone, sulfide, steroids, podocarpanes, fused thiophenes, dihydronaphthyridinedione, furan, S-substituted quinazolines, methylxanthine, and benzothienothiadiazine.

16. The substance as claimed in one or more of claims 1 to 15, wherein the inhibitory agent is a member selected from the group consisting of compounds with the CAS Registry Numbers assigned by the Chemical Abstracts Service (CAS) of 553669-13-9, 553668-77-2, 460346-28-5, 756535-34-9, 873540-36-4, 873537-95-2, 873538-20-6, 873538-81-9, 873541-44-7, 873541-70-9, 873541-14-1, 873541-04-9, 433695-36-4, 1086424-30-7, 16081-93-9, 1062094-00-1, 1062094-26-1, 873541-45-8, 908570-13-8, 908570-12-7, 873541-45-8 (·H2O), 906079-12-7, 1041846-43-8, 1374784-19-6, 1041846-46-1, 1140589-61-2, 1140589-71-4, 1140589-76-9, 1140590-16-4, 1162649-94-6, 1162650-34-1, 1140590-71-1, 1162650-04-5, 1162650-22-7, 1162650-24-9, 1251701-48-0, 1251701-62-8, 1251702-51-8, 1251702-23-4, 1251702-27-8, 1251701-58-2, 1251702-08-5, 1281681-33-1, 1281681-33-1 without 2 HBr, 58-32-2, 6493-05-6, 873539-34-5, 18741-24-7, and 28822-58-4.

17. A pharmaceutical composition, characterized in that it comprises at least one PDE7 inhibitory agent as claimed in one or more of claims 1 to 16.

18. The pharmaceutical composition as claimed in claim 17, characterized in that it comprises (i) at least one PDE7 inhibitory agent according to claim 9, or a pharmaceutically acceptable salt, solvate, hydrate, co-crystal, polymorph, ester, ether, enantiomer, prodrug, or metabolite thereof, and at least one pharmaceutically acceptable diluent or excipient, or (ii) at least one compound of the formulas according to claim 14 or claim 16, or a pharmaceutically acceptable salt, solvate, hydrate, co-crystal, polymorph, ester, ether, enantiomer, prodrug, or metabolite thereof, and at least one pharmaceutically acceptable diluent or excipient.

19. Use of a compound with phosphodiesterase-7 inhibitory activity (PDE7 inhibitors) for the manufacture of a medicament for the treatment and prevention of chronic fatigue, exhaustion and/or exertional intolerance, preferably said PDE7 inhibitory compound inhibits the enzymatic activity of PDE7 (PDE7A, PDE7B, or PDE7A and PDE7B) at an IC₅₀<50 μM, preferrably at an IC₅₀≤1 μM, more preferred at an IC₅₀ of from 0.1 to 600 nM, in particular preferred at an IC₅₀ of from about 0.2 to about 100 nM, and further in particular preferred at an IC₅₀ of from about 1 to about 100 nM.
